(19) United States
(12) Patent Application Publication
(10) Pub. No.: US 2024/0124484 A1
(43) Pub. Date: Apr. 18, 2024

(54) IMIDAZOTHIENOPYRIDINE COMPOUNDS AND METHODS OF USE

(71) Applicant: ZYMEWORKS BC INC., Vacouver (CA)

(72) Inventors: Graham Albert Edwin GARNETT, Vancouver (CA); Michael G. Brant, Vancouver (CA)

(21) Appl. No.: 18/257,850

(22) PCT Filed: Dec. 14, 2021
(86) PCT No.: PCT/CA2021/051809

§ 371 Date: Jun. 15, 2023

Related U.S. Application Data
(60) Provisional application No. 63/126,980, filed on Dec. 17, 2020.

(51) Int. Cl. C07D 495/14; A61K 47/68

(52) U.S. Cl. CPC C07D 495/14; A61K 47/6803

(57) ABSTRACT

Disclosed herein are compounds having Formula I, or a pharmaceutically acceptable salt thereof, wherein R, R¹, R², R³, and Spacer are described herein. Also disclosed are methods of making and using such compounds, as well as pharmaceutical compositions comprising the compounds, for the treatment of a disease such as cancer.

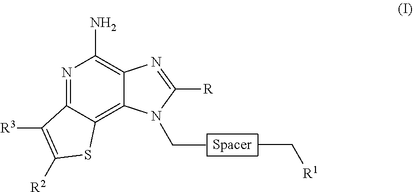

### CROSS-REFERENCE

This application claims the benefit of U.S. Provisional Application No. 63/126,980, filed Dec. 17, 2020, which is incorporated herein by reference in its entirety for all purposes.

### TECHNICAL FIELD

This specification relates generally to compounds for targeted immuno-oncology and methods of making and using the same.

### BACKGROUND

Over a million new cases of cancer will be diagnosed, and over half a million Americans will die from cancer this year. Although surgery can provide definitive treatment of cancer in its early stages, the eradication of metastases is crucial to the cure of more advanced disease. Chemotherapeutic drugs used in combinations provide the standard treatment for metastases and advanced disease. However, the side effects of these treatments seriously diminish the quality of life for cancer patients, and progressions and relapses following surgery and chemotherapy/radiation are common. Thus, despite the expenditure of large amounts of public and private resources over many years, better treatments for cancer are still sorely needed.

Toll-like receptors (TLRs) are a class of pattern recognition receptors that recognize pathogen-associated molecular patterns (PAMPs) and damage-associated molecular patterns, including lipopolysaccharide and free nucleic acids. Activation of a TLR by binding of its cognate molecular pattern stimulates the host's immune system to fight the infection. Based on their role in regulating innate and adaptive immunity, TLRs have been explored for their potential as anti-tumor therapies (see, Chi H, et al. Front. Pharmacol. 2017; 8:304, doi: 10.3389/fphar.2017.00304).

One of the most studied TLRs in humans is TLR7. TLR7 is an immune response sensor sensitive for ligands such as ssRNA and cGMP. TLR7 is expressed primarily on plasmacytoid dendritic cells (pDCs), where it initiates production of IFN-α in response to pathogen or damage signals, thereby playing a pivotal role in the induction of inflammatory response. Activation of TLR7 by a natural or synthetic agonist can beneficially affect the action of vaccines and immunotherapy agents in treating not just pathogen infection, but also various other conditions through stimulation of the immune response overall. As such, there has been considerable interest in TLR7 agonists for use as vaccine adjuvants and in cancer immunotherapy (see review by Patinote, et al., Eur J Med Chem., 2020, 193:112238).

Although clinical data support the use of TLR7 agonists in oncology, such treatment requires local administration. For example, the TLR7 agonist imiquimod has been approved for topical use in dermal oncology applications, including basal cell carcinoma and actin keratosis (see, Geisse et al. J Am Acad Dermatol. 2004; 50(5):722-33, and Korman et al. Arch Dermatol. 2005; 141(4):467-473). Other uses in invasive skin cancers (e.g., squamous cell carcinoma, Bowen's disease, melanoma, and/or lentigo maligna) are similarly efficacious when applied locally on surface lesions (see, Meyer et al. Expert Opin Investig Drugs. 2008; 17(7):1051-65, and Wolf et al. Arch Dermatol. 2003; 139(3):273-6).

Unfortunately, studies have reported that systemic administration of imiquimod and other TLR agonists leads to dose-limiting toxicity below efficacious doses (see, Dudek et al. Clin Cancer Res 2007; 13:7119-7125). Furthermore, overexpression and/or activation of some TLRs (including TLR7) results in conflicting anti-tumor/pro-tumor activity, in some cases contributing to, rather than diminishing, inflammation, tumor growth, cell survival, metastasis, and the upregulation of pro-inflammatory cytokines (see, Kaczanowska S, et al. J Leukoc Biol. 2013; 93(6):847-863). Antibody-drug conjugates (ADCs) combine the selectivity of antibodies with the efficacy of small-molecule chemotherapeutics, allowing for more precise, targeted therapeutic applications. Traditionally, ADCs deliver cytotoxic payloads to antigen-expressing cancer cells with conjugated antibodies that bind to specific targets. Commercially available ADCs, for example, carry cytotoxic payloads for potential treatment of various liquid and solid tumors (Gingrich, J. J ADC. 2020; doi: 10.14229/jadc.2020.04.07.001).

In contrast to traditional ADCs, which selectively deliver a cytotoxic payload to tumor cells, ADCs comprising an immunostimulatory compound, such as a TLR activating compound, deliver an immunostimulatory payload to the tumor where it exerts an indirect anti-tumor effect mediated by immune cells. Such ADCs are often referred to as immunostimulatory antibody-drug conjugates (ISACs).

Several aspects of TLR7 make it attractive for immunostimulatory antibody-drug conjugate (ISAC)-based agonism including its functional role, lysosomal expression, cellular expression profile, chemical properties of its agonists, and status as a clinically validated target (e.g., TLR7 regulates inflammation via production of IFN-α, and its expression is limited to the lysosome in innate immune cells). ISACs comprising TLR7 agonists as payloads have been described (PCT Patent Publication Nos. WO 2015/103989; WO 2017/072662; WO 2017/100305; WO 2018/009916; WO 2019/036023 and WO 2020/056192).

### SUMMARY

The present disclosure relates to compounds and compositions that can act as agonists for a Toll-like receptor (TLR), such as TLR7 and/or TLR8, and can be used, for example, for the treatment of cancer. Certain embodiments relate to such compounds in the form of immunostimulatory antibody-drug conjugates (ISACs).

In vitro agonism of TLR7 and TLR8 has been shown to be induced in peripheral blood mononuclear cells incubated with the disclosed compounds and compositions (see, e.g., Example 11, below). Similarly, in vivo ISAC therapy utilizing antibodies targeting tumor-associated antigens (e.g., Her2) conjugated to at least one of the disclosed compounds generated an anti-tumor response, as evidenced by inhibition of tumor growth rate (see, e.g., Example 13, below). Notably, no negative consequences to normal cells (e.g., off-target effects) were observed during in vivo testing of ISACs comprising the disclosed compounds and compositions.

Accordingly, one aspect of the present disclosure provides a compound having Formula I:

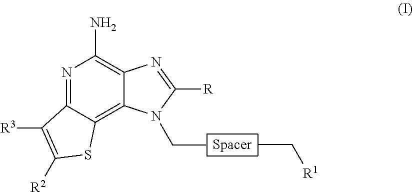

or a pharmaceutically acceptable salt thereof,

wherein:

R is H, C₁-C₆ alkyl, CH₂SR¹⁵ or CH₂OR¹⁵;

R¹ is —OH, —NR⁴R⁵, —OR¹⁰, SR¹¹ or

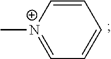

R² and R³ are each independently H or optionally substituted C₁-C₆ alkyl;

Spacer is —(CH₂)ₙ,

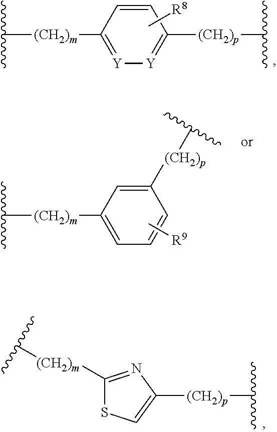

wherein n is an integer between 3 and 10; each m is independently an integer between 0 and 4; each p is independently an integer between 0 and 4; and Y is CH or N;

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;

R⁸ and R⁹ are each independently H, NR¹³R¹⁴, halo, optionally substituted C₁-C₄ alkoxy or optionally substituted C₁-C₄ alkyl;

R¹⁰ and R¹¹ are each independently optionally substituted C₁-C₆ alkyl, optionally substituted aryl or optionally substituted C₃-C₇ cycloalkyl;

R¹³ and R¹⁴ are each independently H or optionally substituted C₁-C₄ alkyl; and

R¹⁵ is C₃-C₄ cycloalkyl or C₁-C₄ alkyl optionally substituted with one or more halo.

In some embodiments, the present disclosure provides a conjugate having Formula X:

T-(L-(D)ᵣ)_{q} (X)

wherein T is a targeting moiety; L is a linker; D is a compound having Formula I; q has a value from about 1 to about 8, and r is an integer from 1 to 4.

In some embodiments, T is an antibody or an antigen-binding antibody fragment.

The present disclosure also provides pharmaceutical compositions comprising a compound having Formula I, or a pharmaceutically acceptable salt thereof, and/or a conjugate having Formula X, in accordance with some embodiments of this disclosure, and a pharmaceutically acceptable carrier or diluent.

The present disclosure also provides methods of agonizing a TLR (e.g., TLR7) comprising contacting a cell that expresses the TLR (e.g., TLR7) with a compound having Formula I, or a pharmaceutically acceptable salt thereof, and/or a conjugate having Formula X, in accordance with some embodiments of this disclosure.

The present disclosure also provides methods of stimulating an immune response in a subject in need thereof, such methods comprising administering to the subject an effective amount of a compound having Formula I, or a pharmaceutically acceptable salt thereof, and/or a conjugate having Formula X, in accordance with some embodiments of this disclosure.

The present disclosure also provides methods of treating a cancer in a subject in need thereof, such methods comprising administering to the subject an effective amount of a compound having Formula I, or a pharmaceutically acceptable salt thereof, and/or a conjugate having Formula X, in accordance with some embodiments of this disclosure.

It is to be understood that any embodiment disclosed herein, when applicable, can be applied to any aspect of this disclosure.

### BRIEF DESCRIPTION OF THE DRAWINGS

The implementations disclosed herein are illustrated by way of example, and not by way of limitation, in the accompanying drawings. The description and drawings are only for the purpose of illustration and as an aid to understanding, and are not intended as a definition of the limits of the compounds, conjugates, compositions and methods of the present disclosure.

FIG. 1 presents a general synthetic scheme (Scheme 1) using General Procedures described herein that may be used in some embodiments to prepare compounds of Formula I.

FIG. 2 presents a general synthetic scheme (Scheme 2) using General Procedures described herein that may be used in some embodiments to prepare compounds of Formula I.

FIG. 3 presents a general synthetic scheme (Scheme 3) using General Procedures described herein that may be used in some embodiments to prepare a drug-linker construct comprising a compound of Formula I (“TLR7 agonist”) coupled to a linker.

FIGS. 4A and 4B show the hydrophobic interaction chromatography (HIC) chromatograms for the antibody-drug conjugates (A) T-MTvcPABC-Compound 111, and (B) T-MTvcPABC-Compound 166, respectively.

FIGS. 5A and 5B show the size exclusion chromatography (SEC) chromatograms for the antibody-drug conjugates (A) T-MTvcPABC-Compound 111, and (B) T-MTvcPABC-Compound 166, respectively.

FIGS. 6A and 6B show (A) the light chain deconvoluted RP-UPLC-MS spectrum, and (B) the heavy chain deconvoluted RP-UPLC-MS spectrum for the antibody-drug conjugate T-MTvcPABC-Compound 111.

FIGS. 7A and 7B show (A) the light chain deconvoluted RP-UPLC-MS spectrum, and (B) the heavy chain deconvoluted RP-UPLC-MS spectrum for the antibody-drug conjugate T-MTvcPABC-Compound 166.

FIG. 8 shows the in vivo efficacy of antibody-drug conjugates comprising the drug-linkers MTvcPABC-Compound 111 or MTvcPABC-Compound 166 conjugated to trastuzumab in decreasing tumor volume in mice implanted with HER2-high NCI-N87 tumors.

FIG. 9 shows the change in body weight (in %) of mice implanted with HER2-high NCI-N87 tumors when treated with antibody-drug conjugates comprising the drug-linkers MTvcPABC-Compound 111 or MTvcPABC-Compound 166.

FIGS. 10A and 10B show, respectively, a schematic of an immunostimulatory antibody-drug conjugate (10B) and its mechanism of action (10A), in accordance with some embodiments of the present disclosure.

### DETAILED DESCRIPTION

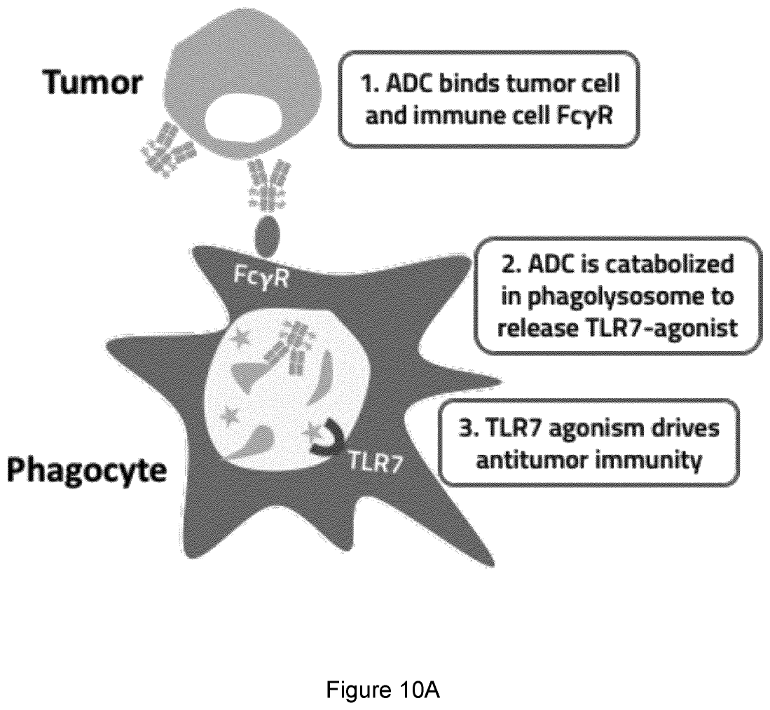

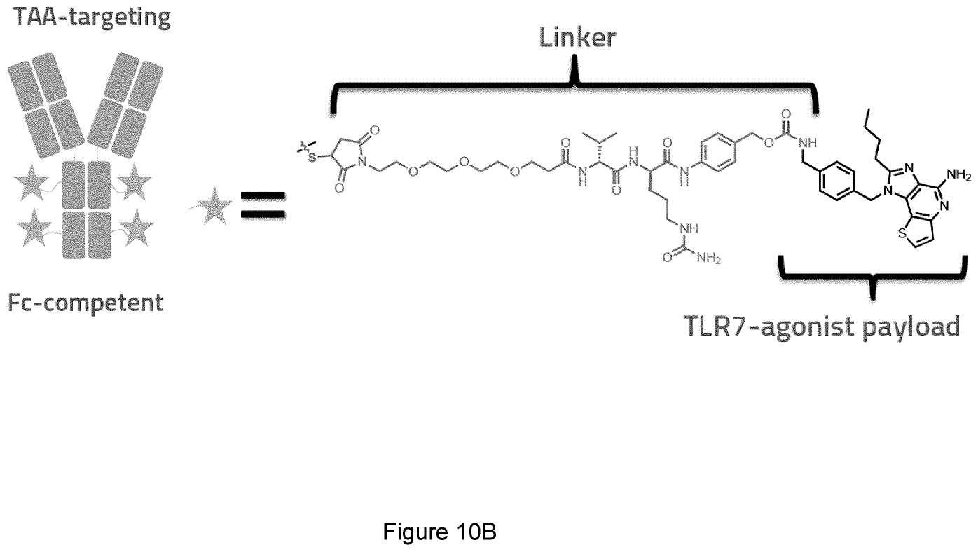

The present disclosure provides compounds capable of agonizing a TLR, conjugates and compositions comprising the same, and methods of making and using the compounds, conjugates and compositions for agonizing the TLR, and methods of stimulating an immune response and/or treating a disease in a subject. In an embodiment, the present disclosure provides immunostimulatory antibody-drug conjugates (ISACs) that target immune cells expressing the TLR to stimulate anti-tumor activity based on antibodies targeted to specific tumor-associated antigens (TAAs). In various embodiments, the TLR is TLR7, TLR8, or a combination thereof. In various instances, the TLR is TLR7. FIG. 10A illustrates a mechanism of action of an ISAC comprising a TLR7 agonist that is mediated by phagocytes, in accordance with some embodiments of the present disclosure.

As illustrated in FIG. 10A, anti-tumor effects can be mediated by innate immune cells. In the embodiment shown in FIG. 10A, an ISAC initially binds the tumor cell via the target TAA and the immune cell via FcTR engagement. Subsequently, the ISAC is catabolized in the phagolysosome to release the TLR7 agonist. Stimulation of TLR7 in the phagolysosome induces cytokine expression, which can drive the anti-tumor response. As a result, TLR7 agonism can drive anti-tumor immunity.

An example ISAC is further illustrated in FIG. 10B. In some embodiments, an ISAC comprises a TAA-targeting antibody, a linker, and an immunostimulatory payload (e.g., a TLR7 agonist of the present disclosure). In some embodiments, the payload is a high potency TLR7 agonist. In some embodiments, the TLR7 agonist can be a compound of Formula I described herein. In some embodiments, the ISAC induces a TLR7-derived immune response, which can include production of regulatory cytokines (e.g., IL-6) from immune cells (e.g., peripheral blood mononuclear cells (PBMCs)). In some embodiments, the ISAC induces a reduction in tumor size, e.g., in a subject following administration of the ISAC to the subject.

Accordingly, the present disclosure provides compounds (e.g., immunostimulatory drugs for immunostimulatory antibody-drug conjugates), conjugates and compositions comprising the same, and methods of making and using any of the same to agonize TLR7, stimulate an immune response, and/or treat disease (e.g., a cancer in a subject). The compounds, conjugates, compositions and methods disclosed herein are generally representative of the compounds, conjugates, and compositions of the present disclosure and the methods in which such compounds, conjugates, and compositions can be used. The following discussion is intended as illustrative of selected aspects and embodiments of the present disclosure and it should not be interpreted as limiting the scope of the present disclosure.

### Definitions

The term “alkyl,” as used herein, refers to a straight or branched saturated hydrocarbon chain containing the specified number of carbon atoms. Examples of alkyl groups include, but are not limited to: methyl, ethyl, n-propyl, isopropyl, n-butyl, sec-butyl, isobutyl, t-butyl, pentyl, isopentyl, t-pentyl, neo-pentyl, 1-methylbutyl, 2-methylbutyl, n-hexyl, and the like.

The term “alkoxy,” as used herein, refers to the group —O-alkyl. Examples of alkoxy groups include, but are not limited to, methoxy, ethoxy, n-propoxy, isopropoxy, n-butoxy, sec-butoxy, isobutoxy, and the like.

The term “alkoxycarbonyl,” as used herein, refers to the group —C(O)O-alkyl, wherein the alkyl may be optionally substituted.

The term “amido,” as used herein, refers to the group —C(O)NR′R″, wherein R′ and R″ may independently be hydrogen, optionally substituted alkyl, optionally substituted aryl or optionally substituted heteroaryl.

The term “amidoalkyl,” as used herein, refers to an alkyl group substituted with one or more amido groups. In certain embodiments, an amidoalkyl refers to an alkyl group substituted with one amido group.

The term “amino,” as used herein, refers to the group —NH₂.

The term “aminoalkyl,” as used herein, refers to an alkyl group substituted with one or more amino groups. In certain embodiments, an aminoalkyl refers to an alkyl group substituted with one amino group.

The term “aryl,” as used herein, refers to a 6- to 12-membered monocyclic or bicyclic hydrocarbon ring system in which at least one ring is aromatic. Examples of aryl groups include, but are not limited to: phenyl, naphthalenyl, 1,2,3,4-tetrahydro-naphthalenyl, 5,6,7,8-tetrahydro-naphthalenyl, indanyl, and the like.

The term “arylalkyl,” as used herein, refers to an alkyl group substituted with one or more optionally substituted aryl group(s). In certain embodiments, arylalkyl refers to an alkyl group substituted with one optionally substituted aryl group. Examples of arylalkyl groups include, but are not limited to: benzyl, phenethyl, phenylpropyl, naphthalenylmethyl, and the like.

The term “carboxyalkyl,” as used herein, refers to an alkyl group substituted with one or more carboxyl groups.

The term “carboxyl” as used herein refers to the group —C(O)OH.

The term “cycloalkyl,” as used herein, refers to a monocyclic or bicyclic saturated hydrocarbon ring system containing the specified number of carbon atoms. Examples of cycloalkyl groups include, but are not limited to: cyclopropyl, cyclobutyl, cyclopentyl, cyclohexyl, and the like.

The term “cycloalkylalkyl,” as used herein, refers to an alkyl group substituted with one or more optionally substituted cycloalkyl group. In certain embodiments, cycloalkylalkyl refers to an alkyl group substituted with one optionally substituted cycloalkyl group. Examples of cycloalkylalkyl groups include, but are not limited to: cyclopropylmethyl, cyclobutylmethyl, cyclopentylmethyl, cyclohexylmethyl, cyclopropylethyl, cyclobutylethyl, cyclopentylethyl, cyclohexylethyl, and the like.

The term “heteroaryl,” as used herein, refers to a 5- to 12-membered monocyclic or bicyclic ring system in which at least one ring atom is a heteroatom and at least one ring is aromatic. Examples of heteroatoms include O, S and N. Examples of heteroaryl groups include, but are not limited to: pyridinyl, triazolyl, benzofuranyl, pyrazinyl, pyridazinyl, pyrimidinyl, triazinyl, quinolinyl, benzoxazolyl, benzothiazolyl, 1H-benzimidazolyl, isoquinolinyl, 3,4-dihydroisoquinolyl, quinazolinyl, quinoxalinyl, pyrrolyl, indolyl, and the like.

The term “heteroarylalkyl,” as used herein, refers to an alkyl group substituted with one or more optionally substituted heteroaryl group(s). In certain embodiments, heteroarylalkyl refers to an alkyl group substituted with one optionally substituted heteroaryl group.

The term “heterocyclyl,” as used herein, refers to a 3- to 12-membered monocyclic or bicyclic non-aromatic ring system in which at least one ring atom is a heteroatom. Examples of heteroatoms include O, S and N. A heterocyclyl substituent can be attached via a ring carbon or a ring heteroatom. Examples of heterocyclyl groups include, but are not limited to: aziridinyl, azetidinyl, piperidinyl, morpholinyl, piperazinyl, pyrrolidinyl, and the like.

The term “heterocyclylalkyl,” as used herein, refers to an alkyl group substituted with one or more optionally substituted heterocyclyl group(s). In certain embodiments, heterocyclylalkyl refers to an alkyl group substituted with one optionally substituted heterocyclyl group.

As used herein with reference to a ring system, the term “bicyclic” includes both fused and spiro ring systems.

The term “substituted,” as used herein, indicates that at least one hydrogen atom of the named group is replaced by a non-hydrogen substituent or group. When a group is “substituted” it may have one substituent or it may have more than one substituent up to the total number of substituents physically allowed by the group. For example, a methyl group can be substituted by 1, 2, or 3 substituents, a phenyl group can be substituted by 1, 2, 3, 4, or 5 substituents, a naphthyl group can be substituted by 1, 2, 3, 4, 5, 6, or 7 substituents, and the like. When a group is substituted with more than one group they can be identical or they can be different. Substituents include groups such as hydroxyl, thiol, halogen, nitro, cyano, acyl, alkoxy, amino, amido, carboxyl, alkyl, alkenyl, alkynyl, alkylthiol, alkoxycarbonyl, aminoalkyl, amidoalkyl, cycloalkyl, cycloalkylalkyl, heterocyclyl, heterocycloalkyl, aryl, arylalkyl, aryloxy, heteroaryl or heteroarylalkyl.

The term “antibody,” as used herein, refers broadly to monoclonal antibodies, polyclonal antibodies, multispecific antibodies (e.g., bispecific antibodies), and/or antibody fragments, so long as they exhibit the desired biological activity. Antibodies may be murine, human, humanized, chimeric, or derived from other species (e.g., camels or sharks). An antibody is a protein generated by the immune system that is capable of recognizing and binding to a specific antigen. (Janeway, et al. (2001) “Immunobiology”, 5th Ed., Garland Publishing, New York). A target antigen generally has numerous binding sites, also called epitopes, recognized by complementary determining regions (CDRs) on multiple antibodies. Each antibody that specifically binds to a different epitope has a different structure. Thus, one antigen may have more than one corresponding antibody.

The term antibody also refers to a full-length immunoglobulin molecule or an immunologically active portion of a full-length immunoglobulin molecule, e.g., a molecule that contains an antigen binding site that immunospecifically binds an antigen of a target of interest or part thereof, such targets including but not limited to, a cancer cell or cells that produce autoimmune antibodies associated with an autoimmune disease. The immunoglobulins disclosed herein can be of any type (e.g., IgG, IgE, IgM, IgD, or IgA), class (e.g., IgG1, IgG2, IgG3, IgG4, IgA1 or IgA2) or subclass of immunoglobulin molecules. The immunoglobulins can be derived from any species. In certain embodiments, the immunoglobulin is of human, murine, or rabbit origin.

The terms “treat” and “treatment,” as used herein, generally refer to a therapeutic treatment, where the object is to slow down (lessen) an undesired physiological change or disorder, such as the growth, development or spread of a hyperproliferative condition, such as cancer. For purposes of the present disclosure, beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The phrase “therapeutically effective amount,” as used herein, refers to an amount of a compound (e.g., a compound of Formula I) or conjugate (e.g., a conjugate of Formula X) disclosed herein that (i) treats the particular disease, condition, or disorder, (ii) attenuates, ameliorates, or eliminates one or more symptoms of the particular disease, condition, or disorder, and/or (iii) delays the onset of one or more symptoms of the particular disease, condition, or disorder described herein. In the case of cancer, the therapeutically effective amount of the drug (e.g., compound of Formula I or a conjugate X thereof) may reduce the number of cancer cells; reduce the tumor size; inhibit (e.g., slow to some extent and preferably stop) cancer cell infiltration into other, e.g., peripheral, organs; inhibit (e.g., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The phrases “therapeutic composition” and “pharmaceutical composition,” as used herein, can be used interchangeably and refer to a mixture of components for therapeutic administration. In some embodiments, a therapeutic composition comprises a therapeutically active agent of the present disclosure and one or more of a buffering agent, solvent, nanoparticle, microcapsule, viral vector and/or other stabilizer(s). In some embodiments, the therapeutically active agent is, for example, a compound that agonizes TLR7 (e.g., a compound of Formula I or a conjugate X thereof). In some embodiments, a therapeutic composition also contains residual levels of chemical agents used during the manufacturing process, e.g., surfactants, buffers, salts, and stabilizing agents, as well as chemical agents used to adjust the pH of the final composition, for example, counter ions contributed by an acid (e.g., hydrochloric acid or acetic acid) or base (e.g., sodium or potassium hydroxide), and/or trace amounts of contaminating proteins.

The terms “dose” and “dosage,” as used interchangeably herein, refer to the amount of active ingredient given to an individual at each administration. The dose may vary depending on a number of factors, including frequency of administration; size and tolerance of the individual; severity of the condition; risk of side effects; and the route of administration. One of skill in the art will recognize that the dose can be modified depending on the above factors or based on therapeutic progress. The term “dosage form,” as used herein, refers to the particular format of the pharmaceutical composition, and depends on the route of administration. For example, a dosage form can be a liquid, formulated for administration via intravenous infusion and/or subcutaneous injection, or a tablet or capsule, formulated for oral administration.

The terms “cancer” and “cancerous,” as used herein, refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

As used herein, the term “disease” generally refers to a state of health of an animal (e.g., a mammal such as a human or a rodent) in which the animal cannot maintain homeostasis, and wherein if the disease is not ameliorated then the animal's health continues to deteriorate.

As used herein, the term “anti-tumor drug” refers to any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radioactive agents. Anti-tumor drugs can also include any agent useful to indirectly combat cancer via the immune system, including immunostimulatory agents that activate a biological response upon recognition by a receptor. In various embodiments of the present disclosure, anti-tumor drugs can include TLR agonists, including TLR7 agonists and/or TLR8 agonists. An anti-tumor drug herein can be a small molecule, e.g., a compound of Formula I. Also encompassed within the scope of the term “anti-tumor drug,” are conjugates of peptides or proteins with anti-tumor activity, e.g., cytokines such as TNF-α. Anti-tumor drug conjugates herein also include, but are not limited to those formed between an antibody, a linker, and a compound of the present disclosure, e.g., a compound of Formula I.

The terms “administration” or “administering,” as used herein, refer to a process of delivering a treatment (e.g., a therapeutic agent and/or a therapeutic composition) to a subject. An administration may be performed using oral administration, administration as a suppository, topical contact, intravenous, intraperitoneal, intramuscular, intralesional, intranasal or subcutaneous administration, intrathecal administration, or the implantation of a slow-release device, e.g., a mini-osmotic pump, to the subject. An administration may be systemic or local, in which the treatment is preferentially delivered to a target location in a subject as compared to a systemic distribution of the agent.

Where a range of values is provided, it is understood that the range encompasses each intervening value, to the tenth of the unit of the lower limit unless the context clearly dictates otherwise, between the upper and lower limit of that range and any other stated or intervening value in that stated range. The upper and lower limits of these smaller ranges may independently be included in the smaller ranges and are also encompassed by the present disclosure, subject to any specifically excluded limit in the stated range. Where the stated range includes one or both of the limits, ranges excluding either or both of those included limits are also included (e.g., through the use of terms such as “between,” the upper and lower limit values are also considered to be included in the recited range).

The term“about,” as used herein in the context of a numerical value or range, generally refers to ±10%, 9%, 8%, 7%, 6%, 5%, 4%, 3%, 2%, or 1% of the numerical value or range recited or claimed, unless otherwise specified.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

Unless defined otherwise, all technical and scientific terms used herein have the same meaning as commonly understood by one of ordinary skill in the art to which the present disclosure belongs.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Although any methods and materials similar or equivalent to those described herein may also be used in the practice or testing of the compounds, conjugates, compositions and methods described herein, representative illustrative methods and materials are described.

### Compounds

The present disclosure provides compounds capable of eliciting an immune response in a subject upon administration. In various cases, such immune response is stimulated by agonizing a TLR (e.g., TLR7) in the subject using one or more compound(s) of the present disclosure.

In various embodiments, the present disclosure provides a compound having Formula I:

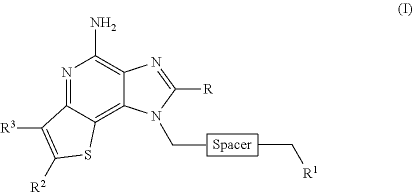

or a pharmaceutically acceptable salt thereof,

wherein:

R is H, C₁-C₆ alkyl, CH₂SR¹⁵ or CH₂OR¹⁵;

R¹ is —OH, —NR⁴R⁵, —OR¹⁰, SR¹¹ or

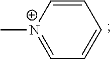

R² and R³ are each independently H or optionally substituted C₁-C₆ alkyl;

Spacer is —(CH₂)ₙ—,

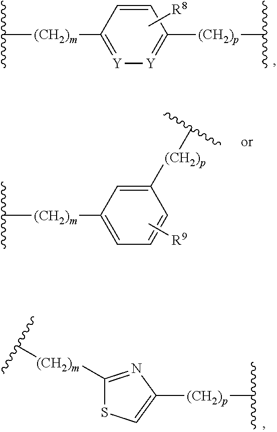

wherein n is an integer between 3 and 10; each m is independently an integer between 0 and 4; each p is independently an integer between 0 and 4; and Y is CH or N;

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;

R⁸ and R⁹ are each independently H, NR¹³R¹⁴, halo, optionally substituted C₁-C₄ alkoxy or optionally substituted C₁-C₄ alkyl;

R¹⁰ and R¹¹ are each independently optionally substituted C₁-C₆ alkyl, optionally substituted aryl or optionally substituted C₃-C₇ cycloalkyl;

R¹³ and R¹⁴ are each independently H or optionally substituted C₁-C₄ alkyl; and

R¹⁵ is C₃-C₄ cycloalkyl or C₁-C₄ alkyl optionally substituted with one or more halo;

In some embodiments, in compounds of Formula I, R² and R³ are each independently H or C₁-C₆ alkyl; R⁸ and R⁹ are each independently H, NR¹³R¹⁴, halo, C₁-C₄ alkoxy or C₁-C₄ alkyl; and R¹³ and R¹⁴ are each independently H or C₁-C₄ alkyl.

In some embodiments, in compounds of Formula I, R is C₁-C₆ alkyl. In some embodiments, R is C₂-C₄ alkyl.

In some embodiments, in compounds of Formula I, R is CH₂OR¹⁵. In some embodiments, R¹⁵ is C₁-C₂ alkyl.

In some embodiments, in compounds of Formula I, Spacer is:

—(CH₂)ₙ—,

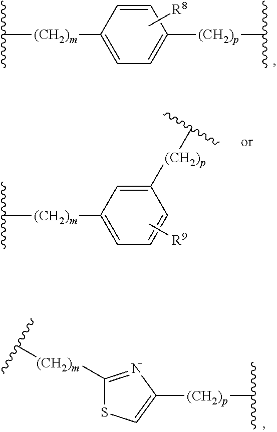

wherein n is an integer between 3 and 10; each m is independently an integer between 0 and 4; each p is independently an integer between 0 and 4, and R⁸ and R⁹ are each independently H, NR¹³R¹⁴, halo, optionally substituted C₁-C₄ alkoxy or optionally substituted C₁-C₄ alkyl, and wherein R¹³ and R¹⁴ are each independently H or optionally substituted C₁-C₄ alkyl.

In some embodiments, in compounds of Formula I, Spacer is:

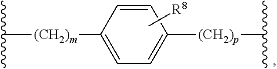

wherein m is an integer between 0 and 4; p is an integer between 0 and 4, and R⁸ is H, NR¹³R¹⁴, halo, optionally substituted C₁-C₄ alkoxy or optionally substituted C₁-C₄ alkyl, wherein R¹³ and R¹⁴ are each independently H or optionally substituted C₁-C₄ alkyl.

In some embodiments, in compounds of Formula I, R⁸ and R⁹ are each independently H, NR¹³R¹⁴, halo, C₁-C₄ alkoxy or C₁-C₄ alkyl, wherein R¹³ and R¹⁴ are each independently H or C₁-C₄ alkyl.

In some embodiments, in compounds of Formula I, R⁸ and R⁹ are each H or halo.

In some embodiments, in compounds of Formula I, m is 0. In some embodiments, in compounds of Formula I, p is 0.

In some embodiments, in compounds of Formula I, Spacer is —(CH₂)ₙ—,

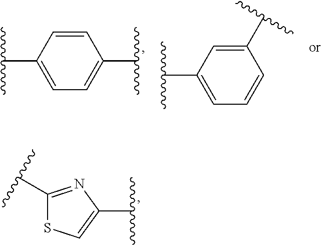

wherein n is an integer between 3 and 10.

In some embodiments, in compounds of Formula I, n is an integer between 3 and 5.

In some embodiments, in compounds of Formula I, Spacer is

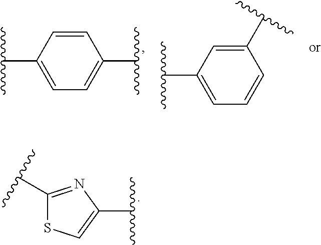

In some embodiments, in compounds of Formula I, R¹ is —OH, —NR⁴R⁵, —OR¹⁰, SR¹¹,

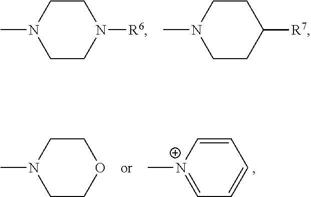

wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

In some embodiments, in compounds of Formula I, R¹ is —OH, —NR⁴R⁵,

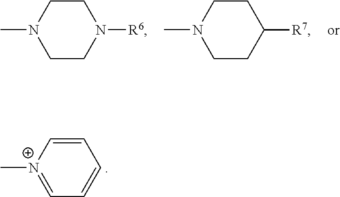

In some embodiments, in compounds of Formula I, R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

In some embodiments, in compounds of Formula I, R⁷ is H, optionally substituted C₁-C₄ alkyl, optionally substituted C₁-C₄ aminoalkyl or optionally substituted heteroaryl.

In some embodiments, in compounds of Formula I, R⁴ is H, optionally substituted C₁-C₄ alkyl, optionally substituted C₁-C₄ carboxyalkyl or optionally substituted C₁-C₄ aminoalkyl.

In some embodiments, in compounds of Formula I, R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

In some embodiments, in compounds of Formula I, R² and R³ are each independently H or C₁-C₆ alkyl.

In some embodiments, in compounds of Formula I, R² and R³ are each independently H or C₁-C₄ alkyl.

In some embodiments, in compounds of Formula I, R³ is H.

In some embodiments, in compounds of Formula I, R² is C₁-C₄ alkyl, and R³ is H.

In some embodiments, in compounds of Formula I, R² and R³ are each H.

Combinations of any of the foregoing embodiments for compounds of Formula I are also contemplated and each combination forms a separate embodiment for the purposes of the present disclosure.

In some embodiments, the compound of Formula I has Formula II:

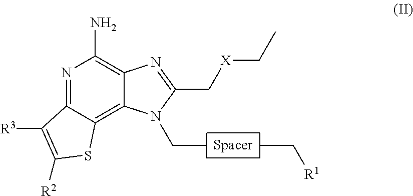

wherein:

X is —CH₂— or —O—; and

R¹, R², R³ and Spacer are as defined for Formula I.

In some embodiments, in compounds of Formula II, Spacer is:

—(CH₂)ₙ—,

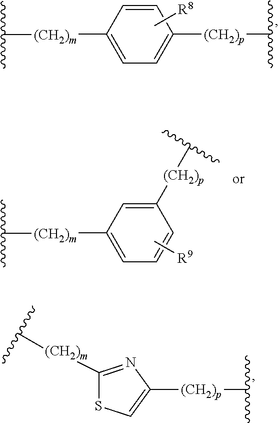

wherein n is an integer between 3 and 10; each m is independently an integer between 0 and 4; each p is independently an integer between 0 and 4, and R⁸ and R⁹ are each independently H, NR¹³R¹⁴, halo, optionally substituted C₁-C₄ alkoxy or optionally substituted C₁-C₄ alkyl, wherein R¹³ and R¹⁴ are each independently H or optionally substituted C₁-C₄ alkyl.

In some embodiments, in compounds of Formula II, Spacer is:

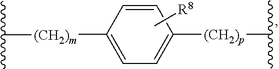

wherein m is an integer between 0 and 4; p is an integer between 0 and 4, and R⁸ is H, NR¹³R¹⁴, halo, optionally substituted C₁-C₄ alkoxy or optionally substituted C₁-C₄ alkyl, wherein R¹³ and R¹⁴ are each independently H or optionally substituted C₁-C₄ alkyl.

In some embodiments, in compounds of Formula II, R⁸ and R⁹ are each independently H, NR¹³R¹⁴, halo, C₁-C₄ alkoxy or C₁-C₄ alkyl, wherein R¹³ and R¹⁴ are each independently H or C₁-C₄ alkyl.

In some embodiments, in compounds of Formula II, R⁸ and R⁹ are each H or halo.

In some embodiments, in compounds of Formula II, m is 0. In some embodiments, in compounds of Formula II, p is 0.

In some embodiments, in compounds of Formula II, Spacer is —(CH₂)ₙ—,

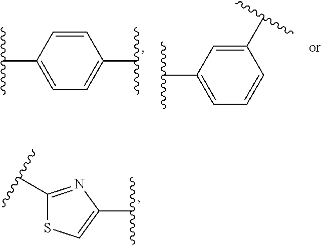

wherein n is an integer between 3 and 10.

In some embodiments, in compounds of Formula II, n is an integer between 3 and 5.

In some embodiments, in compounds of Formula II, Spacer is

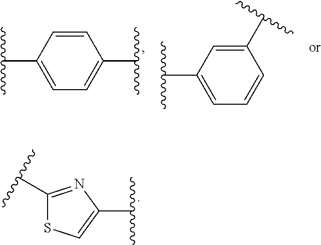

In some embodiments, in compounds of Formula II, R¹ is —OH, —NR⁴R⁵, —OR¹⁰, SR¹¹,

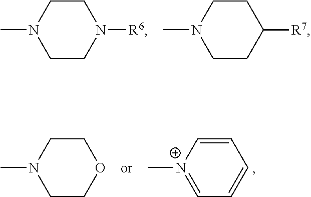

wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

In some embodiments, in compounds of Formula II, R¹ is —OH, —NR⁴R⁵,

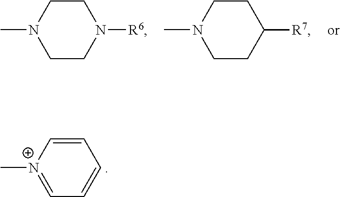

In some embodiments, in compounds of Formula II, R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

In some embodiments, in compounds of Formula II, R⁷ is H, optionally substituted C₁-C₄ alkyl, optionally substituted C₁-C₄ aminoalkyl or optionally substituted heteroaryl.

In some embodiments, in compounds of Formula II, R⁴ is H, optionally substituted C₁-C₄ alkyl, optionally substituted C₁-C₄ carboxyalkyl or optionally substituted C₁-C₄ aminoalkyl.

In some embodiments, in compounds of Formula II, R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

In some embodiments, in compounds of Formula II, R² and R³ are each independently H or C₁-C₆ alkyl.

In some embodiments, in compounds of Formula II, R² and R³ are each independently H or C₁-C₄ alkyl.

In some embodiments, in compounds of Formula II, R³ is H.

In some embodiments, in compounds of Formula II, R² is C₁-C₄ alkyl, and R³ is H.

In some embodiments, in compounds of Formula II, R² and R³ are each H.

Combinations of any of the foregoing embodiments for compounds of Formula II are also contemplated and each combination forms a separate embodiment for the purposes of the present disclosure.

In some embodiments, the compound of Formula I has Formula III:

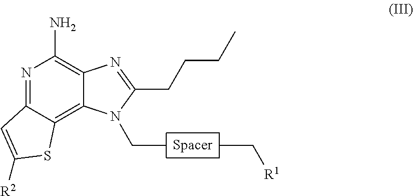

wherein R¹, R² and Spacer are as defined for Formula I.

In some embodiments, in compounds of Formula III, Spacer is: —(CH₂)ₙ—,

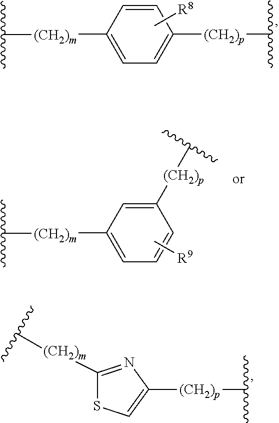

wherein n is an integer between 3 and 10; each m is independently an integer between 0 and 4; each p is independently an integer between 0 and 4, and R⁸ and R⁹ are each independently H, NR¹³R¹⁴, halo, optionally substituted C₁-C₄ alkoxy or optionally substituted C₁-C₄ alkyl, wherein R¹³ and R¹⁴ are each independently H or optionally substituted C₁-C₄ alkyl.

In some embodiments, in compounds of Formula III, Spacer is:

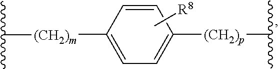

wherein m is an integer between 0 and 4; p is an integer between 0 and 4, and R⁸ is H, NR¹³R¹⁴, halo, optionally substituted C₁-C₄ alkoxy or optionally substituted C₁-C₄ alkyl, wherein R¹³ and R¹⁴ are each independently H or optionally substituted C₁-C₄ alkyl.

In some embodiments, in compounds of Formula III, R⁸ and R⁹ are each independently H, NR¹³R¹⁴, halo, C₁-C₄ alkoxy or C₁-C₄ alkyl, wherein R¹³ and R¹⁴ are each independently H or C₁-C₄ alkyl.

In some embodiments, in compounds of Formula III, R⁸ and R⁹ are each H or halo.

In some embodiments, in compounds of Formula III, m is 0. In some embodiments, in compounds of Formula III, p is 0.

In some embodiments, in compounds of Formula III, Spacer is —(CH₂)ₙ—,

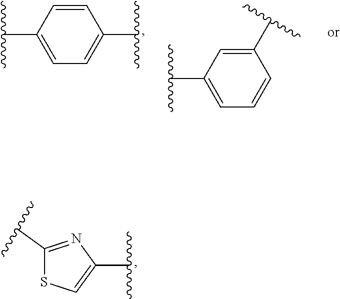

wherein n is an integer between 3 and 10.

In some embodiments, in compounds of Formula III, n is an integer between 3 and 5.

In some embodiments, in compounds of Formula III, Spacer is

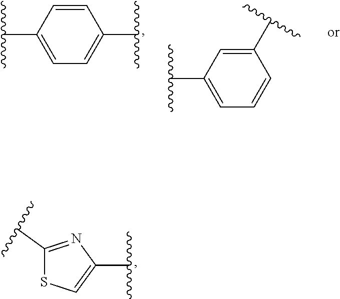 [SMILES: *c1ccc(*)cc1 ; *c1cccc(*)c1 ; *c1csc(*)n1]

In some embodiments, in compounds of Formula III, R¹ is —OH, —NR⁴R⁵, —OR¹⁰, SR¹¹,

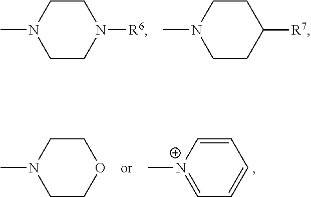

wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

In some embodiments, in compounds of Formula III, R¹ is —OH, —NR⁴R⁵,

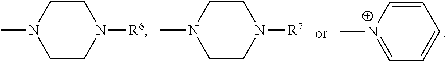

In some embodiments, in compounds of Formula III, R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

In some embodiments, in compounds of Formula III, R⁷ is H, optionally substituted C₁-C₄ alkyl, optionally substituted C₁-C₄ aminoalkyl or optionally substituted heteroaryl.

In some embodiments, in compounds of Formula III, R⁴ is H, optionally substituted C₁-C₄ alkyl, optionally substituted C₁-C₄ carboxyalkyl or optionally substituted C₁-C₄ aminoalkyl.

In some embodiments, in compounds of Formula III, R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

In some embodiments, in compounds of Formula III, R² is H or C₁-C₆ alkyl.

In some embodiments, in compounds of Formula III, R² is H or C₁-C₄ alkyl.

In some embodiments, in compounds of Formula III, R² is C₁-C₄ alkyl.

In some embodiments, in compounds of Formula III, R² is H.

Combinations of any of the foregoing embodiments for compounds of Formula III are also contemplated and each combination forms a separate embodiment for the purposes of the present disclosure.

In some embodiments, the compound of Formula I has Formula IV:

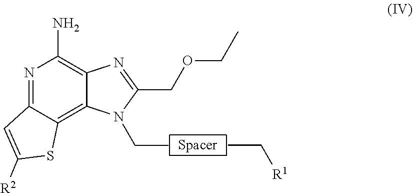

wherein R¹, R² and Spacer are as defined for Formula I.

In some embodiments, in compounds of Formula IV, Spacer is: —(CH₂)ₙ—,

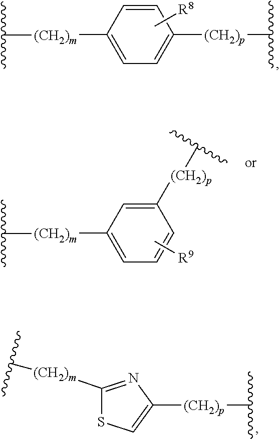

wherein n is an integer between 3 and 10; each m is independently an integer between 0 and 4; each p is independently an integer between 0 and 4, and R⁸ and R⁹ are each independently H, NR¹³R¹⁴, halo, optionally substituted C₁-C₄ alkoxy or optionally substituted C₁-C₄ alkyl, wherein R¹³ and R¹⁴ are each independently H or optionally substituted C₁-C₄ alkyl.

In some embodiments, in compounds of Formula IV, Spacer is:

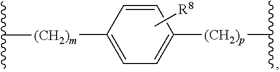

wherein m is an integer between 0 and 4; p is an integer between 0 and 4, and R⁸ is H, NR¹³R¹⁴, halo, optionally substituted C₁-C₄ alkoxy or optionally substituted C₁-C₄ alkyl, wherein R¹³ and R¹⁴ are each independently H or optionally substituted C₁-C₄ alkyl.

In some embodiments, in compounds of Formula IV, R⁸ and R⁹ are each independently H, NR¹³R¹⁴, halo, C₁-C₄ alkoxy or C₁-C₄ alkyl, wherein R¹³ and R¹⁴ are each independently H or C₁-C₄ alkyl.

In some embodiments, in compounds of Formula IV, R⁸ and R⁹ are each H or halo.

In some embodiments, in compounds of Formula IV, m is 0. In some embodiments, in compounds of Formula IV, p is 0.

In some embodiments, in compounds of Formula IV, Spacer is —(CH₂)ₙ—,

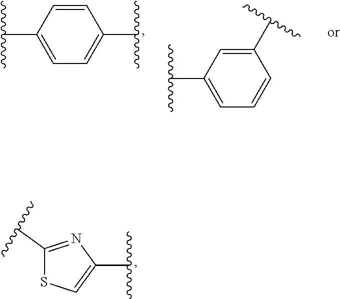

wherein n is an integer between 3 and 10.

In some embodiments, in compounds of Formula IV, n is an integer between 3 and 5.

In some embodiments, in compounds of Formula IV, Spacer is

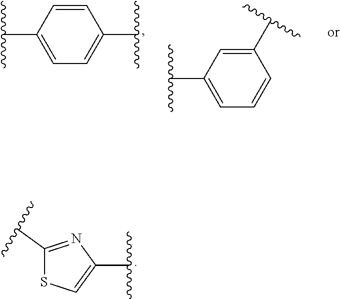 [SMILES: *c1ccc(*)cc1 ; *c1cccc(*)c1 ; *c1csc(*)n1]

In some embodiments, in compounds of Formula IV, R¹ is —OH, —NR⁴R⁵, —OR¹⁰, SR¹¹,

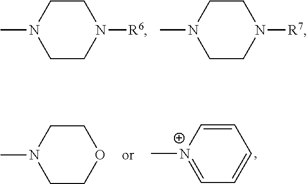

wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

In some embodiments, in compounds of Formula IV, R¹ is —OH, —NR⁴R⁵,

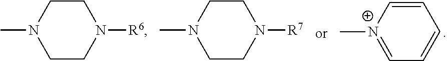

In some embodiments, in compounds of Formula IV, R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

In some embodiments, in compounds of Formula IV, R⁷ is H, optionally substituted C₁-C₄ alkyl, optionally substituted C₁-C₄ aminoalkyl or optionally substituted heteroaryl.

In some embodiments, in compounds of Formula IV, R⁴ is H, optionally substituted C₁-C₄ alkyl, optionally substituted C₁-C₄ carboxyalkyl or optionally substituted C₁-C₄ aminoalkyl.

In some embodiments, in compounds of Formula IV, R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

In some embodiments, in compounds of Formula IV, R² is H or C₁-C₆ alkyl.

In some embodiments, in compounds of Formula IV, R² is H or C₁-C₄ alkyl.

In some embodiments, in compounds of Formula IV, R² is C₁-C₄ alkyl.

In some embodiments, in compounds of Formula IV, R² is H.

Combinations of any of the foregoing embodiments for compounds of Formula IV are also contemplated and each combination forms a separate embodiment for the purposes of the present disclosure.

In some embodiments, the compound of Formula I has Formula V:

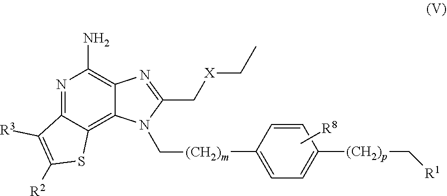

wherein X is —CH₂— or —O—, and R¹, R², R³, R⁸, m and p are as defined for Formula I.

In some embodiments, in compounds of Formula V, m is 0. In some embodiments, in compounds of Formula V, p is 0.

In some embodiments, in compounds of Formula V, R⁸ is H, NR¹³R¹⁴, halo, C₁-C₄ alkoxy or C₁-C₄ alkyl, wherein R¹³ and R¹⁴ are each independently H or C₁-C₄ alkyl.

In some embodiments, in compounds of Formula V, R⁸ is H or halo.

In some embodiments, in compounds of Formula V, R¹ is —OH, —NR⁴R⁵, —OR¹⁰, SR¹¹,

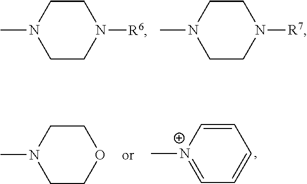

wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

In some embodiments, in compounds of Formula V, R¹ is —OH, —NR⁴R⁵,

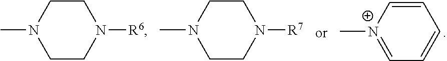

In some embodiments, in compounds of Formula V, R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

In some embodiments, in compounds of Formula V, R⁷ is H, optionally substituted C₁-C₄ alkyl, optionally substituted C₁-C₄ aminoalkyl or optionally substituted heteroaryl.

In some embodiments, in compounds of Formula V, R⁴ is H, optionally substituted C₁-C₄ alkyl, optionally substituted C₁-C₄ carboxyalkyl or optionally substituted C₁-C₄ aminoalkyl.

In some embodiments, in compounds of Formula V, R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

In some embodiments, in compounds of Formula V, R² and R³ are each independently H or C₁-C₆ alkyl.

In some embodiments, in compounds of Formula V, R² and R³ are each independently H or C₁-C₄ alkyl.

In some embodiments, in compounds of Formula V, R³ is H.

In some embodiments, in compounds of Formula V, R² is C₁-C₄ alkyl, and R³ is H.

In some embodiments, in compounds of Formula V, R² and R³ are each H.

Combinations of any of the foregoing embodiments for compounds of Formula V are also contemplated and each combination forms a separate embodiment for the purposes of the present disclosure.

In some embodiments, the compound of Formula I has Formula VI:

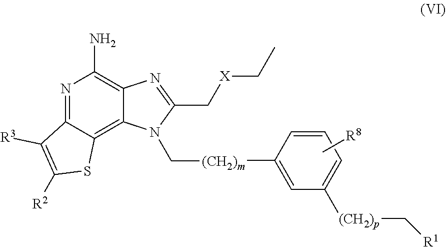

wherein X is —CH₂— or —O—, and R¹, R², R³, R⁹, m and p are as defined for Formula I.

In some embodiments, in compounds of Formula VI, m is 0. In some embodiments, in compounds of Formula VI, p is 0.

In some embodiments, in compounds of Formula VI, R⁹ is H, NR¹³R¹⁴, halo, C₁-C₄ alkoxy or C₁-C₄ alkyl, wherein R¹³ and R¹⁴ are each independently H or C₁-C₄ alkyl.

In some embodiments, in compounds of Formula VI, R⁹ is H or halo.

In some embodiments, in compounds of Formula VI, R¹ is —OH, —NR⁴R⁵, —OR¹⁰, SR¹¹,

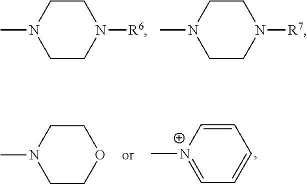

wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

In some embodiments, in compounds of Formula VI, R¹ is —OH, —NR⁴R⁵,

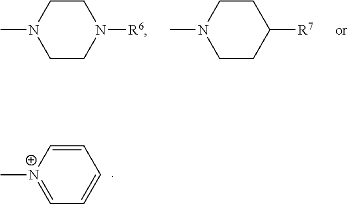

In some embodiments, in compounds of Formula VI, R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

In some embodiments, in compounds of Formula VI, R⁷ is H, optionally substituted C₁-C₄ alkyl, optionally substituted C₁-C₄ aminoalkyl or optionally substituted heteroaryl.

In some embodiments, in compounds of Formula VI, R⁴ is H, optionally substituted C₁-C₄ alkyl, optionally substituted C₁-C₄ carboxyalkyl or optionally substituted C₁-C₄ aminoalkyl.

In some embodiments, in compounds of Formula VI, R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

In some embodiments, in compounds of Formula VI, R² and R³ are each independently H or C₁-C₆ alkyl.

In some embodiments, in compounds of Formula VI, R² and R³ are each independently H or C₁-C₄ alkyl.

In some embodiments, in compounds of Formula VI, R³ is H.

In some embodiments, in compounds of Formula VI, R² is C₁-C₄ alkyl, and R³ is H.

In some embodiments, in compounds of Formula VI, R² and R³ are each H.

Combinations of any of the foregoing embodiments for compounds of Formula VI are also contemplated and each combination forms a separate embodiment for the purposes of the present disclosure.

The present disclosure further provides a compound of Formula I selected from the following compounds listed in Table 1, or a pharmaceutically acceptable salt thereof.

TABLE 1
Example Compounds

| | |
|---|---|
| 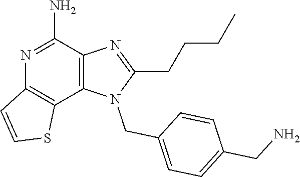 [SMILES: CCCCc1nc2c(N)nc3ccsc3c2n1Cc1ccc(CN)cc1] | 1-(4-(aminomethyl)benzyl)-2-butyl-1H- imidazo[4,5-d]thieno[3,2-b]pyridin-4-amine (Compound 111) |
| 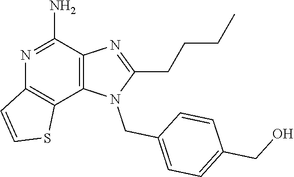 [SMILES: CCCCc1nc2c(N)nc3ccsc3c2n1Cc1ccc(CO)cc1] | (4-((4-amino-2-butyl-1H-imidazo[4,5- d]thieno[3,2-b]pyridin-1- yl)methyl)phenyl)methanol (Compound 141) |
| 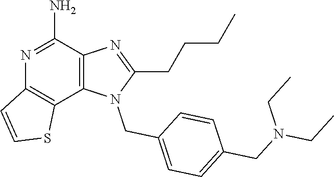 [SMILES: CCCCc1nc2c(N)nc3ccsc3c2n1Cc1ccc(CN(CC)CC)cc1] | 2-butyl-1-(4-((diethylamino)methyl)benzyl)-1H- imidazo[4,5-d]thieno[3,2-b]pyridin-4-amine (Compound 144) |
| 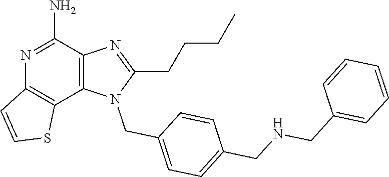 [SMILES: CCCCc1nc2c(N)nc3ccsc3c2n1Cc1ccc(CNCc2ccccc2)cc1] | 1-(4-((benzylamino)methyl)benzyl)-2-butyl-1H- imidazo[4,5-d]thieno[3,2-b]pyridin-4-amine (Compound 145) |
| 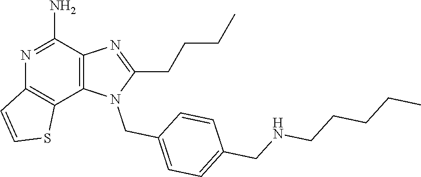 [SMILES: CCCCCNCc1ccc(Cn2c(CCCC)nc3c(N)nc4ccsc4c32)cc1] | 2-butyl-1-(4-((pentylamino)methyl)benzyl)-1H- imidazo[4,5-d]thieno[3,2-b]pyridin-4-amine (Compound 146) |
| 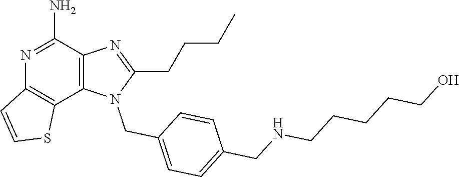 [SMILES: CCCCc1nc2c(N)nc3ccsc3c2n1Cc1ccc(CNCCCCCO)cc1] | 5-((4-((4-amino-2-butyl-1H-imidazo[4,5- d]thieno[3,2-b]pyridin-1- yl)methyl)benzyl)amino)pentan-1-ol (Compound 147) |
| 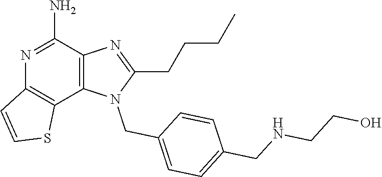 [SMILES: CCCCc1nc2c(N)nc3ccsc3c2n1Cc1ccc(CNCCO)cc1] | 2-((4-((4-amino-2-butyl-1H-imidazo[4,5- d]thieno[3,2-b]pyridin-1- yl)methyl)benzyl)amino)ethan-1-ol (Compound 148) |
| 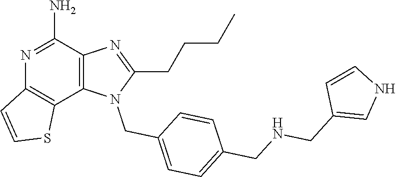 [SMILES: CCCCc1nc2c(N)nc3ccsc3c2n1Cc1ccc(CNCc2cc[nH]c2)cc1] | 1-(4-((((1H-pyrrol-3- yl)methyl)amino)methyl)benzyl)-2-butyl-1H- imidazo[4,5-d]thieno[3,2-b]pyridin-4-amine (Compound 149) |
| 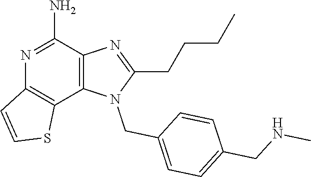 [SMILES: CCCCc1nc2c(N)nc3ccsc3c2n1Cc1ccc(CNC)cc1] | 2-butyl-1-(4-((methylamino)methyl)benzyl)-1H- imidazo[4,5-d]thieno[3,2-b]pyridin-4-amine (Compound 150) |
| 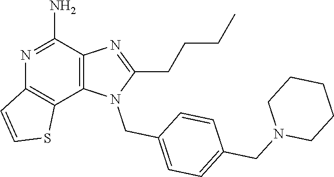 [SMILES: CCCCc1nc2c(N)nc3ccsc3c2n1Cc1ccc(CN2CCCCC2)cc1] | 2-butyl-1-(4-(piperidin-1-ylmethyl)benzyl)-1H- imidazo[4,5-d]thieno[3,2-b]pyridin-4-amine (Compound 151) |
| 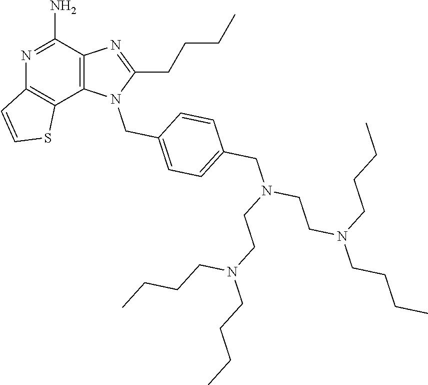 [SMILES: CCCCc1nc2c(N)nc3ccsc3c2n1Cc1ccc(CN(CCN(CCCC)CCCC)CCN(CCCC)CCCC)cc1] | N¹-(4-((4-amino-2-butyl-1H-imidazo[4,5- d]thieno[3,2-b]pyridin-1-yl)methyl)benzyl)- N²,N²-dibutyl-N¹-(2-(dibutylamino)ethyl)ethane- 1,2-diamine (Compound 152) |
| 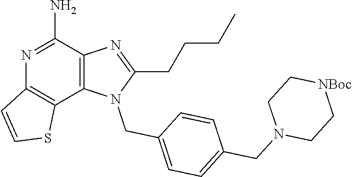 [SMILES: CCCCc1nc2c(N)nc3ccsc3c2n1Cc1ccc(CN2CCN(C(=O)OC(C)(C)C)CC2)cc1] | tert-butyl 4-(4-((4-amino-2-butyl-1H- imidazo[4,5-d]thieno[3,2-b]pyridin-1- yl)methyl)benzyl)piperazine-1-carboxylate (Compound 153) |
| 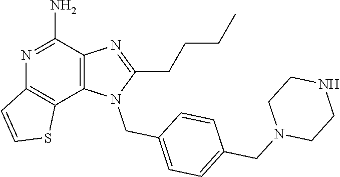 [SMILES: CCCCc1nc2c(N)nc3ccsc3c2n1Cc1ccc(CN2CCNCC2)cc1] | 2-butyl-1-(4-(piperazin-1-ylmethyl)benzyl)-1H- imidazo[4,5-d]thieno[3,2-b]pyridin-4-amine (Compound 154) |
| 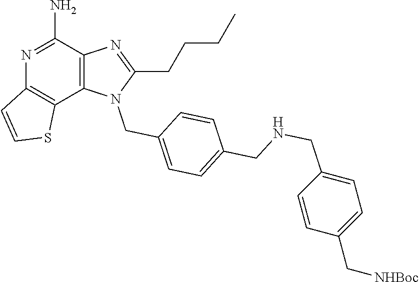 [SMILES: CCCCc1nc2c(N)nc3ccsc3c2n1Cc1ccc(CNCc2ccc(CNC(=O)OC(C)(C)C)cc2)cc1] | tert-butyl (4-(((4-((4-amino-2-butyl-1H- imidazo[4,5-d]thieno[3,2-b]pyridin-1- yl)methyl)benzyl)amino)methyl)benzyl)carbamate (Compound 155) |
| 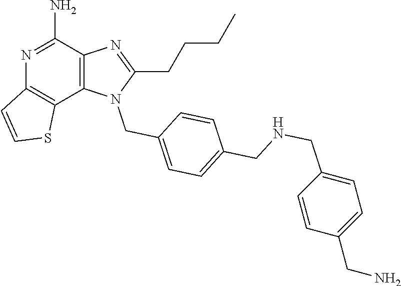 [SMILES: CCCCc1nc2c(N)nc3ccsc3c2n1Cc1ccc(CNCc2ccc(CN)cc2)cc1] | 1-(4-(((4- (aminomethyl)benzyl)amino)methyl)benzyl)-2- butyl-1H-imidazo[4,5-d]thieno[3,2-b]pyridin-4- amine (Compound 156) |
| 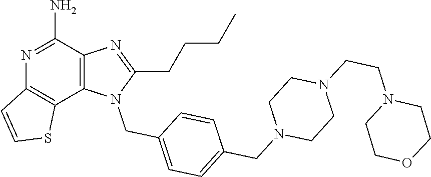 [SMILES: CCCCc1nc2c(N)nc3ccsc3c2n1Cc1ccc(CN2CCN(CCN3CCOCC3)CC2)cc1] | 2-butyl-1-(4-((4-(2-morpholinoethyl)piperazin-1- yl)methyl)benzyl)-1H-imidazo[4,5-d]thieno[3,2- b]pyridin-4-amine (Compound 157) |
| 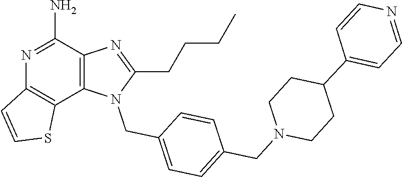 [SMILES: CCCCc1nc2c(N)nc3ccsc3c2n1Cc1ccc(CN2CCC(c3ccncc3)CC2)cc1] | 2-butyl-1-(4-((4-(pyridin-4-yl)piperidin-1- yl)methyl)benzyl)-1H-imidazo[4,5-d]thieno[3,2- b]pyridin-4-amine (Compound 158) |
| 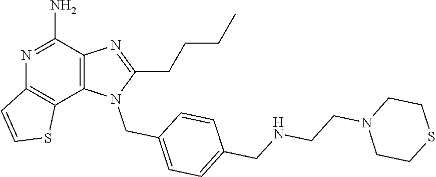 [SMILES: CCCCc1nc2c(N)nc3ccsc3c2n1Cc1ccc(CNCCN2CCSCC2)cc1] | 2-butyl-1-(4-(((2- thiomorpholinoethyl)amino)methyl)benzyl)-1H- imidazo[4,5-d]thieno[3,2-b]pyridin-4-amine (Compound 159) |
| 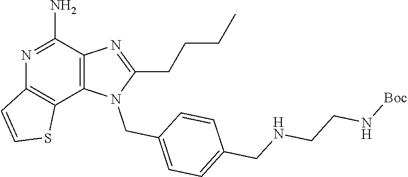 [SMILES: CCCCc1nc2c(N)nc3ccsc3c2n1Cc1ccc(CNCCNC(=O)OC(C)(C)C)cc1] | tert-butyl (2-((4-((4-amino-2-butyl-1H- imidazo[4,5-d]thieno[3,2-b]pyridin-1- yl)methyl)benzyl)amino)ethyl)carbamate (Compound 160) |
| 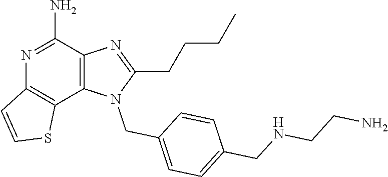 [SMILES: CCCCc1nc2c(N)nc3ccsc3c2n1Cc1ccc(CNCCN)cc1] | N¹-(4-((4-amino-2-butyl-1H-imidazo[4,5- d]thieno[3,2-b]pyridin-1- yl)methyl)benzyl)ethane-1,2-diamine (Compound 161) |
| 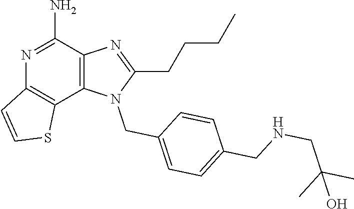 [SMILES: CCCCc1nc2c(N)nc3ccsc3c2n1Cc1ccc(CNCC(C)(C)O)cc1] | 1-((4-((4-amino-2-butyl-1H-imidazo[4,5- d]thieno[3,2-b]pyridin-1- yl)methyl)benzyl)amino)-2-methylpropan-2-ol (Compound 162) |
| 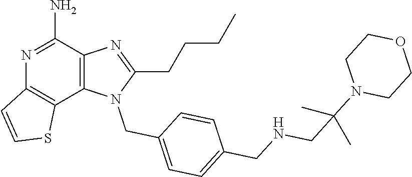 [SMILES: CCCCc1nc2c(N)nc3ccsc3c2n1Cc1ccc(CNCC(C)(C)N2CCOCC2)cc1] | 2-butyl-1-(4-(((2-methyl-2- morpholinopropyl)amino)methyl)benzyl)-1H- imidazo[4,5-d]thieno[3,2-b]pyridin-4-amine (Compound 163) |
| 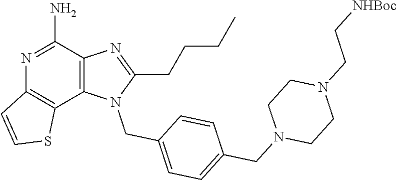 [SMILES: CCCCc1nc2c(N)nc3ccsc3c2n1Cc1ccc(CN2CCN(CCNC(=O)OC(C)(C)C)CC2)cc1] | tert-butyl (2-(4-(4-((4-amino-2-butyl-1H- imidazo[4,5-d]thieno[3,2-b]pyridin-1- yl)methyl)benzyl)piperazin-1-yl)ethyl)carbamate (Compound 165) |
| 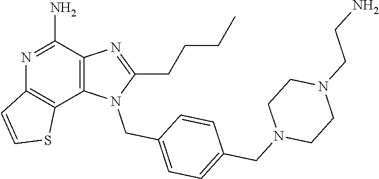 [SMILES: CCCCc1nc2c(N)nc3ccsc3c2n1Cc1ccc(CN2CCN(CCN)CC2)cc1] | 1-(4-((4-(2-aminoethyl)piperazin-1- yl)methyl)benzyl)-2-butyl-1H-imidazo[4,5- d]thieno[3,2-b]pyridin-4-amine (Compound 166) |
| 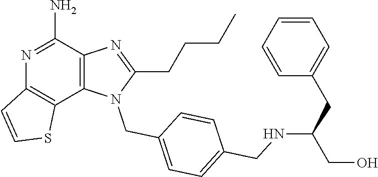 [SMILES: CCCCc1nc2c(N)nc3ccsc3c2n1Cc1ccc(CN[C@H](CO)Cc2ccccc2)cc1] | (S)-2-((4-((4-amino-2-butyl-1H-imidazo[4,5- d]thieno[3,2-b]pyridin-1- yl)methyl)benzyl)amino)-3-phenylpropan-1-ol (Compound 167) |
| 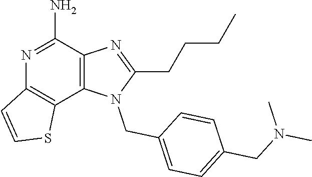 [SMILES: CCCCc1nc2c(N)nc3ccsc3c2n1Cc1ccc(CN(C)C)cc1] | 2-butyl-1-(4-((dimethylamino)methyl)benzyl)- 1H-imidazo[4,5-d]thieno[3,2-b]pyridin-4-amine (Compound 168) |
| 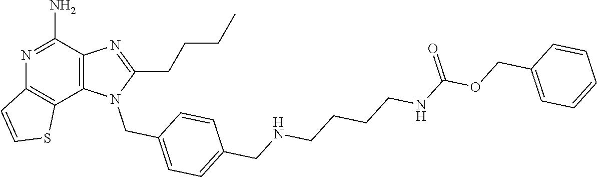 [SMILES: CCCCc1nc2c(N)nc3ccsc3c2n1Cc1ccc(CNCCCCNC(=O)OCc2ccccc2)cc1] | benzyl (4-((4-((4-amino-2-butyl-1H-imidazo[4,5- d]thieno[3,2-b]pyridin-1- yl)methyl)benzyl)amino)butyl)carbamate (Compound 260) |
| 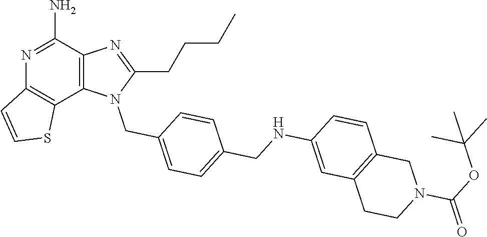 [SMILES: CCCCc1nc2c(N)nc3ccsc3c2n1Cc1ccc(CNc2ccc3c(c2)CCN(C(=O)OC(C)(C)C)C3)cc1] | tert-butyl 6-((4-((4-amino-2-butyl-1H- imidazo[4,5-d]thieno[3,2-b]pyridin-1- yl)methyl)benzyl)amino)-3,4- dihydroisoquinoline-2(1H)-carboxylate (Compound 261) |
| 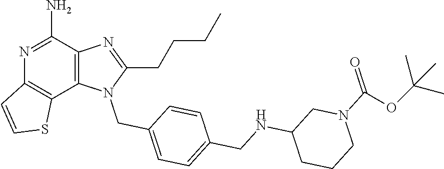 [SMILES: CCCCc1nc2c(N)nc3ccsc3c2n1Cc1ccc(CNC2CCCN(C(=O)OC(C)(C)C)C2)cc1] | tert-butyl 3-((4-((4-amino-2-butyl-1H- imidazo[4,5-d]thieno[3,2-b]pyridin-1- yl)methyl)benzyl)amino)piperidine-1-carboxylate (Compound 262) |
| 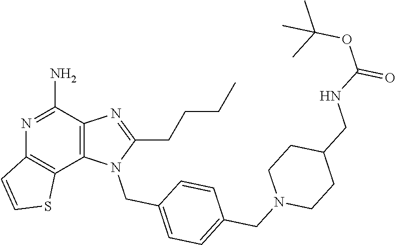 [SMILES: CCCCc1nc2c(N)nc3ccsc3c2n1Cc1ccc(CN2CCC(CNC(=O)OC(C)(C)C)CC2)cc1] | tert-butyl ((1-(4-((4-amino-2-butyl-1H- imidazo[4,5-d]thieno[3,2-b]pyridin-1- yl)methyl)benzyl)piperidin-4- yl)methyl)carbamate (Compound 263) |
| 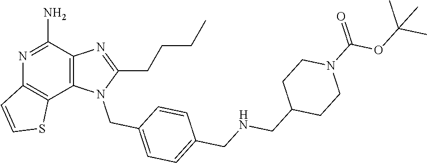 [SMILES: CCCCc1nc2c(N)nc3ccsc3c2n1Cc1ccc(CNCC2CCN(C(=O)OC(C)(C)C)CC2)cc1] | tert-butyl 4-(((4-((4-amino-2-butyl-1H- imidazo[4,5-d]thieno[3,2-b]pyridin-1- yl)methyl)benzyl)amino)methyl)piperidine-1- carboxylate (Compound 264) |
| 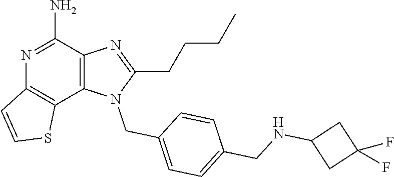 [SMILES: CCCCc1nc2c(N)nc3ccsc3c2n1Cc1ccc(CNC2CC(F)(F)C2)cc1] | 2-butyl-1-(4-(((3,3- difluorocyclobutyl)amino)methyl)benzyl)-1H- imidazo[4,5-d]thieno[3,2-b]pyridin-4-amine (Compound 265) |
| 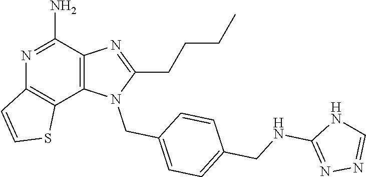 [SMILES: CCCCc1nc2c(N)nc3ccsc3c2n1Cc1ccc(CNc2nnc[nH]2)cc1] | 1-(4-(((4H-1,2,4-triazol-3- yl)amino)methyl)benzyl)-2-butyl-1H- imidazo[4,5-d]thieno[3,2-b]pyridin-4-amine (Compound 272) |
| 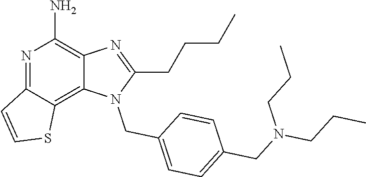 [SMILES: CCCCc1nc2c(N)nc3ccsc3c2n1Cc1ccc(CN(CCC)CCC)cc1] | 2-butyl-1-(4-((dipropylamino)methyl)benzyl)-1H- imidazo[4,5-d]thieno[3,2-b]pyridin-4-amine (Compound 277) |
| 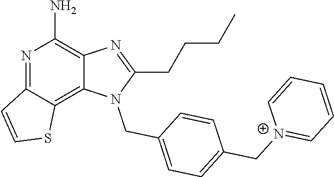 [SMILES: CCCCc1nc2c(N)nc3ccsc3c2n1Cc1ccc(C[n+]2ccccc2)cc1] | 1-(4-((4-amino-2-butyl-1H-imidazo[4,5- d]thieno[3,2-b]pyridin-1- yl)methyl)benzyl)pyridin-1-ium (Compound 279) |
| 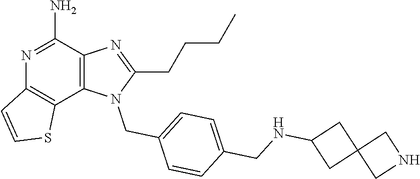 [SMILES: CCCCc1nc2c(N)nc3ccsc3c2n1Cc1ccc(CNC2CC3(CNC3)C2)cc1] | 1-(4-(((2-azaspiro[3.3]heptan-6- yl)amino)methyl)benzyl)-2-butyl-1H- imidazo[4,5-d]thieno[3,2-b]pyridin-4-amine (Compound 286) |
| 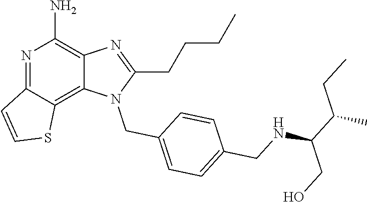 [SMILES: CCCCc1nc2c(N)nc3ccsc3c2n1Cc1ccc(CN[C@H](CO)[C@@H](C)CC)cc1] | (2S,3S)-2-((4-((4-amino-2-butyl-1H-imidazo[4,5- d]thieno[3,2-b]pyridin-1- yl)methyl)benzyl)amino)-3-methylpentan-1-ol (Compound 296) |
| 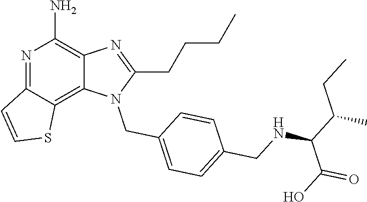 [SMILES: CCCCc1nc2c(N)nc3ccsc3c2n1Cc1ccc(CN[C@H](C(=O)O)[C@@H](C)CC)cc1] | (4-((4-amino-2-butyl-1H-imidazo[4,5- d]thieno[3,2-b]pyridin-1-yl)methyl)benzyl)-L- isoleucine (Compound 297) |
| 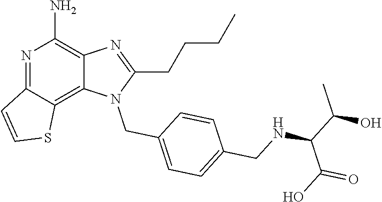 [SMILES: CCCCc1nc2c(N)nc3ccsc3c2n1Cc1ccc(CN[C@H](C(=O)O)[C@@H](C)O)cc1] | (4-((4-amino-2-butyl-1H-imidazo[4,5- d]thieno[3,2-b]pyridin-1-yl)methyl)benzyl)-L- threonine (Compound 298) |
| 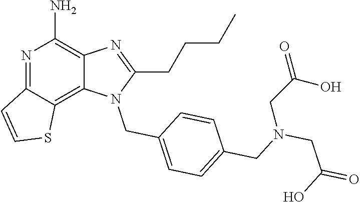 [SMILES: CCCCc1nc2c(N)nc3ccsc3c2n1Cc1ccc(CN(CC(=O)O)CC(=O)O)cc1] | 2,2′-((4-((4-amino-2-butyl-1H-imidazo[4,5- d]thieno[3,2-b]pyridin-1- yl)methyl)benzyl)azanediyl)diacetic acid (Compound 299) |
| 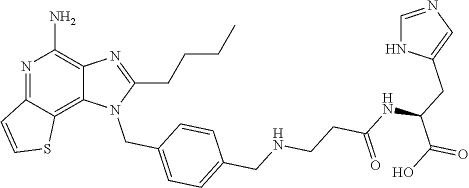 [SMILES: CCCCc1nc2c(N)nc3ccsc3c2n1Cc1ccc(CNCCC(=O)N[C@@H](Cc2cnc[nH]2)C(=O)O)cc1] | (3-((4-((4-amino-2-butyl-1H-imidazo[4,5- d]thieno[3,2-b]pyridin-1- yl)methyl)benzyl)amino)propanoyl)-L-histidine (Compound 301) |
| 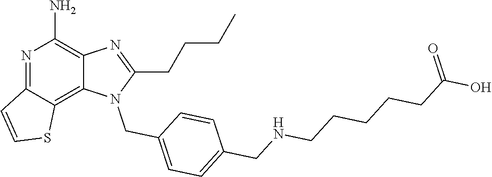 [SMILES: CCCCc1nc2c(N)nc3ccsc3c2n1Cc1ccc(CNCCCCCC(=O)O)cc1] | 6-((4-((4-amino-2-butyl-1H-imidazo[4,5- d]thieno[3,2-b]pyridin-1- yl)methyl)benzyl)amino)hexanoic acid (Compound 303) |
| 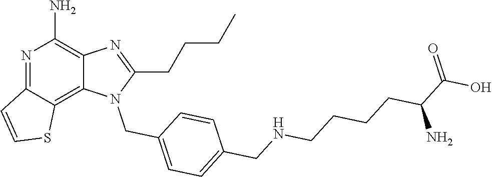 [SMILES: CCCCc1nc2c(N)nc3ccsc3c2n1Cc1ccc(CNCCCC[C@H](N)C(=O)O)cc1] | N⁶-(4-((4-amino-2-butyl-1H-imidazo[4,5- d]thieno[3,2-b]pyridin-1-yl)methyl)benzyl)-L- lysine (Compound 305) |
| 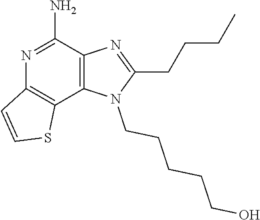 [SMILES: CCCCc1nc2c(N)nc3ccsc3c2n1CCCCCO] | 5-(4-amino-2-butyl-1H-imidazo[4,5-d]thieno[3,2- b]pyridin-1-yl)pentan-1-ol (Compound 172) |
| 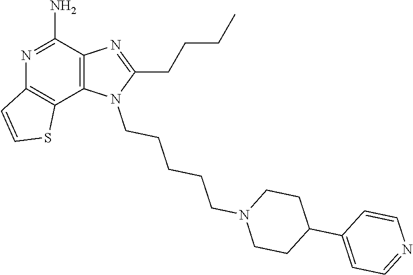 [SMILES: CCCCc1nc2c(N)nc3ccsc3c2n1CCCCCN1CCC(c2ccncc2)CC1] | 2-butyl-1-(5-(4-(pyridin-4-yl)piperidin-1- yl)pentyl)-1H-imidazo[4,5-d]thieno[3,2- b]pyridin-4-amine (Compound 174) |
| 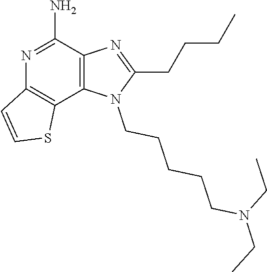 [SMILES: CCCCc1nc2c(N)nc3ccsc3c2n1CCCCCN(CC)CC] | 2-butyl-1-(5-(diethylamino)pentyl)-1H- imidazo[4,5-d]thieno[3,2-b]pyridin-4-amine (Compound 175) |
| 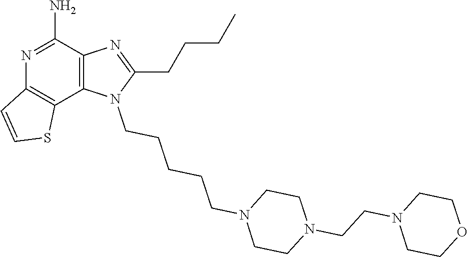 [SMILES: CCCCc1nc2c(N)nc3ccsc3c2n1CCCCCN1CCN(CCN2CCOCC2)CC1] | 2-butyl-1-(5-(4-(2-morpholinoethyl)piperazin-1- yl)pentyl)-1H-imidazo[4,5-d]thieno[3,2- b]pyridin-4-amine (Compound 176) |
| 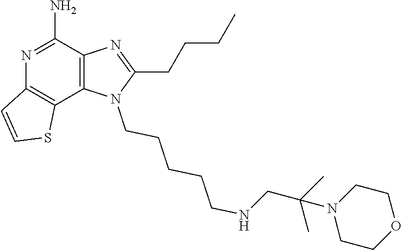 [SMILES: CCCCc1nc2c(N)nc3ccsc3c2n1CCCCCNCC(C)(C)N1CCOCC1] | 2-butyl-1-(5-((2-methyl-2- morpholinopropyl)amino)pentyl)-1H- imidazo[4,5-d]thieno[3,2-b]pyridin-4-amine (Compound 177) |
| 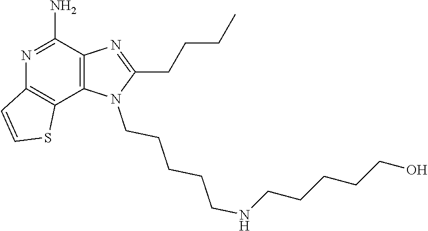 [SMILES: CCCCc1nc2c(N)nc3ccsc3c2n1CCCCCNCCCCCO] | 5-((5-(4-amino-2-butyl-1H-imidazo[4,5- d]thieno[3,2-b]pyridin-1-yl)pentyl)amino)pentan- 1-ol (Compound 178) |
| 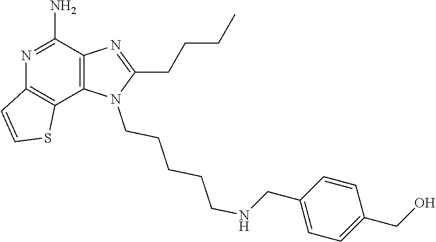 [SMILES: CCCCc1nc2c(N)nc3ccsc3c2n1CCCCCNCc1ccc(CO)cc1] | (4-(((5-(4-amino-2-butyl-1H-imidazo[4,5- d]thieno[3,2-b]pyridin-1- yl)pentyl)amino)methyl)phenyl)methanol (Compound 179) |
| 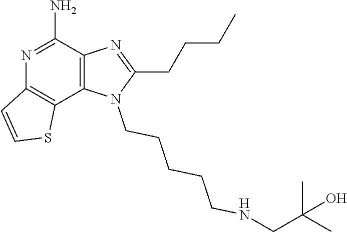 [SMILES: CCCCc1nc2c(N)nc3ccsc3c2n1CCCCCNCC(C)(C)O] | 1-((5-(4-amino-2-butyl-1H-imidazo[4,5- d]thieno[3,2-b]pyridin-1-yl)pentyl)amino)-2- methylpropan-2-ol (Compound 180) |
| 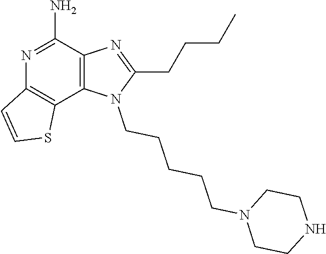 [SMILES: CCCCc1nc2c(N)nc3ccsc3c2n1CCCCCN1CCNCC1] | 2-butyl-1-(5-(piperazin-1-yl)pentyl)-1H- imidazo[4,5-d]thieno[3,2-b]pyridin-4-amine (Compound 182) |
| 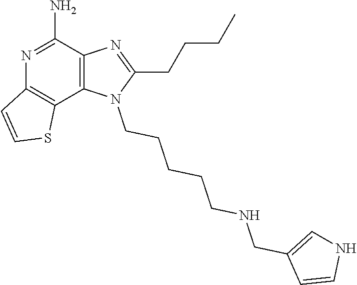 [SMILES: CCCCc1nc2c(N)nc3ccsc3c2n1CCCCCNCc1cc[nH]c1] | 1-(5-(((1H-pyrrol-3-yl)methyl)amino)pentyl)-2- butyl-1H-imidazo[4,5-d]thieno[3,2-b]pyridin-4- amine (Compound 183) |
| 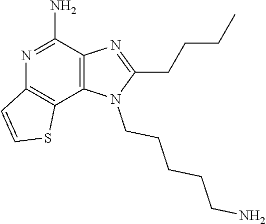 [SMILES: CCCCc1nc2c(N)nc3ccsc3c2n1CCCCCN] | 1-(5-aminopentyl)-2-butyl-1H-imidazo[4,5- d]thieno[3,2-b]pyridin-4-amine (Compound 185) |
| 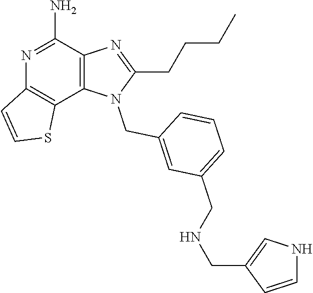 [SMILES: CCCCc1nc2c(N)nc3ccsc3c2n1Cc1cccc(CNCc2cc[nH]c2)c1] | 1-(3-((((1H-pyrrol-3- yl)methyl)amino)methyl)benzyl)-2-butyl-1H- imidazo[4,5-d]thieno[3,2-b]pyridin-4-amine (Compound 197) |
| 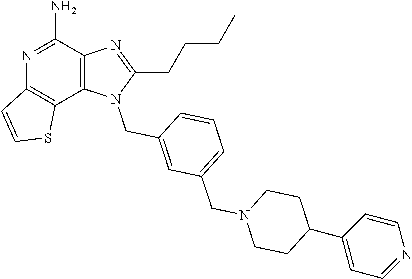 [SMILES: CCCCc1nc2c(N)nc3ccsc3c2n1Cc1cccc(CN2CCC(c3ccncc3)CC2)c1] | 2-butyl-1-(3-((4-(pyridin-4-yl)piperidin-1- yl)methyl)benzyl)-1H-imidazo[4,5-d]thieno[3,2- b]pyridin-4-amine (Compound 198) |
| 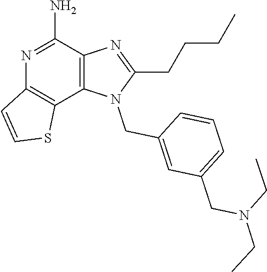 [SMILES: CCCCc1nc2c(N)nc3ccsc3c2n1Cc1cccc(CN(CC)CC)c1] | 2-butyl-1-(3-((diethylamino)methyl)benzyl)-1H- imidazo[4,5-d]thieno[3,2-b]pyridin-4-amine (Compound 199) |
| 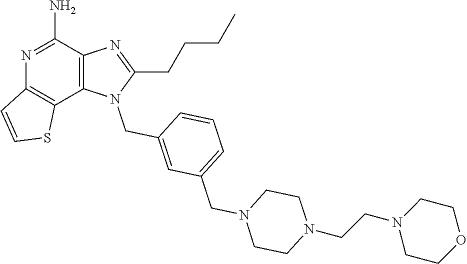 [SMILES: CCCCc1nc2c(N)nc3ccsc3c2n1Cc1cccc(CN2CCN(CCN3CCOCC3)CC2)c1] | 2-butyl-1-(3-((4-(2-morpholinoethyl)piperazin-1- yl)methyl)benzyl)-1H-imidazo[4,5-d]thieno[3,2- b]pyridin-4-amine (Compound 200) |
| 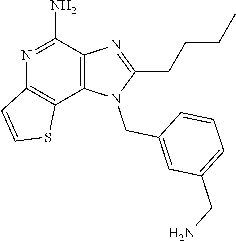 [SMILES: CCCCc1nc2c(N)nc3ccsc3c2n1Cc1cccc(CN)c1] | 1-(3-(aminomethyl)benzyl)-2-butyl-1H- imidazo[4,5-d]thieno[3,2-b]pyridin-4-amine (Compound 201) |
| 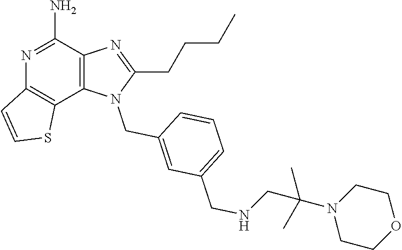 [SMILES: CCCCc1nc2c(N)nc3ccsc3c2n1Cc1cccc(CNCC(C)(C)N2CCOCC2)c1] | 2-butyl-1-(3-(((2-methyl-2- morpholinopropyl)amino)methyl)benzyl)-1H- imidazo[4,5-d]thieno[3,2-b]pyridin-4-amine (Compound 202) |
| 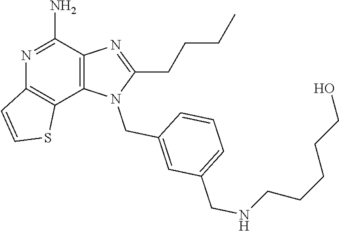 [SMILES: CCCCc1nc2c(N)nc3ccsc3c2n1Cc1cccc(CNCCCCCO)c1] | 5-((3-((4-amino-2-butyl-1H-imidazo[4,5- d]thieno[3,2-b]pyridin-1- yl)methyl)benzyl)amino)pentan-1-ol (Compound 203) |
| 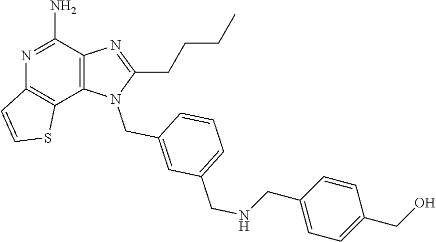 [SMILES: CCCCc1nc2c(N)nc3ccsc3c2n1Cc1cccc(CNCc2ccc(CO)cc2)c1] | (4-(((3-((4-amino-2-butyl-1H-imidazo[4,5- d]thieno[3,2-b]pyridin-1- yl)methyl)benzyl)amino)methyl)phenyl)methanol (Compound 204) |
| 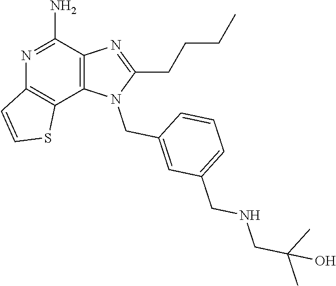 [SMILES: CCCCc1nc2c(N)nc3ccsc3c2n1Cc1cccc(CNCC(C)(C)O)c1] | 1-((3-((4-amino-2-butyl-1H-imidazo[4,5- d]thieno[3,2-b]pyridin-1- yl)methyl)benzyl)amino)-2-methylpropan-2-ol (Compound 205) |
| 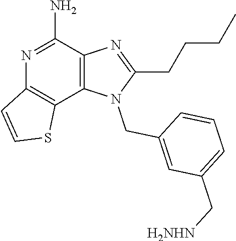 | 2-butyl-1-(3-(hydrazineylmethyl)benzyl)-1H- imidazo[4,5-d]thieno[3,2-b]pyridin-4-amine (Compound 206) |
| 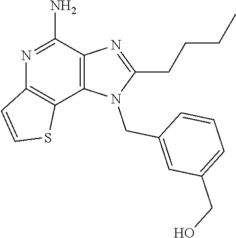 [SMILES: CCCCc1nc2c(N)nc3ccsc3c2n1Cc1cccc(CO)c1] | (3-((4-amino-2-butyl-1H-imidazo[4,5- d]thieno[3,2-b]pyridin-1- yl)methyl)phenyl)methanol (Compound 207) |
| 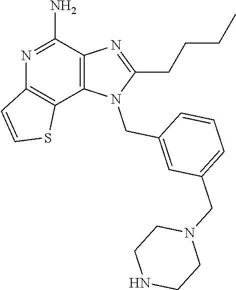 [SMILES: CCCCc1nc2c(N)nc3ccsc3c2n1Cc1cccc(CN2CCNCC2)c1] | 2-butyl-1-(3-(piperazin-1-ylmethyl)benzyl)-1H- imidazo[4,5-d]thieno[3,2-b]pyridin-4-amine (Compound 214) |
| 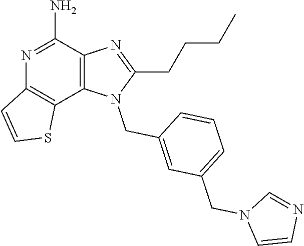 [SMILES: CCCCc1nc2c(N)nc3ccsc3c2n1Cc1cccc(Cn2ccnc2)c1] | 1-(3-((1H-imidazol-1-yl)methyl)benzyl)-2-butyl- 1H-imidazo[4,5-d]thieno[3,2-b]pyridin-4-amine (Compound 229) |
| 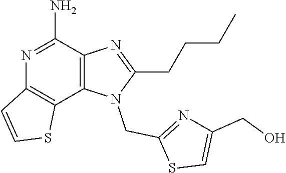 [SMILES: CCCCc1nc2c(N)nc3ccsc3c2n1Cc1nc(CO)cs1] | (2-((4-amino-2-butyl-1H-imidazo[4,5- d]thieno[3,2-b]pyridin-1-yl)methyl)thiazol-4- yl)methanol (Compound 228) |
| 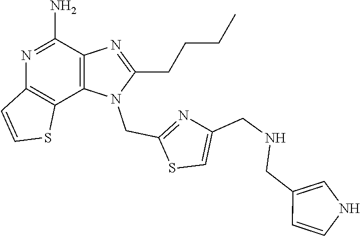 [SMILES: CCCCc1nc2c(N)nc3ccsc3c2n1Cc1nc(CNCc2cc[nH]c2)cs1] | 1-((4-((((1H-pyrrol-3- yl)methyl)amino)methyl)thiazol-2-yl)methyl)-2- butyl-1H-imidazo[4,5-d]thieno[3,2-b]pyridin-4- amine (Compound 231) |
| 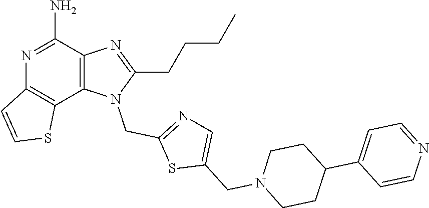 [SMILES: CCCCc1nc2c(N)nc3ccsc3c2n1Cc1ncc(CN2CCC(c3ccncc3)CC2)s1] | 2-butyl-1-((4-((4-(pyridin-4-yl)piperidin-1- yl)methyl)thiazol-2-yl)methyl)-1H-imidazo[4,5- d]thieno[3,2-b]pyridin-4-amine (Compound 232) |
| 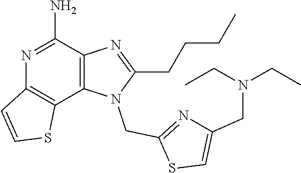 [SMILES: CCCCc1nc2c(N)nc3ccsc3c2n1Cc1nc(CN(CC)CC)cs1] | 2-butyl-1-((4-((diethylamino)methyl)thiazol-2- yl)methyl)-1H-imidazo[4,5-d]thieno[3,2- b]pyridin-4-amine (Compound 233) |
| 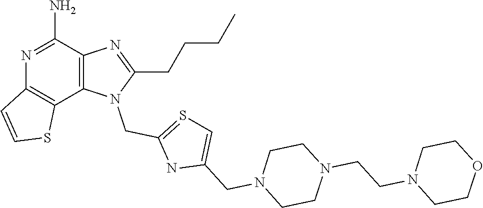 | 2-butyl-1-((4-((4-(2-morpholinoethyl)piperazin-1- yl)methyl)thiazol-2-yl)methyl)-1H-imidazo[4,5- d]thieno[3,2-b]pyridin-4-amine (Compound 234) |
| 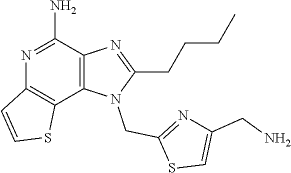 [SMILES: CCCCc1nc2c(N)nc3ccsc3c2n1Cc1nc(CN)cs1] | 1-((4-(aminomethyl)thiazol-2-yl)methyl)-2-butyl- 1H-imidazo[4,5-d]thieno[3,2-b]pyridin-4-amine (Compound 235) |
| 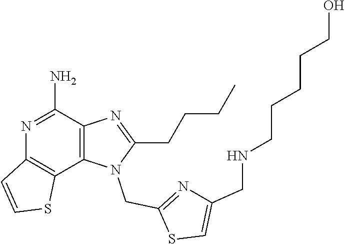 [SMILES: CCCCc1nc2c(N)nc3ccsc3c2n1Cc1nc(CNCCCCCO)cs1] | 5-(((2-((4-amino-2-butyl-1H-imidazo[4,5- d]thieno[3,2-b]pyridin-1-yl)methyl)thiazol-4- yl)methyl)amino)pentan-1-ol (Compound 236) |
| 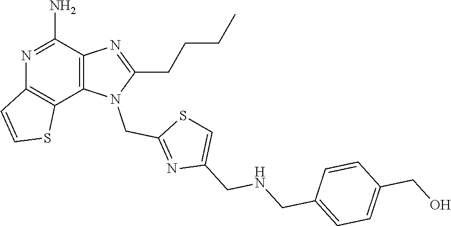 [SMILES: CCCCc1nc2c(N)nc3ccsc3c2n1Cc1nc(CNCc2ccc(CO)cc2)cs1] | (4-((((2-((4-amino-2-butyl-1H-imidazo[4,5- d]thieno[3,2-b]pyridin-1-yl)methyl)thiazol-4- yl)methyl)amino)methyl)phenyl)methanol (Compound 237) |
| 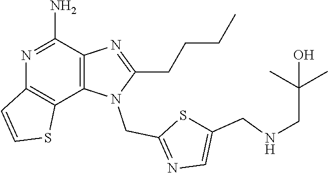 [SMILES: CCCCc1nc2c(N)nc3ccsc3c2n1Cc1ncc(CNCC(C)(C)O)s1] | 1-(((2-((4-amino-2-butyl-1H-imidazo[4,5- d]thieno[3,2-b]pyridin-1-yl)methyl)thiazol-5- yl)methyl)amino)-2-methylpropan-2-ol (Compound 238) |
| 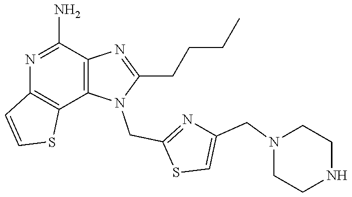 [SMILES: CCCCc1nc2c(N)nc3ccsc3c2n1Cc1nc(CN2CCNCC2)cs1] | 2-butyl-1-((4-(piperazin-1-ylmethyl)thiazol-2- yl)methyl)-1H-imidazo[4,5-d]thieno[3,2- b]pyridin-4-amine (Compound 242) |
| 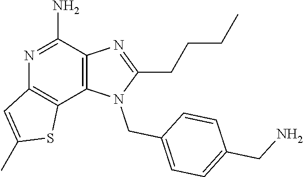 [SMILES: CCCCc1nc2c(N)nc3cc(C)sc3c2n1Cc1ccc(CN)cc1] | 1-(4-(aminomethyl)benzyl)-2-butyl-7-methyl-1H- imidazo[4,5-d]thieno[3,2-b]pyridin-4-amine (Compound 254) |
| 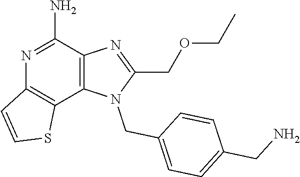 [SMILES: CCOCc1nc2c(N)nc3ccsc3c2n1Cc1ccc(CN)cc1] | 1-(4-(aminomethyl)benzyl)-2-(ethoxymethyl)-1H- imidazo[4,5-d]thieno[3,2-b]pyridin-4-amine (Compound 284) |
| 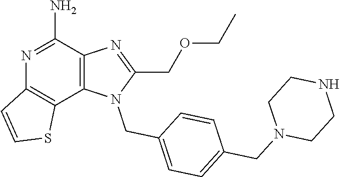 [SMILES: CCOCc1nc2c(N)nc3ccsc3c2n1Cc1ccc(CN2CCNCC2)cc1] | 2-(ethoxymethyl)-1-(4-(piperazin-1- ylmethyl)benzyl)-1H-imidazo[4,5-d]thieno[3,2- b]pyridin-4-amine (Compound 289) |
| 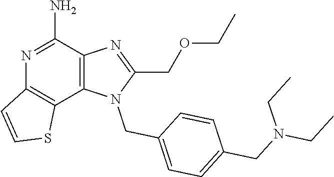 [SMILES: CCOCc1nc2c(N)nc3ccsc3c2n1Cc1ccc(CN(CC)CC)cc1] | 1-(4-((diethylamino)methyl)benzyl)-2- (ethoxymethyl)-1H-imidazo[4,5-d]thieno[3,2- b]pyridin-4-amine (Compound 290) |
| 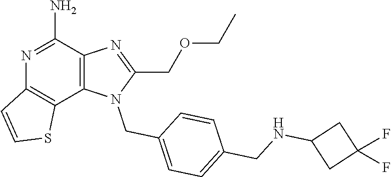 [SMILES: CCOCc1nc2c(N)nc3ccsc3c2n1Cc1ccc(CNC2CC(F)(F)C2)cc1] | 1-(4-(((3,3- difluorocyclobutyl)amino)methyl)benzyl)-2- (ethoxymethyl)-1H-imidazo[4,5-d]thieno[3,2- b]pyridin-4-amine (Compound 291) |
| 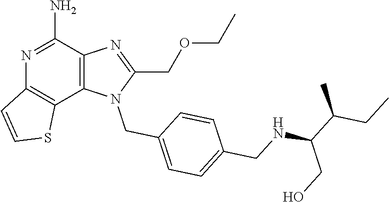 [SMILES: CCOCc1nc2c(N)nc3ccsc3c2n1Cc1ccc(CN[C@H](CO)[C@@H](C)CC)cc1] | (2S,3S)-2-((4-((4-amino-2-(ethoxymethyl)-1H- imidazo[4,5-d]thieno[3,2-b]pyridin-1- yl)methyl)benzyl)amino)-3-methylpentan-1-ol (Compound 292) |
| 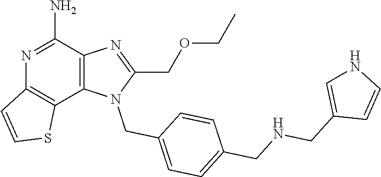 [SMILES: CCOCc1nc2c(N)nc3ccsc3c2n1Cc1ccc(CNCc2cc[nH]c2)cc1] | 1-(4-((((1H-pyrrol-3- yl)methyl)amino)methyl)benzyl)-2- (ethoxymethyl)-1H-imidazo[4,5-d]thieno[3,2- b]pyridin-4-amine (Compound 293) |
| 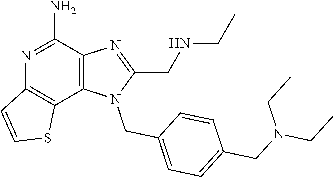 [SMILES: CCNCc1nc2c(N)nc3ccsc3c2n1Cc1ccc(CN(CC)CC)cc1] | 1-(4-((diethylamino)methyl)benzyl)-2- ((ethylamino)methyl)-1H-imidazo[4,5- d]thieno[3,2-b]pyridin-4-amine (Compound 269) |

In certain embodiments, a compound having Formula I, II, III, IV, V, and/or VI, as described herein, may possess a sufficiently acidic group, a sufficiently basic group, or both functional groups, and accordingly react with a number of organic and inorganic bases, or organic and inorganic acids, to form pharmaceutically acceptable salts. The term “pharmaceutically acceptable salt,” as used herein, refers to a salt of a compound having Formula I, II, III, IV, V, and/or VI, which is substantially non-toxic to living organisms. Typical pharmaceutically acceptable salts include those salts prepared by reaction of a compound having Formula I, II, III, IV, V, and/or VI, with a pharmaceutically acceptable mineral or organic acid or an organic or inorganic base. Such salts are known as acid addition and base addition salts.

Acids commonly employed to form acid addition salts are inorganic acids including, but are not limited to, hydrochloric acid, hydrobromic acid, hydroiodic acid, sulphuric acid, phosphoric acid, and organic acids including, but not limited to, p-toluenesulphonic acid, methanesulphonic acid, oxalic acid, p-bromophenylsulphonic acid, carbonic acid, succinic acid, citric acid, benzoic acid and acetic acid. Examples of pharmaceutically acceptable salts include, but are not limited to, sulphates, pyrosulphates, bisulphates, sulphites, phosphates, monohydrogenphosphates, dihydrogenphosphates, metaphosphates, pyrophosphates, bromides, iodides, acetates, propionates, decanoates, caprylates, acrylates, formates, hydrochlorides, dihydrochlorides, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, hydroxybenzoates, methoxybenzoates, phthalates, xylenesulphonates, phenylacetates, phenylpropionates, phenylbutyrates, citrates, lactates, gamma-hydroxybutyrates, glycolates, tartrates, methanesulphonates, propanesulphonates, naphthalene-1-sulfonates, napththalene-2-sulfonates and mandelates. Pharmaceutically acceptable acid addition salts of particular interest are those formed with mineral acids such as hydrochloric acid and hydrobromic acid, and those formed with organic acids such as maleic acid and methanesulphonic acid.

Salts of amine groups may also comprise quarternary ammonium salts in which the amino nitrogen carries a suitable organic group such as an alkyl, lower alkenyl, lower alkynyl or aralkyl moiety.

Base addition salts include those derived from inorganic bases, such as ammonium or alkali or alkaline earth metal hydroxides, carbonates, bicarbonates, and the like. Bases useful in preparing pharmaceutically acceptable salts include, but are not limited to, sodium hydroxide, potassium hydroxide, ammonium hydroxide, potassium carbonate, sodium carbonate, sodium bicarbonate, potassium bicarbonate, calcium hydroxide and calcium carbonate.

One skilled in the art will understand that the particular counterion forming a part of a pharmaceutically acceptable salt is usually not of a critical nature, so long as the salt as a whole is pharmacologically acceptable and as long as the counterion does not contribute undesired qualities to the salt as a whole.

Certain embodiments relate to pharmaceutically acceptable solvates of a compound having Formula I, II, III, IV, V, and/or VI. One skilled in the art will appreciate that certain compounds having Formula I, II, III, IV, V, and/or VI, may combine with solvents such as water, methanol, ethanol or acetonitrile to form pharmaceutically acceptable solvates such as the corresponding hydrate, methanolate, ethanolate or acetonitrilate. Other examples of solvents that may be used to prepare solvates include isopropanol, dimethyl sulfoxide, ethyl acetate, acetic acid, ethanolamine and acetone, as well as miscible formulations of solvate mixtures as would be known by the skilled artisan.

In some embodiments, a compound having Formula I, II, III, IV, V, and/or VI, and/or any embodiments or combinations thereof, is an agonist of TLR7. In some embodiments, a compound having Formula I, II, III, IV, V, and/or VI, and/or any embodiments or combinations thereof, is an agonist of TLR7 and TLR8. In some embodiments, a compound having Formula I, II, III, IV, V, and/or VI, and/or any embodiments or combinations thereof, induces production of cytokine(s) in immune cells (e.g., PBMCs). In some embodiments, a compound having Formula I, II, III, IV, V, and/or VI, and/or any embodiments or combinations thereof, induces production of IL-6 and/or TNF-α in immune cells (e.g., PBMCs).

In some embodiments, a compound of Table 1 is an agonist of TLR7. In some embodiments, a compound of Table 1 is an agonist of TLR7 and TLR8. In some embodiments, a compound of Table 1 induces production of cytokines in immune cells (e.g., PBMCs). For instance, Examples 1-9 and Tables 11.1 and 11.2 describe example methods for synthesizing, using, and testing compounds for in vitro activity as TLR7 and/or TLR8 agonists. Furthermore, Examples 1-9 and Tables 11.1 and 11.2 describe example methods for synthesizing, using, and testing compounds for in vitro induction of cytokine production in immune cells (e.g., PBMCs).

In some embodiments, a compound having Formula I, II, III, IV, V, and/or VI, a compound of Table 1, and/or any embodiments or combinations thereof, has an EC₅₀ of 200 nM or less for agonism of TLR7. As used herein, EC₅₀ can refer to the half maximal effective concentration of the respective compound, where the value of the EC₅₀ indicates the concentration of the compound that induces a biological response (e.g., TLR7 agonism, TLR8 agonism, stimulation of the immune system, production of cytokines by immune cells, and/or rejection of tumor cells) halfway between the baseline and the maximum after a defined duration of exposure.

In some embodiments, a compound having Formula I, II, III, IV, V, and/or VI, a compound of Table 1, and/or any embodiments or combinations thereof, has an EC₅₀ of 750 nM or less, 650 nM or less, 500 nM or less, 300 nM or less, 275 nM or less, 250 nM or less, 225 nM or less, 200 nM or less, 175 nM or less, 150 nM or less, 125 nM or less, 100 nM or less, 75 nM or less, 50 nM or less, 25 nM or less, 20 nM or less, 15 nM or less or 10 nM or less for agonism of TLR7. In some embodiments, a compound having Formula I, II, III, IV, V, and/or VI, a compound of Table 1, and/or any embodiments or combinations thereof, has an EC₅₀ of between 1 and 750 nM, between 1 and 500 nM, between 1 and 300 nM, between 1 and 200 nM, or between 1 and 100 nM for agonism of TLR7.

In some embodiments, the EC₅₀ value for agonism of TLR7 by a compound having Formula I, II, III, IV, V, and/or VI, or a compound of Table 1, is determined in vitro using a reporter gene assay employing TLR7 reporter cells. In some embodiments, the EC₅₀ value for agonism of TLR7 of a compound having Formula I, II, III, IV, V, and/or VI, or a compound of Table 1, is determined in vitro using a reporter gene assay employing HEK-Blue™ TLR7 reporter cells (available from Invivogen, San Diego, CA). An exemplary method for determining EC₅₀ values for agonism of TLR7 is provided in Example 11 herein.

In some embodiments, a compound having Formula I, II, III, IV, V, and/or VI, a compound of Table 1, and/or any embodiments or combinations thereof, has an EC₅₀ of 750 nM or less, 500 nM or less, 300 nM or less, 275 nM or less, 250 nM or less, 225 nM or less, 200 nM or less, 175 nM or less, 150 nM or less, 125 nM or less or 100 nM or less for inducing production of a cytokine from PBMCs. In some embodiments, a compound having Formula I, II, III, IV, V, and/or VI, a compound of Table 1, and/or any embodiments or combinations thereof, has an EC₅₀ of between 50 and 750 nM, 50 and 500 nM, between 50 and 300 nM or between 50 and 200 nM for inducing production of a cytokine from PBMCs. In some embodiments, the cytokine is TNF-α. In some embodiments, the cytokine is IL-6.

In certain embodiments, the EC₅₀ for inducing production of a cytokine from PBMCs by a compound having Formula I, II, III, IV, V, and/or VI, or a compound of Table 1, is determined in vitro by treating PBMCs isolated from peripheral blood with titrating concentrations of the compound followed by assaying for cytokines by homogeneous time resolved fluorescence (HTRF). An exemplary method for determining EC₅₀ values for inducing production of a cytokine from PBMCs is provided in Example 11 herein.

It is to be understood that reference to compounds of Formula I throughout the disclosure, includes in various embodiments, compounds of Formula II, III, IV, V and VI, and compounds of Table 1, to the same extent as if embodiments individually reciting each of these formulae or compounds were specifically recited.

### Conjugates

Certain embodiments of the present disclosure relate to conjugates of compounds of Formula I in which the compound is conjugated to a targeting moiety via a linker.

The conjugates of the present disclosure may comprise one or multiple compounds of Formula I (either of the same or different structure) conjugated to the targeting moiety. Multiple compounds may be conjugated to the targeting moiety, for example, by attaching the compounds at different sites on the targeting moiety each via a linker and/or by employing a linker that allows for attachment of multiple compounds to a single site (e.g., functional group) on the targeting moiety.

Accordingly, in some embodiments, the present disclosure provides a conjugate having Formula X:

T-(L-(D)ᵣ)_{q} (X)

wherein:

T is a targeting moiety;

L is a linker;

D is a compound according to any one of the herein described compounds of Formula I, II, III, IV, V, and/or VI, or a compound of Table 1 (see the foregoing section; “Compounds”);

q has a value from about 1 to about 8; and

r is an integer from 1 to 4.

As described herein, in certain embodiments, the compounds disclosed herein can be used for the preparation of immunostimulatory antibody-drug conjugates (ISACs) that comprise at least a conjugated antibody that recognizes and binds to a tumor-associated antigen (e.g., a protein that is expressed on the surface of a tumor cell to be targeted) and an immunostimulatory payload (e.g., a compound of Formula I) that is released inside an immune cell and/or within a tumor microenvironment, thereby causing an indirect anti-tumor effect via activation of the immune system (e.g., via TLR7-mediated signaling and/or cytokine production).

FIG. 10B illustrates a schematic of an example of a conjugate comprising a targeting moiety (e.g., a TAA-targeting antibody), a linker L, and a compound D (e.g., a TLR7-agonist payload according to Formula I).

A. Targeting Moieties

Targeting moieties (T) comprised by the conjugates described herein are molecules that bind, reactively associate or complex with a receptor, antigen or other receptive moiety associated with a given target cell population. Examples of targeting moieties include, but are not limited to, proteins (such as antibodies, antibody fragments and growth factors), glycoproteins, peptides (such as bombesin and gastrin-releasing peptide), lectins, vitamins (such as folic acid) and nutrient-transport molecules (such as transferrin).

As described herein, antibody-drug conjugates, including immunostimulatory antibody-drug conjugates, generally comprise a targeting moiety (e.g., an antibody specific for an antigen expressed on a tumor cell), which allows an ADC or ISAC to mount a direct or indirect effect against the target cell. For example, where the conjugate is an ISAC, the effect can be performed indirectly by stimulating an immune cell with an immunostimulatory payload (e.g., a compound of Formula I) conjugated to the antibody.

Thus, in some embodiments, the conjugate herein having Formula X comprises an antibody or an antigen-binding antibody fragment as the targeting moiety T. In some embodiments, the targeting moiety T is an antibody or antigen-binding antibody fragment that recognizes and/or binds to a tumor-associated antigen (TAA).

In some embodiments, the targeting moiety T is a protein comprising one or more members of a subset of portions and/or domains present in an antibody (e.g., scFv, Fab, Fc, or combinations of such portions or domains).

In some embodiments, the targeting moiety T comprises one or more of a full size antibody (such as an IgG) and/or an antibody fragment, such as a one armed antibody, a half antibody, a Fab domain of an antibody, an scFv or a domain antibody, an Fc domain of an antibody, and/or a heterodimeric Fc domain of an antibody. In some embodiments, any two or more of these antibody fragments can be combined (e.g., chemically or through expression of a fusion protein) to form a targeting moiety, as will be apparent to one skilled in the art.

For example, in some embodiments, the targeting moiety T is a one-armed (monovalent) antibody. Typically, one-armed antibody-drug conjugates bind cell-surface antigen targets at a 1:1 ADC:antigen ratio, whereas full sized-antibody-drug conjugates can bind antigens at a ratio of 1:2. Using similar stoichiometric reasoning, and without being bound to any one theory of operation, a greater number of surface bound ADC or ISAC molecules can result in faster efficacy and/or more potent immunological responses when using one-armed antibodies compared to full sized antibodies. Thus, in some embodiments, conjugates comprising one-armed antibody targeting moieties have advantages of mass action, where more antibody decoration on the target cell surface can increase the effect of the biological response.

In some embodiments, the targeting moiety T is a single chain antibody (“SCA”). In some embodiments, single chain antibodies comprise single chain Fv fragments (“scFv”), in which the variable light (“VL”) and variable heavy (“VH”) domains are linked by a peptide bridge or by disulfide bonds. In some embodiments, the targeting moiety T comprises single VH domains (dAbs) that possess antigen-binding activity. See, e.g., G. Winter and C. Milstein, Nature, 349, 295 (1991); R. Glockshuber et al. Biochemistry 29, 1362 (1990); and, E. S. Ward et al. Nature 341, 544 (1989).

In some embodiments, the targeting moiety T is a chimeric antibody (e.g., a humanized antibody). In some embodiments, the targeting moiety is a “bifunctional,” “bispecific” or “hybrid” antibody, such as an antibody comprising a first arm having a specificity for a first antigenic site (e.g., a first TAA) and a second arm having a specificity for a second antigenic site (e.g., a second TAA). In some embodiments, the targeting moiety is a biparatopic antibody, such as an antibody comprising a first arm having a specificity for a first epitope of a target antigen (e.g., a first epitope of a TAA) and a second arm having a specificity for a second epitope of a target antigen (e.g., a second epitope of the TAA), which is different from the first epitope, of the cell targeted for therapeutic or biological response. For example, in some instances, biparatopic ADCs have been observed to increase drug activity in vivo. In some embodiments, the targeting moiety has dual specificity (e.g., for two target antigens or two epitopes of a target antigen).

In some embodiments, hybrid or bifunctional antibodies are derived, as noted, either biologically, by cell fusion techniques, or chemically, e.g., with cross-linking agents or disulfide bridge-forming reagents, and can be comprised of whole antibodies and/or fragments thereof. Methods for obtaining such hybrid antibodies are disclosed, for example, in PCT Patent Application No. PCT/CA2014/050486, entitled “Modular Protein Drug Conjugate Therapeutic,” filed May 23, 2014, which is hereby incorporated herein by reference in its entirety. Bifunctional antibodies include those biologically prepared from a “polydoma” or “quadroma,” and/or those that are synthetically prepared with cross-linking agents such as bis-(maleimido)-methyl ether (“BMME”), or with other cross-linking agents familiar to those skilled in the art.

As described herein, in some embodiments, “bifunctional”, “bispecific”, “hybrid”, “chimeric,” and/or “biparatopic” antibody architectures also include, within their individual contexts, architectures comprising antigen-recognizing fragments. In some embodiments, such fragments can be prepared by traditional enzymatic cleavage of intact bifunctional, bispecific, hybrid, chimeric, and/or biparatopic antibodies. Where intact antibodies are not susceptible to cleavage, antibody architectures can be prepared from immunoglobulin fragments; or, if recombinant techniques are used, DNA sequences can be tailored to encode a desired antigen-recognizing fragment that can be combined in vivo or in vitro, by chemical or biological means, with one or more additional antigen-recognizing fragments.

Additional antibody architectures for use as targeting moieties in conjugates described herein are possible, and can be selected based on the specific function or activity desired, as will be apparent to one skilled in the art. See, e.g., Bates A and Power C, Antibodies 2019; 8:28; doi: 10.3390/antib8020028, and Akiba H and Tsumoto K, Trans Reg Sci. 2020; 2(1):1-6; doi: 10.33611/trs.2_1, each of which is hereby incorporated herein by reference in its entirety.

In certain embodiments, the antibody included in a conjugate described herein may be a bispecific antibody. Methods for making bispecific antibodies are known in the art (see, e.g., Milstein et al., 1983, Nature, 305:537-539; Traunecker et al., 1991, EMBO J., 10:3655-3659; Suresh et al., 1986, Meth. Enzymol., 121:210; Rodrigues et al., 1993, J. Immunol., 151:6954-6961; Carter et al., 1992, Bio/Technology, 10:163-167; Carter et al., 1995, J. Hematotherapy, 4:463-470; Merchant et al., 1998, Nature Biotechnology, 16:677-681, and International (PCT) Publication Nos. WO 94/04690, WO 2012/032080, WO 2012/058768 and WO 2013/063702).

In some embodiments, the targeting moiety T is an immunoglobulin antibody. In some embodiments, the targeting moiety T is an immunoglobulin antibody that recognizes a tumor-associated antigen (TAA). As used herein, “immunoglobulin” includes any recognized class or subclass of immunoglobulins such as IgG, IgA, IgM, IgD, or IgE. In some embodiments, the targeting moiety is an IgG immunoglobulin. In some embodiments, the targeting moiety is an immunoglobulin of human, murine, or rabbit origin. In some embodiments, the targeting moiety is an immunoglobulin that is polyclonal, monoclonal, or in fragment form. In some instances, immunoglobulin fragments include the Fab′, F(ab′)₂, Fv or Fab fragments, or other antigen recognizing immunoglobulin fragments. Immunoglobulin fragments can be prepared, for example, by proteolytic enzyme digestion (e.g., by pepsin or papain digestion), reductive alkylation, or recombinant techniques. The materials and methods for preparing such immunoglobulin fragments are well-known to those skilled in the art (Parham, (1983) J. Immunology, 131:2895; Lamoyi et al. (1983) J. Immunological Methods, 56:235; Parham, (1982) J. Immunological Methods, 53:133; and Matthew et al. (1982) J. Immunological Methods, 50:239).

Furthermore, as described herein, in some embodiments, the immunoglobulin (antibody), or fragment thereof, is polyclonal or monoclonal in nature. Methods for preparation of polyclonal or monoclonal antibodies are known to those skilled in the art. See, e.g., G. Kohler and C. Milstein, Nature 256, 495 (1975).

In some embodiments, the targeting moiety T is an antibody that targets a cancer cell. In some embodiments, the targeting moiety T is an antibody used for the treatment of a cancer. For example, antibodies used for the treatment of cancers include, but are not limited to, alemtuzumab, apolizumab, aselizumab, atlizumab, bapineuzumab, bevacizumab, cedelizumab, cidfusituzumab, cidtuzumab, daclizumab, eculizumab, efalizumab, epratuzumab, erlizumab, felvizumab, fontolizumab, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tadocizumab, talizumab, tefibazumab, tocilizumab, toralizumab, trastuzumab, tucusituzumab, umavizumab, urtoxazumab, and/or visilizumab.

In some embodiments, the targeting moiety T is an antibody that is immunospecific for a tumor-associated antigen (TAA) (e.g., for the treatment or prevention of cancer). For example, in some embodiments, the targeting moiety is immunospecific for an antigen expressed by and/or on the surface of a tumor cell, including, but not limited to, anti-Her2, anti-Liv-1, anti-CD52, anti-CD30, anti-CTLA-4, anti-CD20, anti-EGFR, anti-CD33, anti-CD22, anti-HLA-DR, anti-HLA-Dr10, anti-CD2, anti-VEGF, and/or anti-CEA. See, e.g., Salsano and Treglia, Res Rep Nuc Med. 2013; 3:9-17; doi: 10.2147/RRNM.S35186, which is hereby incorporated herein by reference in its entirety.

Antibodies immunospecific for a tumor-associated antigen (TAA) can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody for the treatment of patients with metastatic breast cancer; RITUXAN® (rituximab; Genentech), which is a chimeric anti-CD20 monoclonal antibody for the treatment of patients with non-Hodgkin's lymphoma; OvaRex (AltaRex Corporation, MA), which is a murine antibody for the treatment of ovarian cancer; Panorex (Glaxo Wellcome, N.C.), which is a murine IgG₂ₐ antibody for the treatment of colorectal cancer; Cetuximab Erbitux (Imclone Systems Inc., NY), which is an anti-EGFR IgG chimeric antibody for the treatment of epidermal growth factor positive cancers, such as head and neck cancer; Vitaxin (MedImmune, Inc., MD), which is a humanized antibody for the treatment of sarcoma; Campath I/H (Leukosite, MA), which is a humanized IgG₁ antibody for the treatment of chronic lymphocytic leukemia (CLL); Smart MI95 (Protein Design Labs, Inc., CA), which is a humanized anti-CD33 IgG antibody for the treatment of acute myeloid leukemia (AML); LymphoCide (Immunomedics, Inc., NJ), which is a humanized anti-CD22 IgG antibody for the treatment of non-Hodgkin's lymphoma; Smart ID10 (Protein Design Labs, Inc., CA), which is a humanized anti-HLA-DR antibody for the treatment of non-Hodgkin's lymphoma; Oncolym (Techniclone, Inc., CA), which is a radiolabeled murine anti-HLA-Dr10 antibody for the treatment of non-Hodgkin's lymphoma; Allomune (BioTransplant, CA), which is a humanized anti-CD2 MAb for the treatment of Hodgkin's Disease or non-Hodgkin's lymphoma; Avastin (Genentech, Inc., CA), which is an anti-VEGF humanized antibody for the treatment of lung and colorectal cancers; Epratuzamab (Immunomedics, Inc., NJ and Amgen, CA), which is an anti-CD22 antibody for the treatment of non-Hodgkin's lymphoma; and CEAcide (Immunomedics, NJ), which is a humanized anti-CEA antibody for the treatment of colorectal cancer.

In some embodiments, the targeting moiety T is a deglycosylated antibody.

In some embodiments, the targeting moiety T further encompasses derivatives, mutants, and variants of any of the targeting moieties and/or embodiments described herein, which derivatives, mutants, and variants are altered in one or more amino acids (or, when referring to the nucleotide sequence encoding the same, are altered in one or more base pairs) such that the amino acid (or DNA) sequence of the resulting targeting moiety is not identical to that of the targeting moieties recited herein, but the biological and/or biochemical properties are substantially the same.

In some embodiments, the targeting moiety T further comprises fragments of antibodies and/or truncated antibodies that retain the desired biological activity of the antibody irrespective of the length of the fragmented or truncated antibody.

B. Linkers

As described herein, in some embodiments, the conjugate having Formula X comprises one or more linkers L. In some embodiments, the conjugate comprises a plurality of linkers L.

In some embodiments, a linker L links a compound D (e.g., an immunostimulatory drug, such as a compound of Formula I as described herein) to a targeting moiety T, such that, upon binding of the targeting moiety T to one or more antigens on the surface of the target cell (e.g., a tumor cell), the compound D is subsequently presented on the surface of the target cell for recognition by an effector cell and activation of a biological response (e.g., an immune response mediated by an immune cell). See, e.g., the schematic illustrated in FIG. 10A.

A linker L comprised by the conjugates disclosed herein can be a bifunctional or multifunctional molecule capable of linking one or more compounds of Formula I (D) to targeting moiety T. In some embodiments, a linker L may be bifunctional such that it links a single compound D to a single site (e.g., a single functional group) on targeting moiety T. In some embodiments, the linker is a hetero-bifunctional linker. In some embodiments, a linker L may be multifunctional (or polyvalent) such that it links more than one (e.g., 2, 3, 4 or more) of compound D to a single site (e.g., a single functional group) on targeting moiety T. Multifunctional linkers may, in some embodiments, also be used to link one compound D to more than one site (e.g., more than one functional group) on targeting moiety T.

A linker L can include a first functional group (or first set of functional groups) capable of reacting with a target functional group (or groups) on targeting moiety T, and a second functional group (or second set of functional groups) capable of reacting with a target functional group (or groups) on compound D of Formula I. Suitable functional groups are known in the art and include those described, for example, in Bioconjugate Techniques (G. T. Hermanson, 2013, Academic Press). Functional groups on targeting moiety T and compound D that may serve as target groups for linker attachment can include, but are not limited to, thiol, hydroxyl, carboxyl, amine, aldehyde and ketone groups.

In some embodiments, each linker of the one or more linkers comprised by the conjugate is bound to one or more compounds D of Formula I. In some embodiments, each linker of the one or more linkers is bound to 1, 2, 3, or 4 compounds D (e.g., r in Formula X can be 1, 2, 3, or 4) of Formula I.

In some embodiments, a linker comprises or consists of a substituted or unsubstituted alkyl or a substituted or unsubstituted heteroalkyl chain comprising a first and a second terminal functional group. Hence, in some embodiments, the first terminal functional group of the linker forms a first linkage or bond with a first reactive functional group on a first conjugation partner (e.g., a targeting moiety T), and the second terminal functional group of the linker forms a second linkage or bond with a second reactive functional group of a second conjugation partner (e.g., a compound D of Formula I).

In some embodiments, the linker comprises a substituted or unsubstituted hydrocarbon backbone. In some embodiments, the substituted or unsubstituted hydrocarbon backbone is interrupted by one or more heteroatoms (e.g., O, N, S, P), thereby forming, e.g., a heteroalkyl linker.

In some embodiments, the linker includes an alkylene oxide, e.g., a poly(alkylene oxide), or a poly(ethylene glycol).

A linker L may be a cleavable or a non-cleavable linker. A cleavable linker is a linker that is susceptible to cleavage under specific conditions, e.g., intracellular conditions (such as in an endosome or lysosome) or within the vicinity of a target cell (such as in the tumor microenvironment). Examples include linkers that are protease-sensitive, acid-sensitive or reduction-sensitive. Non-cleavable linkers by contrast, rely on the degradation of the antibody in the cell, which typically results in the release of one or more amino acid-linker-drug species. Hence, in some embodiments, the linker comprised by the conjugate of Formula X is a cleavable linker. In other embodiments, the linker comprised by the conjugate of Formula X is a non-cleavable linker.

In some embodiments, the linker is stable in the extracellular environment. Such linker can be characterized in that at least about 90%, about 80%, about 70%, about 60%, about 50% or at least about 40% of the conjugates of Formula X are intact (e.g., the DAR of the conjugate remains substantially the same (e.g., ±5%) compared the conjugate at the time of administration) upon delivery to and/or localization on a target cell surface and after a certain period of time. In other words, in some embodiments, the linker remains essentially uncleaved in the extracellular environment during the time the conjugate is resident in this environment (e.g., in systemic circulation and/or a non-target tissue or organ). Hence, such linker may be cleaved in the extracellular environment but not to a degree that prevents a useful dosage of the intact conjugate being delivered to a target cell. Whether a linker is not substantially sensitive to the extracellular environment can be determined, e.g., by incubating the antibody-drug conjugate with plasma for a predetermined period of time (e.g., 2, 4, 8, 16, or 24 hours) and then quantitating the amount of free drug present in the plasma.

In some embodiments, the linker is cleavable by a cleaving agent (e.g., an enzyme) that is present in the intracellular environment (e.g., within a lysosome or endosome or caveolea). In some embodiments, the linker comprises a peptide sequence that is preferentially cleaved by an intracellular peptidase or protease enzyme, including, but not limited to, a lysosomal or endosomal protease. In some embodiments, the peptide sequence comprised by the linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). In some embodiments, the linker cleavable by an intracellular protease comprises the dipeptide Val-Cit or Phe-Lys. Other examples of cathepsin B cleavable peptide sequences include Val-Ala, Val-Cit, Val-Gly, Val-Gln, Val-Lys, Ala-Val-Cit, Asp-Val-Ala, Asp-Val-Cit, Lys-Val-Ala and Lys-Val-Cit. Intracellular proteolytic release mechanisms may be advantageous in some instances, e.g., when the concentration of cleaving agent is relatively high in a target cell.

In some embodiments, a cleavable linker is pH-sensitive, e.g., sensitive to hydrolysis at certain pH values. Typically, the pH-sensitive linker is hydrolyzable under acidic conditions. For example, an acid-labile linker that is hydrolyzable in the lysosome (e.g., those comprising a hydrazone, semicarbazone, thiosemicarbazone, cis-aconitic amide, orthoester, acetal, ketal moiety, or the like) can be used. See, e.g., U.S. Pat. Nos. 5,122,368; 5,824,805; 5,622,929; Dubowchik and Walker, 1999, Pharm. Therapeutics 83:67-123; and Neville et al. 1989, Biol. Chem. 264:14653-14661. Such linkers are relatively stable under neutral pH conditions, such as those found in the blood, but are unstable at pH 5.5 or 5.0 or below, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929)).

In some embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene). See, e.g., Thorpe et al. 1987, Cancer Res. 47:5924-5931; Wawrzynczak et al. In Immunoconjugates: Antibody Conjugates in Radioimagery and Therapy of Cancer (C. W. Vogel ed., Oxford U. Press, 1987). See also U.S. Pat. No. 4,880,935.

A further example of a cleavable linker is a linker comprising a β-glucuronide, which is cleavable by β-glucuronidase, an enzyme present in lysosomes and tumor interstitium (see, for example, De Graaf et al., 2002, Curr. Pharm. Des. 8:1391-1403). β-glucuronide may also function to improve the hydrophilicity of linker L.

Another example of a linker that is cleaved internally within a cell and improves hydrophilicity is a linker comprising a pyrophosphate diester moiety (see, for example, Kern et al., 2016, J Am Chem Soc., 138:2430-1445).

In some embodiments, the linker comprises malonate (Johnson et al. 1995, Anticancer Res. 15:1387-93), maleimidobenzoyl (Lau et al. 1995, Bioorg-Med-Chem. 3(10):1299-1304), or a 3′-N-amide analogue (Lau et al. 1995, Bioorg-Med-Chem. 3(10):1305-12).

In some embodiments, the linker unit is not cleavable and the drug is released by antibody degradation (U.S. Publication No. 2005/0238649).

In some embodiments, the linker is cleaved upon uptake of the conjugate (or a portion thereof) by a cell. For example, in some embodiments, the linker comprises PABC or PAB (para-aminobenzyloxycarbonyl) (Carl et al. (1981) J. Med. Chem. 24:479-480; and Chakravarty et al. (1983) J. Med. Chem. 26:638-644). In some embodiments, the linker comprises PABC or PAB and a peptide sequence that is cleaved by an intracellular peptidase or protease enzyme. The PAB/PABC linker unit is also referred to as an electronic cascade spacer. The amide bond linking the carboxy terminus of a peptide unit and the para-aminobenzyl of PAB/PABC can be a substrate and cleavable by certain proteases. The aromatic amine becomes electron-donating and initiates an electronic cascade that leads to the expulsion of the leaving group, which releases the free drug (e.g., a compound of Formula I) after elimination of carbon dioxide (see, de Groot et al. (2001) Journal of Organic Chemistry 66(26):8815-8830). Cathepsin B is a ubiquitous cysteine protease. It is an intracellular enzyme, except in pathological conditions, such as metastatic tumors (Sinha et al. (2001) Prostate 49:172-184) or rheumatoid arthritis (Hashimoto et al. (2001) Biochem. Biophys. Res. Commun. 283:334-339). Therefore, conjugates produced with cathepsin B-cleavable linkers are likely to be stable in circulation. Upon cleavage of a peptide bond adjacent to the PAB/PABC, e.g., by an intracellular enzyme, the drug is released from the ligand whereby no remaining portion of the linker is bound (de Groot, et al. (2002) Molecular Cancer Therapeutics 1(11):901-911; de Groot, et al. (1999) J. Med. Chem. 42(25):5277-5283).

Linkers containing the para-aminobenzyloxycarbonyl (PAB or PABC) unit, in conjunction with a peptide unit, have been developed with a “self-immolating” or “self-immolative” mechanism of 1,6 elimination and fragmentation under enzymatic, hydrolytic, or other metabolic conditions to release a drug molecule from a targeting moiety, such as an antibody (U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). The 2-nitroimidazol-5-ylmethyl group has been reported as a fragmenting prodrug unit (Hay et al. (1999) Bioorg. Med. Chem. Lett. 9:2237). For the use of the PAB unit in prodrugs and conjugates, see also: Walker, et al. (2004) Bioorganic &Medicinal Chemistry Letters 14(16):4323-4327; Devy, et al. (2004) FASEB Journal 18(3):565-567, 10.1096/fj.03-0462fje; Francisco, et al. Blood (2003) 102(4):1458-1465; Doronina, et al. (2003) Nature Biotechnology 21(7):778-784; King, et al. (2002) Journal of Medicinal Chemistry 45(19):4336-4343; Dubowchik, et al. (2002) Bioconjugate Chemistry 13(4):855-869; Dubowchik, et al. (2002) Bioorganic & Medicinal Chemistry Letters 12(11):1529-1532.

Additional linkers are possible, as will be apparent to one skilled in the art. Linkers and methods of producing the same are further described, for example, in PCT Patent Application No. PCT/CA2014/050486, entitled “Modular Protein Drug Conjugate Therapeutic,” filed May 23, 2014, which is hereby incorporated herein by reference in its entirety.

C. Properties of Conjugates

The drug-antibody ratio (DAR) of a conjugate (or “q” in Formula X), as provided herein, refers to the ratio of the number of drug compounds (e.g., compound(s) D of Formula I) conjugated to any one targeting moiety T. Thus, as illustrated in FIG. 10B, r can be 1 and q can be 4, such that each linker binds 1 compound to the targeting moiety, and the targeting moiety is bound to 4 drug-linkers to result in a drug-antibody ratio (DAR) of 4.

Those skilled in the art will appreciate that, while any particular targeting moiety T is conjugated to compound(s) D, analysis of a preparation of the conjugate to determine the ratio of compound D to targeting moiety T (DAR) may give a non-integer result, reflecting a statistical average. Accordingly, conjugate preparations having non-integer DARs are intended to be encompassed by Formula X. One skilled in the art will appreciate that the term “DAR” may also be employed to define conjugates comprising targeting moieties other than antibodies.

The DAR of the conjugates described herein may be determined by standard techniques such as UV/VIS spectroscopic analysis, ELISA-based techniques, chromatography techniques such as hydrophobic interaction chromatography (HIC), UV-MALDI mass spectrometry (MS) and MALDI-TOF MS. In addition, the distribution of conjugates with different DARs (e.g., the fraction of conjugates comprising zero, one, two, three, four, etc. compounds D) may also optionally be analyzed. Various techniques are known in the art to measure such distribution, including MS (with or without an accompanying chromatographic separation step), hydrophobic interaction chromatography, reverse-phase HPLC or iso-electric focusing gel electrophoresis (IEF) (see, for example, Wakankar et al., 2011, mAbs, 3:161-172).

In some embodiments, the DAR of the conjugates of Formula X is between 1 and 32. In some embodiments, the DAR of the conjugates of Formula X is between 1 and 24, between 1 and 16, between 1 and 8, between 3 and 5, or between 1 and 4. In some embodiments, the DAR of the conjugates of Formula X is between 2 and 32, between 2 and 24, between 2 and 16, between 2 and 8 or between 2 and 4.

In some embodiments, the DAR of the conjugates of Formula X herein (or “q” in Formula X) can have any numeric value from about 1 to about 8, and thus can have a value of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or about 8.0. In some cases, q can have a value from about 2 to about 8, from about 2 to about 6, from about 2 to about 5, from about 3 to about 5, or from about 2 to about 4.

In some embodiments, the DAR of a conjugate herein is obtained by any combination of linker-to-antibody ratio and/or drug-to-linker ratio (e.g., where q is the ratio of drug-linker constructs to antibody T, and r is the number of drug molecules D per linker). For example, where q=8 and r=4, the number of compounds D of Formula Iin the respective antibody-drug conjugate is 32 (e.g., each tumor-targeting antibody T is coupled to 8 drug-linker constructs, with each construct comprising 4 immunostimulatory drugs of Formula I). In another example, where q=1 and r=8, the number of compounds in the respective antibody-drug conjugate is 8 (e.g., each tumor-targeting antibody T is coupled to a single drug-linker construct comprising 8 immunostimulatory drugs).

Hence, in some embodiments, the product of q and r is about 32 or less, 24 or less, 16 or less, 8 or less, or 4 or less. In some embodiments, the product of q and r is about 8. In some embodiments, the product of q and r is about 4. In some embodiments, the product of q and r is about 2.

In some embodiments, in which the conjugate comprises a plurality of drug-linker constructs coupled to a single antibody T, each respective drug-linker construct of the plurality of drug-linker constructs can comprise the same or a different number of compounds compared to any other drug-linker construct of the respective conjugate.

In some embodiments, in which a conjugate X comprises two or more compounds D of Formula I, all compounds of such conjugate are identical, e.g., have an identical chemical structure.

In other embodiments, in which a conjugate X comprises two or more compounds D of Formula I, at least two of the two or more compounds of that conjugate are different, e.g., have a different chemical structure. In other instances, every compound D of a conjugate is different, e.g., has a different chemical structure compared to all other compounds of the conjugate. In some embodiments, the payload of a conjugate of Formula X can be tailored to target a specific immune target, by selecting a compound (e.g., a compound of Formula I) that is an agonist for the respective immune target (e.g., a TLR such as TLR7 and/or TLR8).

In some embodiments, the payload of a conjugate of Formula X can be tailored to target a plurality of specific immune targets, e.g., by selecting a plurality of compounds (e.g., including one or more compounds of Formula I), each capable of interacting (e.g., agonizing) a respective immune target of the plurality of immune targets (e.g., one or more TLRs such as TLR7, TLR8, etc.). In other embodiments, the payload of a conjugate of Formula X can be tailored to target a plurality of specific immune targets using a single species compound D of Formula I capable of interacting, e.g., agonizing, all of the immune targets of the plurality of immune targets. Examples of such multi-targeting compounds are the TLR7/8 agonists described herein which can be characterized as dual-targeting TLR agonists.

In some embodiments in which a conjugate X comprises a plurality of drug-linker constructs associated with (e.g., covalently or non-covalently coupled to) a single targeting moiety T (e.g., antibody), each drug-linker construct comprises only a single species of compound D (e.g., a compound of Formula I). In some embodiments in which a conjugate X comprises a plurality of drug-linker constructs associated with a single targeting moiety T, each drug-linker construct comprises a different species of compound D, e.g., a first drug-linker construct comprises a first species of compound D, a second drug-linker construct comprises a second species of compound D, a third drug-linker construct comprises a third species of compound D, etc.). In some embodiments in which a conjugate X comprises a plurality of drug-linker constructs associated with a single targeting moiety T, at least two drug-linker constructs comprise different species of compound D. In some embodiments in which a conjugate X comprises a plurality of drug-linker constructs associated with a single targeting moiety T, each drug-linker construct comprises the same species of compound D.

In further embodiments, a conjugate of Formula X herein comprises a drug-linker construct comprising two or more species of compound D (e.g., two or more species of compound D are coupled to a targeting moiety T via the same linker).

Additional embodiments comprising other configurations of targeting moieties, linkers, and compounds of Formula I that have not been explicitly described herein, as well as combinations thereof, are encompassed by the present disclosure, as will be apparent to one skilled in the art.

Methods of conjugating compound(s) D of Formula I to a targeting moiety via linker(s) are known in the art. For example, a variety of chemical strategies have been employed to conjugate compounds to targeting moieties, including reactions between amines and N-hydroxysuccinimide (NHS) esters, and sulfhydryls with maleimides. The latter reaction may be useful for conjugation chemistry as it can provide high selectivity and rapid reaction rates under aqueous conditions compatible with maintaining protein structure and activity. See, e.g., Sussman D et al. Prot Eng Des Select. 2018; 31(2):47-54; doi: 10.1093/protein/gzx067, which is hereby incorporated herein by reference in its entirety. Additional methods for conjugation are further described in, e.g., Bioconjugate Techniques (G. T. Hermanson, 2013, Academic Press). Various linkers and linker components are commercially available or may be prepared using standard synthetic organic chemistry techniques (see, e.g., March's Advanced Organic Chemistry (Smith & March, 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press)). In addition, various antibody drug conjugation services are available commercially from companies such as Lonza Inc. (Allendale, NJ), Abzena PLC (Cambridge, UK), ADC Biotechnology (St. Asaph, UK), Baxter BioPharma Solutions (Baxter Healthcare Corporation, Deerfield, IL) and Piramel Pharma Solutions (Grangemouth, UK).

In some embodiments, synthesis of a conjugate of Formula X can comprise maleimide cysteine conjugation.

Additional conjugates comprising any combination of the various embodiments of antibodies, linkers, and compounds, as described in the foregoing sections, are contemplated, as will be apparent to one skilled in the art. Also possible are any substitutions, deletions, additions, and/or modifications thereof, as will be apparent to one skilled in the art.

### Pharmaceutical Compositions

The present disclosure further provides pharmaceutical compositions comprising a compound according to any one of Formula I, II, III, IV, V, and/or VI, or a compound of Table 1 described herein (see, e.g., section above; “Compounds”), or a pharmaceutically acceptable salt thereof. Such pharmaceutical composition can further comprise a pharmaceutically acceptable carrier or diluent. In some embodiments, the pharmaceutical composition is a therapeutic composition for the treatment of a disorder, e.g., an immune disorder. In some embodiments, the pharmaceutical composition is a therapeutic composition for the treatment of a cancer.

Another aspect of the present disclosure provides pharmaceutical compositions comprising a conjugate according to Formula X described herein (see, e.g., the foregoing section; “Conjugates”), and a pharmaceutically acceptable carrier or diluent. In some embodiments, the pharmaceutical composition is a therapeutic composition for the treatment of a disorder, e.g., an immune disorder. In some embodiments, the pharmaceutical composition is a therapeutic composition for the treatment of a cancer.

In some embodiments, a pharmaceutical composition herein includes a drug and/or a pharmaceutically active agent (e.g., any one or more of a compound of Formula I, a conjugate of Formula X, and/or any combinations of the same), and one or more pharmaceutically acceptable carriers, glidants, diluents, or excipients.

In some embodiments, a pharmaceutical composition is formulated in accordance with standard pharmaceutical practice for use in therapeutic treatment of hyperproliferative disorders (e.g., cancer) in mammals including humans.

In some embodiments, the pharmaceutical composition is formulated in accordance with standard pharmaceutical practice for use in a therapeutic combination for therapeutic treatment of hyperproliferative disorders (e.g., cancer) in mammals including humans.

In some embodiments, the pharmaceutical composition encompasses a bulk composition and/or individual dosage units comprised of one or more drugs and/or pharmaceutically active agents including, for example, a conjugate comprising at least an antibody and a compound as provided herein (e.g., a conjugate according to Formula X), along with any pharmaceutically inactive excipients, diluents, carriers, or glidants. In some embodiments, the bulk composition and each individual dosage unit contain fixed amounts of the respective one or more pharmaceutically active agents (e.g., compound(s) of Formula I and/or conjugate(s) of Formula X). As used herein, a bulk composition refers to material that has not yet been formed into individual dosage units. For example, an illustrative dosage unit is an oral dosage unit such as a tablet, a pills, a capsule, and the like. Similarly, in some embodiments, a method of treating a subject (e.g., a human) in need thereof by administering a pharmaceutical composition includes the administration of the bulk composition and/or individual dosage units.

Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used may depend upon the means and purpose for which the compound or conjugate is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of a drug and/or a pharmaceutically active agent (e.g., any one or more of a compound as described herein, a conjugate as described herein, and/or any combination of the same) or aid in the manufacturing of a pharmaceutical product (e.g., a medicament).

In some embodiments, the pharmaceutical compositions described herein include formulations comprising a carrier suitable for the desired delivery method. Suitable carriers include any material that when combined with the pharmaceutical composition retains the function of the pharmaceutical composition and is generally non-reactive with the subject's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980). In some embodiments, the pharmaceutical composition includes formulations suitable for a specific administration route (e.g., any one or more of the methods of administration provided herein). Techniques and formulations are known in the art (see, Remington's Pharmaceutical Sciences 18th Edition, Mack Publishing Co., Easton, Pa., 1995).

For example, a formulation for a pharmaceutical composition suitable for oral administration can be prepared as discrete units such as pills, hard or soft capsules, e.g., gelatin capsules, cachets, troches, lozenges, aqueous or oil suspensions, dispersible powders or granules, emulsions, syrups or elixirs, each containing a predetermined amount of a compound and/or a conjugate disclosed herein. In some embodiments, such formulations are prepared according to any method known to the art for the manufacture of pharmaceutical compositions, where such compositions contain one or more agents including sweetening agents, flavoring agents, coloring agents and preserving agents, in order to provide a palatable preparation. In some embodiments, compressed tablets are prepared by compressing in a suitable machine a drug and/or pharmaceutically active agent (e.g., any one or more of a compound as described herein, a conjugate as described herein, and/or any combination of the same) in a free-flowing form such as a powder or granules, optionally mixed with a binder, lubricant, inert diluent, preservative, surface active and/or dispersing agent. In some embodiments, molded tablets are made by molding in a suitable machine a mixture of the powdered drug and/or pharmaceutically active agent moistened with an inert liquid diluent. The tablets can optionally be coated or scored and optionally are formulated so as to provide slow or controlled release of the drug and/or pharmaceutically active agent therefrom.

In some embodiments, a formulation for a pharmaceutical composition suitable for treatment of the eye or other external tissues (e.g., mouth and skin) can be applied as a topical ointment or cream containing the drug and/or pharmaceutically active agent (e.g., any one or more of a compound as described herein, a conjugate as described herein, and/or any combination of the same). In some embodiments, the formulation is an ointment, where the drug and/or pharmaceutically active agent is employed with either a paraffinic or a water-miscible ointment base. Alternatively, in some embodiments, the drug and/or pharmaceutically active agent is formulated in a cream with an oil-in-water cream base.

In some embodiments, a formulation for a pharmaceutical composition is an aqueous suspension comprising the drug and/or pharmaceutically active agent (e.g., any one or more of a compound as described herein, a conjugate as described herein, and/or any combination of the same) and excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). In some embodiments, the aqueous suspension further comprises one or more preservatives such as ethyl or n-propyl p-hydroxybenzoate, one or more coloring agents, one or more flavoring agents, and/or one or more sweetening agents, such as sucrose or saccharin.

In some embodiments, the pharmaceutical composition is in the form of a sterile injectable preparation, such as a sterile injectable aqueous or oleaginous suspension. In some embodiments, the suspension is formulated according to the known art using suitable dispersing or wetting agents and suspending agents as described herein. In some embodiments, the sterile injectable preparation is a solution or a suspension in a non-toxic parenterally acceptable diluent or solvent, such as a solution in 1,3-butanediol or prepared from a lyophilized powder. Suitable vehicles and solvents include water, Ringer's solution and isotonic sodium chloride solution. In addition, the sterile injectable preparation can comprise sterile fixed oils as a solvent or suspending medium, any bland fixed oil including synthetic mono- or diglycerides, and/or fatty acids such as oleic acid.

Additional embodiments of pharmaceutical compositions are possible, including any additions, deletions, substitutions, and/or modifications or combinations of the foregoing examples, as will be apparent to one skilled in the art.

### Methods of Agonizing a TLR

Another aspect of the present disclosure provides a method of agonizing a TLR (e.g., TLR7) comprising contacting a cell that expresses the TLR with a compound according to any one of the compounds disclosed herein, e.g., a compound of Formula I (see the above sections; “Compounds”), or a pharmaceutically acceptable salt thereof.

Another aspect of the present disclosure provides a method of agonizing a TLR (e.g., TLR7) comprising contacting a cell that expresses the TLR with a conjugate according to any one of the conjugates disclosed herein, e.g., a conjugate of Formula X (see the above section; “Conjugates”).

In some embodiments, the TLR is TLR7.

In some embodiments, the cell is a mammalian cell. In some embodiments, the cell is a human cell. In some embodiments, the cell is an immune cell (e.g., a phagocyte). In some embodiments, the cell is selected from the group consisting of macrophages, dendritic cells, natural killer (NK) cells and epithelial cells.

In some embodiments, agonizing TLR7 using a compound and/or conjugate of the present disclosure comprises activation of the TLR7 signaling pathway. In some embodiments, agonizing TLR7 comprises an activation or a repression of one or more intermediates in the TLR7 signaling pathway. In some embodiments, agonizing TLR7 comprises a change in the expression level of one or more intermediates in the TLR7 signaling pathway. Intermediates of the TLR7 signaling pathway include, e.g., MyD88, IRAK4, IRAK1, IRAK2, TRAF6, TAK1, IKK, NF-1<B, FADD, Caspase 8, Caspase 3, and/or IRF7. See, e.g., Chi H et al. Front Pharmacol. 2017; 8:304; doi: 10.3389/fphar.2017.00304, which is hereby incorporated herein by reference in its entirety.

In some embodiments, agonizing TLR7 induces an immune response. In some embodiments, the immune response can comprise any of the changes in a physiological parameter as described herein (e.g., production/secretion of cytokines, small molecules, co-stimulatory molecules, and/or factors involved in inflammation cascade or regulation, and/or a change in immune cell populations) (see the following section; “Stimulation of the Immune Response”).

In some embodiments, the changes in TLR7 signaling intermediates and/or other physiological parameters affected by TLR7 agonism and/or stimulation of the immune responses are measured as described herein, and/or by using any suitable method known in the art, as described below (see the following section; “Stimulation of the Immune Response”).

### Immunological and Clinical Applications of the Disclosed Compounds and Conjugates

A. Stimulation of an Immune Response

Another aspect of the present disclosure provides a method of stimulating an immune response (e.g., a TLR7-mediated response) in a subject in need thereof comprising administering to the subject an effective amount of a compound according to any one of the compounds disclosed herein, e.g., a compound of Formula I (see the above section, “Compounds”), or a pharmaceutically acceptable salt thereof.

In some embodiments, the present disclosure provides a method of stimulating an immune response (e.g., a TLR7-mediated response) in a subject in need thereof comprising administering to the subject an effective amount of a conjugate according to any one of the conjugates disclosed herein, e.g., a conjugate of Formula X (see the above section, “Conjugates”).

In some embodiments, the present disclosure provides a method of stimulating an immune response (e.g., a TLR7-mediated response) in a subject in need thereof comprising administering to the subject a pharmaceutical composition comprising an effective amount of a compound of Formula I, or a pharmaceutically acceptable salt thereof, and/or a conjugate of Formula X, as disclosed herein.

In some embodiments, administering a compound (e.g., one of Formula I), a conjugate (e.g., one of Formula X), or a pharmaceutical composition disclosed herein to a subject in need thereof can result in agonizing TLR7 in a cell of the subject that expresses TLR7. In some embodiments, stimulating an immune response comprises agonizing TLR7 in a cell that expresses TLR7. In such embodiments, stimulating the immune response comprises contacting a cell that expresses TLR7 with a compound (e.g., one of Formula I), a conjugate (e.g., one of Formula X), or a pharmaceutical composition as disclosed herein.

In some embodiments, stimulating an immune response comprises activating an immune cell, including but not limited to macrophages, dendritic cells, natural killer (NK) cells and epithelial cells.

In some embodiments, stimulating an immune response using the compounds, conjugates, and/or pharmaceutical compositions described herein comprises inducing a change in a physiological parameter in the subject, wherein the change in the physiological parameter can comprise one or more of the following: (i) a change in a level of one or more (pro)-inflammatory cytokine(s); (ii) a change in a level of anti-inflammatory cytokine(s) and/or pro-resolving mediators, (iii) changes in immune cell population(s) or immune cell surface co-stimulatory molecules, (iv) a change in a level of factors involved in the inflammation cascade; and/or (v) a change in a level of immune response mediators. In some embodiments, the change in the physiological parameter comprises one or more of an increase in pro-inflammatory cytokine(s), a decrease in anti-inflammatory cytokines and/or pro-resolving mediators, an increase in immune cell population(s) or immune cell surface co-stimulatory molecules, an increase in factors involved in the inflammation cascade, and/or an increase in immune response mediators.

Examples of inflammatory cytokines that can be modulated in response to a compound and/or a conjugate of the present disclosure include tumor necrosis factor (TNF; also known as TNFα or cachectin), interleukin (IL)-1α, IL-1β, IL-2; IL-5, IL-6, IL-8, IL-15, IL-18, interferon γ (IFN-γ); platelet-activating factor (PAF), thromboxane; soluble adhesion molecules; vasoactive neuropeptides; phospholipase A2; plasminogen activator inhibitor (PAI-1); free radical generation; neopterin; CD14; prostacyclin; neutrophil elastase; protein kinase; monocyte chemotactic proteins 1 and 2 (MCP-1, MCP-2); macrophage migration inhibitory factor (MIF), high mobility group box protein 1 (HMGB-1), and other known factors.

Anti-inflammatory cytokines are also known in the art. Examples of these include IL-4, IL-10, IL-17, IL-13, IL-1α, and TNFα receptor. Examples of pro-resolving mediators include Lipoxins, Resolvins, Protectins and Maresins.

In some embodiments, some pro-inflammatory cytokines can act as anti-inflammatory cytokines in certain circumstances, and vice versa. Such cytokines are typically referred to as pleiotropic cytokines.

In some embodiments, factors involved in immune responses can be useful measurable parameters for assessing stimulation of an immune response by a compound and/or a conjugate of the present disclosure, e.g., TGF, PDGF, VEGF, EGF, FGF, I-CAM, and/or nitric oxide.

In some embodiments, chemokines can also be useful measurable parameters of immunomodulation, such as 6cKine and MIP3beta, and chemokine receptors, including CCR7 receptor.

Changes in immune cell population(s) (Langerhans cells, dendritic cells, lymphocytes, monocytes, macrophages), or immune cell surface co-stimulatory molecules (Major Histocompatibility, CD80, CD86, CD28, CD40) can also be useful measurable parameters for assessing stimulation of an immune response by a compound and/or a conjugate of the present disclosure.

Factors involved in the inflammatory cascade can also be used as measurable parameters for stimulation of the immune response. For example, the signal transduction cascades include factors such as NFκ-B, Egr-1, Smads, toll-like receptors, and MAP kinases.

Methods of assessing one or more of these physiological parameters are known in the art. For example, a cytokine can be directly detected, e.g., by ELISA. Other suitable methods include liquid chromatography and tandem mass spectrometry. Quantitative changes of the biological molecules (e.g., cytokines) can be measured in a biological sample such as organ, tissue, urine or plasma. Detection of the biological molecules can be performed directly on a sample taken from a subject, or the sample can be treated between sample collection and analysis.

B. Treatment of Hyperproliferative Disorders

Another aspect of the present disclosure provides a method of treating a hyperproliferative disorder (e.g., a cancer) in a subject in need thereof comprising administering to the subject an effective amount of a compound according to any one of the compounds disclosed herein, e.g., a compound of Formula I (see the above section; “Compounds”), or a pharmaceutically acceptable salt thereof.

In some embodiments, the present disclosure provides a method of treating a hyperproliferative disorder (e.g., a cancer) in a subject in need thereof comprising administering to the subject an effective amount of a conjugate according to any one of conjugates disclosed herein, e.g., a conjugate of Formula X (see the above section; “Conjugates”).

In some embodiments, the present disclosure provides a method of treating a hyperproliferative disorder (e.g., a cancer) in a subject in need thereof comprising administering to the subject a pharmaceutical composition comprising an effective amount of a compound, or a pharmaceutically acceptable salt thereof, and/or a conjugate according to any one of the compounds and/or conjugates disclosed herein.

In some embodiments, the treating comprises agonizing a TLR in a cell that expresses the TLR. In some embodiments, the treating comprises agonizing TLR7 in a cell that expresses TLR7. In some embodiments, the treating comprises contacting a cell that expresses TLR7 with a compound, conjugate, and/or pharmaceutical composition as disclosed herein.

In some embodiments, the subject being treated with a compound, conjugate, and/or pharmaceutical composition disclosed herein is a human. In some embodiments, the subject is a human that has been diagnosed with a hyperproliferative disorder. In some embodiments, the subject has been diagnosed with a cancer.

In some embodiments, the cancer to be treated using a compound, conjugate, and/or pharmaceutical composition disclosed herein includes, but is not limited to, breast cancer, ovarian cancer, cervical cancer, prostate cancer, testicular cancer, genitourinary tract cancer, esophageal cancer, larynx cancer, glioblastoma, neuroblastoma, stomach cancer, skin cancer, keratoacanthoma, lung cancer, epidermoid carcinoma, large cell carcinoma, non-small cell lung carcinoma (NSCLC), small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma and biliary passage cancer, kidney carcinoma, myeloid disorders, lymphoid disorders, hairy cell carcinoma, buccal cavity and pharynx (oral) cancer, lip cancer, tongue cancer, mouth cancer, pharynx cancer, cancer of the small intestine, colon-rectum cancer, large intestinal cancer, rectal cancer, brain cancer and cancer of the central nervous system, (non)-Hodgkin's lymphoma, and leukemia.

### C. Methods of Administration

In some embodiments, an effective amount of a compound, conjugate, and/or a pharmaceutical composition comprising the same, is administered to a subject in need thereof by any suitable means to stimulate an immune response and/or to treat a hyperproliferative disorder (e.g., a cancer). For example, in certain embodiments, the compound, conjugate, and/or pharmaceutical composition can be administered by intravenous, intraocular, subcutaneous, and/or intramuscular means. The compound, conjugate, and/or pharmaceutical composition can be administered by parenteral (including intravenous, intradermal, intraperitoneal, intramuscular and subcutaneous) routes or by other delivery routes, including oral, nasal, buccal, sublingual, intra-tracheal, transdermal, transmucosal, and pulmonary. In certain embodiments, the compound, conjugate, and/or pharmaceutical composition can be administered either systemically or locally. Systemic administration can includes oral, transdermal, subdermal, intraperitioneal, subcutaneous, transnasal, sublingual, or rectal administration. Alternatively, the compound, conjugate, and/or pharmaceutical composition can be delivered via a sustained delivery device implanted, for example, subcutaneously or intramuscularly. The compound, conjugate, and/or pharmaceutical composition can be administered by continuous release or delivery, using, for example, an infusion pump, continuous infusion, controlled release formulations utilizing polymer, oil or water insoluble matrices.

In certain embodiments, the effective amount of a compound and/or a conjugate of the present disclosure to be administered to the subject can be determined by a physician with consideration of individual differences in age, weight, the disease or condition being treated, disease severity and response to the therapy. In certain embodiments, the compound, conjugate, and/or pharmaceutical composition described herein can be administered to a subject alone or in combination with other compositions. In some embodiments, the compound, conjugate, and/or pharmaceutical composition is administered at periodic intervals, over multiple time points, and/or for a specific duration of treatment (e.g., one or several days, weeks, or months). In some embodiments, the compound, conjugate, and/or pharmaceutical composition is administered at a single time point. In some embodiments, the time needed to complete a course of the treatment is determined by a physician.

According to some embodiments, the compound, conjugate, and/or pharmaceutical composition is administered in extended release form, which is capable of releasing the compound, conjugate, and/or pharmaceutical composition over a predetermined release period, such that a therapeutically effective plasma level of the compound, conjugate, and/or pharmaceutical composition is maintained for at least 24 hours, such as at least 48 hours, at least 72 hours, at least one week, or at least one month.

In some embodiments, the compound, conjugate, and/or pharmaceutical composition comprises a formulation that is selected for the mode of delivery, e.g., intravenous, intraocular, subcutaneous, oral, and/or intramuscular means.

According to some embodiments of the present invention, the compound, conjugate, and/or pharmaceutical composition can be administered in combination with one or more active therapeutic agents for treating a specific disease, a co-infection, and/or potential complications or side-effects associated with a treatment regimen.

### D. Combination Therapy

In some embodiments, a compound, conjugate, and/or pharmaceutical composition as described herein is employed in combination with other chemotherapeutic agents for the treatment of a hyperproliferative disorder (e.g., a cancer). In some embodiments, a compound, conjugate, and/or pharmaceutical composition is combined in a pharmaceutical combination formulation, or dosing regimen as combination therapy, with a second compound that has anti-hyperproliferative properties or that is useful for treating the hyperproliferative disorder. The second compound of the pharmaceutical combination formulation or dosing regimen can have complementary activities to a compound or conjugate disclosed herein, and such that they do not adversely affect each other. Such compounds are suitably present in a combination in amounts that are effective for the purpose intended.

In some embodiments, therapeutic combinations include a formulation, dosing regimen, and/or other course of treatment comprising the administration of a compound, conjugate, and/or pharmaceutical composition, and a chemotherapeutic agent. In some embodiments, the therapeutic combination is a combined preparation for separate, simultaneous or sequential use in the treatment of a hyperproliferative disorder.

In some embodiments, the combination therapy is administered as a simultaneous or sequential regimen. When administered sequentially, the combination can be administered in two or more administrations. The combined administration includes coadministration, using separate formulations or a single pharmaceutical formulation, and consecutive administration in either order, in which there may be a time period while both (or all) active agents simultaneously exert their biological activities.

Suitable dosages for any of the above coadministered agents can be optimized based on the combined action (synergy) of one or both of the coadministered agents.

In some embodiments, a compound, conjugate, and/or pharmaceutical composition of the present disclosure is combined with surgical therapy and/or radiation therapy. In some embodiments, the amount of the compound, conjugate, and/or pharmaceutical composition and the relative timings of administration can be selected and modified in order to achieve the desired combined and maximum therapeutic effect.

Dosages and administration protocols for the treatment of cancers using the foregoing methods may vary with the method and the cancer to be treated, and may generally depend on a number of other factors appreciated in the art. Additional methods of administration of compounds, conjugates, and/or pharmaceutical compositions for the stimulation of immune response and/or the treatment of hyperproliferative disorders are possible, as will be apparent to one skilled in the art.

### SELECTED EMBODIMENTS OF THE DISCLOSURE

In various aspects, the present disclosure provides an embodiment according to any one of the embodiments 1-172, or a combination thereof.

Embodiment 1. A compound having Formula I:

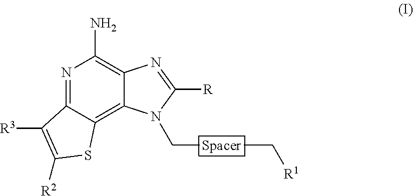

or a pharmaceutically acceptable salt thereof,
wherein:

R is H, C₁-C₆ alkyl, CH₂SR¹⁵ or CH₂OR¹⁵;

R¹ is —OH, —NR⁴R⁵, —OR¹⁰, SR¹¹ or

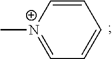

R² and R³ are each independently H or optionally substituted C₁-C₆ alkyl;

Spacer is —(CH₂)ₙ—,

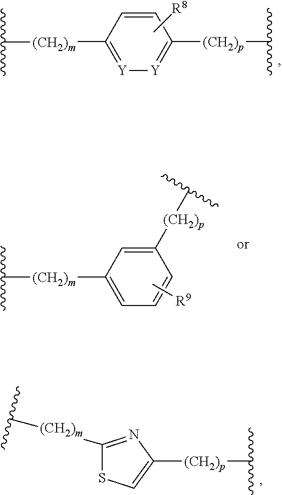

wherein n is an integer between 3 and 10; each m is independently an integer between 0 and 4; each p is independently an integer between 0 and 4; and Y is CH or N;

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;

R⁸ and R⁹ are each independently H, NR¹³R¹⁴, halo, optionally substituted C₁-C₄ alkoxy or optionally substituted C₁-C₄ alkyl;

R¹⁰ and R¹¹ are each independently optionally substituted C₁-C₆ alkyl, optionally substituted aryl or optionally substituted C₃-C₇ cycloalkyl;

R¹³ and R¹⁴ are each independently H or optionally substituted C₁-C₄ alkyl; and

R¹⁵ is C₃-C₄ cycloalkyl or C₁-C₄ alkyl optionally substituted with one or more halo.

Embodiment 2. The compound according to embodiment 1, wherein R is C₁-C₆ alkyl.

Embodiment 3. The compound according to embodiment 1 or embodiment 2, wherein R is C₂-C₄ alkyl.

Embodiment 4. The compound according to embodiment 1, wherein R is CH₂OR¹⁵.

Embodiment 5. The compound according to embodiment 4, wherein R¹⁵ is C₁-C₂ alkyl.

Embodiment 6. The compound according to any one of embodiments 1-5, wherein R¹ and R⁹ are each H or halo.

Embodiment 7. The compound according to any one of embodiments 1-6, wherein Spacer is

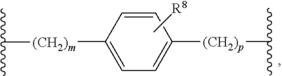

m in an integer between 0 and 4; p is an integer between 0 and 4; R⁸ is H, NR¹³R¹⁴, halo, optionally substituted C₁-C₄ alkoxy or optionally substituted C₁-C₄ alkyl, and wherein R¹³ and R¹⁴ are each independently H or C₁-C₄ alkyl.

Embodiment 8. The compound according to any one of embodiments 1-7, wherein m is 0.

Embodiment 9. The compound according to any one of embodiments 1-8, wherein p is 0.

Embodiment 10. The compound according to any one of embodiments 1-6, wherein Spacer is

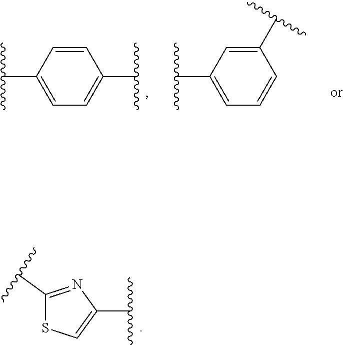

Embodiment 11. The compound according to any one of embodiments 1-6, wherein n is an integer between 3 and 5.

Embodiment 12. The compound according to any one of embodiments 1-11, wherein R¹ is —OH, —NR⁴R⁵, —OR¹⁰, SR¹¹,

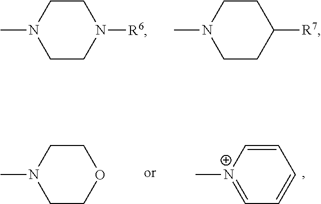

and wherein

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

Embodiment 13. The compound according to embodiment 12, wherein R¹ is —OH, —NR⁴R⁵,

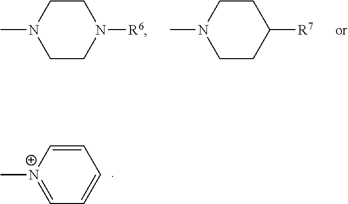

Embodiment 14. The compound according to embodiment 12 or embodiment 13, wherein R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

Embodiment 15. The compound according to embodiment 12 or embodiment 13, wherein R⁷ is H, optionally substituted C₁-C₄ alkyl, optionally substituted C₁-C₄ aminoalkyl or optionally substituted heteroaryl.

Embodiment 16. The compound according to any one of embodiments 1-13, wherein R⁴ is H, optionally substituted C₁-C₄ alkyl, optionally substituted C₁-C₄ carboxyalkyl or optionally substituted C₁-C₄ aminoalkyl.

Embodiment 17. The compound according to any one of embodiments 1-13, or 16, wherein R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

Embodiment 18. The compound according to any one of embodiments 1-17, wherein R² and R³ are each independently H or C₁-C₄ alkyl.

Embodiment 19. The compound according to any one of embodiments 1-18, wherein R³ is H.

Embodiment 20. The compound according to any one of embodiments 1-19, wherein R² is C₁-C₄ alkyl, and R³ is H.

Embodiment 21. The compound according to any one of embodiments 1-18, wherein R² and R³ are each H.

Embodiment 22. The compound according to embodiment 1, wherein R² and R³ are each independently H or C₁-C₆ alkyl, R⁸ and R⁹ are each independently H, NR¹³R¹⁴, halo, C₁-C₄ alkoxy or C₁-C₄ alkyl, and R¹³ and R¹⁴ are each independently H or C₁-C₄ alkyl.

Embodiment 23. The compound according to embodiment 1, having Formula II:

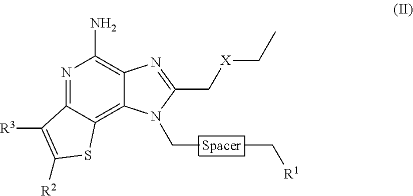

wherein:

X is —CH₂— or —O—;

R¹ is —OH, —NR⁴R⁵, —OR¹⁰, SR¹¹ or

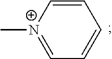

R² and R³ are each independently H or optionally substituted C₁-C₆ alkyl;

Spacer is —(CH₂)ₙ—,

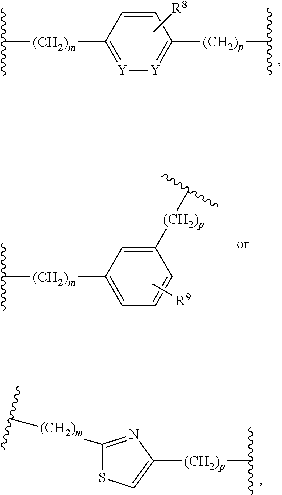

wherein n is an integer between 3 and 10; each m is independently an integer between 0 and 4; each p is independently an integer between 0 and 4; and Y is CH or N;

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;

R⁸ and R⁹ are each independently H, NR¹³R¹⁴, halo, optionally substituted C₁-C₄ alkoxy or optionally substituted C₁-C₄ alkyl;

R¹⁰ and R¹¹ are each independently optionally substituted C₁-C₆ alkyl, optionally substituted aryl or optionally substituted C₃-C₇ cycloalkyl; and

R¹³ and R¹⁴ are each independently H or optionally substituted C₁-C₄ alkyl.

Embodiment 24. The compound according to embodiment 23, wherein R² and R³ are each independently H or C₁-C₆ alkyl, or R² and R³ are each independently H or C₁-C₄ alkyl.

Embodiment 25. The compound according to embodiment 23 or embodiment 24, wherein R³ is H.

Embodiment 26. The compound according to any one of embodiments 23-25, wherein R² is C₁-C₄ alkyl, and R³ is H.

Embodiment 27. The compound according to any one of embodiments 23-25, wherein R² and R³ are each H.

Embodiment 28. The compound according to any one of embodiments 23-27, wherein Spacer is:

—(CH₂)ₙ—,

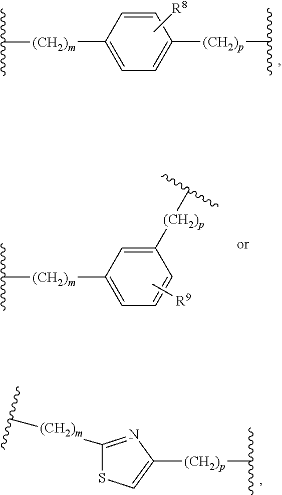

wherein n is an integer between 3 and 10; each m is independently an integer between 0 and 4; each p is independently an integer between 0 and 4, and R⁸ and R⁹ are each independently H, NR¹³R¹⁴, halo, optionally substituted C₁-C₄ alkoxy or optionally substituted C₁-C₄ alkyl, and wherein R¹³ and R¹⁴ are each independently H or optionally substituted C₁-C₄ alkyl.

Embodiment 29. The compound according to any one of embodiments 23-28, wherein

### Spacer is:

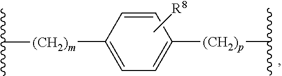

wherein m is an integer between 0 and 4; p is an integer between 0 and 4, and R⁸ is H, NR¹³R¹⁴, halo, optionally substituted C₁-C₄ alkoxy or optionally substituted C₁-C₄ alkyl, and wherein R¹³ and R¹⁴ are each independently H or optionally substituted C₁-C₄ alkyl.

Embodiment 30. The compound according to embodiment 23, wherein R² and R³ are each independently H or C₁-C₆ alkyl, R⁸ and R⁹ are each independently H, NR¹³R¹⁴, halo, C₁-C₄ alkoxy or C₁-C₄ alkyl, and R¹³ and R¹⁴ are each independently H or C₁-C₄ alkyl.

Embodiment 31. The compound according to any one of embodiments 23-28 or embodiment 30, wherein R⁸ and R⁹ are each H or halo.

Embodiment 32. The compound according to any one of embodiments 23-31, wherein m is 0.

Embodiment 33. The compound according to any one of embodiments 23-32, p is 0.

Embodiment 34. The compound according to any one of embodiments 23-27, wherein Spacer is —(CH₂)ₙ—,

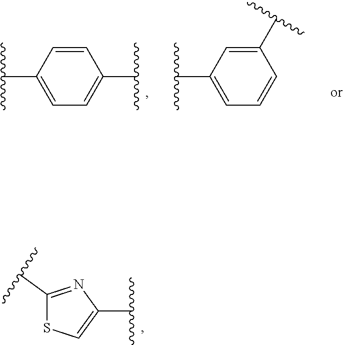

wherein n is an integer between 3 and 10.

Embodiment 35. The compound according to any one of embodiments 23-28, or 34, wherein n is an integer between 3 and 5.

Embodiment 36. The compound according to any one of embodiments 23-27, or 34, wherein Spacer is

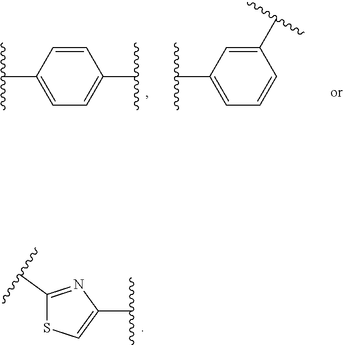

Embodiment 37. The compound according to any one of embodiments 23-36 wherein R¹ is —OH, —NR⁴R⁵, —OR¹⁰, SR¹¹,

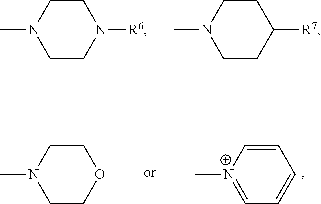

and wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

Embodiment 38. The compound according to any one of embodiments 23-37, wherein R¹ is —OH, —NR⁴R⁵,

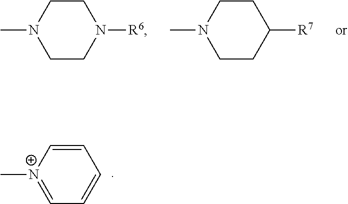

Embodiment 39. The compound according to any one of embodiments 23-38, wherein R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

Embodiment 40. The compound according to any one of embodiments 23-38, wherein R⁷ is H, optionally substituted C₁-C₄ alkyl, optionally substituted C₁-C₄ aminoalkyl or optionally substituted heteroaryl.

Embodiment 41. The compound according to any one of embodiments 23-38, wherein R⁴ is H, optionally substituted C₁-C₄ alkyl, optionally substituted C₁-C₄ carboxyalkyl or optionally substituted C₁-C₄ aminoalkyl.

Embodiment 42. The compound according to any one of embodiments 23-38, or 41, wherein R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

Embodiment 43. The compound according to embodiment 1, having Formula III:

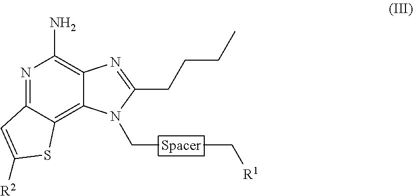

wherein:

R¹ is —OH, —NR⁴R⁵, —OR¹⁰, SR¹¹ or

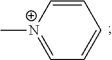

R² is H or optionally substituted C₁-C₆ alkyl;

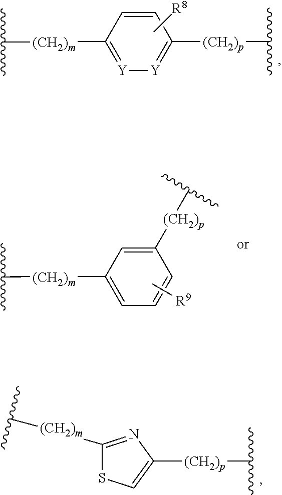

wherein n is an integer between 3 and 10; each m is independently an integer between 0 and 4; each p is independently an integer between 0 and 4; and Y is CH or N;

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;

R⁸ and R⁹ are each independently H, NR¹³R¹⁴, halo, optionally substituted C₁-C₄ alkoxy or optionally substituted C₁-C₄ alkyl;

R¹⁰ and R¹¹ are each independently optionally substituted C₁-C₆ alkyl, optionally substituted aryl or optionally substituted C₃-C₇ cycloalkyl; and

R¹³ and R¹⁴ are each independently H or optionally substituted C₁-C₄ alkyl.

Embodiment 44. The compound according to embodiment 43, wherein Spacer is: —(CH₂)ₙ—,

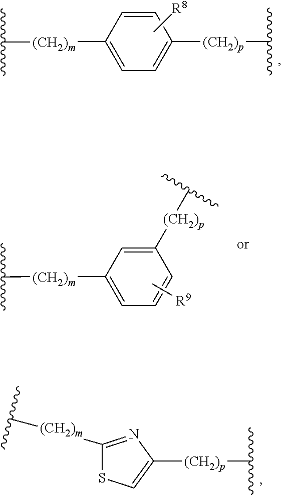

wherein n is an integer between 3 and 10; each m is independently an integer between 0 and 4; each p is independently an integer between 0 and 4, and R⁸ and R⁹ are each independently H, NR¹³R¹⁴, halo, optionally substituted C₁-C₄ alkoxy or optionally substituted C₁-C₄ alkyl, wherein R¹³ and R¹⁴ are each independently H or optionally substituted C₁-C₄ alkyl.

Embodiment 45. The compound according to embodiment 43 or embodiment 44, wherein Spacer is:

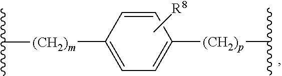

wherein m is an integer between 0 and 4; p is an integer between 0 and 4, and R⁸ is H, NR¹³R¹⁴, halo, optionally substituted C₁-C₄ alkoxy or optionally substituted C₁-C₄ alkyl, and wherein R¹³ and R¹⁴ are each independently H or optionally substituted C₁-C₄ alkyl.

Embodiment 46. The compound according to embodiment 43 or embodiment 44, wherein R⁸ and R⁹ are each independently H, NR¹³R¹⁴, halo, C₁-C₄ alkoxy or C₁-C₄ alkyl, wherein R¹³ and R¹⁴ are each independently H or C₁-C₄ alkyl.

Embodiment 47. The compound according to embodiment 43 or embodiment 44, wherein R⁸ and R⁹ are each H or halo.

Embodiment 48. The compound according to any one of embodiments 43-47, wherein m is 0.

Embodiment 49. The compound according to any one of embodiments 43-48, wherein p is 0.

Embodiment 50. The compound according to embodiment 43 or embodiment 44, wherein Spacer is —(CH₂)ₙ—,

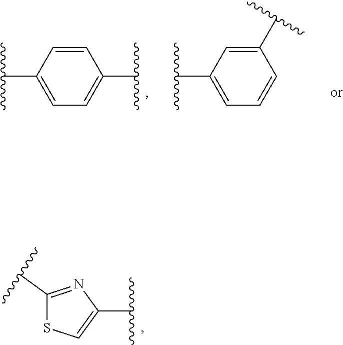

wherein n is an integer between 3 and 10.

Embodiment 51. The compound according to embodiment 43 or embodiment 50, wherein n is an integer between 3 and 5.

Embodiment 52. The compound according to embodiment 43 or embodiment 44, wherein Spacer is

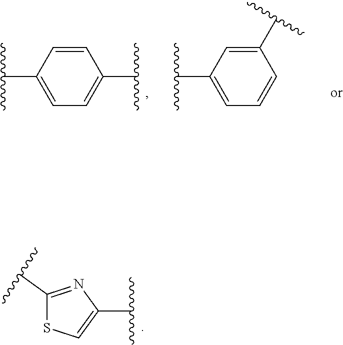

Embodiment 53. The compound according to any one of embodiments 43-52 wherein R¹ is —OH, —NR⁴R⁵, —OR¹⁰, SR¹¹,

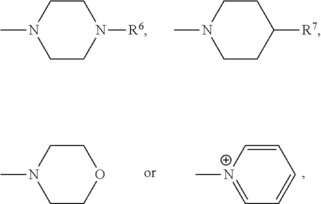

and wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

Embodiment 54. The compound according to any one of embodiments 43-53, wherein R¹ is —OH, —NR⁴R⁵,

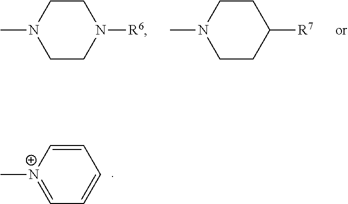

Embodiment 55. The compound according to any one of embodiments 43-54, wherein R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

Embodiment 56. The compound according to any one of embodiments 43-54, wherein R⁷ is H, optionally substituted C₁-C₄ alkyl, optionally substituted C₁-C₄ aminoalkyl or optionally substituted heteroaryl.

Embodiment 57. The compound according to any one of embodiments 43-54, wherein R⁴ is H, optionally substituted C₁-C₄ alkyl, optionally substituted C₁-C₄ carboxyalkyl or optionally substituted C₁-C₄ aminoalkyl.

Embodiment 58. The compound according to any one of embodiments 43-54, wherein R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

Embodiment 59. The compound according to any one of embodiments 43-58, wherein R² is H or C₁-C₆ alkyl.

Embodiment 60. The compound according to any one of embodiments 43-59, wherein R² is H or C₁-C₄ alkyl.

Embodiment 61. The compound according to any one of embodiments 43-60, wherein R² is H.

Embodiment 62. The compound according to any one of embodiments 43-60, wherein R² is C₁-C₄ alkyl.

Embodiment 63. The compound according to embodiment 1, having Formula IV:

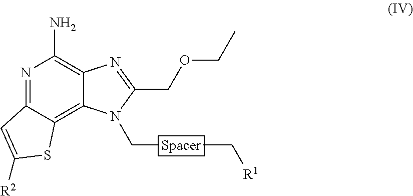

wherein:

R¹ is —OH, —NR⁴R⁵, —OR¹⁰, SR¹¹ or

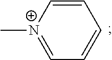

R² is H or optionally substituted C₁-C₆ alkyl, and

Spacer is —(CH₂)ₙ—,

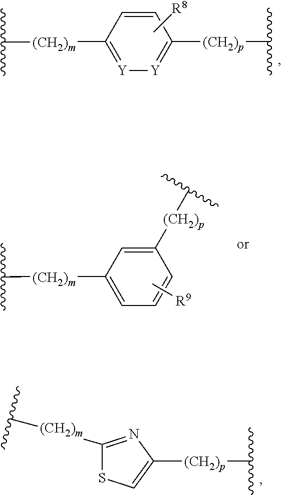

wherein n is an integer between 3 and 10; each m is independently an integer between 0 and 4; each p is independently an integer between 0 and 4; and Y is CH or N;

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;

R⁸ and R⁹ are each independently H, NR¹³R¹⁴, halo, optionally substituted C₁-C₄ alkoxy or optionally substituted C₁-C₄ alkyl;

R¹⁰ and R¹¹ are each independently optionally substituted C₁-C₆ alkyl, optionally substituted aryl or optionally substituted C₃-C₇ cycloalkyl; and

R¹³ and R¹⁴ are each independently H or optionally substituted C₁-C₄ alkyl.

Embodiment 64. The compound according to embodiment 63, wherein Spacer is —(CH₂)ₙ—,

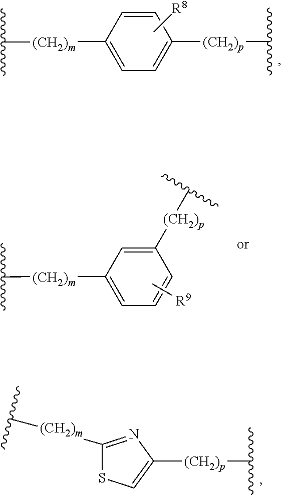

wherein n is an integer between 3 and 10; each m is independently an integer between 0 and 4; each p is independently an integer between 0 and 4, and R⁸ and R⁹ are each independently H, NR¹³R¹⁴, halo, optionally substituted C₁-C₄ alkoxy or optionally substituted C₁-C₄ alkyl, and wherein R¹³ and R¹⁴ are each independently H or optionally substituted C₁-C₄ alkyl.

Embodiment 65. The compound according to embodiment 63 or embodiment 64, wherein Spacer is:

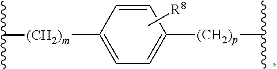

wherein m is an integer between 0 and 4; p is an integer between 0 and 4, and R⁸ is H, NR¹³R¹⁴, halo, optionally substituted C₁-C₄ alkoxy or optionally substituted C₁-C₄ alkyl, and wherein R¹³ and R¹⁴ are each independently H or optionally substituted C₁-C₄ alkyl.

Embodiment 66. The compound according to embodiment 63 or embodiment 64, wherein R⁸ and R⁹ are each independently H, NR¹³R¹⁴, halo, C₁-C₄ alkoxy or C₁-C₄ alkyl, wherein R¹³ and R¹⁴ are each independently H or C₁-C₄ alkyl.

Embodiment 67. The compound according to embodiment 63 or embodiment 64, wherein R⁸ and R⁹ are each H or halo.

Embodiment 68. The compound according to any one of embodiments 63-67, wherein m is 0.

Embodiment 69. The compound according to any one of embodiments 63-68, wherein p is 0.

Embodiment 70. The compound according to embodiment 63 or embodiment 64, wherein Spacer is —(CH₂)ₙ—,

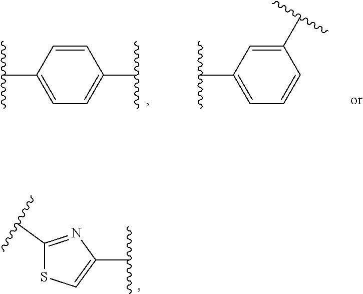

wherein n is an integer between 3 and 10.

Embodiment 71. The compound according to embodiment 63, embodiment 64 or embodiment 70, wherein n is an integer between 3 and 5.

Embodiment 72. The compound according to embodiment 63 or embodiment 64, wherein Spacer is

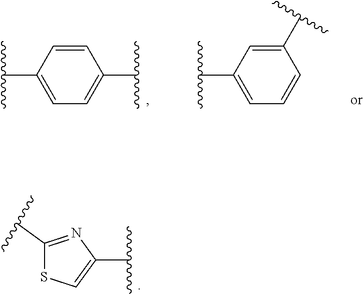 [SMILES: *c1ccc(*)cc1 ; *c1cccc(*)c1 ; *c1csc(*)n1]

Embodiment 73. The compound according to any one of embodiments 63-72, wherein R¹ is —OH, —NR⁴R⁵, —OR¹⁰, SR¹¹,

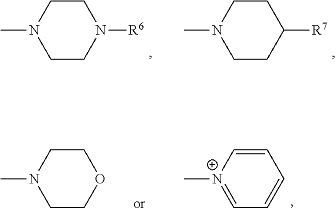

and wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

Embodiment 74. The compound according to any one of embodiments 63-73, wherein R¹ is —OH, —NR⁴R⁵,

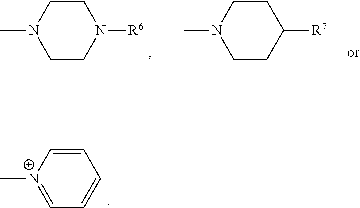

Embodiment 75. The compound according to any one of embodiments 63-74, wherein R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

Embodiment 76. The compound according to any one of embodiments 63-74, wherein R⁷ is H, optionally substituted C₁-C₄ alkyl, optionally substituted C₁-C₄ aminoalkyl or optionally substituted heteroaryl.

Embodiment 77. The compound according to any one of embodiments 63-74, wherein R⁴ is H, optionally substituted C₁-C₄ alkyl, optionally substituted C₁-C₄ carboxyalkyl or optionally substituted C₁-C₄ aminoalkyl.

Embodiment 78. The compound according to any one of embodiments 63-74, or 77, wherein R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

Embodiment 79. The compound according to any one of embodiments 63-78, wherein R² is H or C₁-C₆ alkyl.

Embodiment 80. The compound according to any one of embodiments 63-79, wherein R² is H or C₁-C₄ alkyl.

Embodiment 81. The compound according to any one of embodiments 63-80, wherein R² is H.

Embodiment 82. The compound according to any one of embodiments 63-80, wherein R² is C₁-C₄ alkyl.

Embodiment 83. The compound according to embodiment 1, having Formula V:

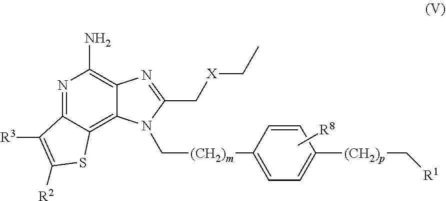

wherein:

X is —CH₂— or —O—;

R¹ is —OH, —NR⁴R⁵, —OR¹⁰, SR¹¹ or

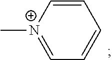

R² and R³ are each independently H or optionally substituted C₁-C₆ alkyl;

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;

R⁸ is H, NR¹³R¹⁴, halo, optionally substituted C₁-C₄ alkoxy or optionally substituted C₁-C₄ alkyl;

R¹⁰ and R¹¹ are each independently optionally substituted C₁-C₆ alkyl, optionally substituted aryl or optionally substituted C₃-C₇ cycloalkyl;

R¹³ and R¹⁴ are each independently H or optionally substituted C₁-C₄ alkyl;

each m is independently an integer between 0 and 4; and

each p is independently an integer between 0 and 4.

Embodiment 84. The compound according to embodiment 83, wherein m is 0.

Embodiment 85. The compound according to embodiment 83 or embodiment 84, wherein p is 0.

Embodiment 86. The compound according to any one of embodiments 83-85, wherein R⁸ is H, NR¹³R¹⁴, halo, C₁-C₄ alkoxy or C₁-C₄ alkyl, and wherein R¹³ and R¹⁴ are each independently H or C₁-C₄ alkyl.

Embodiment 87. The compound according to any one of embodiments 83-86, wherein R⁸ is H or halo.

Embodiment 88. The compound according to any one of embodiments 83-87, wherein R¹ is —OH, —NR⁴R⁵, —OR¹⁰, SR¹¹,

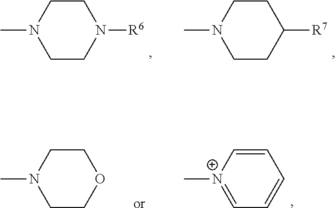

and wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

Embodiment 89. The compound according to any one of embodiments 83-88, wherein R¹ is —OH, —NR⁴R⁵,

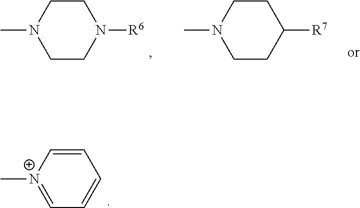

Embodiment 90. The compound according to any one of embodiments 83-89, wherein R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

Embodiment 91. The compound according to any one of embodiments 83-89, wherein R⁷ is H, optionally substituted C₁-C₄ alkyl, optionally substituted C₁-C₄ aminoalkyl or optionally substituted heteroaryl.

Embodiment 92. The compound according to any one of embodiments 83-89, wherein R⁴ is H, optionally substituted C₁-C₄ alkyl, optionally substituted C₁-C₄ carboxyalkyl or optionally substituted C₁-C₄ aminoalkyl.

Embodiment 93. The compound according to any one of embodiments 83-89, or 92, wherein R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

Embodiment 94. The compound according to any one of embodiments 83-93, wherein R² and R³ are each independently H or C₁-C₆ alkyl.

Embodiment 95. The compound according to any one of embodiments 83-94, wherein R² and R³ are each independently H or C₁-C₄ alkyl.

Embodiment 96. The compound according to any one of embodiments 83-95, wherein R³ is H.

Embodiment 97. The compound according to any one of embodiments 83-96, wherein R² is C₁-C₄ alkyl, and R³ is H.

Embodiment 98. The compound according to any one of embodiments 83-96, wherein R² and R³ are each H.

Embodiment 99. The compound according to any one of embodiments 83-98, wherein X is —CH₂—.

Embodiment 100. The compound according to any one of embodiments 83-98, wherein X is —O—.

Embodiment 101. The compound according to embodiment 1, having Formula VI:

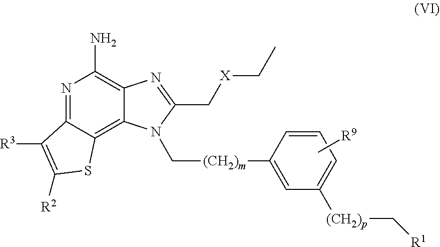

wherein:

X is —CH₂— or —O—;

R¹ is —OH, —NR⁴R⁵, —OR¹⁰, SR¹¹ or

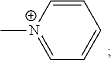

R² and R³ are each independently H or optionally substituted C₁-C₆ alkyl; R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;

R⁹ is H, NR¹³R¹⁴, halo, optionally substituted C₁-C₄ alkoxy or optionally substituted C₁-C₄ alkyl;

R¹⁰ and R¹¹ are each independently optionally substituted C₁-C₆ alkyl, optionally substituted aryl or optionally substituted C₃-C₇ cycloalkyl;

R¹³ and R¹⁴ are each independently H or optionally substituted C₁-C₄ alkyl;

each m is independently an integer between 0 and 4; and

each p is independently an integer between 0 and 4.

Embodiment 102. The compound according to embodiment 101, wherein m is 0.

Embodiment 103. The compound according to embodiment 101 or embodiment 102, wherein p is 0.

Embodiment 104. The compound according to any one of embodiments 101-103, wherein R⁹ is H, NR¹³R¹⁴, halo, C₁-C₄ alkoxy or C₁-C₄ alkyl, and wherein R¹³ and R¹⁴ are each independently H or C₁-C₄ alkyl.

Embodiment 105. The compound according to any one of embodiments 101-104, wherein R⁹ is H or halo.

Embodiment 106. The compound according to any one of embodiments 101-105, wherein R¹ is —OH, —NR⁴R⁵, —OR¹⁰, SR¹¹,

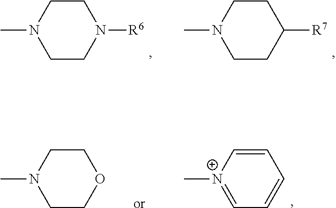

and wherein:

R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;

R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and

R¹⁰ and R¹¹ are each independently optionally substituted C₁-C₆ alkyl, optionally substituted aryl or optionally substituted C₃-C₇ cycloalkyl.

Embodiment 107. The compound according to any one of embodiments 101-106, wherein R¹ is —OH, —NR⁴R⁵,

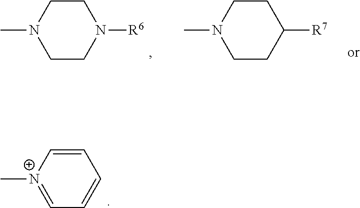

Embodiment 108. The compound according to any one of embodiments 101-107, wherein R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

Embodiment 109. The compound according to any one of embodiments 101-107, wherein R⁷ is H, optionally substituted C₁-C₄ alkyl, optionally substituted C₁-C₄ aminoalkyl or optionally substituted heteroaryl.

Embodiment 110. The compound according to any one of embodiments 101-107, wherein R⁴ is H, optionally substituted C₁-C₄ alkyl, optionally substituted C₁-C₄ carboxyalkyl or optionally substituted C₁-C₄ aminoalkyl.

Embodiment 111. The compound according to any one of embodiments 101-107, or 110, wherein R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

Embodiment 112. The compound according to any one of embodiments 101-111, wherein R² and R³ are each independently H or C₁-C₆ alkyl.

Embodiment 113. The compound according to any one of embodiments 101-112, wherein R² and R³ are each independently H or C₁-C₄ alkyl.

Embodiment 114. The compound according to any one of embodiments 101-113, wherein R³ is H.

Embodiment 115. The compound according to any one of embodiments 101-114, wherein R² is C₁-C₄ alkyl, and R³ is H.

Embodiment 116. The compound according to any one of embodiments 101-114, wherein R² and R³ are each H.

Embodiment 117. The compound according to any one of embodiments 101-116, wherein X is —CH₂—.

Embodiment 118. The compound according to any one of embodiments 101-116, wherein X is —O—.

Embodiment 119. A compound that is selected from any one of the compounds listed in Table 1.

Embodiment 120. The compound according to any one of embodiments 1-119, wherein the compound has an EC₅₀ value for agonizing TLR7 of <500 nM, <250 nM, or <100 nM.

Embodiment 121. A pharmaceutical composition comprising a compound according to any one of embodiments 1-120, or a pharmaceutically acceptable salt thereof, and a pharmaceutically acceptable carrier or diluent.

Embodiment 122. A method of agonizing a TLR, the method comprising contacting a cell that expresses the TLR with a compound according to any one of embodiments 1-120, or a pharmaceutically acceptable salt thereof, thereby agonizing the TLR.

Embodiment 123. The method of embodiment 122, wherein the cell is an immune cell.

Embodiment 124. The method of embodiment 123, wherein the immune cell is a dendritic cell or a macrophage.

Embodiment 125. The method of any one of embodiments 122-124, wherein the TLR is a TLR7, a TLR8, or a combination thereof.

Embodiment 126. The method of any one of embodiments 122-125, wherein the TLR is a TLR7.

Embodiment 127. The method of embodiment 126, wherein the compound agonizes the TLR7 with an EC₅₀ value of <500 nM, <250 nM, or <100 nM.

Embodiment 128. A method of stimulating an immune response in a subject in need thereof, the method comprising administering to the subject an effective amount of a compound according to any one of embodiments 1-120, or a pharmaceutically acceptable salt thereof.

Embodiment 129. The method of embodiment 128, wherein the compound agonizes a TLR in the subject, thereby stimulating the immune response in the subject.

Embodiment 130. The method of embodiment 129, wherein the TLR is a TLR7, a TLR8, or a combination thereof.

Embodiment 131. The method of embodiment 129 or embodiment 130, wherein the TLR is a TLR7.

Embodiment 132. A method of treating a cancer in a subject in need thereof, the method comprising administering to the subject an effective amount of a compound according to any one of embodiments 1-120, or a pharmaceutically acceptable salt thereof.

Embodiment 133. The method of embodiment 132, wherein the compound agonizes a TLR in the subject, thereby treating the cancer in the subject.

Embodiment 134. The method of embodiment 133, wherein the TLR is a TLR7, a TLR8, or a combination thereof.

Embodiment 135. The method of embodiment 133 or embodiment 134, wherein the TLR is a TLR7.

Embodiment 136. The method of any one of embodiments 132-135, wherein the cancer is selected from the group consisting of hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, and head and neck cancer.

Embodiment 137. A conjugate having Formula X:

T-(L-(D)ᵣ)_{q} (X)

wherein:

T is a targeting moiety;

L is a linker;

D is a compound according to any one of embodiments 1-120;

q is a value from about 1 to about 8, and

r is an integer from 1 to 4.

Embodiment 138. The conjugate according to embodiment 137, wherein q is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

Embodiment 139. The conjugate according to embodiment 137 or embodiment 138, wherein r is 1 or 2.

Embodiment 140. The conjugate according to any one of embodiments 137-139, wherein the product of q and r is 24 or less.

Embodiment 141. The conjugate according to any one of embodiments 137-140, wherein the product of q and r is 4.

Embodiment 142. The conjugate according to any one of embodiments 137-141, wherein T is an antibody or antigen-binding antibody fragment.

Embodiment 143. The conjugate according to embodiment 142, wherein the antibody or antigen-binding antibody fragment is capable of binding a tumor associated antigen (TAA).

Embodiment 144. The conjugate according to any one of embodiments 137-143, wherein the linker is a cleavable linker.

Embodiment 145. The conjugate according to any one of embodiments 137-144, wherein the linker comprises a peptide sequence comprising at least 2 amino acids.

Embodiment 146. The conjugate according to any one of embodiments 137-143, wherein the linker comprises compound 10.c or 10.d.

Embodiment 147. A pharmaceutical composition comprising a conjugate according to any one of embodiments 137-146, and a pharmaceutically acceptable carrier or diluent.

Embodiment 148. A method of agonizing a TLR, the method comprising contacting a cell that expresses the TLR with a conjugate according to any one of embodiments 137-146, thereby agonizing the TLR.

Embodiment 149. The method of embodiment 148, wherein the cell is an immune cell.

Embodiment 150. The method of embodiment 149, wherein the immune cell is a dendritic cell or a macrophage.

Embodiment 151. The method of any one of embodiments 148-150, wherein the TLR is a TLR7, a TLR8, or a combination thereof.

Embodiment 152. The method of any one of embodiments 148-151, wherein the TLR is a TLR7.

Embodiment 153. A method of stimulating an immune response in a subject in need thereof, the method comprising administering to the subject an effective amount of a conjugate according to any one of embodiments 137-146.

Embodiment 154. The method of embodiment 153, wherein the conjugate agonizes a TLR in the subject, thereby stimulating the immune response in the subject.

Embodiment 155. The method of embodiment 154, wherein the TLR is a TLR7, a TLR8, or a combination thereof.

Embodiment 156. The method of embodiment 154 or embodiment 155, wherein the TLR is a TLR7.

Embodiment 157. A method of treating a cancer in a subject in need thereof, the method comprising administering to the subject an effective amount of a conjugate according to any one of embodiments 137-146.

Embodiment 158. The method of embodiment 157, wherein the conjugate agonizes a TLR in the subject, thereby treating the cancer in the subject.

Embodiment 159. The method of embodiment 158, wherein the TLR is a TLR7, a TLR8, or a combination thereof.

Embodiment 160. The method of embodiment 158 or embodiment 159, wherein the TLR is a TLR7.

Embodiment 161. The method of any one of embodiments 157-160, wherein the cancer is selected from the group consisting of hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, and head and neck cancer.

Embodiment 162. A compound according to any one of embodiments 1-120 for use in therapy.

Embodiment 163. A compound according to any one of embodiments 1-120, or a pharmaceutically acceptable salt thereof, for use to stimulate an immune response in a subject in need thereof.

Embodiment 164. Use of a compound according to any one of embodiments 1-120, or a pharmaceutically acceptable salt thereof, in the manufacture of a medicament for stimulating an immune response in a subject in need thereof.

Embodiment 165. A compound according to any one of embodiments 1-120, or a pharmaceutically acceptable salt thereof, for use to treat a cancer in a subject in need thereof.

Embodiment 166. Use of a compound according to any one of embodiments 1-120, or a pharmaceutically acceptable salt thereof, in the manufacture of a medicament for treating a cancer in a subject in need thereof.

Embodiment 167. The compound for use according to embodiment 165, or the use according to embodiment 166, wherein the cancer is selected from the group consisting of hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, and head and neck cancer.

Embodiment 168. A conjugate according to any one of embodiments 137-146 for use to stimulate an immune response in a subject in need thereof.

Embodiment 169. Use of a conjugate according to any one of embodiments 137-146 in the manufacture of a medicament for stimulating an immune response in a subject in need thereof.

Embodiment 170. A conjugate according to any one of embodiments 137-146 for use to treat a cancer in a subject in need thereof.

Embodiment 171. Use of a conjugate according to any one of embodiments 137-146 in the manufacture of a medicament for treating a cancer in a subject in need thereof.

Embodiment 172. The conjugate for use according to embodiment 170, or the use according to embodiment 171, wherein the cancer is selected from the group consisting of hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, and head and neck cancer.

### EXAMPLES

The following Examples provide illustrative methods of making and using compounds of the present disclosure, e.g., those of general Formula I. It is understood that one skilled in the art may be able to synthesize these compounds by similar methods or by combining other methods known in the art. Preparation of other compounds of general Formula I not specifically illustrated herein could be achieved by one skilled in the art using the methods described herein or similar methods with appropriate starting components and modification of the parameters of the synthesis as needed. In general, starting components may be obtained from commercial sources such as Sigma Aldrich (Merck KGaA), Alfa Aesar and Maybridge (Thermo Fisher Scientific Inc.), Matrix Scientific, Tokyo Chemical Industry Ltd. (TCI) and Fluorochem Ltd., and/or synthesized according to sources known to those skilled in the art (see, for example, March's Advanced Organic Chemistry: Reactions, Mechanisms, and Structure, 7th edition, John Wiley & Sons, Inc., 2013) or prepared as described herein.

The following abbreviations are used throughout the Examples. DCM=dichloromethane; DIPEA=N,N-diisopropylethylamine; DMA=dimethylacetamide; DMF=dimethylformamide; DMSO=dimethylsulphoxide; IL-6=interleukin 6; LC/MS=Liquid Chromatography/Mass Spectrometry; LC/MSD=Liquid Chromatography/Mass Selective Detector; SEC=Size-exclusion chromatography; HIC=hydrophobic interaction chromatography; RP-UPLC=reverse-phase ultra performance liquid chromatography; HPLC=high-performance liquid chromatography; MT=maleimidotriethylene glycolate; PABC=p-aminobenzyloxycarbonyl; PBMC=peripheral blood mononuclear cell; PNP=p-nitrophenol; rt=room temperature; TCEP=tris(2-carboxyethyl)phosphine; TFA=trifluoroacetic acid; TNF-α=tumor necrosis factor α; VC=valine-citrulline; UHPLC=ultra high-performance liquid chromatograph.

### General Procedures

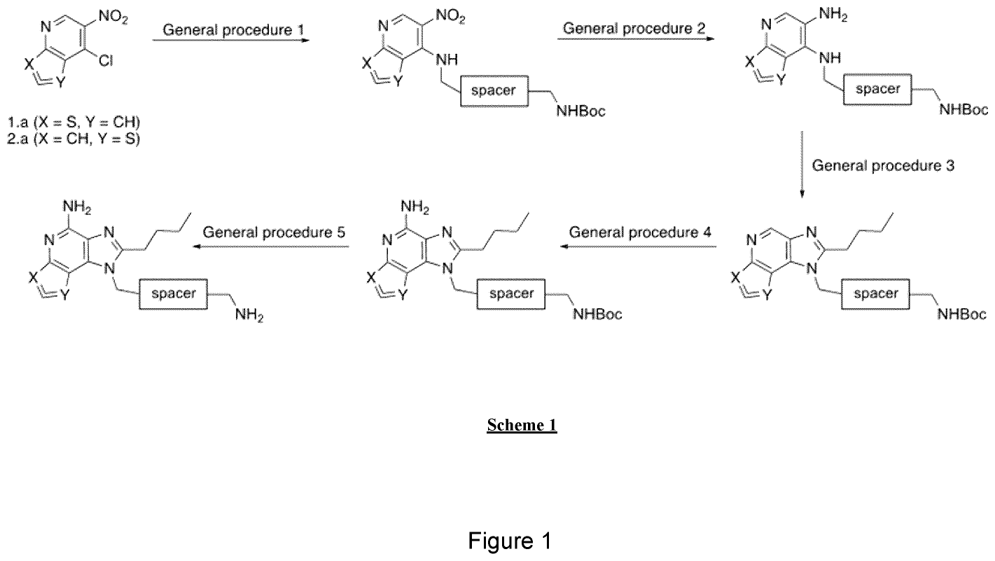

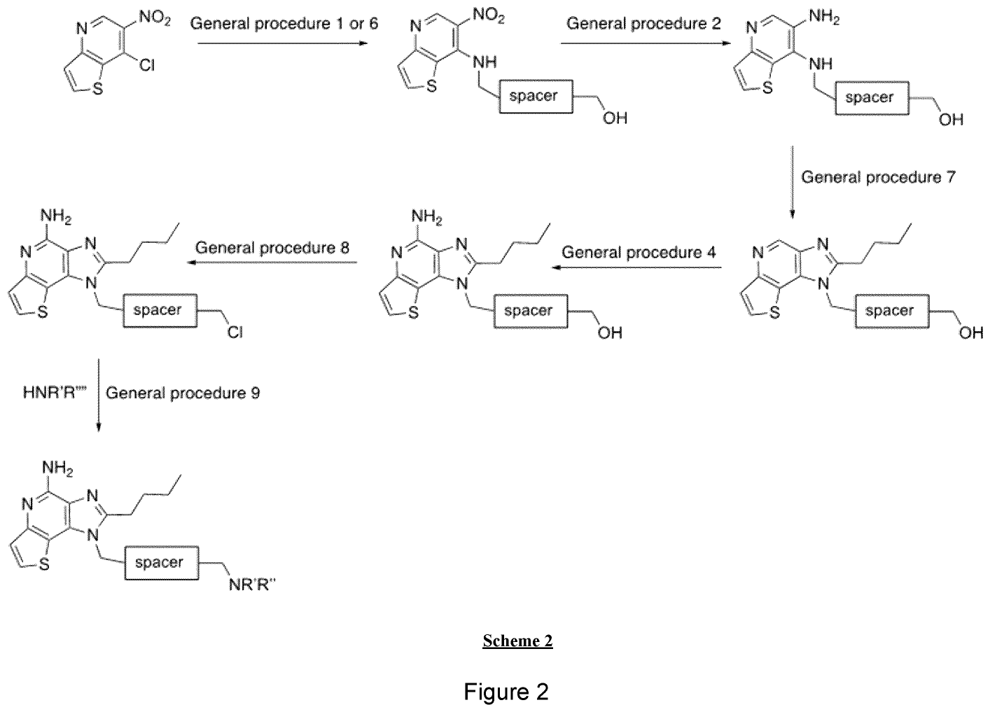

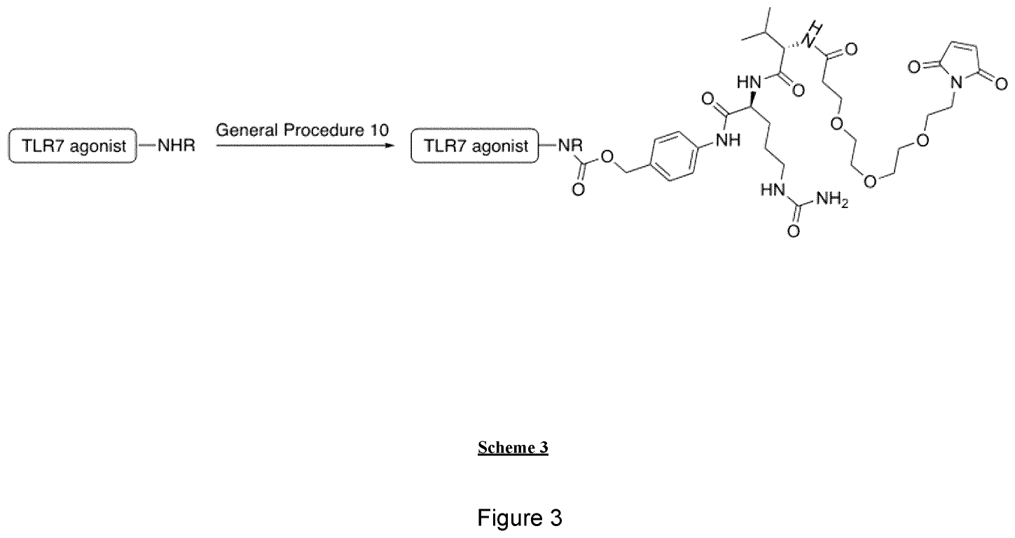

The following general synthetic procedures were employed in the preparation of compounds of general Formula I. Examples of synthetic schemes employing these procedures to prepare compound of general Formula I are shown in FIGS. 1 to 3 (e.g., Schemes 1 to 3).

### General Procedure 1: Installation of Amino-Boc Spacer

To a stirring solution of the chloronitrothienopyridine in dimethylformamide (0.2-0.5 M) was added the mono-Boc-diamine or the amino-alcohol (1.5 eq.) followed by anhydrous potassium carbonate (2.0 eq.). Upon completion (by LCMS, typically 1-3 h), the reaction mixture was partially concentrated in vacuo. Water was added and the mixture was extracted twice with ethyl acetate or 10% methanol in dichloromethane. The combined organic extracts were dried over sodium sulfate and concentrated in vacuo to afford product as a dimethylformamide laden crude liquid. The crude residue was typically taken to the next step without additional purification.

### General Procedure 2: Reduction of Nitro-Group

To a stirring solution of the nitro compound in a 9:1 mixture of methanol:water (0.1 M) was added NiCl₂·6H₂O (0.1 eq.) followed by NaBH₄ (2 eq.) portion wise over 30 minutes. The solution was stirred until complete consumption of starting material was observed by LCMS. Additional NaBH₄ (0.5 eq.) was added as necessary. Upon completion, the reaction mixture was quenched carefully with acetic acid (1 mL/g of nitro compound) prior to partial concentration in vacuo. The resulting crude residue was purified by reverse-phase flash chromatography to provide the desired product after lyophilization.

### General Procedure 3: Imidazole Formation for Amino-Boc Spacers

To a stirring solution of the bis-anilino compound in toluene (0.05 M) was added p-toluenesulfonic acid (0.1 eq.) and trimethylorthovalerate (1.1 eq.). The reaction mixture was heated at 120° C. The reaction was monitored by LCMS and additional trimethylorthovalerate (0.5 eq.) was added as necessary. Upon completion (typically about 1 h), the reaction mixture was cooled to room temperature (rt) then concentrated in vacuo. The resulting crude residue was purified by flash chromatography to provide the desired product.

### General Procedure 4: Amidine Formation

To the TFA salt of the pyridine compound was added 1% concentrated aqueous ammonium hydroxide in methanol (v./v., 5 mL per mmol). The solution was concentrated to dryness in vacuo to provide the free base. To a stirring solution of the pyridine compound free base in 1,2-dichloroethane, dichloromethane or chloroform (0.05-0.1 M) was added 3-chloroperbenzoic acid (2.0 eq., 77%). The solution was stirred until complete conversion to the N-oxide compound was observed by LCMS (typically 1-3 h). Upon completion, the reaction mixture was diluted with dichloromethane and washed with a 10% concentrated aqueous ammonium hydroxide (v./v.) in water solution. The organic extract was concentrated in vacuo and dissolved in 1,2-dichloroethane (0.01-0.1 M) with rapid stirring. An equal volume of concentrated aqueous ammonium hydroxide was subsequently added, followed by 4-toluenesulfonyl chloride (1.1 eq.). Upon completion (typically <1 h), the reaction mixture was partitioned between dichloromethane and saturated aqueous sodium bicarbonate. The organic layer was concentrated in vacuo then purified by reverse-phase flash chromatography to provide the desired amidine product after lyophilization.

### General Procedure 5: Deprotection of Boc-Amine

To a stirring solution of the Boc protected amine compound in dichloromethane (0.1 M) was added TFA (20% by volume). Upon completion (typically 1 h), the reaction mixture was concentrated in vacuo to provide a crude solid or was purified by preparative HPLC to provide the desired product after lyophilization.

### General Procedure 6: Installation of Hydroxyl-Containing Spacer

To a stirring solution of the chloronitrothienopyridine in dimethylformamide (0.2-0.5 M) was added the amino-alcohol (1.5 eq.) followed by anhydrous potassium carbonate or DIPEA (2.0 eq.). Upon completion, the reaction mixture was diluted with water (5 times the volume of dimethylformamide) and the yellow precipitate was collected via vacuum filtration. The crude solid was washed once with water then dried under vacuum. The crude residue was typically taken to the next step with no additional purification.

### General Procedure 7: Imidazole Formation for Hydroxyl-Containing Spacers

To a stirring solution of the bis-anilino compound in acetic acid (0.1-0.2 M) was added trimethylorthovalerate (1.1 eq.). The reaction mixture was heated at 60° C. for 1 h. The reaction was monitored by LCMS and additional trimethylorthovalerate (0.5 eq.) was added as necessary. Upon completion (typically <1 h), the reaction mixture was cooled to rt prior to concentration in vacuo to afford the crude product. The crude residue was co-evaporated (2×50 mL toluene) then purified by reverse-phase flash chromatography to provide the desired product after lyophilization. In some cases, the desired product was found to contain a pentanoic or methyl ester moiety on the free alcohol group. In these cases, the residue was dissolved in tetrahydrofuran (0.2 M) and an equal volume of 1 M aqueous lithium hydroxide was added. Upon complete ester cleavage, the reaction mixture was partially concentrated in vacuo and extracted twice with ethyl acetate. The combined organic layers were concentrated in vacuo then purified by reverse-phase flash chromatography to provide the desired product after lyophilization.

### General Procedure 8: Formation of Alkyl/Benzyl Chlorides

To the alcohol compound was added thionyl chloride (20-50 eq.). The solution was heated at 60° C. for 1 h, then concentrated in vacuo. The residue was co-evaporated (2×5 mL dichloromethane) to give the crude chloride compound, which was typically used without further purification.

### General Procedure 9: Nucleophilic Displacement of Chloride

To the chloride compound in DMF or DMA (0.05 M) was added a primary or secondary amine (3 eq.) followed by DIPEA (3 eq.), and the solution was heated at 70° C. Upon completion (typically 1-18 h), the reaction mixture was purified by reverse-phase HPLC to provide the desired product after lyophilization.

### General Procedure 10: Drug-Linker Synthesis

To the primary or secondary amine compound in dimethylformamide (0.05 M) was added Compound 10.e (MT-VC-PABC-PNP) (1 eq.) followed by DIPEA (3 eq.). Upon completion (typically 1-4 h), the reaction mixture was acidified with 1 M HCl then purified by reverse-phase HPLC to provide the desired drug-linker after lyophilization.

### General Procedure 11: Compound Purification

Flash Chromatography: Crude reaction products were purified with Biotage® Snap Ultra columns (10, 25, 50, or 100 g) (Biotage, Charlotte, NC), and eluting with linear gradients of ethyl acetate/hexanes or methanol/dichloromethane on a Biotage® Isolera™ automated flash system (Biotage, Charlotte, NC). Alternatively, reverse-phase flash purification was conducting using Biotage® Snap Ultra C18 columns (12, 30, 60, or 120 g) and eluting with linear gradients of 0.1% TFA in acetonitrile/0.1% TFA in water. Purified compounds were isolated by either removal of organic solvents by rotavap or lyophilization of acetonitrile/water mixtures.

Preparative HPLC: Reverse-phase HPLC of crude compounds was performed using a Kinetex® 5-μm EVO C18 100 Å (250×21.2 mm) column (Phenomenex Inc., Torrance, CA) on an Agilent 1260 Infinity II preparative LC/MSD system (Agilent Technologies, Inc., Santa Clara, CA), and eluting with linear gradients of 0.1% TFA in acetonitrile/0.1% TFA in water. Purified compounds were isolated by lyophilization of acetonitrile/water mixtures.

### General Procedure 12: Compound Analysis

LC/MS: Purified compounds were analyzed using a Kinetex® 2.6-μm C18 100 Å (30×3 mm) column (Phenomenex Inc., Torrance, CA) on an Agilent 1290 HPLC/6120 single quad LC/MS system (Agilent Technologies, Inc., Santa Clara, CA), and eluting with a linear gradient of 10 to 100+0.1% formic acid in acetonitrile/0.1% formic acid in water.

NMR: ¹H NMR spectra were collected with a Bruker AVANCE III 300 Spectrometer (300 MHz) (Bruker Corporation, Billerica, MA). Chemical shifts are in parts per million (ppm).

### General Procedure 13: Preparation of Antibody-Drug Conjugates Using Maleimide-Containing Drug-Linkers

The antibody (1-10 mg/mL in phosphate buffered saline, pH 7.4) was reduced with TCEP (1-10 mM in dH₂O) (2.0-3.0 molar equivalents) in the presence of 1 mM DTPA. The solution was mixed thoroughly and incubated at 37° C. for 120 min before cooling on ice. In some instances, the reduced antibody solution was further buffer exchanged into 10 mM sodium acetate buffer, pH 4.5 by passage over a Zeba™ Spin Desalting Column (40 KDa MWCO) (Thermo Scientific, Waltham, MA). To the reduced protein solution stored on ice was added the maleimide functionalized drug-linker (10 mM in DMSO) (10-12 molar equivalents). The conjugation reaction was immediately mixed thoroughly by pipetting and conjugation was allowed to proceed at room temperature for 90 to 120 min. The reaction mixture was then purified by passage over Zeba™ Spin Desalting Columns (40 KDa MWCO) pre-equilibrated with phosphate buffered saline, pH 7.4 or 10 mM sodium acetate, pH 4.5. The purified conjugates were stored at 4° C. and analyzed for total protein content by bicinchonic acid assay (Pierce microBCA™ Protein Assay Kit; Thermo Scientific, Waltham, MA), characterized by HPLC-HIC, SEC and RP-UPLC-MS. The average DAR and drug distribution were derived from interpretation of HIC and LC-MS data. Endotoxin levels were assessed using the ToxinSensor™ Single Test Kit (Genscript USA Inc. Piscataway, NJ), with a threshold set at 0.5 EU/mg. Residual free drug and drug-linker levels were assessed by RP-UPLC-MS, with a threshold set at 1% ((free drug+free drug-linker)/(conjugated drug-linker)).

### General Procedure 14: Determination of Drug-Antibody Ratio (DAR) by RP-UPLC-MS

10 μL samples of antibody-drug conjugate at 1 mg/ml were deglycosylated using EndoS (1 μg). After a 1 h incubation at room temperature, 2 μL of 500 mM TCEP (in water) was added to 11 μL sample and incubated at 70° C. for 30 min for reduction and denaturation.

Samples were then run on LC MS Quadrupole Time-of-Flight (QTOF) mass spectrometer, 1 μl injection each following the analytical method described below:
Column: Agilent PLRP-S 1000A 8 μM 50x2.1 MM
Column Temperature: 70° C.
Flow Rate: 0.3 mL/min
Mobile Phase C: 0.1% Formic Acid (FA) 0.025% Trifluoroacetic Acid (TFA) and 10% Isopropyl Alcohol (IPA) in water
Mobile Phase D: 0.1% FA and 10% IPA in acetonitrile (ACN)
Detection: Signal A (280 nm, 4.0 bandwidth), Signal B (220 nm, 4.0 bandwidth)
Gradient:

| Time | C | D |
|---|---|---|
| 0 | 80 | 20 |
| 20 | 60 | 40 |
| 22 | 10 | 90 |
| 22.5 | 1 | 99 |
| 24 | 1 | 99 |

Post Run Time: 2 minutes
   MS data analysis was performed as follows: For cysteine conjugates, the total ion chromatogram (TIC) was integrated at two regions (Light Chain (LC) region between 3 to 8 minutes, Heavy Chain (HC) region between 6.5 to 13 minutes). Deconvolution Parameters:
Deconvolution Algorithm: Maximum Entropy
Mass Range: 20000-60000
Mass Step: 1.0
Used limited m/z range: 1000-7000
Subtract baseline: 7.0
Adduct: Proton
Isotope width: Automatic
Height Filter: Peak signal to noise >=30.0
Maximum number of peaks: Limited by height 100
Calculate average mass using top 90% of peak height
   Average DAR was calculated by the peak height for each DAR species.

### General Procedure 15: Determination of DAR by Hydrophobic Interaction Chromatography (HIC)

Determination of DAR for the antibody-drug conjugates was assessed by hydrophobic interaction chromatography (HIC) following the general procedures outlined in Antibody Drug Conjugates, Methods in Molecular Biology, 2013, vol. 1045, pp. 275-284. L. Ducry, Ed. Briefly, ADCs were subjected to HIC on a TSKgel® Butyl-NPR column (4.6 mm×3 mm i.d.; 2.5 m particle size) (Tosoh Bioscience LLC, Tokyo, Japan) connected to an Agilent 1100 series HPLC. Samples were injected (5 μL) at or above 2 mg/mL. A linear gradient elution was employed starting at 95% mobile phase A/5% mobile phase B, transitioning to 5% mobile phase A/95% mobile phase B over a period of 12 min (mobile phase A: 1.5 M ammonium sulfate+25 mM sodium phosphate, pH 6.95; mobile phase B: 25% isopropanol, 75% 25 mM sodium phosphate, pH 6.95). Injection of unmodified antibody provided a means of identifying the peak with DAR=0. Antibodies were detected on the basis of absorbance at 280 nm.

### General Procedure 16: Size Exclusion Chromatography (SEC) Analysis of ADCs

Samples were run on an Agilent 1290 UHPLC system equipped with a quaternary pump and a diode array detection (DAD) detector, 5 μl injection each following the analytical method described below:
Column: Agilent AdvanceBio SEC, 300 Å, 2.7 μm, 7.8×150 mm
Column Temperature: 25° C.
Flow Rate: 1.0 mL/min
Mobile Phase: 150 mM NaₓPO₄ pH6.95
Detection: Absorbance at 280 nm, 4.0 bandwidth
Isocratic run time: 7 min

### Example 1: Preparation of Series 1 Compounds

### 1.1 4-chloro-5-nitrothieno[2,3-b]pyridine (Compound 1.a)

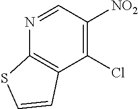 [SMILES: O=[N+]([O-])c1cnc2sccc2c1Cl]

The title compound was prepared according to the procedure described in U.S. Patent Application Publication No. US 2014/0200227.

### 1.2 tert-butyl (4-(((5-nitrothieno[2,3-b]pyridin-4-yl)amino)methyl)benzyl)carbamate (Compound 1.b)

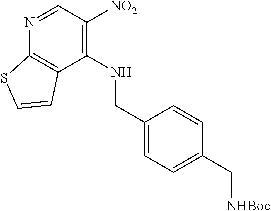 [SMILES: CC(C)(C)OC(=O)NCc1ccc(CNc2c([N+](=O)[O-])cnc3sccc23)cc1]

The title compound was prepared according to General Procedure 1 starting from Compound 1.a (330 mg) and tert-butyl (4-(aminomethyl)benzyl)carbamate. The crude compound was taken to the next step with no additional purification.

LC/MS: Calc'd m/z=414.1 for C₂₀H₂₂N₄O₄S, found [M+H]⁺=415.1.

### 1.3 tert-butyl (4-(((5-aminothieno[2,3-b]pyridin-4-yl)amino)methyl)benzyl)carbamate (Compound 1.c)

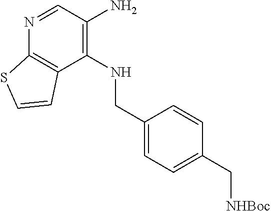 [SMILES: CC(C)(C)OC(=O)NCc1ccc(CNc2c(N)cnc3sccc23)cc1]

The title compound was prepared according to General Procedure 2 starting from crude Compound 1.b. After quenching with AcOH, the reaction mixture was partially concentrated in vacuo. The residue was partitioned between ethyl acetate and saturated aqueous sodium bicarbonate. The organic layer was dried over sodium sulfate then concentrated in vacuo. Purification was accomplished as described in General Procedure 11, eluting with a 0 to 15% DCM/MeOH+1% NH₄OH gradient to give Compound 1.c as a yellow solid (143 mg, 24% yield over two steps).

LC/MS: Calc'd m/z=384.2 for C₂₀H₂₄N₄O₂S, found [M+H]⁺=385.2.

### 1.4 tert-butyl (4-((2-butyl-1H-imidazo[4,5-d]thieno[2,3-b]pyridin-1-yl)methyl)benzyl) carbamate (Compound 1.d)

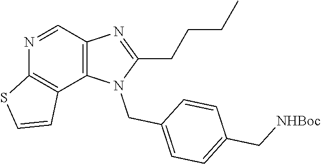 [SMILES: CCCCc1nc2cnc3sccc3c2n1Cc1ccc(CNC(=O)OC(C)(C)C)cc1]

The title compound was prepared according to General Procedure 3 starting from Compound 1.c (140 mg). Purification was accomplished as described in General Procedure 11, eluting with a 0 to 10% DCM/MeOH+1% NH₄OH gradient to give Compound 1.d as an off-white solid (132 mg, 82% yield).

LC/MS: Calc'd m/z=450.2 for C₂₅H₃₀N₄O₂S, found [M+H]⁺=451.4.

### 1.5 tert-butyl (4-((4-amino-2-butyl-1H-imidazo[4,5-d]thieno[2,3-b]pyridin-1-yl)methyl) benzyl)carbamate (Compound 1.e)

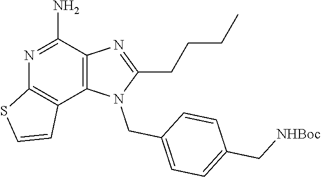 [SMILES: CCCCc1nc2c(N)nc3sccc3c2n1Cc1ccc(CNC(=O)OC(C)(C)C)cc1]

The title compound was prepared according to General Procedure 4 starting from Compound 1.d (60 mg) using 1,2-dichloroethane as the solvent. The crude product was taken to the next step with no additional purification.

LC/MS: Calc'd m/z=465.2 for C₂₅H₃₁N₅O₂S, found [M+H]⁺=466.2.

### 1.6 1-(4-(aminomethyl)benzyl)-2-butyl-1H-imidazo[4,5-d]thieno[2,3-b]pyridin-4-amine (Compound 112)

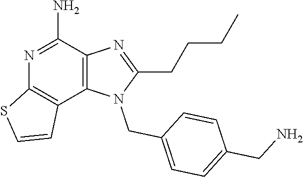 [SMILES: CCCCc1nc2c(N)nc3sccc3c2n1Cc1ccc(CN)cc1]

The title compound was prepared according to General Procedure 5 starting from Compound 1.e (60 mg). Purification was accomplished by preparative HPLC as described in General Procedure 11, eluting with a 0 to 40% CH₃CN/H₂O+0.1% TFA gradient to give Compound 112 as a white solid (4.5 mg, 6% yield, assumed 2×TFA salt).

LC/MS: Calc'd m/z=365.2 for C₂₅H₃₁N₅O₂S, found [M+H]⁺=366.2. ¹H NMR (300 MHz, methanol-d4) δ 7.47 (d, J=8.2 Hz, 2H), 7.36 (d, J=5.9 Hz, 1H), 7.31 (d, J=5.9 Hz, 1H), 7.18 (d, J=8.0 Hz, 2H), 5.86 (s, 2H), 4.11 (s, 2H), 3.03-2.92 (m, 2H), 1.84 (p, J=7.5 Hz, 2H), 1.47 (h, J=7.4 Hz, 2H), 0.96 (t, J=7.4 Hz, 3H).

### Example 2: Preparation of Series 2 Compounds

### 2.1 7-chloro-6-nitrothieno[3,2-b]pyridine (Compound 2.a)

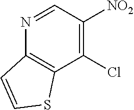 [SMILES: O=[N+]([O-])c1cnc2ccsc2c1Cl]

The title compound was prepared according to the procedure described in U.S. Patent Application Publication No. US 2014/0121198A1.

### 2.2 tert-butyl (4-(((6-nitrothieno[3,2-b]pyridin-7-yl)amino)methyl)benzyl)carbamate (Compound 2.b)

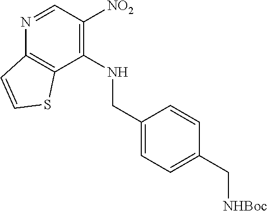 [SMILES: CC(C)(C)OC(=O)NCc1ccc(CNc2c([N+](=O)[O-])cnc3ccsc23)cc1]

The title compound was prepared according to General Procedure 1 starting from Compound 2.a (330 mg) and tert-butyl (4-(aminomethyl)benzyl)carbamate. Crude Compound 2.b was taken to the next step with no additional purification.

LC/MS: Calc'd m/z=414.1 for C₂₀H₂₂N₄O₄S, found [M+H]⁺=415.1.

### 2.3 tert-butyl (4-(((6-aminothieno[3,2-b]pyridin-7-yl)amino)methyl)benzyl)carbamate (Compound 2.c)

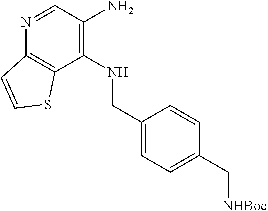 [SMILES: CC(C)(C)OC(=O)NCc1ccc(CNc2c(N)cnc3ccsc23)cc1]

The title compound was prepared according to General Procedure 2 starting from crude Compound 2.b. Purification was accomplished as described in General Procedure 11, eluting with a 10 to 50% CH₃CN/H₂O+0.1% TFA gradient to give Compound 2.c as a yellow solid (449 mg, 48% yield over two steps, assumed 2×TFA salt).

LC/MS: Calc'd m/z=384.2 for C₂₀H₂₄N₄O₂S, found [M+H]⁺=385.2.

### 2.4 tert-butyl (4-((2-butyl-1H-imidazo[4,5-d]thieno[3,2-b]pyridin-1-yl)methyl)benzyl) carbamate (Compound 2.d)

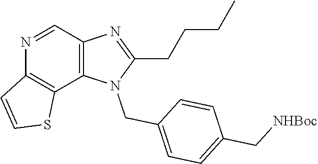 [SMILES: CCCCc1nc2cnc3ccsc3c2n1Cc1ccc(CNC(=O)OC(C)(C)C)cc1]

The title compound was prepared according to General Procedure 3 starting from Compound 2.c (180 mg). Purification was accomplished as described in General Procedure 11, eluting with a 10 to 50% CH₃CN/H₂O+0.1% TFA gradient to give Compound 2.d as an off-white solid (120 mg, 72% yield, assumed 1×TFA salt).

LC/MS: Calc'd m/z=450.2 for C₂₅H₃₀N₄O₂S, found [M+H]⁺=451.4.

### 2.5 tert-butyl (4-((4-amino-2-butyl-1H-imidazo[4,5-d]thieno[3,2-b]pyridin-1-yl)methyl) benzyl)carbamate (Compound 2.e)

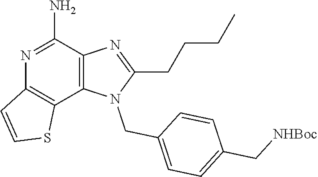 [SMILES: CCCCc1nc2c(N)nc3ccsc3c2n1Cc1ccc(CNC(=O)OC(C)(C)C)cc1]

The title compound was prepared according to General Procedure 4 starting from Compound 2.d (60 mg) using 1,2-dichloroethane as the solvent. The crude product was taken to the next step with no additional purification.

LC/MS: Calc'd m/z=465.2 for C₂₅H₃₁N₅O₂S, found [M+H]⁺=466.2.

### 2.6 1-(4-(aminomethyl)benzyl)-2-butyl-1H-imidazo[4,5-d]thieno[2,3-b]pyridin-4-amine (Compound 111)

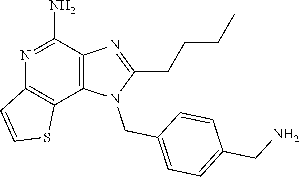 [SMILES: CCCCc1nc2c(N)nc3ccsc3c2n1Cc1ccc(CN)cc1]

The title compound was prepared according to General Procedure 5 starting from crude Compound 2.e. Purification was accomplished by preparative HPLC as described in General Procedure 11, eluting with a 0 to 40% CH₃CN/H₂O+0.1% TFA gradient to give Compound 111 as a white solid (8.6 mg, 14% yield over two steps, assumed 2×TFA salt).

LC/MS: Calc'd m/z=365.2 for C₂₅H₃₁N₅O₂S, found [M+H]⁺=366.2. ¹H NMR (300 MHz, methanol-d4) δ 7.90 (d, J=5.5 Hz, 1H), 7.48 (d, J=8.1 Hz, 2H), 7.45 (d, J=5.4 Hz, 1H), 7.22 (d, J=8.0 Hz, 2H), 5.78 (s, 2H), 4.12 (s, 2H), 2.96 (t, J=7.6 Hz, 2H), 1.85 (p, J=7.5 Hz, 2H), 1.47 (h, J=7.4 Hz, 2H), 0.96 (t, J=7.3 Hz, 3H).

### 2.7 (4-(((6-nitrothieno[3,2-b]pyridin-7-yl)amino)methyl)phenyl)methanol (Compound 2.f)

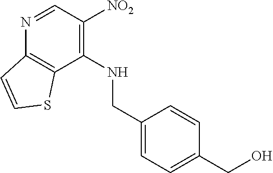 [SMILES: O=[N+]([O-])c1cnc2ccsc2c1NCc1ccc(CO)cc1]

The title compound was prepared according to General Procedure 1 starting from Compound 2.a (3.30 g) and (4-(aminomethyl)phenyl)methanol. The crude compound was taken to the next step with no additional purification.

LC/MS: Calc'd m/z=315.1 for C₁₅H₁₃N₃O₃S, found [M+H]⁺=316.1.

### 2.8 (4-(((6-aminothieno[3,2-b]pyridin-7-yl)amino)methyl)phenyl)methanol (Compound 2.g)

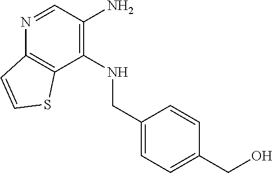 [SMILES: Nc1cnc2ccsc2c1NCc1ccc(CO)cc1]

The title compound was prepared according General Procedure 2 starting from crude Compound 2.f. Purification was accomplished as described in General Procedure 11, eluting with a 5 to 40% CH₃CN/H₂O+0.1% TFA gradient to give Compound 2.g as a yellow solid (3.10 g, 39% yield over two steps, assumed 2×TFA salt).

LC/MS: Calc'd m/z=285.1 for C₁₅H₁₅N₃OS, found [M+H]⁺=286.2.

### 2.9 (4-((2-butyl-1H-imidazo[4,5-d]thieno[3,2-b]pyridin-1-yl)methyl)phenyl)methanol (Compound 2.h)

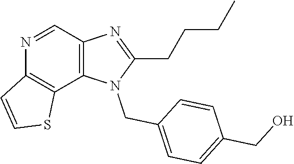 [SMILES: CCCCc1nc2cnc3ccsc3c2n1Cc1ccc(CO)cc1]

The title compound was prepared according to General Procedure 7 starting from Compound 2.g (1.00 g). Purification was accomplished as described in General Procedure 11, eluting with a 0 to 10% DCM/MeOH+1% NEt₃ gradient to give Compound 2.h as an off-white solid (640 mg, 94% yield).

LC/MS: Calc'd m/z=351.1 for C₂₀H₂₁N₃OS, found [M+H]⁺=352.2.

### 2.10 (4-((4-amino-2-butyl-1H-imidazo[4,5-d]thieno[3,2-b]pyridin-1-yl)methyl)phenyl) methanol (Compound 2.i)

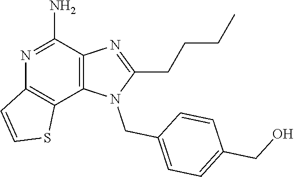 [SMILES: CCCCc1nc2c(N)nc3ccsc3c2n1Cc1ccc(CO)cc1]

The title compound was prepared according to General Procedure 4 starting from Compound 2.h (640 mg) using chloroform as the solvent. Purification was accomplished as described in General Procedure 11, eluting with a 10 to 50% CH₃CN/H₂O+0.1% TFA gradient to give Compound 2.i as an off-white solid (230 mg, 26% yield, assumed 1×TFA salt).

LC/MS: Calc'd m/z=366.2 for C₂₀H₂₂N₄OS, found [M+H]⁺=367.2. ¹H NMR (300 MHz, methanol-d₄) δ 7.91 (d, J=5.4 Hz, 1H), 7.43 (d, J=5.5 Hz, 1H), 7.37 (d, J=8.0 Hz, 2H), 7.11 (d, J=8.0 Hz, 2H), 5.73 (s, 2H), 4.60 (s, 2H), 3.02-2.91 (m, 2H), 1.83 (p, J=7.6 Hz, 2H), 1.46 (h, J=7.3 Hz, 2H), 0.95 (t, J=7.4 Hz, 3H).

### 2.11 2-butyl-1-(4-(chloromethyl)benzyl)-1H-imidazo[4,5-d]thieno[3,2-b]pyridin-4-amine (Compound 2.j)

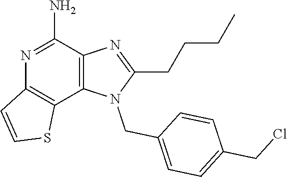 [SMILES: CCCCc1nc2c(N)nc3ccsc3c2n1Cc1ccc(CCl)cc1]

The title compound was prepared according to General Procedure 8 starting from Compound 2.i (230 mg). The crude compound was taken to the next step with no additional purification.

LC/MS: Calc'd m/z=384.1 for C₂₀H₂₁ClN₄S, found [M+H]⁺=385.2.

### 2.12 2-butyl-1-(4-((diethylamino)methyl)benzyl)-1H-imidazo[4,5-d]thieno[3,2-b]pyridin-4-amine (Compound 144)

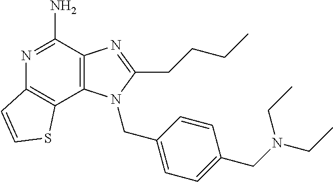 [SMILES: CCCCc1nc2c(N)nc3ccsc3c2n1Cc1ccc(CN(CC)CC)cc1]

The title compound was prepared according to General Procedure 9 using Compound 2.j (10 mg), DMF and diethylamine. Purification was accomplished by preparative HPLC as described in General Procedure 11, eluting with a 10 to 40% CH₃CN/H₂O+0.1% TFA gradient to give Compound 144 as a white solid (9.0 mg, 53% yield, assumed 2×TFA salt).

LC/MS: Calc'd m/z=421.2 for C₂₄H₃₁N₅S, found [M+H]⁺=422.2. ¹H NMR (300 MHz, methanol-d₄) δ 7.90 (d, J=5.5 Hz, 1H), 7.55 (d, J=8.3 Hz, 2H), 7.46 (d, J=5.4 Hz, 1H), 7.27 (d, J=8.2 Hz, 2H), 5.80 (s, 2H), 4.34 (s, 2H), 3.19 (q, J=7.2 Hz, 4H), 3.03-2.92 (m, 2H), 1.85 (p, J=7.5 Hz, 2H), 1.56-1.40 (m, 2H), 1.33 (t, J=7.3 Hz, 6H), 0.95 (t, J=7.3 Hz, 3H).

### 2.13 1-(4-((benzylamino)methyl)benzyl)-2-butyl-1H-imidazo[4,5-d]thieno[3,2-b]pyridin-4-amine (Compound 145)

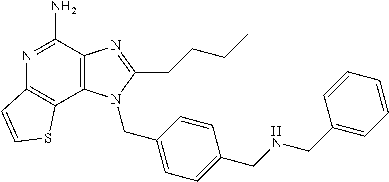 [SMILES: CCCCc1nc2c(N)nc3ccsc3c2n1Cc1ccc(CNCc2ccccc2)cc1]

The title compound was prepared according to General Procedure 9 using Compound 2.j (10 mg), DMF and benzylamine. Purification was accomplished by preparative HPLC as described in General Procedure 11, eluting with a 10 to 40% CH₃CN/H₂O+0.1% TFA gradient to give Compound 145 as a white solid (8.7 mg, 49% yield, assumed 2×TFA salt).

LC/MS: Calc'd m/z=455.2 for C₂₇H₂₉N₅S, found [M+H]⁺=456.2. ¹H NMR (300 MHz, methanol-d₄) δ 7.90 (d, J=5.5 Hz, 1H), 7.51 (d, J=8.2 Hz, 2H), 7.47-7.45 (m, 5H), 7.45 (d, J=5.5 Hz, 1H), 7.23 (d, J=8.2 Hz, 2H), 5.78 (s, 2H), 4.24 (s, 2H), 4.23 (s, 2H), 3.02-2.91 (m, 2H), 1.85 (p, J=7.5 Hz, 2H), 1.47 (h, J=7.4 Hz, 2H), 0.96 (t, J=7.4 Hz, 3H).

### 2.14 2-butyl-1-(4-((pentylamino)methyl)benzyl)-1H-imidazo[4,5-d]thieno[3,2-b]pyridin-4-amine (Compound 146)

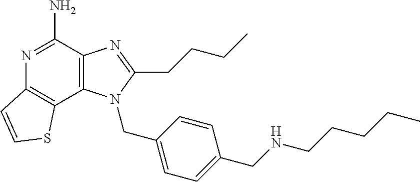 [SMILES: CCCCCNCc1ccc(Cn2c(CCCC)nc3c(N)nc4ccsc4c32)cc1]

The title compound was prepared according to General Procedure 9 using Compound 2.j (10 mg), DMF and pentylamine. Purification was accomplished by preparative HPLC as described in General Procedure 11, eluting with a 10 to 40% CH₃CN/H₂O+0.1% TFA gradient to give Compound 146 as a white solid (7.9 mg, 46% yield, assumed 2×TFA salt).

LC/MS: Calc'd m/z=435.2 for C₂₅H₃₃N₅S, found [M+H]⁺=436.2. ¹H NMR (300 MHz, methanol-d₄) δ 7.90 (d, J=5.5 Hz, 1H), 7.51 (d, J=8.2 Hz, 2H), 7.45 (d, J=5.4 Hz, 1H), 7.23 (d, J=8.1 Hz, 2H), 5.78 (s, 2H), 4.19 (s, 2H), 3.04-2.93 (m, 4H), 1.85 (p, J=7.6 Hz, 2H), 1.69 (m, 2H), 1.56-1.35 (m, 6H), 0.95 (m, 6H).

### 2.15 5-((4-((4-amino-2-butyl-1H-imidazo[4,5-d]thieno[3,2-b]pyridin-1-yl)methyl)benzyl) amino)pentan-1-ol (Compound 147)

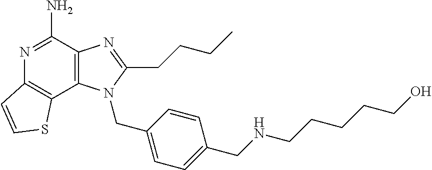 [SMILES: CCCCc1nc2c(N)nc3ccsc3c2n1Cc1ccc(CNCCCCCO)cc1]

The title compound was prepared according to General Procedure 9 using Compound 2.j (10 mg) DMF and 5-aminopentan-1-ol. Purification was accomplished by preparative HPLC as described in General Procedure 11, eluting with a 10 to 40% CH₃CN/H₂O+0.1% TFA gradient to give Compound 147 as a white solid (5.9 mg, 33% yield, assumed 2×TFA salt).

LC/MS: Calc'd m/z=451.2 for C₂₅H₃₃N₅OS, found [M+H]⁺=452.2. ¹H NMR (300 MHz, methanol-d₄) δ 7.90 (d, J=5.5 Hz, 1H), 7.56-7.48 (m, 2H), 7.45 (d, J=5.5 Hz, 1H), 7.23 (d, J=8.1 Hz, 2H), 5.78 (s, 2H), 4.20 (s, 2H), 3.57 (t, J=6.2 Hz, 2H), 3.12-3.00 (m, 2H), 2.97 (t, J=7.6 Hz, 2H), 1.85 (p, J=7.5 Hz, 2H), 1.72 (p, J=7.6 Hz, 2H), 1.64-1.34 (m, 6H), 0.96 (t, J=7.3 Hz, 3H).

### 2.16 2-((4-((4-amino-2-butyl-1H-imidazo[4,5-d]thieno[3,2-b]pyridin-1-yl)methyl) benzyl)amino)ethan-1-ol (Compound 148)

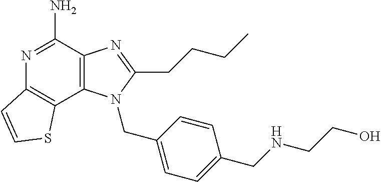 [SMILES: CCCCc1nc2c(N)nc3ccsc3c2n1Cc1ccc(CNCCO)cc1]

The title compound was prepared according to General Procedure 9 using Compound 2.j (10 mg), DMF and ethanolamine. Purification was accomplished by preparative HPLC as described in General Procedure 11, eluting with a 10 to 40% CH₃CN/H₂O+0.1% TFA gradient to give Compound 148 as a white solid (5.9 mg, 33% yield, assumed 2×TFA salt).

LC/MS: Calc'd m/z=409.2 for C₂₂H₂₇N₅OS, found [M+H]⁺=410.2. ¹H NMR (300 MHz, methanol-d₄) δ 7.90 (d, J=5.4 Hz, 1H), 7.53 (d, J=8.2 Hz, 2H), 7.45 (d, J=5.4 Hz, 1H), 7.23 (d, J=8.1 Hz, 2H), 5.78 (s, 2H), 4.24 (s, 2H), 3.85-3.75 (m, 2H), 3.17-3.07 (m, 2H), 3.02-2.91 (m, 2H), 1.85 (p, J=7.6 Hz, 2H), 1.56-1.38 (m, 2H), 0.96 (t, J=7.4 Hz, 3H).

### 2.17 1-(4-((((1H-pyrrol-3-yl)methyl)amino)methyl)benzyl)-2-butyl-1H-imidazo[4,5-d]thieno[3,2-b]pyridin-4-amine (Compound 149)

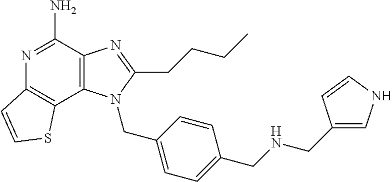 [SMILES: CCCCc1nc2c(N)nc3ccsc3c2n1Cc1ccc(CNCc2cc[nH]c2)cc1]

The title compound was prepared according to General Procedure 9 using Compound 2.j (10 mg), DMF and (1H-pyrrol-3-yl)methanamine. Purification was accomplished by preparative HPLC as described in General Procedure 11, eluting with a 10 to 40% CH₃CN/H₂O+0.1% TFA gradient to give Compound 149 as a white solid (4.4 mg, 25% yield, assumed 2×TFA salt).

LC/MS: Calc'd m/z=444.2 for C₂₅H₂₈N₆S, found [M+H]⁺=445.2. ¹H NMR (300 MHz, methanol-d₄) δ 10.65 (s, 1H), 7.90 (d, J=5.5 Hz, 1H), 7.47 (d, J=8.2 Hz, 2H), 7.45 (d, J=5.5 Hz, 1H), 7.22 (d, J=8.0 Hz, 2H), 6.92 (s, 1H), 6.81 (d, J=2.6 Hz, 1H), 6.22 (d, J=2.9 Hz, 1H), 5.78 (s, 2H), 4.15 (s, 2H), 4.10 (s, 2H), 2.96 (t, J=7.6 Hz, 2H), 1.85 (p, J=7.6 Hz, 2H), 1.47 (h, J=7.3 Hz, 2H), 0.96 (t, J=7.4 Hz, 3H).

### 2.18 2-butyl-1-(4-((methylamino)methyl)benzyl)-1H-imidazo[4,5-d]thieno[3,2-b]pyridin-4-amine (Compound 150)

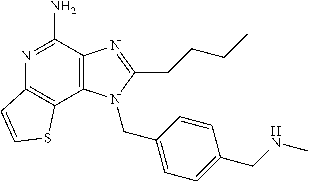 [SMILES: CCCCc1nc2c(N)nc3ccsc3c2n1Cc1ccc(CNC)cc1]

The title compound was prepared according to General Procedure 9 using Compound 2.j (10 mg), DMF and methylamine hydrochloride. Purification was accomplished by preparative HPLC as described in General Procedure 11, eluting with a 10 to 40% CH₃CN/H₂O+0.1% TFA gradient to give Compound 150 as a white solid (4.4 mg, 25% yield, assumed 2×TFA salt).

LC/MS: Calc'd m/z=379.2 for C₂₁H₂₅N₅S, found [M+H]⁺=380.2. ¹H NMR (300 MHz, methanol-d₄) δ 7.90 (d, J=5.5 Hz, 1H), 7.50 (d, J=8.1 Hz, 2H), 7.44 (d, J=5.4 Hz, 1H), 7.24 (d, J=8.0 Hz, 2H), 5.79 (s, 2H), 4.18 (s, 2H), 2.96 (t, J=7.6 Hz, 2H), 2.71 (s, 3H), 1.85 (p, J=7.6 Hz, 2H), 1.45 (h, J=7.3 Hz, 2H), 0.96 (t, J=7.4 Hz, 3H).

### 2.19 2-butyl-1-(4-(piperidin-1-ylmethyl)benzyl)-1H-imidazo[4,5-d]thieno[3,2-b]pyridin-4-amine (Compound 151)

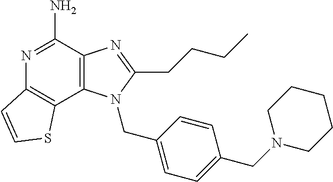 [SMILES: CCCCc1nc2c(N)nc3ccsc3c2n1Cc1ccc(CN2CCCCC2)cc1]

The title compound was prepared according to General Procedure 9 using Compound 2.j (10 mg) DMF and piperidine. Purification was accomplished by preparative HPLC as described in General Procedure 11, eluting with a 10 to 40% CH₃CN/H₂O+0.1% TFA gradient to give Compound 151 as a white solid (9.9 mg, 58% yield, assumed 2×TFA salt).

LC/MS: Calc'd m/z=433.2 for C₂₅H₃₁N₅S, found [M+H]⁺=434.2. ¹H NMR (300 MHz, methanol-d₄) δ 7.90 (d, J=5.5 Hz, 1H), 7.53 (d, J=8.2 Hz, 2H), 7.45 (d, J=5.5 Hz, 1H), 7.26 (d, J=8.1 Hz, 2H), 5.79 (s, 2H), 4.29 (s, 2H), 3.47-3.37 (m, 2H), 3.02-2.87 (m, 4H), 1.99-1.63 (m, 7H), 1.58-1.37 (m, 3H), 0.95 (t, J=7.4 Hz, 3H).

### 2.20 N¹-(4-((4-amino-2-butyl-1H-imidazo[4,5-d]thieno[3,2-b]pyridin-1-yl)methyl)benzyl)-N²,N²-dibutyl-N¹-(2-(dibutylamino)ethyl)ethane-1,2-diamine (Compound 152)

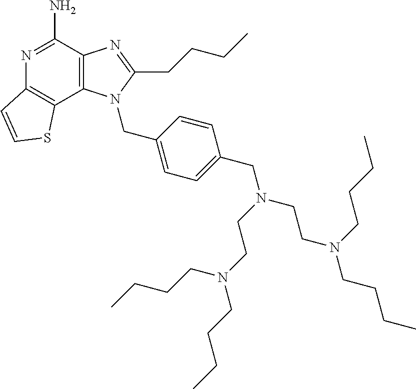 [SMILES: CCCCc1nc2c(N)nc3ccsc3c2n1Cc1ccc(CN(CCN(CCCC)CCCC)CCN(CCCC)CCCC)cc1]

The title compound was prepared according to General Procedure 9 using Compound 2.j (10 mg), DMF and N¹,N¹-dibutyl-N²-(2-(dibutylamino)ethyl)ethane-1,2-diamine. Purification was accomplished by preparative HPLC as described in General Procedure 11, eluting with a 10 to 40% CH₃CN/H₂O+0.1% TFA gradient to give Compound 152 as a white solid (4.9 mg, 17% yield, assumed 4×TFA salt).

LC/MS: Calc'd m/z=675.5 for C₄₀H₆₅N₇S, found [M+H]⁺=676.5. ¹H NMR (300 MHz, methanol-d₄) δ 7.93 (d, J=5.5 Hz, 1H), 7.47 (d, J=5.5 Hz, 1H), J=7.46 (d, J=8.1 Hz, 2H), 7.18 (d, J=8.0 Hz, 2H), 5.73 (s, 2H), 3.73 (s, 2H), 3.11-2.86 (m, 16H), 1.88 (p, J=7.6 Hz, 2H), 1.69-1.56 (m, 8H), 1.53-1.42 (m, 4H), 1.40-1.25 (m, 8H), 1.08-0.90 (m, 15H).

### 2.21 tert-butyl 4-(4-((4-amino-2-butyl-1H-imidazo[4,5-d]thieno[3,2-b]pyridin-1-yl)methyl)benzyl)piperazine-1-carboxylate (Compound 153)

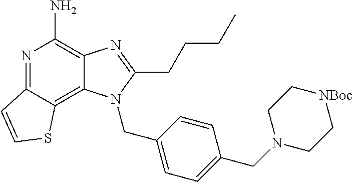 [SMILES: CCCCc1nc2c(N)nc3ccsc3c2n1Cc1ccc(CN2CCN(C(=O)OC(C)(C)C)CC2)cc1]

The title compound was prepared according to General Procedure 9 using Compound 2.j (10 mg), DMF and tert-butyl piperazine-1-carboxylate. Purification was accomplished by preparative HPLC as described in General Procedure 11, eluting with a 10 to 40% CH₃CN/H₂O+0.1% TFA gradient to give Compound 153 as a white solid (14 mg, 77% yield, assumed 2×TFA salt).

LC/MS: Calc'd m/z=534.3 for C₂₉H₃₈N₆O₂S, found [M+H]⁺=535.4. ¹H NMR (300 MHz, acetonitrile-d₃) δ 7.82 (d, J=5.5 Hz, 1H), 7.49-7.40 (m, 3H), 7.20 (d, J=8.1 Hz, 2H), 5.67 (s, 2H), 4.24 (s, 2H), 2.93-2.84 (m, 2H), 1.74 (p, J=7.5 Hz, 2H), 1.43 (s, 9H), 1.44-1.33 (m, 2H), 0.89 (t, J=7.3 Hz, 3H).

### 2.22 2-butyl-1-(4-(piperazin-1-ylmethyl)benzyl)-1H-imidazo[4,5-d]thieno[3,2-b]pyridin-4-amine (Compound 154)

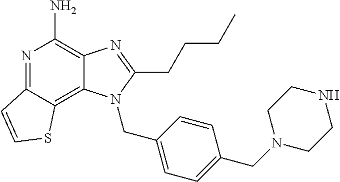 [SMILES: CCCCc1nc2c(N)nc3ccsc3c2n1Cc1ccc(CN2CCNCC2)cc1]

The title compound was prepared according to General Procedure 5 using Compound 153 (13.1 mg). Purification was accomplished by preparative HPLC as described in General Procedure 11, eluting with a 10 to 40% CH₃CN/H₂O+0.1% TFA gradient to give Compound 154 as a white solid (10.1 mg, 72% yield, assumed 3×TFA salt).

LC/MS: Calc'd m/z=434.2 for C₂₄H₃₀N₆S, found [M+H]⁺=435.4. ¹H NMR (300 MHz, acetonitrile-d₃) δ 7.80 (d, J=5.5 Hz, 1H), 7.48-7.39 (m, 3H), 7.16 (d, J=8.0 Hz, 2H), 5.64 (s, 2H), 4.13 (s, 2H), 3.40 (dd, J=6.9, 3.9 Hz, 4H), 2.96-2.79 (m, 2H), 1.75 (p, J=7.6 Hz, 2H), 1.49-1.31 (m, 2H), 0.90 (t, J=7.3 Hz, 3H).

### 2.23 tert-butyl (4-(((4-((4-amino-2-butyl-1H-imidazo[4,5-d]thieno[3,2-b]pyridin-1-yl)methyl)benzyl)amino)methyl)benzyl)carbamate (Compound 155)

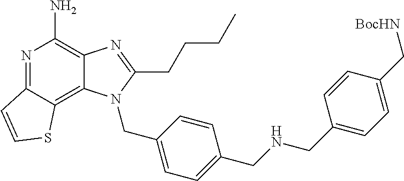 [SMILES: CCCCc1nc2c(N)nc3ccsc3c2n1Cc1ccc(CNCc2ccc(CNC(=O)OC(C)(C)C)cc2)cc1]

The title compound was prepared according to General Procedure 9 using Compound 2.j (10 mg), DMF and tert-butyl (4-(aminomethyl)benzyl)carbamate. Purification was accomplished by preparative HPLC as described in General Procedure 11, eluting with a 10 to 40% CH₃CN/H₂O+0.1% TFA gradient to give Compound 155 as a white solid (15.2 mg, 84% yield, assumed 2×TFA salt).

LC/MS: Calc'd m/z=584.3 for C₃₃H₄₀N₆O₂S, found [M+H]⁺=585.4. ¹H NMR (300 MHz, acetonitrile-d₃) δ 7.79 (d, J=5.5 Hz, 1H), 7.43 (d, J=9.6 Hz, 2H), 7.41 (d, J=3.1 Hz, 1H), 7.36 (d, J=8.2 Hz, 2H), 7.30 (d, J=8.1 Hz, 2H), 7.15 (d, J=8.1 Hz, 2H), 5.64 (s, 2H), 4.21 (s, 2H), 4.15 (s, 2H), 4.11 (s, 2H), 2.96-2.83 (m, 2H), 1.75 (p, J=7.4 Hz, 2H), 1.40 (s, 9H), 1.47-1.33 (m, 2H), 0.90 (t, J=7.3 Hz, 3H).

### 2.24 1-(4-(((4-(aminomethyl)benzyl)amino)methyl)benzyl)-2-butyl-1H-imidazo[4,5-d]thieno[3,2-b]pyridin-4-amine (Compound 156)

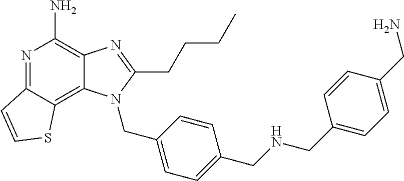 [SMILES: CCCCc1nc2c(N)nc3ccsc3c2n1Cc1ccc(CNCc2ccc(CN)cc2)cc1]

The title compound was prepared according to General Procedure 5 using Compound 155 (13.0 mg). Purification was accomplished by preparative HPLC as described in General Procedure 11, eluting with a 10 to 40% CH₃CN/H₂O+0.1% TFA gradient to give Compound 156 as a white solid (11.4 mg, 84% yield, assumed 3×TFA salt).

LC/MS: Calc'd m/z=484.2 for C₂₈H₃₂N₆S, found [M+H]⁺=485.2. ¹H NMR (300 MHz, acetonitrile-d₃) δ 7.80 (d, J=5.4 Hz, 1H), 7.49-7.40 (m, 3H), 7.18 (d, J=7.9 Hz, 2H), 5.66 (s, 2H), 4.22 (s, 2H), 3.94-3.85 (m, 4H), 2.89 (t, J=7.6 Hz, 2H), 2.77 (d, J=5.9 Hz, 2H), 1.83-1.67 (m, 2H), 1.40 (h, J=7.4 Hz, 2H), 0.90 (t, J=7.3 Hz, 3H).

### 2.25 2-butyl-1-(4-((4-(2-morpholinoethyl)piperazin-1-yl)methyl)benzyl)-1H-imidazo[4,5-d]thieno[3,2-b]pyridin-4-amine (Compound 157)

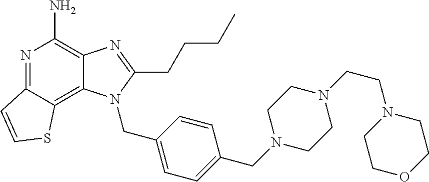 [SMILES: CCCCc1nc2c(N)nc3ccsc3c2n1Cc1ccc(CN2CCN(CCN3CCOCC3)CC2)cc1]

The title compound was prepared according to General Procedure 9 using Compound 2.j (10 mg), DMF and 4-(2-(piperazin-1-yl)ethyl)morpholine. Purification was accomplished by preparative HPLC as described in General Procedure 11, eluting with a 10 to 40% CH₃CN/H₂O+0.1% TFA gradient to give Compound 157 as a white solid (5.0 mg, 23% yield, assumed 4×TFA salt).

LC/MS: Calc'd m/z=547.31 for C₃₀H₄₁N₇OS, found [M+H]⁺=548.4. ¹H NMR (300 MHz, acetonitrile-d₃) δ 7.80 (d, J=5.4 Hz, 1H), 7.49-7.40 (m, 3H), 7.18 (d, J=7.9 Hz, 2H), 5.66 (s, 2H), 4.22 (s, 2H), 3.94-3.85 (m, 4H), 3.28 (d, J=4.7 Hz, 4H), 2.89 (t, J=7.6 Hz, 2H), 2.77 (d, J=11.8 Hz, 2H), 1.83-1.67 (m, 2H), 1.40 (h, J=7.4 Hz, 2H), 0.90 (t, J=7.3 Hz, 3H).

### 2.26 2-butyl-1-(4-((4-(pyridin-4-yl)piperidin-1-yl)methyl)benzyl)-1H-imidazo[4,5-d]thieno[3,2-b]pyridin-4-amine (Compound 158)

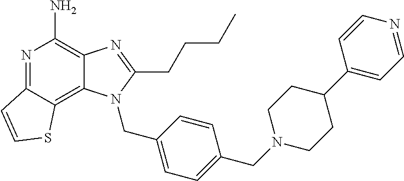 [SMILES: CCCCc1nc2c(N)nc3ccsc3c2n1Cc1ccc(CN2CCC(c3ccncc3)CC2)cc1]

The title compound was prepared according to General Procedure 9 using Compound 2.j (10 mg), DMF and 4-(piperidin-4-yl)pyridine. Purification was accomplished by preparative HPLC as described in General Procedure 11, eluting with a 10 to 40% CH₃CN/H₂O+0.1% TFA gradient to give Compound 158 as a white solid (12.3 mg, 60% yield, assumed 3×TFA salt).

LC/MS: Calc'd m/z=510.3 for C₃₀H₃₄N₆S, found [M+H]⁺=511.4. ¹H NMR (300 MHz, acetonitrile-d₃) δ 8.67 (d, J=6.0 Hz, 2H), 7.88 (d, J=6.2 Hz, 2H), 7.80 (d, J=5.5 Hz, 1H), 7.50 (d, J=8.2 Hz, 2H), 7.44 (d, J=5.5 Hz, 1H), 7.20 (d, J=8.0 Hz, 2H), 5.66 (s, 2H), 4.28 (s, 2H), 3.50 (d, J=12.3 Hz, 2H), 3.10-2.99 (m, 2H), 2.95-2.81 (m, 2H), 2.18-2.07 (m, 3H), 1.75 (qd, J=8.5, 7.7, 6.4 Hz, 2H), 1.40 (h, J=7.3 Hz, 2H), 0.90 (t, J=7.3 Hz, 3H).

### 2.27 2-butyl-1-(4-(((2-thiomorpholinoethyl)amino)methyl)benzyl)-1H-imidazo[4,5-d]thieno [3,2-b]pyridin-4-amine (Compound 159)

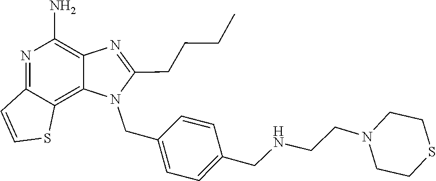 [SMILES: CCCCc1nc2c(N)nc3ccsc3c2n1Cc1ccc(CNCCN2CCSCC2)cc1]

The title compound was prepared according to General Procedure 9 using Compound 2.j (10 mg), DMF and 2-thiomorpholinoethan-1-amine. Purification was accomplished by preparative HPLC as described in General Procedure 11, eluting with a 10 to 40% CH₃CN/H₂O+0.1% TFA gradient to give Compound 159 as a white solid (11.7 mg, 58% yield, assumed 3×TFA salt).

LC/MS: Calc'd m/z=494.2 for C₂₆H₃₄N₆S₂, found [M+H]⁺=495.4. ¹H NMR (300 MHz, acetonitrile-d₃) δ 7.80 (d, J=5.5 Hz, 1H), 7.51-7.39 (m, 3H), 7.15 (d, J=8.0 Hz, 2H), 5.64 (s, 2H), 4.18 (s, 2H), 3.47-3.30 (m, 8H), 2.94-2.83 (m, 6H), 1.76 (p, J=7.5 Hz, 2H), 1.48-1.34 (m, 2H), 0.91 (t, J=7.3 Hz, 3H).

### 2.28 tert-butyl (2-((4-((4-amino-2-butyl-1H-imidazo[4,5-d]thieno[3,2-b]pyridin-1-yl)methyl) benzyl)amino)ethyl)carbamate (Compound 160)

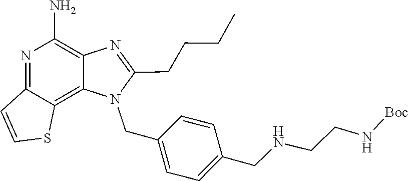 [SMILES: CCCCc1nc2c(N)nc3ccsc3c2n1Cc1ccc(CNCCNC(=O)OC(C)(C)C)cc1]

The title compound was prepared according to General Procedure 9 using Compound 2.j (10 mg), DMF and tert-butyl (2-aminoethyl)carbamate. Purification was accomplished by preparative HPLC as described in General Procedure 11, eluting with a 10 to 40% CH₃CN/H₂O+0.1% TFA gradient to give Compound 160 as a white solid (11.8 mg, 71% yield, assumed 2×TFA salt). LC/MS: Calc'd m/z=508.3 for C₂₇H₃₆N₆O₂S, found [M+H]⁺=509.4. ¹H NMR (300 MHz, acetonitrile-d₃) δ 7.80 (d, J=5.5 Hz, 1H), 7.48-7.39 (m, 3H), 7.16 (d, J=8.0 Hz, 2H), 5.64 (s, 2H), 4.15 (s, 2H), 3.31 (t, J=5.7 Hz, 2H), 3.05 (t, J=5.7 Hz, 2H), 2.95-2.84 (m, 2H), 1.75 (p, J=7.4 Hz, 2H), 1.47-1.32 (m, 2H), 1.37 (s, 9H), 0.90 (t, J=7.3 Hz, 3H).

### 2.29 N¹-(4-((4-amino-2-butyl-1H-imidazo[4,5-d]thieno[3,2-b]pyridin-1-yl)methyl)benzyl) ethane-1,2-diamine (Compound 161)

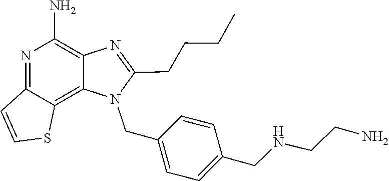 [SMILES: CCCCc1nc2c(N)nc3ccsc3c2n1Cc1ccc(CNCCN)cc1]

The title compound was prepared according to General Procedure 5 using Compound 160 (10 mg). Purification was accomplished by preparative HPLC as described in General Procedure 11, eluting with a 10 to 40% CH₃CN/H₂O+0.1% TFA gradient to give Compound 161 as a white solid (8.9 mg, 86% yield, assumed 2×TFA salt).

LC/MS: Calc'd m/z=408.2 for C₂₂H₂₈N₆S, found [M+H]⁺=409.2. ¹H NMR (300 MHz, acetonitrile-d₃) δ 7.80 (d, J=5.5 Hz, 1H), 7.47 (d, J=8.3 Hz, 2H), 7.43 (d, J=5.5 Hz, 1H), 7.17 (d, J=8.0 Hz, 2H), 5.65 (s, 2H), 4.19 (s, 2H), 2.95-2.85 (m, 2H), 2.84 (s, 2H), 1.83-1.67 (m, 2H), 1.41 (h, J=7.3 Hz, 2H), 0.90 (t, J=7.3 Hz, 3H).

### 2.30 1-((4-((4-amino-2-butyl-1H-imidazo[4,5-d]thieno[3,2-b]pyridin-1-yl)methyl)benzyl) amino)-2-methylpropan-2-ol (Compound 162)

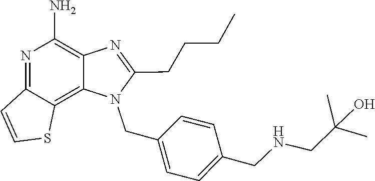 [SMILES: CCCCc1nc2c(N)nc3ccsc3c2n1Cc1ccc(CNCC(C)(C)O)cc1]

The title compound was prepared according to General Procedure 9 using Compound 2.j (10 mg), DMF and 1-amino-2-methylpropan-2-ol. Purification was accomplished by preparative HPLC as described in General Procedure 11, eluting with a 10 to 40% CH₃CN/H₂O+0.1% TFA gradient to give Compound 162 as a white solid (8.4 mg, 52% yield, assumed 2×TFA salt).

LC/MS: Calc'd m/z=437.2 for C₂₄H₃₁N₅OS, found [M+H]⁺=438.4. ¹H NMR (300 MHz, acetonitrile-d₃) δ 7.80 (d, J=5.5 Hz, 1H), 7.51-7.40 (m, 3H), 7.17 (d, J=8.1 Hz, 2H), 5.65 (s, 2H), 4.19 (s, 2H), 2.95-2.81 (m, 4H), 1.83-1.67 (m, 2H), 1.48-1.32 (m, 2H), 1.18 (s, 6H), 0.90 (t, J=7.3 Hz, 3H).

### 2.31 2-butyl-1-(4-(((2-methyl-2-morpholinopropyl)amino)methyl)benzyl)-1H-imidazo[4,5-d]thieno[3,2-b]pyridin-4-amine (Compound 163)

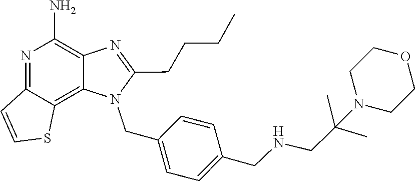 [SMILES: CCCCc1nc2c(N)nc3ccsc3c2n1Cc1ccc(CNCC(C)(C)N2CCOCC2)cc1]

The title compound was prepared according to General Procedure 9 using Compound 2.j (10 mg), DMF and 2-methyl-2-morpholinopropan-1-amine. Purification was accomplished by preparative HPLC as described in General Procedure 11, eluting with a 10 to 40% CH₃CN/H₂O+0.1% TFA gradient to give Compound 163 as a white solid (11.8 mg, 62% yield, assumed 2×TFA salt).

LC/MS: Calc'd m/z=506.3 for C₂₈H₃₈N₆OS, found [M+H]⁺=507.4. ¹H NMR (300 MHz, acetonitrile-d₃) δ 7.80 (d, J=5.4 Hz, 1H), 7.51-7.45 (m, 2H), 7.43 (d, J=5.5 Hz, 1H), 7.15 (d, J=8.0 Hz, 2H), 5.64 (s, 2H), 4.19 (s, 2H), 3.68 (t, J=4.7 Hz, 4H), 3.14 (s, 2H), 2.95-2.84 (m, 2H), 2.82 (t, J=4.7 Hz, 4H), 1.77 (p, J=7.5 Hz, 2H), 1.41 (h, J=7.4 Hz, 2H), 1.27 (s, 6H), 0.92 (t, J=7.3 Hz, 3H).

### 2.32 4-((4-amino-2-butyl-1H-imidazo[4,5-d]thieno[3,2-b]pyridin-1-yl)methyl)benzaldehyde (Compound 164)

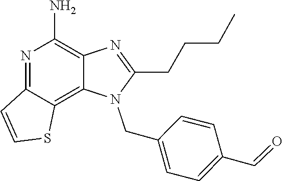 [SMILES: CCCCc1nc2c(N)nc3ccsc3c2n1Cc1ccc(C=O)cc1]

The title compound was prepared according to General Procedure 9 using Compound 2.j (10 mg), DMF and 1,3,5-triazaadamantan-7-amine. Compound 164 was isolated as the major product. Purification was accomplished by preparative HPLC as described in General Procedure 11, eluting with a 10 to 40% CH₃CN/H₂O+0.1% TFA gradient to give Compound 164 as a white solid (5.3 mg, 50% yield, assumed 2×TFA salt).

LC/MS: Calc'd m/z=364.1 for C₂₀H₂₀N₄OS, found [M+H]⁺=365.2. ¹H NMR (300 MHz, acetonitrile-d₃) δ 9.96 (s, 1H), 7.92-7.83 (m, 2H), 7.80 (d, J=5.5 Hz, 1H), 7.42 (d, J=5.5 Hz, 1H), 7.29 (d, J=8.1 Hz, 2H), 5.72 (s, 2H), 2.97-2.84 (m, 2H) 1.83-1.67 (m, 2H), 1.49-1.31 (m, 2H), 0.89 (t, J=7.3 Hz, 3H).

### 2.33 tert-butyl (2-(4-(4-((4-amino-2-butyl-1H-imidazo[4,5-d]thieno[3,2-b]pyridin-1-yl)methyl) benzyl)piperazin-1-yl)ethyl)carbamate (Compound 165)

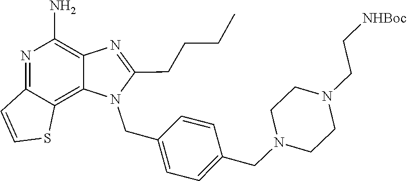 [SMILES: CCCCc1nc2c(N)nc3ccsc3c2n1Cc1ccc(CN2CCN(CCNC(=O)OC(C)(C)C)CC2)cc1]

The title compound was prepared according to General Procedure 9 using Compound 2.j (10 mg), DMF and tert-butyl (2-(piperazin-1-yl)ethyl)carbamate. Purification was accomplished by preparative HPLC as described in General Procedure 11, eluting with a 10 to 40% CH₃CN/H₂O+0.1% TFA gradient to give Compound 165 as a white solid (10.3 mg, 43% yield, assumed 3×TFA salt).

LC/MS: Calc'd m/z=577.3 for C₃₁H₄₃N₇O₂S, found [M+H]⁺=578.4. ¹H NMR (300 MHz, acetonitrile-d₃) δ 7.80 (d, J=5.4 Hz, 1H), 7.47-7.38 (m, 3H), 7.16 (d, J=8.0 Hz, 2H), 5.64 (s, 2H), 4.14 (s, 2H), 3.46-3.32 (m, 6H), 3.28 (d, J=4.8 Hz, 2H), 3.15 (t, J=6.0 Hz, 2H), 2.94-2.83 (m, 2H), 1.75 (p, J=7.5 Hz, 2H), 1.54-1.29 (m, 2H), 1.40 (s, 9H), 0.90 (t, J=7.3 Hz, 3H).

### 2.34 1-(4-((4-(2-aminoethyl)piperazin-1-yl)methyl)benzyl)-2-butyl-1H-imidazo[4,5-d]thieno [3,2-b]pyridin-4-amine (Compound 166)

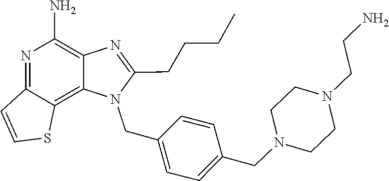 [SMILES: CCCCc1nc2c(N)nc3ccsc3c2n1Cc1ccc(CN2CCN(CCN)CC2)cc1]

The title compound was prepared according to General Procedure 5 from Compound 165 (7.5 mg). Purification was accomplished by preparative HPLC as described in General Procedure 11, eluting with a 10 to 40% CH₃CN/H₂O+0.1% TFA gradient to give Compound 166 as a white solid (4.9 mg, 65% yield, assumed 4×TFA salt).

LC/MS: Calc'd m/z=477.3 for C₂₆H₃₅N₇S, found [M+H]⁺=478.4. ¹H NMR (300 MHz, acetonitrile-d₃) δ 7.81 (d, J=5.5 Hz, 1H), 7.50-7.40 (m, 3H), 7.19 (d, J=7.9 Hz, 2H), 5.66 (s, 2H), 4.22 (s, 2H), 3.01 (t, J=4.8 Hz, 2H), 2.95-2.85 (m, 2H), 2.83-2.67 (m, 8H), 2.66 (t, J=4.8 Hz, 2H), 1.75 (p, J=7.6 Hz, 2H), 1.49-1.31 (m, 2H), 0.90 (t, J=7.3 Hz, 3H).

### 2.35 (S)-2-((4-((4-amino-2-butyl-1H-imidazo[4,5-d]thieno[3,2-b]pyridin-1-yl)methyl)benzyl) amino)-3-phenylpropan-1-ol (Compound 167)

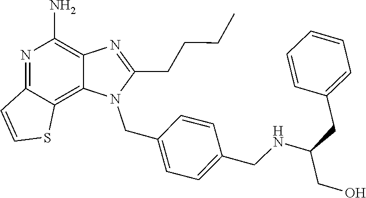 [SMILES: CCCCc1nc2c(N)nc3ccsc3c2n1Cc1ccc(CN[C@H](CO)Cc2ccccc2)cc1]

The title compound was prepared according to General Procedure 9 using Compound 2.j (10 mg), DMF and L-phenylalaninol. Purification was accomplished by preparative HPLC as described in General Procedure 11, eluting with a 10 to 40% CH₃CN/H₂O+0.1% TFA gradient to give Compound 167 as a white solid (11.8 mg, 68% yield, assumed 2×TFA salt).

LC/MS: Calc'd m/z=499.2 for C₂₉H₃₃N₅OS, found [M+H]⁺=500.4. ¹H NMR (300 MHz, acetonitrile-d₃) δ 7.74 (d, J=5.4 Hz, 1H), 7.45 (d, J=8.2 Hz, 2H), 7.41 (d, J=5.5 Hz, 1H), 7.37-7.21 (m, 4H), 7.21-7.11 (m, 5H), 5.65 (s, 2H), 4.25 (s, 2H), 3.68 (dd, J=12.5, 3.3 Hz, 1H), 3.50 (dd, J=12.5, 5.2 Hz, 1H), 3.02 (dd, J=13.7, 5.0 Hz, 1H), 2.96-2.82 (m, 4H), 1.75 (p, J=7.5 Hz, 2H), 1.40 (h, J=7.4 Hz, 2H), 0.90 (t, J=7.3 Hz, 3H).

### 2.36 2-butyl-1-(4-((dimethylamino)methyl)benzyl)-1H-imidazo[4,5-d]thieno[3,2-b]pyridin-4-amine (Compound 168)

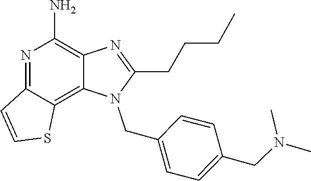 [SMILES: CCCCc1nc2c(N)nc3ccsc3c2n1Cc1ccc(CN(C)C)cc1]

The title compound was prepared according to General Procedure 9 using Compound 2.j (10 mg), DMF and dimethylamine. Purification was accomplished by preparative HPLC as described in General Procedure 11, eluting with a 10 to 40% CH₃CN/H₂O+0.1% TFA gradient to give Compound 168 as a white solid (0.8 mg, 5% yield, assumed 2×TFA salt).

LC/MS: Calc'd m/z=393.20 for C₂₂H₂₇N₅S, found [M+H]⁺=394.2. ¹H NMR (300 MHz, acetonitrile-d₃) δ 7.82 (d, J=5.5 Hz, 1H), 7.44 (dd, J=6.9, 1.4 Hz, 3H), 7.20 (d, J=8.0 Hz, 2H), 5.67 (s, 2H), 4.19 (s, 2H), 2.95-2.84 (m, 2H), 2.72 (s, 6H), 1.82-1.66 (m, 2H), 1.49-1.31 (m, 2H), 0.89 (t, J=7.4 Hz, 3H).

### 2.37 benzyl (4-((4-((4-amino-2-butyl-1H-imidazo[4,5-d]thieno[3,2-b]pyridin-1-yl)methyl) benzyl)amino)butyl)carbamate (Compound 260)

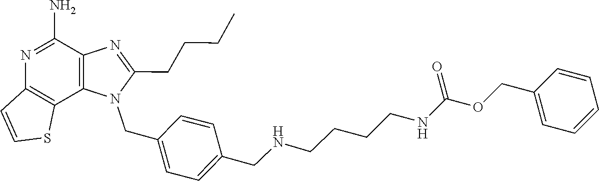 [SMILES: CCCCc1nc2c(N)nc3ccsc3c2n1Cc1ccc(CNCCCCNC(=O)OCc2ccccc2)cc1]

The title compound was prepared according to General Procedure 9 using Compound 2.j (17 mg), DMA and benzyl N-(4-aminobutyl)carbamate. Purification was accomplished by preparative HPLC as described in General Procedure 11, to give Compound 260 as a white solid (2.4 mg, 12% yield, assumed 2×TFA salt).

LC/MS: Calc'd m/z=570.3 for C₃₂H₃₈N₆O₂S, found [M+H]⁺=571.4.

### 2.38 tert-butyl 6-((4-((4-amino-2-butyl-1H-imidazo[4,5-d]thieno[3,2-b]pyridin-1-yl)methyl) benzyl)amino)-3,4-dihydroisoquinoline-2(1H)-carboxylate (Compound 261)

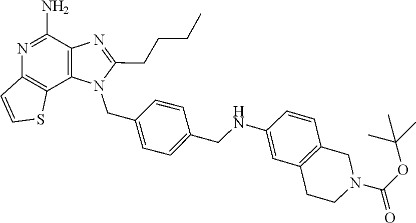 [SMILES: CCCCc1nc2c(N)nc3ccsc3c2n1Cc1ccc(CNc2ccc3c(c2)CCN(C(=O)OC(C)(C)C)C3)cc1]

The title compound was prepared according to General Procedure 9 using Compound 2.j (10 mg), DMA and tert-butyl 6-amino-3,4-dihydro-1H-isoquinoline-2-carboxylate. Purification was accomplished by preparative HPLC as described in General Procedure 11 to give Compound 261 as a white solid (4.5 mg, 21% yield, assumed 2×TFA salt).

LC/MS: Calc'd m/z=596.3 for C₃₄H₄₀N₆O₂S, found [M+H]⁺=597.4.

### 2.39 tert-butyl 3-((4-((4-amino-2-butyl-1H-imidazo[4,5-d]thieno[3,2-b]pyridin-1-yl)methyl) benzyl)amino)piperidine-1-carboxylate (Compound 262)

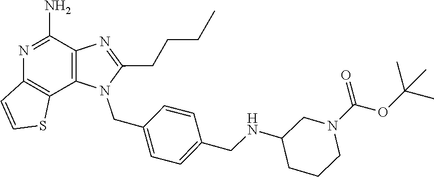 [SMILES: CCCCc1nc2c(N)nc3ccsc3c2n1Cc1ccc(CNC2CCCN(C(=O)OC(C)(C)C)C2)cc1]

The title compound was prepared according to General Procedure 9 using Compound 2.j (10 mg), DMA and tert-butyl 3-aminopiperidine-1-carboxylate. Purification was accomplished by preparative HPLC as described in General Procedure 11 to give Compound 262 as a white solid (4.5 mg, 14% yield, assumed 2×TFA salt).

LC/MS: Calc'd m/z=548.3 for C₃₀H₄₀N₆O₂S, found [M+H]⁺=549.4.

### 2.40 tert-butyl ((1-(4-((4-amino-2-butyl-1H-imidazo[4,5-d]thieno[3,2-b]pyridin-1-yl)methyl) benzyl)piperidin-4-yl)methyl)carbamate (Compound 263)

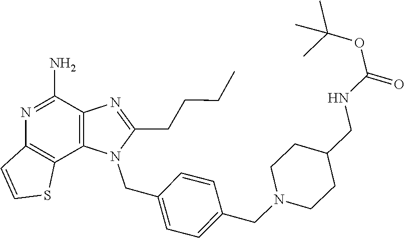 [SMILES: CCCCc1nc2c(N)nc3ccsc3c2n1Cc1ccc(CN2CCC(CNC(=O)OC(C)(C)C)CC2)cc1]

The title compound was prepared according to General Procedure 9 using Compound 2.j (10 mg), DMA and tert-butyl N-(piperidin-4-ylmethyl)carbamate. Purification was accomplished by preparative HPLC as described in General Procedure 11 to give Compound 263 as a white solid (2.8 mg, 14% yield, assumed 2×TFA salt).

LC/MS: Calc'd m/z=562.3 for C₃₁H₄₂N₆O₂S, found [M+H]⁺=563.4.

### 2.41 tert-butyl 4-(((4-((4-amino-2-butyl-1H-imidazo[4,5-d]thieno[3,2-b]pyridin-1-yl)methyl) benzyl)amino)methyl)piperidine-1-carboxylate (Compound 264)

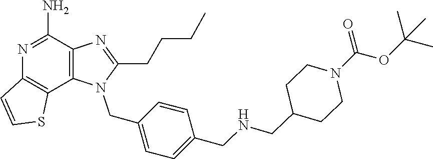 [SMILES: CCCCc1nc2c(N)nc3ccsc3c2n1Cc1ccc(CNCC2CCN(C(=O)OC(C)(C)C)CC2)cc1]

The title compound was prepared according to General Procedure 9 using Compound 2.j (10 mg), DMA and tert-butyl N-(piperidin-4-ylmethyl)carbamate. Purification was accomplished by preparative HPLC as described in General Procedure 11 to give Compound 264 as a white solid (4.1 mg, 19% yield, assumed 2×TFA salt).

LC/MS: Calc'd m/z=562.3 for C₃₁H₄₂N₆O₂S, found [M+H]⁺=563.4.

### 2.42 2-butyl-1-(4-(((3,3-difluorocyclobutyl)amino)methyl)benzyl)-1H-imidazo[4,5-d]thieno[3,2-b]pyridin-4-amine (Compound 265)

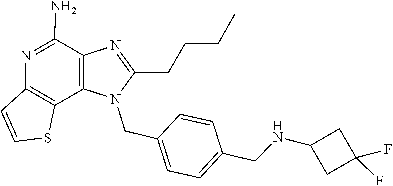 [SMILES: CCCCc1nc2c(N)nc3ccsc3c2n1Cc1ccc(CNC2CC(F)(F)C2)cc1]

The title compound was prepared according to General Procedure 9 using Compound 2.j (17 mg), DMA and 3,3-difluorocyclobutan-1-amine hydrochloride. Purification was accomplished by preparative HPLC as described in General Procedure 11 to give Compound 265 as a white solid (4.9 mg, 17% yield, assumed 2×TFA salt).

LC/MS: Calc'd m/z=455.2 for C₂₄H₂₇F₂N₅S, found [M+H]⁺=456.4. ¹H NMR (300 MHz, acetonitrile-d₃) δ 7.81 (d, J=5.5 Hz, 1H), 7.47-7.38 (m, 3H), 7.16 (d, J=7.9 Hz, 2H), 5.65 (s, 2H), 4.10 (s, 2H), 3.74-3.52 (m, 1H), 3.12 t, J=7.6 Hz, 2H), 1.75 (p, J=7.7 Hz, 2H), 1.41 (h, J=7.5 Hz, 2H), 1.35-1.12 (m, 4H), 0.90 (t, J=7.3 Hz, 3H).

### 2.43 1-(4-(((4H-1,2,4-triazol-3-yl)amino)methyl)benzyl)-2-butyl-1H-imidazo[4,5-d]thieno [3,2-b]pyridin-4-amine (Compound 272)

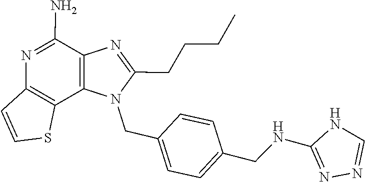 [SMILES: CCCCc1nc2c(N)nc3ccsc3c2n1Cc1ccc(CNc2nnc[nH]2)cc1]

The title compound was prepared according to General Procedure 9 using Compound 2.j (10 mg), DMA and 2-amino-1,3,4-triazole. Purification was accomplished by preparative HPLC as described in General Procedure 11 to give Compound 272 as a white solid (3.0 mg, 14% yield, assumed 3×TFA salt).

LC/MS: Calc'd m/z=432.2 for C₂₂H₂₄N₈S, found [M+2H]²⁺=217.2. ¹H NMR (300 MHz, acetonitrile-d₃) δ 7.89 (s, 1H), 7.81 (d, J=5.5 Hz, 1H), 7.42 (d, J=5.4 Hz, 1H), 7.29 (d, J=8.1 Hz, 3H), 7.15 (d, J=8.0 Hz, 2H), 5.64 (s, 2H), 5.05 (s, 2H), 2.88 (t, J=7.6 Hz, 3H), 1.73 (p, J=7.7 Hz, 3H), 1.39 (h, J=7.1 Hz, 3H), 0.88 (t, J=7.3 Hz, 4H).

### 2.44 2-butyl-1-(4-((4-(2-(pyridin-2-yl)ethyl)piperazin-1-yl)methyl)benzyl)-1H-imidazo[4,5-d]thieno[3,2-b]pyridin-4-amine (Compound 275)

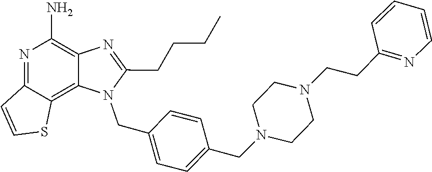 [SMILES: CCCCc1nc2c(N)nc3ccsc3c2n1Cc1ccc(CN2CCN(CCc3ccccn3)CC2)cc1]

The title compound was prepared according to General Procedure 9 using Compound 2.j (10 mg), DMA and 1-(2-(pyridin-2-yl)ethyl)piperazine. Purification was accomplished by preparative HPLC as described in General Procedure 11 to give Compound 275 as a white solid (13 mg, 50% yield, assumed 4×TFA salt).

LC/MS: Calc'd m/z=539.3 for C₃₁H₃₇N₇S, found [M+H]⁺=540.4. ¹H NMR (300 MHz, acetonitrile-d₃) δ 8.61 (dd, J=5.6, 1.7 Hz, 1H), 8.28 (td, J=7.9, 1.7 Hz, 1H), 7.81 (d, J=5.5 Hz, 1H), 7.48-7.38 (m, 3H), 7.17 (d, J=8.0 Hz, 2H), 5.65 (s, 2H), 4.12 (s, 2H), 3.27-3.19 (m, 2H), 3.13 (td, J=6.2, 2.9 Hz, 6H), 3.05 (s, 4H), 2.89 (t, J=7.6 Hz, 2H), 1.74 (p, J=7.6 Hz, 2H), 1.49-1.30 (m, 2H), 0.89 (t, J=7.3 Hz, 3H).

### 2.45 2-butyl-1-(4-((dipropylamino)methyl)benzyl)-1H-imidazo[4,5-d]thieno[3,2-b]pyridin-4-amine (Compound 277)

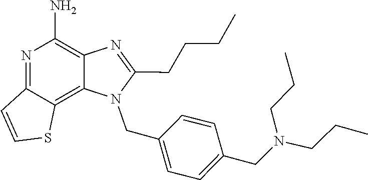 [SMILES: CCCCc1nc2c(N)nc3ccsc3c2n1Cc1ccc(CN(CCC)CCC)cc1]

The title compound was prepared according to General Procedure 9 using Compound 2.j (15 mg), DMA and dipropylamine. Purification was accomplished by preparative HPLC as described in General Procedure 11 to give Compound 277 as a white solid (32.7 mg, 123% yield, assumed 2×TFA salt).

LC/MS: Calc'd m/z=449.3 for C₂₆H₃₅N₅S, found [M+H]⁺=450.4.

### 2.46 1-(4-((4-amino-2-butyl-1H-imidazo[4,5-d]thieno[3,2-b]pyridin-1-yl)methyl)benzyl) pyridin-1-ium (Compound 279)

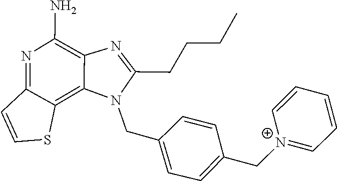 [SMILES: CCCCc1nc2c(N)nc3ccsc3c2n1Cc1ccc(C[n+]2ccccc2)cc1]

The title compound was prepared according to General Procedure 9 using Compound 2.j (10 mg), DMA and pyridine. Purification was accomplished by preparative HPLC as described in General Procedure 11 to give Compound 279 as a white solid (4.5 mg, 22% yield, assumed 2×TFA salt).

LC/MS: Calc'd m/z=428.2 for C₂₅H₂₆N₅S, found [M+H]⁺=428.2. ¹H NMR (300 MHz, acetonitrile-d₃) δ 8.89-8.61 (m, 2H), 8.58-8.46 (m, 1H), 8.03 (t, J=7.1 Hz, 2H), 7.81 (d, J=5.5 Hz, 1H), 7.47-7.38 (m, 3H), 7.18 (d, J=8.1 Hz, 2H), 5.70 (s, 2H), 5.64 (s, 2H), 2.92-2.81 (m, 2H), 1.71 (p, J=7.5 Hz, 2H), 1.37 (h, J=7.3 Hz, 3H), 0.86 (t, J=7.4 Hz, 3H).

### 2.47 1-(4-(((2-azaspiro[3.3]heptan-6-yl)amino)methyl)benzyl)-2-butyl-1H-imidazo[4,5-d]thieno[3,2-b]pyridin-4-amine (Compound 286)

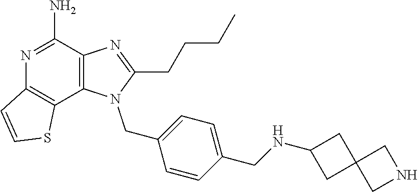 [SMILES: CCCCc1nc2c(N)nc3ccsc3c2n1Cc1ccc(CNC2CC3(CNC3)C2)cc1]

The title compound was prepared according to General Procedure 9 followed by General Procedure 5 using Compound 2.j (10 mg), DMA and tert-butyl 6-amino-2-azaspiro[3.3]heptane-2-carboxylate. Purification was accomplished by preparative HPLC as described in General Procedure 11 to give Compound 286 as a white solid (3.7 mg, 17% yield, assumed 3×TFA salt).

LC/MS: Calc'd m/z=460.2 for C₂₆H₃₂N₆S, found [M+H]⁺=461.4. ¹H NMR (300 MHz, acetonitrile-d₃) δ 7.82 (d, J=5.5 Hz, 1H), 7.43 (d, J=5.5 Hz, 1H), 7.39 (d, J=8.1 Hz, 2H), 7.15 (d, J=8.0 Hz, 2H), 5.65 (s, 2H), 4.06 (s, 2H), 4.01 (s, 2H), 3.99 (s, 2H), 3.60 (p, J=8.1 Hz, 1H), 2.89 (t, J=7.6 Hz, 2H), 2.70-2.52 (m, 2H), 2.52-2.32 (m, 2H), 1.75 (p, J=7.6 Hz, 2H), 1.40 (h, J=7.5 Hz, 2H), 0.90 (t, J=7.3 Hz, 3H).

### 2.48 1-(4-((4-amino-2-butyl-1H-imidazo[4,5-d]thieno[3,2-b]pyridin-1-yl)methyl)benzyl)-1,4-diazabicyclo[2.2.2]octan-1-ium (Compound 295)

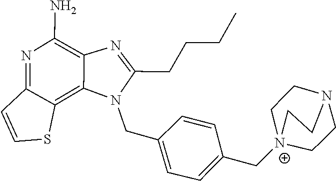 [SMILES: CCCCc1nc2c(N)nc3ccsc3c2n1Cc1ccc(C[N+]23CCN(CC2)CC3)cc1]

The title compound was prepared according to a modified version of General Procedure 9 using Compound 2.j (10 mg), 3:1 CH₃CN:H₂O instead of DMA; 2 eq. of sodium iodide and 1.5 eq of potassium carbonate instead of DIPEA, and 1,4-diazabicyclo[2.2.2]octane. Purification was accomplished by preparative HPLC as described in General Procedure 11 to give Compound 295 as a white solid (9.5 mg, 46% yield, assumed 3×TFA salt).

LC/MS: Calc'd m/z=461.2 for C₂₆H₃₃N₆S, found [M+H]⁺=461.4. ¹H NMR (300 MHz, acetonitrile-d₃) δ 7.82 (d, J=5.4 Hz, 1H), 7.61-7.39 (m, 3H), 7.24 (d, J=7.9 Hz, 2H), 5.68 (s, 2H), 4.46 (s, 2H), 3.49-3.38 (m, 6H), 3.35-3.28 (m, 6H), 2.89 (t, J=7.6 Hz, 2H), 1.76 (p, J=7.6 Hz, 2H), 1.41 (h, J=7.4 Hz, 2H), 0.91 (t, J=7.3 Hz, 3H).

### 2.49 (2S,3S)-2-((4-((4-amino-2-butyl-1H-imidazo[4,5-d]thieno[3,2-b]pyridin-1-yl)methyl) benzyl)amino)-3-methylpentan-1-ol (Compound 296)

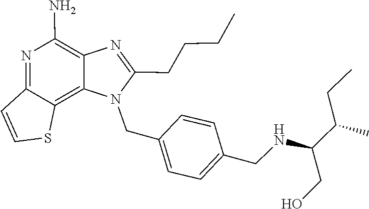 [SMILES: CCCCc1nc2c(N)nc3ccsc3c2n1Cc1ccc(CN[C@H](CO)[C@@H](C)CC)cc1]

The title compound was prepared according to General Procedure 9 using Compound 2.j (10 mg), DMA and L-isoleucinol. Purification was accomplished by preparative HPLC as described in General Procedure 11 to give Compound 296 as a white solid (2.9 mg, 16% yield, assumed 2×TFA salt).

LC/MS: Calc'd m/z=465.2 for C₂₆H₃₅N₅OS, found [M+H]⁺=466.4. ¹H NMR (300 MHz, acetonitrile-d₃) δ 7.81 (d, J=5.5 Hz, 1H), 7.59-7.36 (m, 3H), 7.17 (d, J=7.9 Hz, 2H), 5.66 (s, 2H), 4.26 (d, J=2.7 Hz, 2H), 3.82-3.54 (m, 2H), 3.08-2.96 (m, 2H), 1.75 (p, J=7.4 Hz, 3H), 1.47-1.34 (m, 4H), 1.35-1.10 (m, 2H), 0.96-0.83 (m, 7H), 0.76 (t, J=7.3 Hz, 3H).

### 2.50 (4-((4-amino-2-butyl-1H-imidazo[4,5-d]thieno[3,2-b]pyridin-1-yl)methyl)benzyl)-L-isoleucine (Compound 297)

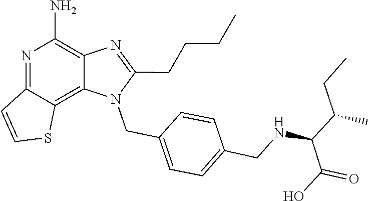 [SMILES: CCCCc1nc2c(N)nc3ccsc3c2n1Cc1ccc(CN[C@H](C(=O)O)[C@@H](C)CC)cc1]

The title compound was prepared according to a modified General Procedure 9 using Compound 2.j (10 mg); 3:1 CH₃CN:H₂O instead of DMA; 2 eq. sodium iodide and 2.5 eq. potassium carbonate instead of DIPEA, and L-isoleucine. Purification was accomplished by preparative HPLC as described in General Procedure 11 to give Compound 297 as a white solid (3.0 mg, 16% yield, assumed 2×TFA salt).

LC/MS: Calc'd m/z=479.2 for C₂₆H₃₃N₅O₂S, found [M+H]⁺=480.4. ¹H NMR (300 MHz, acetonitrile-d₃) δ 7.80 (d, J=5.5 Hz, 1H), 7.49-7.37 (m, 3H), 7.16 (d, J=7.8 Hz, 2H), 5.65 (s, 2H), 4.38-4.01 (m, 2H), 3.59 (d, J=3.4 Hz, 1H), 2.90 (t, J=7.6 Hz, 2H), 1.75 (p, J=7.6 Hz, 2H), 1.42 (h, J=7.4 Hz, 2H), 1.36-1.19 (m, 1H), 0.96-0.77 (m, 10H).

### 2.51 (4-((4-amino-2-butyl-1H-imidazo[4,5-d]thieno[3,2-b]pyridin-1-yl)methyl)benzyl)-L-threonine (Compound 298)

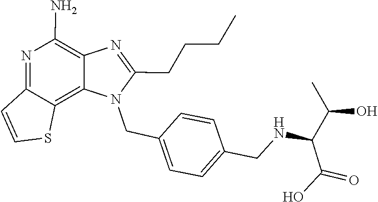 [SMILES: CCCCc1nc2c(N)nc3ccsc3c2n1Cc1ccc(CN[C@H](C(=O)O)[C@@H](C)O)cc1]

The title compound was prepared according to a modified General Procedure 9 using Compound 2.j (10 mg); 3:1 CH₃CN:H₂O instead of DMA; 2 eq. sodium iodide and 2.5 eq. potassium carbonate instead of DIPEA, and L-threonine. Purification was accomplished by preparative HPLC as described in General Procedure 11 to give Compound 298 as a white solid (2.5 mg, 14% yield, assumed 2×TFA salt).

LC/MS: Calc'd m/z=467.2 for C₂₄H₂₉N₅O₃S, found [M+H]⁺=468.4. ¹H NMR (300 MHz, acetonitrile-d₃) δ 7.80 (d, J=5.5 Hz, 1H), 7.58-7.32 (m, 3H), 7.17 (d, J=7.8 Hz, 2H), 5.65 (s, 2H), 4.33-4.12 (m, 2H), 4.06 (t, J=6.4 Hz, 1H), 3.36 (d, J=6.3 Hz, 1H), 2.90 (t, J=7.7 Hz, 2H), 2.42 (s, 1H), 1.75 (p, J=7.7 Hz, 2H), 1.41 (h, J=7.4 Hz, 2H), 1.18 (d, J=6.4 Hz, 3H), 0.91 (t, J=7.3 Hz, 3H).

### 2.52 2,2′-((4-((4-amino-2-butyl-1H-imidazo[4,5-d]thieno[3,2-b]pyridin-1-yl)methyl)benzyl) azanediyl)diacetic acid (Compound 299)

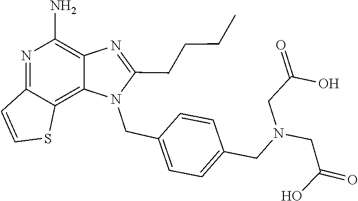 [SMILES: CCCCc1nc2c(N)nc3ccsc3c2n1Cc1ccc(CN(CC(=O)O)CC(=O)O)cc1]

The title compound was prepared according to a modified General Procedure 9 using Compound 2.j (10 mg); 3:1 CH₃CN:H₂O instead of DMA; 2 eq. sodium iodide and 2.5 eq. potassium carbonate instead of DIPEA, and iminodiacetic acid. Purification was accomplished by preparative HPLC as described in General Procedure 11 to give Compound 299 as a white solid (2.6 mg, 10% yield, assumed 2×TFA salt).

LC/MS: Calc'd m/z=481.2 for C₂₄H₂₇N₅O₄S, found [M+H]⁺=482.2. ¹H NMR (300 MHz, acetonitrile-d₃) δ 7.81 (d, J=5.5 Hz, 1H), 7.47-7.37 (m, 3H), 7.16 (d, J=7.9 Hz, 2H), 5.65 (s, 2H), 4.09 (s, 2H), 2.96-2.85 (m, 5H), 1.74 (p, J=7.5 Hz, 3H), 1.61 (h, J=7.6 Hz, 2H), 0.90 (t, J=7.4 Hz, 3H).

### 2.53 (4-((4-amino-2-butyl-1H-imidazo[4,5-d]thieno[3,2-b]pyridin-1-yl)methyl)benzyl)-L-proline (Compound 300)

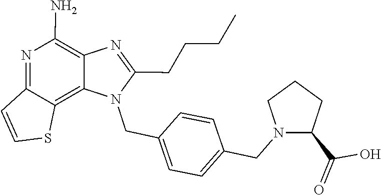 [SMILES: CCCCc1nc2c(N)nc3ccsc3c2n1Cc1ccc(CN2CCC[C@H]2C(=O)O)cc1]

The title compound was prepared according to a modified General Procedure 9 using Compound 2.j (10 mg); 1:3 H2O:CH3CN instead of DMA; 2 eq. sodium iodide and 2.5 eq. potassium carbonate instead of DIPEA, and L-proline. Purification was accomplished by preparative HPLC as described in General Procedure 11 to give Compound 300 as a white solid (5.4 mg, 30% yield, assumed 2×TFA salt).

LC/MS: Calc'd m/z=463.2 for C₂₅H₂₉N₅O₂S, found [M+H]⁺=464.4. ¹H NMR (300 MHz, acetonitrile-d₃) δ 7.79 (d, J=5.4 Hz, 1H), 7.48 (d, J=7.9 Hz, 2H), 7.37 (d, J=5.5 Hz, 1H), 7.18 (d, J=7.8 Hz, 2H), 5.65 (s, 2H), 4.37 (d, J=13 Hz, 1H), 4.28 (d, J=13.0 Hz, 1H), 4.05 (dd, J=9.5, 6.2 Hz, 1H), 3.24-3.06 (m, 1H), 2.89 (t, J=7.6 Hz, 2H), 2.52-2.34 (m, 1H), 2.22-2.02 (m, 2H), 1.94-1.83 (m, 1H), 1.73 (p, J=7.6 Hz, 2H), 1.39 (h, J=7.4 Hz, 2H), 0.89 (t, J=7.3 Hz, 3H).

### 2.54 (3-((4-((4-amino-2-butyl-1H-imidazo[4,5-d]thieno[3,2-b]pyridin-1-yl)methyl)benzyl) amino)propanoyl)-L-histidine (Compound 301)

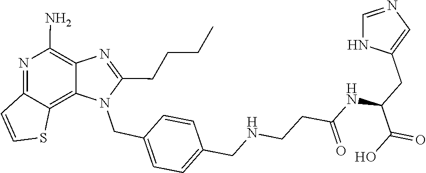 [SMILES: CCCCc1nc2c(N)nc3ccsc3c2n1Cc1ccc(CNCCC(=O)N[C@@H](Cc2cnc[nH]2)C(=O)O)cc1]

The title compound was prepared according to a modified General Procedure 9 using Compound 2.j (10 mg); 1:3 H2O:CH3CN instead of DMA; 2 eq. sodium iodide and 2.5 eq. potassium carbonate instead of DIPEA, and carnosine. Purification was accomplished by preparative HPLC as described in General Procedure 11 to give Compound 301 as a white solid (0.9 mg, 4% yield, assumed 3×TFA salt).

LC/MS: Calc'd m/z=574.2 for C₂₉H₃₄N₈O₃S, found [M+H]⁺=575.4. ¹H NMR (300 MHz, acetonitrile-d₃) δ 8.50 (s, 1H), 7.81 (d, J=5.5 Hz, 1H), 7.54-7.32 (m, 3H), 7.23 (s, 1H), 7.17 (d, J=7.8 Hz, 2H), 5.66 (s, 2H), 4.62 (t, J=6.6 Hz, 1H), 4.14 (s, 2H), 3.15 (t, J=7.2 Hz, 2H), 2.90 (t, J=7.6 Hz, 2H), 2.63 (t, J=6.4 Hz, 2H), 1.75 (p, J=7.4 Hz, 2H), 1.40 (h, J=7.4 Hz, 2H), 0.90 (t, J=7.3 Hz, 3H).

### 2.55 6-((4-((4-amino-2-butyl-1H-imidazo[4,5-d]thieno[3,2-b]pyridin-1-yl)methyl)benzyl) amino)hexanoic acid (Compound 303)

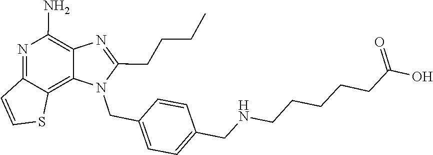 [SMILES: CCCCc1nc2c(N)nc3ccsc3c2n1Cc1ccc(CNCCCCCC(=O)O)cc1]

The title compound was prepared according to a modified General Procedure 9 using Compound 2.j (10 mg); 3:1 CH₃CN:H₂O instead of DMA; 2 eq. sodium iodide and 2.5 eq. potassium carbonate instead of DIPEA, and aminocaproic acid. Purification was accomplished by preparative HPLC as described in General Procedure 11 to give Compound 303 as a white solid (1.8 mg, 10% yield, assumed 3×TFA salt).

LC/MS: Calc'd m/z=479.2 for C₂₆H₃₃N₅O₂S, found [M+H]⁺=¹H NMR (300 MHz, acetonitrile-d₃) δ 7.81 (d, J=5.5 Hz, 1H), 7.51-7.33 (m, 3H), 7.16 (d, J=8.0 Hz, 2H), 5.65 (s, 2H), 4.09 (s, 2H), 2.98-2.64 (m, 4H), 2.27 (t, J=7.3 Hz, 2H), 1.74 (p, J=7.7 Hz, 2H), 1.67-1.45 (m, 4H), 1.47-1.20 (m, 4H), 0.89 (t, J=7.4 Hz, 3H).

### 2.56 N⁶-(4-((4-amino-2-butyl-1H-imidazo[4,5-d]thieno[3,2-b]pyridin-1-yl)methyl)benzyl)-L-lysine (Compound 305)

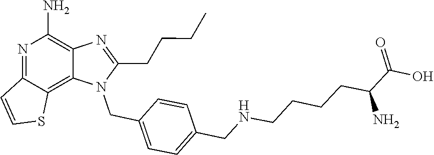 [SMILES: CCCCc1nc2c(N)nc3ccsc3c2n1Cc1ccc(CNCCCC[C@H](N)C(=O)O)cc1]

The title compound was prepared according to a modified General Procedure 9 followed by General Procedure 5 using Compound 2.j (10 mg); 3:1 CH₃CN:H₂O instead of DMA; 2 eq. sodium iodide and 2.5 eq. potassium carbonate instead of DIPEA, and (2S)-6-amino-2-[(tert-butoxycarbonyl)amino]hexanoic acid. Purification was accomplished by preparative HPLC as described in General Procedure 11 to give Compound 305 as a white solid (2.0 mg, 9% yield, assumed 3×TFA salt).

LC/MS: Calc'd m/z=494.2 for C₂₆H₃₄N₆O₂S, found [M+H]⁺=495.4. ¹H NMR (300 MHz, acetonitrile-d₃) δ 7.81 (d, J=5.5 Hz, 1H), 7.50-7.35 (m, 3H), 7.16 (d, J=7.8 Hz, 2H), 5.65 (s, 2H), 4.10 (s, 2H), 3.89 (t, J=6.3 Hz, 1H), 3.01-2.84 (m, 4H), 1.93-1.82 (m, 2H), 1.81-1.60 (m, 3H), 1.50-1.31 (m, 3H), 0.90 (t, J=7.3 Hz, 3H).

### Example 3: Preparation of Series 3 Compounds

### 3.1 5-((6-nitrothieno[3,2-b]pyridin-7-yl)amino)pentan-1-ol (Compound 3.a)

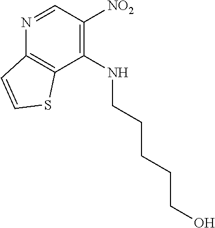 [SMILES: O=[N+]([O-])c1cnc2ccsc2c1NCCCCCO]

The title compound was prepared according to General Procedure 6 starting from Compound 2.a (4.70 g) and 5-aminopentanol. The crude compound was taken to the next step with no additional purification.

LC/MS: Calc'd m/z=315.1 for C₁₅H₁₃N₃O₃S, found [M+H]⁺=316.2.

### 3.2 5-((6-aminothieno[3,2-b]pyridin-7-yl)amino)pentan-1-ol (Compound 3.b)

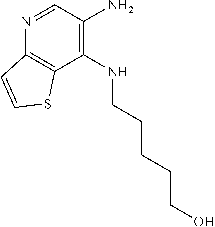 [SMILES: Nc1cnc2ccsc2c1NCCCCCO]

The title compound was prepared according to General Procedure 2 starting from crude Compound 3.a. Purification was accomplished as described in General Procedure 11, eluting with a 0 to 20% CH₃CN/H₂O+0.1% TFA gradient to give Compound 3.b as a yellow solid (5.01 g, 48% yield, assumed 2×TFA salt).

LC/MS: Calc'd m/z=251.1 for C₁₂H₁₇N₃OS, found [M+H]⁺=252.2.

### 3.3 5-(2-butyl-1H-imidazo[4,5-d]thieno[3,2-b]pyridin-1-yl)pentan-1-ol (Compound 3.c)

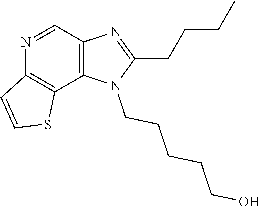 [SMILES: CCCCc1nc2cnc3ccsc3c2n1CCCCCO]

The title compound was prepared according to General Procedure 7 starting from Compound 3.b (5.00 g). Purification was accomplished as described in General Procedure 11, eluting with a 0 to 10% DCM/MeOH+1% NEt₃ gradient to give Compound 3.c as an off-white solid (2.65, 80% yield).

LC/MS: Calc'd m/z=317.2 for C₁₇H₂₃N₃OS, found [M+H]⁺=318.2.

### 3.4 5-(4-amino-2-butyl-1H-imidazo[4,5-d]thieno[3,2-b]pyridin-1-yl)pentan-1-ol (Compound 172)

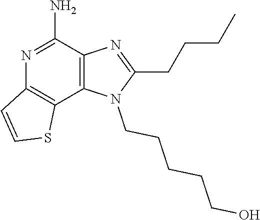 [SMILES: CCCCc1nc2c(N)nc3ccsc3c2n1CCCCCO]

The title compound was prepared according to General Procedure 4 starting from Compound 3.c (600 mg) using chloroform as the solvent. Purification was accomplished as described in General Procedure 11, eluting with 10 to 50% CH₃CN/H₂O+0.1% TFA to give Compound 172 as an off-white solid (252 mg, 30% yield, assumed 1×TFA salt).

LC/MS: Calc'd m/z=332.2 for C₁₇H₂₄N₃OS, found [M+H]⁺=333.2. ¹H NMR (300 MHz, acetone-d6) δ 7.63 (d, J=5.3 Hz, 1H), 7.28 (d, J=5.4 Hz, 1H), 5.68 (s, 1H), 5.64 (s, 1H), 4.42-4.31 (m, 2H), 3.57 (t, J=5.8 Hz, 2H), 3.32 (s, 1H), 3.01-2.90 (m, 2H), 2.02-1.81 (m, 4H), 1.67-1.43 (m, 6H), 1.00 (t, J=7.4 Hz, 3H).

### 3.5 2-butyl-1-(5-chloropentyl)-1H-imidazo[4,5-d]thieno[3,2-b]pyridin-4-amine (Compound 3.d)

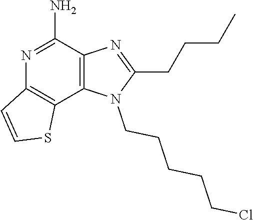 [SMILES: CCCCc1nc2c(N)nc3ccsc3c2n1CCCCCCl]

The title compound was prepared according to General Procedure 8 starting from Compound 172 (250 mg). The crude compound was taken to the next step with no additional purification.

LC/MS: Calc'd m/z=350.1 for C₁₇H₂₃ClN₄S, found [M+H]⁺=351.2.

### 3.6 1-(5-azidopentyl)-2-butyl-1H-imidazo[4,5-d]thieno[3,2-b]pyridin-4-amine (Compound 173)

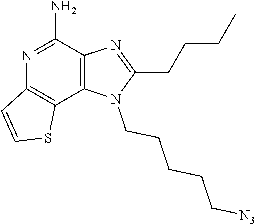 [SMILES: CCCCc1nc2c(N)nc3ccsc3c2n1CCCCCN=[N+]=[N-]]

The title compound was prepared according to General Procedure 9 using Compound 3.d (20 mg), DMA and sodium azide. Purification was accomplished by preparative HPLC as described in General Procedure 11, eluting with a 20 to 55% CH₃CN/H₂O+0.1% TFA gradient to give Compound 173 as a white solid (12 mg, 45% yield, assumed 1×TFA salt).

LC/MS: Calc'd m/z=357.2 for C₁₇H₂₃N₇S, found [M+H]⁺=358.2. ¹H NMR (300 MHz, acetonitrile-d₃) δ 8.37 (d, J=5.5 Hz, 1H), 8.06-7.51 (m, 1H), 4.76 (t, J=7.7 Hz, 2H), 3.72 (t, J=6.5 Hz, 2H), 3.37 (t, J=7.6 Hz, 2H), 2.36-2.20 (m, 4H), 2.11-2.00 (m, 2H), 1.99-1.84 (m, 4H), 1.40 (t, J=7.4 Hz, 3H).

### 3.7 2-butyl-1-(5-(4-(pyridin-4-yl)piperidin-1-yl)pentyl)-1H-imidazo[4,5-d]thieno[3,2-b]pyridin-4-amine (Compound 174)

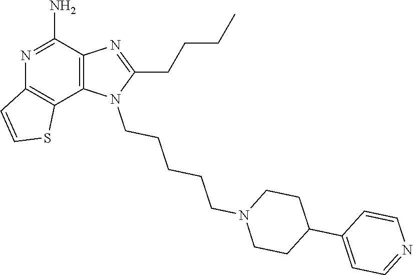 [SMILES: CCCCc1nc2c(N)nc3ccsc3c2n1CCCCCN1CCC(c2ccncc2)CC1]

The title compound was prepared according to General Procedure 9 using Compound 3.d (10 mg), DMA and 4-(piperidin-4-yl)pyridine. Purification was accomplished by preparative HPLC as described in General Procedure 11, eluting with a 10 to 40% CH₃CN/H₂O+0.1% TFA gradient to give Compound 174 as a white solid (9.6 mg, 49% yield, assumed 3×TFA salt).

LC/MS: Calc'd m/z=476.3 for C₂₇H₃₆N₆S, found [M+H]⁺=477.4.

### 3.8 2-butyl-1-(5-(diethylamino)pentyl)-1H-imidazo[4,5-d]thieno[3,2-b]pyridin-4-amine (Compound 175)

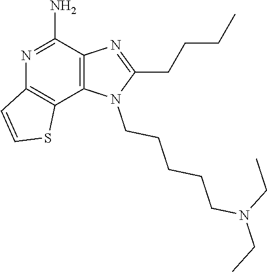 [SMILES: CCCCc1nc2c(N)nc3ccsc3c2n1CCCCCN(CC)CC]

The title compound was prepared according to General Procedure 9 using Compound 3.d (10 mg), DMA and diethylamine. Purification was accomplished by preparative HPLC as described in General Procedure 11, eluting with a 10 to 40% CH₃CN/H₂O+0.1% TFA gradient to give Compound 175 as a white solid (2.8 mg, 16% yield, assumed 2×TFA salt).

LC/MS: Calc'd m/z=387.2 for C₂₁H₃₃N₅S, found [M+H]⁺=388.2. ¹H NMR (300 MHz, acetonitrile-d₃) δ 8.39 (d, J=5.6 Hz, 1H), 7.90 (d, J=5.5 Hz, 1H), 4.78 (t, J=7.8 Hz, 2H), 3.54 (q, J=7.3 Hz, 4H), 3.48-3.34 (m, 4H), 2.37-2.17 (m, 3H), 2.16-2.02 (m, 2H), 1.98-1.79 (m, 5H), 1.64 (t, J=7.3 Hz, 6H), 1.40 (t, J=7.3 Hz, 3H).

### 3.9 2-butyl-1-(5-(4-(2-morpholinoethyl)piperazin-1-yl)pentyl)-1H-imidazo[4,5-d]thieno[3,2-b]pyridin-4-amine (Compound 176)

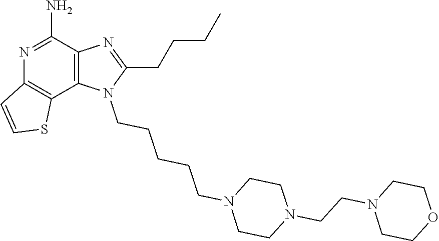 [SMILES: CCCCc1nc2c(N)nc3ccsc3c2n1CCCCCN1CCN(CCN2CCOCC2)CC1]

The title compound was prepared according to General Procedure 9 using Compound 3.d (10 mg), DMA and 4-(2-(piperazin-1-yl)ethyl)morpholine. Purification was accomplished by preparative HPLC as described in General Procedure 11, eluting with a 10 to 40% CH₃CN/H₂O+0.1% TFA gradient to give Compound 176 as a white solid (7.4 mg, 27% yield, assumed 4×TFA salt).

LC/MS: Calc'd m/z=513.3 for C₂₇H₄₃N₇OS, found [M+H]⁺=514.4. ¹H NMR (300 MHz, acetonitrile-d₃) δ 8.39 (d, J=5.5 Hz, 1H), 7.90 (d, J=5.5 Hz, 1H), 4.76 (t, J=7.8 Hz, 2H), 4.34 (s, 4H), 4.04-3.53 (m, 13H), 3.56-3.43 (m, 2H), 3.37 (t, J=7.6 Hz, 2H), 3.23 (t, J=6.1 Hz, 2H), 2.38-2.07 (m, 6H), 1.88 (h, J=7.4 Hz, 5H), 1.39 (t, J=7.3 Hz, 3H).

### 3.10 2-butyl-1-(5-((2-methyl-2-morpholinopropyl)amino)pentyl)-1H-imidazo[4,5-d]thieno [3,2-b]pyridin-4-amine (Compound 177)

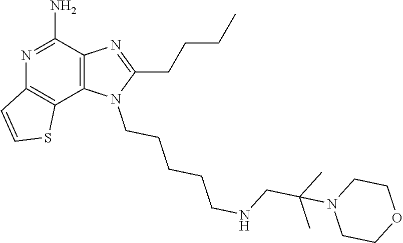 [SMILES: CCCCc1nc2c(N)nc3ccsc3c2n1CCCCCNCC(C)(C)N1CCOCC1]

The title compound was prepared according to General Procedure 9 using Compound 3.d (10 mg), DMA and 2-methyl-2-morpholinopropan-1-amine. Purification was accomplished by preparative HPLC as described in General Procedure 11, eluting with a 10 to 40% CH₃CN/H₂O+0.1% TFA gradient to give Compound 177 as a white solid (7.4 mg, 32% yield, assumed 3×TFA salt).

LC/MS: Calc'd m/z=472.3 for C₂₅H₄₀N₆OS, found [M+H]⁺=473.4. ¹H NMR (300 MHz, acetonitrile-d₃) δ 8.39 (d, J=5.6 Hz, 1H), 7.90 (d, J=5.5 Hz, 1H), 4.77 (t, J=7.7 Hz, 2H), 4.36 (s, 4H), 3.74 (s, 2H), 3.70 (s, 5H), 3.52-3.44 (m, 2H), 3.38 (t, J=7.7 Hz, 2H), 2.37-2.07 (m, 4H), 1.92-1.86 (m, 6H), 1.40 (t, J=7.3 Hz, 3H).

### 3.11 5-((5-(4-amino-2-butyl-1H-imidazo[4,5-d]thieno[3,2-b]pyridin-1-yl)pentyl)amino) pentan-1-ol (Compound 178)

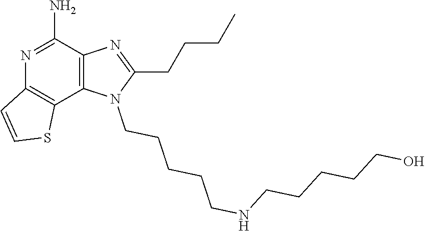 [SMILES: CCCCc1nc2c(N)nc3ccsc3c2n1CCCCCNCCCCCO]

The title compound was prepared according to General Procedure 9 using Compound 3.d (10 mg), DMA and 5-aminopentan-1-ol. Purification was accomplished by preparative HPLC as described in General Procedure 11, eluting with a 10 to 40% CH₃CN/H₂O+0.1% TFA gradient to give Compound 178 as a white solid (7.1 mg, 38% yield, assumed 2×TFA salt).

LC/MS: Calc'd m/z=417.3 for C₂₂H₃₅N₅OS, found [M+H]⁺=418.4. ¹H NMR (300 MHz, acetonitrile-d₃) δ 8.38 (d, J=5.5 Hz, 1H), 7.89 (d, J=5.5 Hz, 1H), 3.94 (t, J=6.4 Hz, 2H), 3.57-3.23 (m, 8H), 2.73-2.36 (m, 5H), 2.37-2.20 (m, 2H), 2.20-1.64 (m, 9H), 1.39 (t, J=7.4 Hz, 3H).

### 3.12 (4-(((5-(4-amino-2-butyl-1H-imidazo[4,5-d]thieno[3,2-b]pyridin-1-yl)pentyl)amino) methyl)phenyl)methanol (Compound 179)

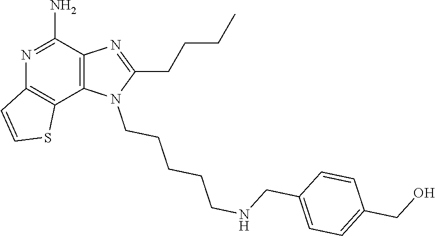 [SMILES: CCCCc1nc2c(N)nc3ccsc3c2n1CCCCCNCc1ccc(CO)cc1]

The title compound was prepared according to General Procedure 9 using Compound 3.d (10 mg), DMA and (4-(aminomethyl)phenyl)methanol. Purification was accomplished by preparative HPLC as described in General Procedure 11, eluting with a 10 to 40% CH₃CN/H₂O+0.1% TFA gradient to give Compound 179 as a white solid (4.0 mg, 21% yield, assumed 2×TFA salt).

LC/MS: Calc'd m/z=451.2 for C₂₅H₃₃N₅OS, found [M+H]⁺=452.2. ¹H NMR (300 MHz, acetonitrile-d₃) δ 8.38 (d, J=5.5 Hz, 1H), 7.90 (d, J=5.5 Hz, 1H), 7.84-7.81 (m, 4H), 5.03 (s, 2H), 4.76 (t, J=7.7 Hz, 2H), 4.55 (s, 2H), 3.36 (t, J=7.7 Hz, 4H), 2.36-2.19 (m, 4H), 2.17-2.04 (m, 2H), 1.98-1.78 (m, 5H), 1.39 (t, J=7.3 Hz, 3H).

### 3.13 1-((5-(4-amino-2-butyl-1H-imidazo[4,5-d]thieno[3,2-b]pyridin-1-yl)pentyl)amino)-2-methylpropan-2-ol (Compound 180)

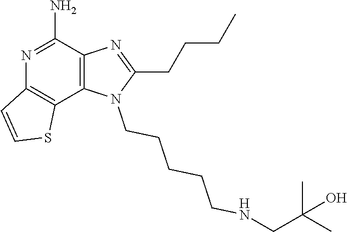 [SMILES: CCCCc1nc2c(N)nc3ccsc3c2n1CCCCCNCC(C)(C)O]

The title compound was prepared according to General Procedure 9 using Compound 3.d (10 mg), DMA and 1-amino-2-methylpropan-2-ol. Purification was accomplished by preparative HPLC as described in General Procedure 11, eluting with a 10 to 40% CH₃CN/H₂O+0.1% TFA gradient to give Compound 180 as a white solid (5.2 mg, 29% yield, assumed 2×TFA salt).

LC/MS: Calc'd m/z=403.2 for C₂₁H₃₃N₅OS, found [M+H]⁺=404.2. ¹H NMR (300 MHz, acetonitrile-d₃) δ 8.39 (d, J=5.5 Hz, 1H), 7.90 (d, J=5.5 Hz, 1H), 4.78 (t, J=7.7 Hz, 2H), 4.35 (s, 2H), 3.86-3.76 (m, 2H), 3.75-3.64 (m, 2H), 3.38 (t, J=7.6 Hz, 2H), 2.37-2.12 (m, 5H), 1.96-1.81 (m, 4H), 1.40 (t, J=7.4 Hz, 3H).

### 3.14 1-((5-(4-amino-2-butyl-1H-imidazo[4,5-d]thieno[3,2-b]pyridin-1-yl)pentyl)amino)-2-methylpropan-2-ol (Compound 182)

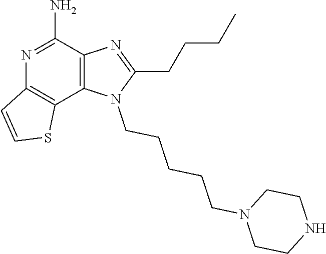 [SMILES: CCCCc1nc2c(N)nc3ccsc3c2n1CCCCCN1CCNCC1]

The title compound was prepared according to General Procedure 9 followed by General Procedure 5 using Compound 3.d (10 mg), DMA and tert-butyl piperazine-1-carboxylate. Purification of the intermediate Boc-protected compound was accomplished by preparative HPLC as described in General Procedure 11, eluting with a 20 to 50% CH₃CN/H₂O+0.1% TFA gradient. Purification was accomplished by preparative HPLC as described in General Procedure 11, eluting with a 10 to 40% CH₃CN/H₂O+0.1% TFA gradient to give Compound 182 as a white solid (2.1 mg, 11% yield, assumed 3×TFA salt).

LC/MS: Calc'd m/z=400.2 for C₂₁H₃₂N₆S, found [M+H]⁺=401.2. ¹H NMR (300 MHz, acetonitrile-d₃) δ 8.55 (d, J=5.5 Hz, 1H), 8.06 (d, J=5.5 Hz, 1H), 4.93 (t, J=6.2 Hz, 2H), 4.03 (s, 4H), 3.95 (s, 4H), 3.62 (t, J=8.2 Hz, 2H), 3.54 (t, J=7.5 Hz, 2H), 2.56-2.19 (m, 6H), 2.15-1.92 (m, 4H), 1.56 (t, J=7.4 Hz, 3H).

### 3.15 1-(5-(((1H-pyrrol-3-yl)methyl)amino)pentyl)-2-butyl-1H-imidazo[4,5-d]thieno[3,2-b]pyridin-4-amine (Compound 183)

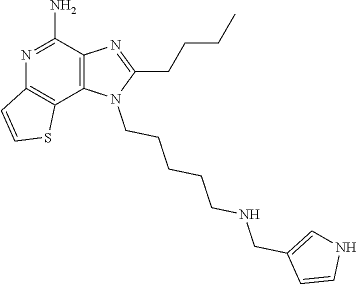 [SMILES: CCCCc1nc2c(N)nc3ccsc3c2n1CCCCCNCc1cc[nH]c1]

The title compound was prepared according to General Procedure 9 using Compound 3.d (10 mg), DMA and (1H-pyrrol-3-yl)methanamine. Purification was accomplished by preparative HPLC as described in General Procedure 11, eluting with a 10 to 40% CH₃CN/H₂O+0.1% TFA gradient to give Compound 183 as a white solid (1.0 mg, 5% yield, assumed 2×TFA salt).

LC/MS: Calc'd m/z=410.2 for C₂₂H₃₀N₆S, found [M+H]⁺=411.2. ¹H NMR (300 MHz, acetonitrile-d₃) δ 10.56 (s, 1H), 8.44 (d, J=5.5 Hz, 2H), 7.96 (d, J=5.5 Hz, 2H), 7.38 (s, 2H), 7.28 (s, 2H), 6.66 (s, 2H), 4.80 (t, J=7.7 Hz, 4H), 4.50 (s, 4H), 3.45-3.33 (m, 9H), 2.38-2.23 (m, 7H), 2.14 (q, J=7.9 Hz, 4H), 2.00-1.89 (m, 9H), 1.46 (t, J=7.4 Hz, 6H).

### 3.16 1-(5-aminopentyl)-2-butyl-1H-imidazo[4,5-d]thieno[3,2-b]pyridin-4-amine (Compound 185)

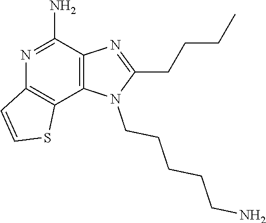 [SMILES: CCCCc1nc2c(N)nc3ccsc3c2n1CCCCCN]

To a solution of Compound 173 (8.0 mg, 0.022 mmol) in 0.4 mL THF/H₂O (1:1) was added triphenylphosphine (9 mg, 1.5 eq.). After stirring for 16 hr at rt the reaction mixture was purified by preparative HPLC as described in General Procedure 11, eluting with a 10 to 40% CH₃CN/H₂O+0.1% TFA gradient to give Compound 185 as a white solid (1.0 mg, 5% yield, assumed 2×TFA salt).

LC/MS: Calc'd m/z=331.2 for C₁₇H₂₅N₅S, found [M+H]⁺=332.2. ¹H NMR (300 MHz, methanol-d₄) δ 8.03 (d, J=5.5 Hz, 1H), 7.48 (d, J=5.5 Hz, 1H), 4.50-4.39 (m, 2H), 3.64-3.54 (m, 2H), 3.08-2.97 (m, 2H), 2.09-1.82 (m, 4H), 1.76-1.48 (m, 7H), 1.05 (t, J=7.3 Hz, 3H).

### Example 4: Preparation of Series 4 Compounds

### 4.1 (3-(((6-nitrothieno[3,2-b]pyridin-7-yl)amino)methyl)phenyl)methanol (Compound 4.a)

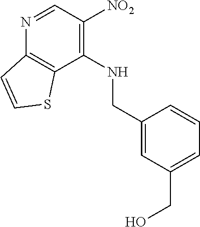 [SMILES: O=[N+]([O-])c1cnc2ccsc2c1NCc1cccc(CO)c1]

The title compound was prepared according to General Procedure 6 starting from Compound 2.a (3.00 g) and (3-(aminomethyl)phenyl)methanol. An aqueous extraction was performed using 10% methanol in dichloromethane. The crude compound was taken to the next step with no additional purification.

LC/MS: Calc'd m/z=315.1 for C₁₅H₁₃N₃O₃S, found [M+H]⁺=316.1.

### 4.2 (3-(((6-aminothieno[3,2-b]pyridin-7-yl)amino)methyl)phenyl)methanol (Compound 4.b)

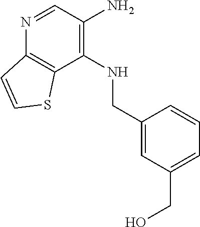 [SMILES: Nc1cnc2ccsc2c1NCc1cccc(CO)c1]

The title compound was prepared according General Procedure 2 starting from crude Compound 4.a. Purification was accomplished as described in General Procedure 11, eluting with a 5 to 40% CH₃CN/H₂O+0.1% TFA gradient to give Compound 4.b as a yellow solid (3.10 g, 43% yield, assumed 2×TFA salt).

LC/MS: Calc'd m/z=285.1 for C₁₅H₁₅N₃OS, found [M+H]⁺=286.2.

### 4.3 (3-((2-butyl-1H-imidazo[4,5-d]thieno[3,2-b]pyridin-1-yl)methyl)phenyl)methanol (Compound 4.c)

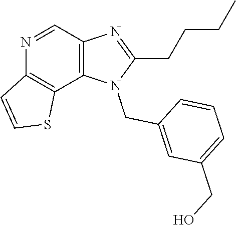 [SMILES: CCCCc1nc2cnc3ccsc3c2n1Cc1cccc(CO)c1]

The title compound was prepared according to General Procedure 7 starting from Compound 4.b (3.10 g). Purification was accomplished as described in General Procedure 11, eluting with 10 to 40% CH₃CN/H₂O+0.1% TFA to give Compound 4.c as an off-white solid (2.95 g, 95% yield, assumed 1×TFA salt).

LC/MS: Calc'd m/z=351.1 for C₂₀H₂₁N₃OS, found [M+H]⁺=352.2.

### 4.4 (3-((4-amino-2-butyl-1H-imidazo[4,5-d]thieno[3,2-b]pyridin-1-yl)methyl)phenyl) methanol (Compound 4.d)

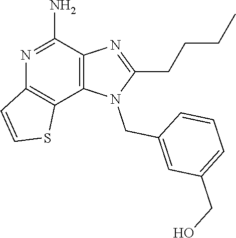 [SMILES: CCCCc1nc2c(N)nc3ccsc3c2n1Cc1cccc(CO)c1]

The title compound was prepared according to General Procedure 4 starting from Compound 4.c (1.30 g) using dichloromethane as the solvent. Purification was accomplished as described in General Procedure 11, eluting with eluting with 10 to 50% CH₃CN/H₂O+0.1% TFA to give Compound 4.d as a white solid (171 mg, 13% yield, assumed 1×TFA salt).

LC/MS: Calc'd m/z=366.2 for C₂₀H₂₂N₄OS, found [M+H]⁺=367.2.

### 4.5 2-butyl-1-(3-(chloromethyl)benzyl)-1H-imidazo[4,5-d]thieno[3,2-b]pyridin-4-amine (Compound 4.e)

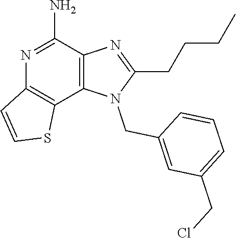 [SMILES: CCCCc1nc2c(N)nc3ccsc3c2n1Cc1cccc(CCl)c1]

The title compound was prepared according to General Procedure 8 starting from Compound 4.d (165 mg). The crude compound was taken to the next step with no additional purification.

LC/MS: Calc'd m/z=384.1 for C₂₀H₂₁ClN₄S, found [M+H]⁺=385.2.

### 4.6 tert-butyl 4-(3-((4-amino-2-butyl-1H-imidazo[4,5-d]thieno[3,2-b]pyridin-1-yl)methyl) benzyl)piperazine-1-carboxylate (Compound 196)

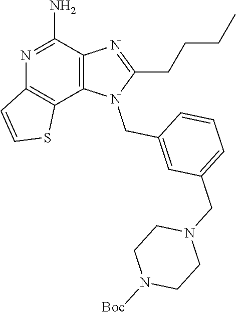 [SMILES: CCCCc1nc2c(N)nc3ccsc3c2n1Cc1cccc(CN2CCN(C(=O)OC(C)(C)C)CC2)c1]

The title compound was prepared according to General Procedure 9 using Compound 4.e (10 mg), DMA and tert-butyl piperazine-1-carboxylate. Purification was accomplished by preparative HPLC as described in General Procedure 11, eluting with a 10 to 50% CH₃CN/H₂O+0.1% TFA gradient to give Compound 196 as a white solid (10.9 mg, 41% yield, assumed 2×TFA salt).

LC/MS: Calc'd m/z=534.3 for C₂₉H₃₈N₆O₂S, found [M+H]⁺=534.8. ¹H NMR (300 MHz, methanol-d₄) δ 7.90 (d, J=5.5 Hz, 1H), 7.61-7.48 (m, 2H), 7.46 (d, J=5.4 Hz, 1H), 7.35-7.24 (m, 2H), 5.79 (s, 2H), 4.32 (s, 2H), 3.97-3.38 (m, 4H), 3.13 (s, 4H), 3.04-2.93 (m, 2H), 1.94-1.77 (m, 2H), 1.51-1.43 (m, 2H), 1.51-1.39 (m, 2H), 1.49 (s, 9H), 0.96 (t, J=7.3 Hz, 3H).

### 4.7 1-(3-((((1H-pyrrol-3-yl)methyl)amino)methyl)benzyl)-2-butyl-1H-imidazo[4,5-d]thieno [3,2-b]pyridin-4-amine (Compound 197)

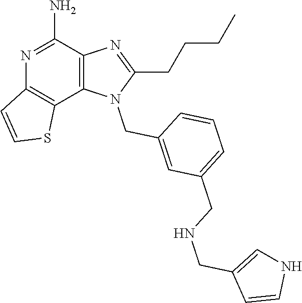 [SMILES: CCCCc1nc2c(N)nc3ccsc3c2n1Cc1cccc(CNCc2cc[nH]c2)c1]

The title compound was prepared according to General Procedure 9 using Compound 4.e (10 mg), DMA and (1H-pyrrol-3-yl)methanamine. Purification was accomplished by preparative HPLC as described in General Procedure 11, eluting with a 10 to 50% CH₃CN/H₂O+0.1% TFA gradient to give Compound 197 as a white solid (8.8 mg, 34% yield, assumed 2×TFA salt).

LC/MS: Calc'd m/z=444.2 for C₂₅H₂₈N₆S, found [M+H]⁺=444.8. ¹H NMR (300 MHz, methanol-d₄) δ 10.64 (s, 1H), 7.89 (d, J=5.5 Hz, 1H), 7.54-7.46 (m, 2H), 7.45 (d, J=5.5 Hz, 1H), 7.29-7.18 (m, 2H), 6.91-6.83 (m, 1H), 6.83-6.75 (m, 1H), 6.24-6.02 (m, 1H), 5.77 (s, 2H), 4.13 (s, 2H), 4.01 (s, 2H), 3.02-2.91 (m, 2H), 1.86 (p, J=7.6 Hz, 2H), 1.62-1.34 (m, 2H), 0.96 (t, J=7.3 Hz, 3H).

### 4.8 2-butyl-1-(3-((4-(pyridin-4-yl)piperidin-1-yl)methyl)benzyl)-1H-imidazo[4,5-d]thieno [3,2-b]pyridin-4-amine (Compound 198)

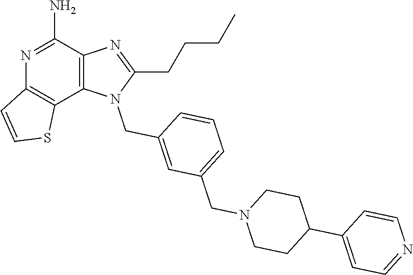 [SMILES: CCCCc1nc2c(N)nc3ccsc3c2n1Cc1cccc(CN2CCC(c3ccncc3)CC2)c1]

The title compound was prepared according to General Procedure 9 using Compound 4.e (10 mg), DMA and 4-(piperidin-4-yl)pyridine. Purification was accomplished by preparative HPLC as described in General Procedure 11, eluting with a 10 to 50% CH₃CN/H₂O+0.1% TFA gradient to give Compound 198 as a white solid (13.9 mg, 42% yield, assumed 3×TFA salt).

LC/MS: Calc'd m/z=510.3 for C₃₀H₃₄N₆S, found [M+H]⁺=510.8. ¹H NMR (300 MHz, methanol-d₄) δ 8.82-8.73 (m, 2H), 7.96-7.85 (m, 2H), 7.61-7.48 (m, 2H), 7.45 (d, J=5.4 Hz, 1H), 7.36 (s, 1H), 7.28 (dt, J=5.0, 2.0 Hz, 1H), 5.80 (s, 2H), 4.37 (s, 2H), 3.58-3.48 (m, 2H), 3.24-3.10 (m, 1H), 3.05-2.94 (m, 2H), 2.44-1.96 (m, 4H), 1.94-1.78 (m, 2H), 1.57-1.38 (m, 2H), 0.96 (t, J=7.4 Hz, 3H).

### 4.9 2-butyl-1-(3-((diethylamino)methyl)benzyl)-1H-imidazo[4,5-d]thieno[3,2-b]pyridin-4-amine (Compound 199)

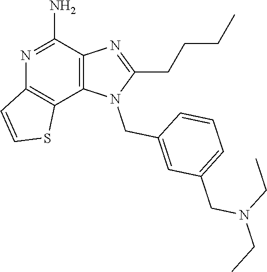 [SMILES: CCCCc1nc2c(N)nc3ccsc3c2n1Cc1cccc(CN(CC)CC)c1]

The title compound was prepared according to General Procedure 9 using Compound 4.e (10 mg), DMA and diethylamine. Purification was accomplished by preparative HPLC as described in General Procedure 11, eluting with a 10 to 50% CH₃CN/H₂O+0.1% TFA gradient to give Compound 199 as a white solid (9.9 mg, 39% yield, assumed 2×TFA salt).

LC/MS: Calc'd m/z=421.2 for C₂₄H₃₁N₅S, found [M+H]⁺=421.8. ¹H NMR (300 MHz, methanol-d₄) δ 7.90 (d, J=5.4 Hz, 1H), 7.61-7.48 (m, 2H), 7.46 (d, J=5.4 Hz, 1H), 7.30 (dt, J=7.0, 1.9 Hz, 1H), 7.26 (s, 1H), 5.80 (s, 2H), 4.31 (s, 2H), 3.10 (qd, J=7.3, 4.9 Hz, 4H), 2.98 (dd, J=8.5, 6.8 Hz, 2H), 1.85 (p, J=7.8 Hz, 2H), 1.46 (h, J=7.6 Hz, 2H), 1.24 (t, J=7.3 Hz, 6H), 0.96 (t, J=7.4 Hz, 3H).

### 4.10 2-butyl-1-(3-((4-(2-morpholinoethyl)piperazin-1-yl)methyl)benzyl)-1H-imidazo[4,5-d]thieno[3,2-b]pyridin-4-amine (Compound 200)

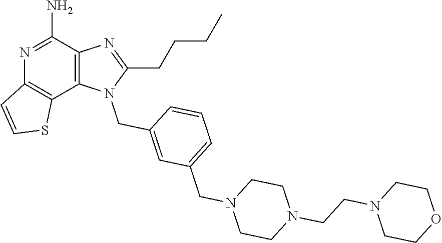 [SMILES: CCCCc1nc2c(N)nc3ccsc3c2n1Cc1cccc(CN2CCN(CCN3CCOCC3)CC2)c1]

The title compound was prepared according to General Procedure 9 using Compound 4.e (10 mg), DMA and 4-(2-(piperazin-1-yl)ethyl)morpholine. Purification was accomplished by preparative HPLC as described in General Procedure 11, eluting with a 10 to 50% CH₃CN/H₂O+0.1% TFA gradient to give Compound 200 as a white solid (1.3 mg, 14% yield, assumed 4×TFA salt).

LC/MS: Calc'd m/z=421.2 for C₃₀H₄₁N₇OS, found [M+H]⁺=421.8. ¹H NMR (300 MHz, methanol-d₄) δ 7.91 (d, J=5.5 Hz, 1H), 7.50 (d, J=6.2 Hz, 2H), 7.45 (d, J=5.5 Hz, 1H), 7.32 (s, 1H), 7.23-7.18 (m, 1H), 5.78 (s, 2H), 4.28-4.08 (m, 2H), 3.95-3.89 (m, 5H), 3.31-3.21 (m, 8H), 3.03-2.92 (m, 2H), 2.90 (s, 3H), 1.84 (dd, J=15.2, 7.5 Hz, 2H), 1.54-1.40 (m, 2H), 0.96 (t, J=7.4 Hz, 3H).

### 4.11 1-(3-(aminomethyl)benzyl)-2-butyl-1H-imidazo[4,5-d]thieno[3,2-b]pyridin-4-amine (Compound 201)

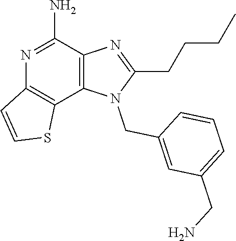 [SMILES: CCCCc1nc2c(N)nc3ccsc3c2n1Cc1cccc(CN)c1]

The title compound was prepared according to General Procedure 9 using Compound 4.e (10 mg), DMA and 0.5 mL 2 M ammonia in methanol. Purification was accomplished by preparative HPLC as described in General Procedure 11, eluting with a 10 to 50% CH₃CN/H₂O+0.1% TFA gradient to give Compound 201 as a white solid (3.2 mg, 14% yield, assumed 3×TFA salt).

LC/MS: Calc'd m/z=365.2 for C₂₀H₂₃N₅S, found [M+H]⁺=365.8. ¹H NMR (300 MHz, methanol-d₄) δ 7.91 (d, J=5.4 Hz, 1H), 7.54-7.41 (m, 3H), 7.26 (s, 1H), 7.23-7.13 (m, 1H), 5.77 (s, 2H), 4.10 (s, 2H), 3.02-2.91 (m, 2H), 1.87 (p, J=7.6 Hz, 2H), 1.48 (h, J=7.4 Hz, 2H), 0.97 (t, J=7.3 Hz, 3H).

### 4.12 2-butyl-1-(3-(((2-methyl-2-morpholinopropyl)amino)methyl)benzyl)-1H-imidazo[4,5-d]thieno[3,2-b]pyridin-4-amine (Compound 202)

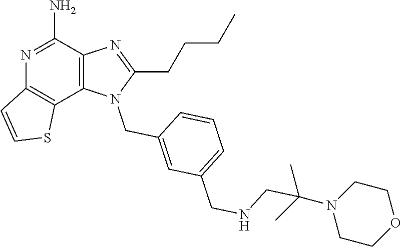 [SMILES: CCCCc1nc2c(N)nc3ccsc3c2n1Cc1cccc(CNCC(C)(C)N2CCOCC2)c1]

The title compound was prepared according to General Procedure 9 using Compound 4.e (10 mg), DMA and 2-methyl-2-morpholinopropan-1-amine. Purification was accomplished by preparative HPLC as described in General Procedure 11, eluting with a 10 to 50% CH₃CN/H₂O+0.1% TFA gradient to give Compound 202 as a white solid (12.5 mg, 38% yield, assumed 3×TFA salt).

LC/MS: Calc'd m/z=506.3 for C₂₈H₃₈N₆OS, found [M+H]⁺=507.0. ¹H NMR (300 MHz, methanol-d₄) δ 7.92 (d, J=5.5 Hz, 1H), 7.60-7.48 (m, 2H), 7.47 (d, J=5.5 Hz, 1H), 7.34-7.20 (m, 2H), 5.79 (s, 2H), 4.19 (s, 2H), 3.63 (t, J=4.7 Hz, 4H), 3.04-2.92 (m, 4H), 2.61 (s, 4H), 1.88 (p, J=7.6 Hz, 2H), 1.58-1.40 (m, 2H), 1.15 (s, 6H), 0.97 (t, J=7.4 Hz, 3H).

### 4.13 5-((3-((4-amino-2-butyl-1H-imidazo[4,5-d]thieno[3,2-b]pyridin-1-yl)methyl)benzyl) amino)pentan-1-ol (Compound 203)

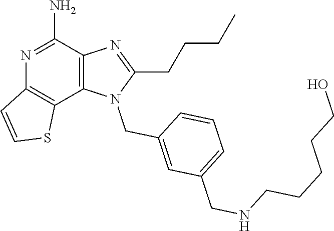 [SMILES: CCCCc1nc2c(N)nc3ccsc3c2n1Cc1cccc(CNCCCCCO)c1]

The title compound was prepared according to General Procedure 9 using Compound 4.e (10 mg), DMA and 5-aminopentan-1-ol. Purification was accomplished by preparative HPLC as described in General Procedure 11, eluting with a 10 to 50% CH₃CN/H₂O+0.1% TFA gradient to give Compound 203 as a white solid (11.2 mg, 42% yield, assumed 2×TFA salt).

LC/MS: Calc'd m/z=451.2 for C₂₅H₃₃N₅OS, found [M+H]⁺=451.8. ¹H NMR (300 MHz, methanol-d₄) δ 7.91 (d, J=5.5 Hz, 1H), 7.56-7.48 (m, 2H), 7.46 (d, J=5.5 Hz, 1H), 7.32-7.21 (m, 2H), 5.78 (s, 2H), 4.18 (s, 2H), 3.58 (t, J=6.2 Hz, 2H), 3.05-2.87 (m, 4H), 1.87 (p, J=7.6 Hz, 2H), 1.67 (p, J=7.6 Hz, 2H), 1.60-1.30 (m, 6H), 0.97 (t, J=7.3 Hz, 3H).

### 4.14 (4-(((3-((4-amino-2-butyl-1H-imidazo[4,5-d]thieno[3,2-b]pyridin-1-yl)methyl)benzyl) amino)methyl)phenyl)methanol (Compound 204)

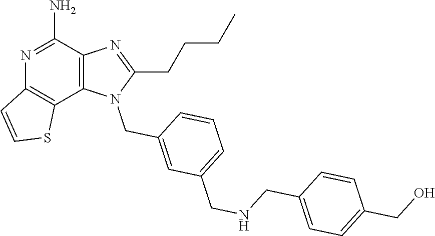 [SMILES: CCCCc1nc2c(N)nc3ccsc3c2n1Cc1cccc(CNCc2ccc(CO)cc2)c1]

The title compound was prepared according to General Procedure 9 using Compound 4.e (10 mg), DMA and (4-(aminomethyl)phenyl)methanol. Purification was accomplished by preparative HPLC as described in General Procedure 11, eluting with a 10 to 50% CH₃CN/H₂O+0.1% TFA gradient to give Compound 204 as a white solid (10.3 mg, 55% yield, assumed 2×TFA salt).

LC/MS: Calc'd m/z=485.2 for C₂₈H₃₁N₅OS, found [M+H]⁺=485.8. ¹H NMR (300 MHz, methanol-d₄) δ 7.89 (d, J=5.5 Hz, 1H), 7.54-7.46 (m, 2H), 7.47-7.41 (m, 3H), 7.36 (d, J=8.2 Hz, 2H), 7.31-7.25 (m, 1H), 7.23 (s, 1H), 5.78 (s, 2H), 4.65 (s, 2H), 4.21 (s, 2H), 4.12 (s, 2H), 2.97 (t, J=7.4 Hz, 2H), 1.87 (p, J=7.6 Hz, 2H), 1.47 (h, J=7.4 Hz, 2H), 0.96 (t, J=7.4 Hz, 3H).

### 4.15 1-((3-((4-amino-2-butyl-1H-imidazo[4,5-d]thieno[3,2-b]pyridin-1-yl)methyl)benzyl) amino)-2-methylpropan-2-ol (Compound 205)

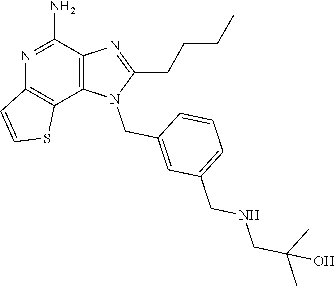 [SMILES: CCCCc1nc2c(N)nc3ccsc3c2n1Cc1cccc(CNCC(C)(C)O)c1]

The title compound was prepared according to General Procedure 9 using Compound 4.e (10 mg), DMA and 1-amino-2-methylpropan-2-ol. Purification was accomplished by preparative HPLC as described in General Procedure 11, eluting with a 10 to 50% CH₃CN/H₂O+0.1% TFA gradient to give Compound 205 as a white solid (10.3 mg, 59% yield, assumed 2×TFA salt).

LC/MS: Calc'd m/z=437.2 for C₂₄H₃₁N₅OS, found [M+H]⁺=437.8. ¹H NMR (300 MHz, methanol-d₄) δ 7.89 (d, J=5.5 Hz, 1H), 7.54-7.48 (m, 2H), 7.46 (d, J=5.5 Hz, 1H), 7.31-7.24 (m, 2H), 5.78 (s, 2H), 4.22 (s, 2H), 3.04-2.93 (m, 2H), 2.76 (s, 2H), 1.87 (d, J=7.1 Hz, 2H), 1.48 (d, J=7.5 Hz, 2H), 1.20 (s, 6H), 0.97 (t, J=7.3 Hz, 3H).

### 4.16 2-butyl-1-(3-(hydrazineylmethyl)benzyl)-1H-imidazo[4,5-d]thieno[3,2-b]pyridin-4-amine (Compound 206)

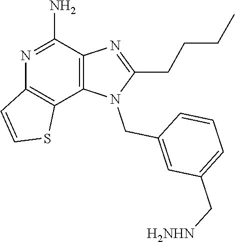

The title compound was prepared according to General Procedure 9 using Compound 4.e (10 mg), DMA and hydrazine monohydrate. Purification was accomplished by preparative HPLC as described in General Procedure 11, eluting with a 10 to 50% CH₃CN/H₂O+0.1% TFA gradient to give Compound 206 as a white solid (7.2 mg, 30% yield, assumed 2×TFA salt).

LC/MS: Calc'd m/z=380.2 for C₂₀H₂₄N₆S, found [M+H]⁺=380.8. ¹H NMR (300 MHz, methanol-d₄) δ 7.91 (d, J=5.5 Hz, 1H), 7.46 (d, J=5.4 Hz, 1H), 7.45-7.41 (m, 2H), 7.23 (s, 1H), 7.20-7.11 (m, 1H), 5.76 (s, 2H), 4.11 (s, 2H), 3.02-2.91 (m, 2H), 1.87 (p, J=7.6 Hz, 2H), 1.45 (h, J=7.6 Hz, 2H), 0.96 (t, J=7.3 Hz, 3H).

### 4.17 2-butyl-1-(3-(piperazin-1-ylmethyl)benzyl)-1H-imidazo[4,5-d]thieno[3,2-b]pyridin-4-amine (Compound 214)

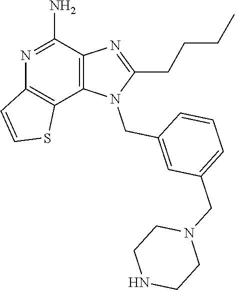 [SMILES: CCCCc1nc2c(N)nc3ccsc3c2n1Cc1cccc(CN2CCNCC2)c1]

The title compound was prepared according to General Procedure 5 starting from Compound 196 (8.1 mg). Purification was accomplished by preparative HPLC as described in General Procedure 11, eluting with a 10 to 40% CH₃CN/H₂O+0.1% TFA gradient to give Compound 214 as a white solid (5.9 mg, 73% yield, assumed 3×TFA salt).

LC/MS: Calc'd m/z=434.2 for C₂₄H₃₀N₆S, found [M+H]⁺=434.8. ¹H NMR (300 MHz, methanol-d₄) δ 7.91 (d, J=5.5 Hz, 1H), 7.46 (d, J=5.5 Hz, 1H), 7.42-7.32 (m, 2H), 7.18 (s, 1H), 7.12-7.03 (m, 1H), 5.74 (s, 2H), 3.69 (s, 2H), 3.25-3.15 (m, 4H), 2.97 (t, J=7.6 Hz, 2H), 2.78-2.68 (m, 4H), 1.83 (p, J=7.6 Hz, 2H), 1.46 (h, J=7.3 Hz, 2H), 0.95 (t, J=7.3 Hz, 3H).

### 4.18 1-(3-((1H-imidazol-1-yl)methyl)benzyl)-2-butyl-1H-imidazo[4,5-d]thieno[3,2-b]pyridin-4-amine (Compound 229)

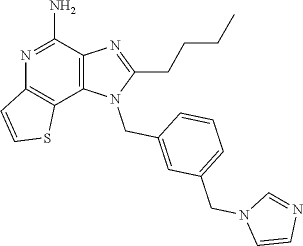 [SMILES: CCCCc1nc2c(N)nc3ccsc3c2n1Cc1cccc(Cn2ccnc2)c1]

The title compound was prepared according to General Procedure 9 using Compound 4.e (10 mg), DMA, potassium carbonate in place of DIPEA, and imidazole. Purification was accomplished by preparative HPLC as described in General Procedure 11, eluting with a 10 to 50% CH₃CN/H₂O+0.1% TFA gradient to give Compound 229 as a white solid (3.0 mg, 18% yield, assumed 2×TFA salt).

LC/MS: Calc'd m/z=416.2 for C₂₃H₂₄N₆S, found [M+H]⁺=416.8. ¹H NMR (300 MHz, methanol-d₄) δ 8.97 (d, J=1.5 Hz, 1H), 7.90 (d, J=5.6 Hz, 1H), 7.62-7.47 (m, 3H), 7.46 (d, J=5.5 Hz, 1H), 7.43-7.35 (m, 1H), 7.21-7.13 (m, 2H), 5.75 (s, 2H), 5.43 (s, 2H), 3.01-2.90 (m, 2H), 1.81 (p, J=7.6 Hz, 2H), 1.44 (h, J=7.4 Hz, 2H), 0.93 (t, J=7.4 Hz, 3H).

### Example 5: Preparation of Series 5 Compounds

### 5.1 (2-(((6-nitrothieno[3,2-b]pyridin-7-yl)amino)methyl)phenyl)methanol (Compound 5.a)

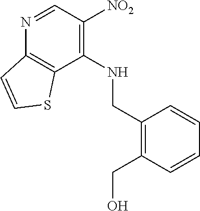 [SMILES: O=[N+]([O-])c1cnc2ccsc2c1NCc1ccccc1CO]

The title compound was prepared according to General Procedure 1 starting from Compound 2.a (3.00 g) and (2-(aminomethyl)phenyl)methanol. Aqueous extraction was performed using 10% methanol in dichloromethane. The crude compound was taken to the next step with no additional purification.

LC/MS: Calc'd m/z=315.1 for C₁₅H₁₃N₃O₃S, found [M+H]⁺=316.1.

### 5.2 (2-(((6-aminothieno[3,2-b]pyridin-7-yl)amino)methyl)phenyl)methanol (Compound 5.b)

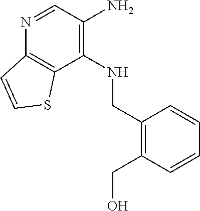 [SMILES: Nc1cnc2ccsc2c1NCc1ccccc1CO]

The title compound was prepared according General Procedure 2 starting from crude Compound 5.a. Purification was accomplished as described in General Procedure 11, eluting with 5 to 40% CH₃CN/H₂O+0.1% TFA to give Compound 5.b as a yellow solid (1.60 g, 22% yield, assumed 2×TFA salt).

LC/MS: Calc'd m/z=285.1 for C₁₅H₁₅N₃OS, found [M+H]⁺=286.2

### 5.3 (2-((2-butyl-1H-imidazo[4,5-d]thieno[3,2-b]pyridin-1-yl)methyl)phenyl)methanol (Compound 5.c)

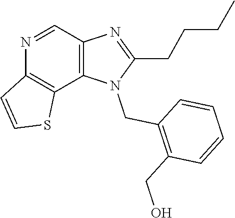 [SMILES: CCCCc1nc2cnc3ccsc3c2n1Cc1ccccc1CO]

The title compound was prepared according to General Procedure 7 starting from Compound 5.b (1.60 g). Purification was accomplished as described in General Procedure 11, eluting with 10 to 50% CH₃CN/H₂O+0.1% TFA to give Compound 5.c as a grey solid (1.38 g, 95% yield, assumed 1×TFA salt).

LC/MS: Calc'd m/z=351.1 for C₂₀H₂₁N₃OS, found [M+H]⁺=352.2.

### 5.4 (2-((4-amino-2-butyl-1H-imidazo[4,5-d]thieno[3,2-b]pyridin-1-yl)methyl)phenyl) methanol (Compound 5.d)

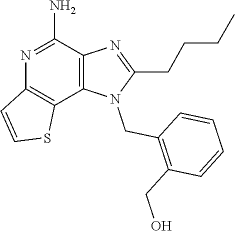 [SMILES: CCCCc1nc2c(N)nc3ccsc3c2n1Cc1ccccc1CO]

The title compound was prepared according to General Procedure 4 starting from Compound 5.c (1.38 g) using dichloromethane as the solvent. Purification was accomplished as described in General Procedure 11, eluting with eluting with 10 to 50% CH₃CN/H₂O+0.1% TFA to give Compound 5.d as a grey solid (170 mg, 12% yield, assumed 1×TFA salt).

LC/MS: Calc'd m/z=366.1 for C₂₀H₂₂N₄OS, found [M+H]⁺=367.2.

### 5.5 2-butyl-1-(2-(chloromethyl)benzyl)-1H-imidazo[4,5-d]thieno[3,2-b]pyridin-4-amine (Compound 5.e)

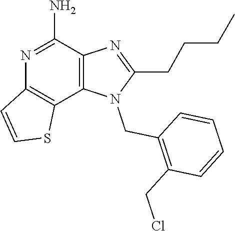 [SMILES: CCCCc1nc2c(N)nc3ccsc3c2n1Cc1ccccc1CCl]

The title compound was prepared according to General Procedure 8 starting from Compound 5.d (110 mg). The crude compound was taken to the next step with no additional purification.

LC/MS: Calc'd m/z=384.1 for C₂₀H₂₁ClN₄S, found [M+H]⁺=385.2.

### 5.6 1-(2-(((3-(4-(3-aminopropyl)piperazin-1-yl)propyl)amino)methyl)benzyl)-2-butyl-1H-imidazo[4,5-d]thieno[3,2-b]pyridin-4-amine (Compound 215)

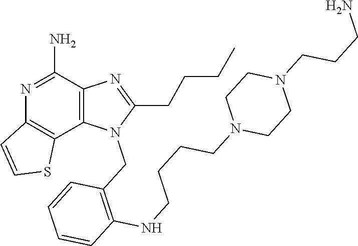 [SMILES: CCCCc1nc2c(N)nc3ccsc3c2n1Cc1ccccc1NCCCCN1CCN(CCCN)CC1]

The title compound was prepared according to General Procedure 9 using Compound 5.e (10 mg), DMA and 3,3′-(piperazine-1,4-diyl)bis(propan-1-amine). Purification was accomplished by preparative HPLC as described in General Procedure 11, eluting with a 10 to 40% CH₃CN/H₂O+0.1% TFA gradient to give Compound 215 as a white solid (3.9 mg, 13% yield, assumed 5×TFA salt).

LC/MS: Calc'd m/z=548.3 for C₃₀H₄₄N₈S, found [M+H]⁺=549.4. ¹H NMR (300 MHz, acetonitrile-d₃) δ 7.77 (d, J=5.5 Hz, 1H), 7.62 (d, J=7.9 Hz, 1H), 7.41 (t, J=7.5 Hz, 1H), 7.40 (d, J=5.4 Hz, 1H), 7.22 (t, J=7.2 Hz, 1H), 6.43 (d, J=7.8 Hz, 1H), 5.77 (s, 2H), 4.49 (s, 2H), 3.31-3.17 (m, 10H), 3.12-2.95 (m, 6H), 2.91 (t, J=7.6 Hz, 2H), 2.18 (p, J=8.3 Hz, 2H), 2.05-1.90 (m, 2H) 1.84-1.68 (m, 2H), 1.39 (h, J=7.4 Hz, 2H), 0.90 (t, J=7.3 Hz, 3H).

### 5.7 1-(2-(aminomethyl)benzyl)-2-butyl-1H-imidazo[4,5-d]thieno[3,2-b]pyridin-4-amine (Compound 216)

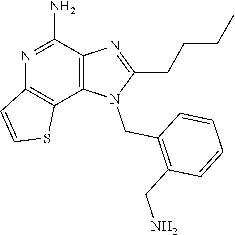 [SMILES: CCCCc1nc2c(N)nc3ccsc3c2n1Cc1ccccc1CN]

The title compound was prepared according to General Procedure 9 using Compound 5.e (10 mg), DMA and 0.5 mL 2 M ammonia in methanol. Purification was accomplished by preparative HPLC as described in General Procedure 11, eluting with a 10 to 40% CH₃CN/H₂O+0.1% TFA gradient to give Compound 216 as a white solid (1.4 mg, 9% yield, assumed 2×TFA salt).

LC/MS: Calc'd m/z=365.1 for C₂₀H₂₃N₅S, found [M+H]⁺=366.2. ¹H NMR (300 MHz, acetonitrile-d₃) δ 7.77 (d, J=5.4 Hz, 1H), 7.56 (d, J=7.9 Hz, 1H), 7.41 (t, J=7.5 Hz, 1H), 7.40 (d, J=5.4 Hz, 1H), 7.20 (t, J=7.7 Hz, 1H), 6.41 (d, J=7.8 Hz, 1H), 5.71 (s, 2H), 4.41 (s, 2H), 2.90 (t, J=7.6 Hz, 2H), 1.76 (p, J=7.5 Hz, 2H), 1.41 (h, J=7.4 Hz, 2H), 0.90 (t, J=7.4 Hz, 3H).

### 5.8 tert-butyl 4-(2-((4-amino-2-butyl-1H-imidazo[4,5-d]thieno[3,2-b]pyridin-1-yl)methyl) benzyl)piperazine-1-carboxylate (Compound 217)

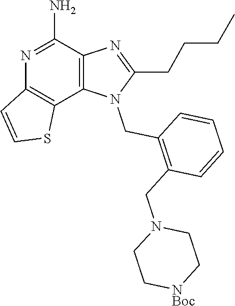 [SMILES: CCCCc1nc2c(N)nc3ccsc3c2n1Cc1ccccc1CN1CCN(C(=O)OC(C)(C)C)CC1]

The title compound was prepared according to General Procedure 9 using Compound 5.e (20 mg), DMA and tert-butyl piperazine-1-carboxylate. Purification was accomplished by preparative HPLC as described in General Procedure 11, eluting with a 10 to 50% CH₃CN/H₂O+0.1% TFA gradient to give Compound 217 as a white solid (6.0 mg, 13% yield, assumed 3×TFA salt).

LC/MS: Calc'd m/z=534.3 for C₂₉H₃₈N₆O₂S, found [M+H]⁺=535.4. ¹H NMR (300 MHz, acetonitrile-d₃) δ 7.77 (d, J=5.4, Hz, 1H), 7.61 (d, J=7.7 Hz, 1H), 7.40 (t, J=7.5 Hz, 1H), 7.40 (d, J=5.4 Hz, 1H), 7.24 (td, J=7.7, 1.4 Hz, 1H), 6.45 (d, J=7.6 Hz, 1H), 5.79 (s, 2H), 4.43 (s, 2H), 3.74-3.58 (m, 4H), 3.22-3.11 (m, 4H), 2.89 (t, J=7.6 Hz, 2H), 1.85-1.66 (m, 2H), 1.46 (s, 9H), 1.42 (h, J=7.4 Hz, 2H), 0.89 (t, J=7.3 Hz, 3H).

### 5.9 2-butyl-1-(2-(piperazin-1-ylmethyl)benzyl)-1H-imidazo[4,5-d]thieno[3,2-b]pyridin-4-amine (Compound 218)

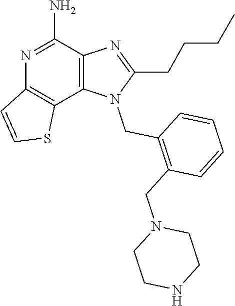 [SMILES: CCCCc1nc2c(N)nc3ccsc3c2n1Cc1ccccc1CN1CCNCC1]

The title compound was prepared according to General Procedure 5 starting from Compound 217 (3.2 mg). The crude product was taken up in 50% CH₃CN/H₂O and lyophilized to give Compound 218 as a white solid (3.2 mg, 100% yield, assumed 4×TFA salt).

LC/MS: Calc'd m/z=434.2 for C₂₄H₃₀N₆S, found [M+H]⁺=435.4.

### 5.10 2-butyl-1-(2-((4-(pyridin-4-yl)piperidin-1-yl)methyl)benzyl)-1H-imidazo[4,5-d]thieno[3,2-b]pyridin-4-amine (Compound 219)

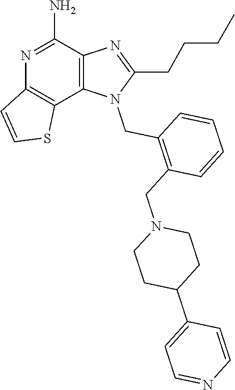 [SMILES: CCCCc1nc2c(N)nc3ccsc3c2n1Cc1ccccc1CN1CCC(c2ccncc2)CC1]

The title compound was prepared according to General Procedure 9 using Compound 5.e (10 mg), DMA and 4-(piperidin-4-yl)pyridine. Purification was accomplished by preparative HPLC as described in General Procedure 11, eluting with a 10 to 40% CH₃CN/H₂O+0.1% TFA gradient to give Compound 219 as a white solid (3.0 mg, 14% yield, assumed 3×TFA salt).

LC/MS: Calc'd m/z=510.3 for C₃₀H₃₄N₆S, found [M+H]⁺=511.4. ¹H NMR (300 MHz, acetonitrile-d₃) δ 8.68 (d, J=6.5 Hz, 2H), 7.90 (d, J=7.2 Hz, 2H), 7.77 (d, J=5.5 Hz, 1H), 7.72 (d, J=7.7, Hz, 1H), 7.45 (t, J=7.0 Hz, 1H), 7.42 (d, J=5.3 Hz, 1H), 7.28 (t, J=7.7 Hz, 1H), 6.49 (d, J=7.8 Hz, 1H), 5.82 (s, 2H), 4.63 (s, 2H), 3.73 (d, J=12.3 Hz, 2H), 2.90 (t, J=7.7 Hz, 2H), 2.32-2.04 (m, 4H), 1.77 (p, J=7.4 Hz, 2H), 1.41 (h, J=7.4 Hz, 2H), 0.90 (t, J=7.4 Hz, 3H).

### 5.11 1-(2-((((1H-pyrrol-3-yl)methyl)amino)methyl)benzyl)-2-butyl-1H-imidazo[4,5-d]thieno [3,2-b]pyridin-4-amine (Compound 220)

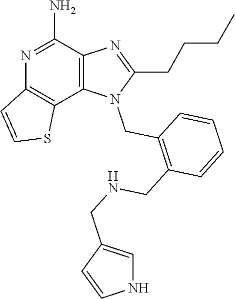 [SMILES: CCCCc1nc2c(N)nc3ccsc3c2n1Cc1ccccc1CNCc1cc[nH]c1]

The title compound was prepared according to General Procedure 9 using Compound 5.e (10 mg), DMA and (1H-pyrrol-3-yl)methanamine. Purification was accomplished by preparative HPLC as described in General Procedure 11, eluting with a 10 to 40% CH₃CN/H₂O+0.1% TFA gradient to give Compound 220 as a white solid (1.8 mg, 10% yield, assumed 2×TFA salt).

LC/MS: Calc'd m/z=444.2 for C₂₅H₂₈N₆S, found [M+H]⁺=444.4. ¹H NMR (300 MHz, acetonitrile-d₃) δ 10.0 (s, 1H), 7.77 (d, J=5.5 Hz, 1H), 7.55 (d, J=7.7 Hz, 1H), 7.46-7.32 (m, 2H), 7.21 (t, J=7.7 Hz, 1H), 7.03 (s, 1H), 6.80-6.76 (m, 1H), 6.42 (d, J=7.8 Hz, 1H), 6.34-6.29 (m, 1H), 5.58 (s, 2H), 4.41 (s, 2H), 4.27 (s, 2H), 2.86 (t, J=7.6 Hz, 2H), 1.75 (p, J=7.4 Hz, 2H), 1.40 (h, J=7.5 Hz, 2H), 0.90 (t, J=7.3 Hz, 3H).

### 5.12 2-butyl-1-(2-((diethylamino)methyl)benzyl)-1H-imidazo[4,5-d]thieno[3,2-b]pyridin-4-amine (Compound 221)

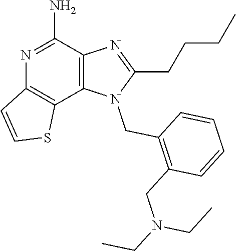 [SMILES: CCCCc1nc2c(N)nc3ccsc3c2n1Cc1ccccc1CN(CC)CC]

The title compound was prepared according to General Procedure 9 using Compound 5.e (10 mg), DMA and diethylamine. Purification was accomplished by preparative HPLC as described in General Procedure 11, eluting with a 10 to 40% CH₃CN/H₂O+0.1% TFA gradient to give Compound 221 as a white solid (2.2 mg, 11% yield, assumed 3×TFA salt).

LC/MS: Calc'd m/z=421.2 for C₂₄H₃₁N₅S, found [M+H]⁺=422.2. ¹H NMR (300 MHz, acetonitrile-d₃) δ 7.77 (d, J=5.5 Hz, 1H), 7.64 d, J=7.6 Hz, 1H), 7.54 (t, J=7.3 Hz, 1H), 7.41 (d, J=5.2 Hz, 1H), 7.27 (t, J=7.6, Hz, 1H), 6.47 (d, J=8.0 Hz, 1H), 5.74 (s, 2H), 4.54 (s, 2H), 2.89 (t, J=7.0 Hz, 2H), 1.99-1.91 (m, 4H), 1.75 (p, J=7.5 Hz, 2H), 1.47-1.27 (m, 8H), 0.89 (t, J=7.4 Hz, 3H).

### 5.13 2-butyl-1-(2-((4-(2-morpholinoethyl)piperazin-1-yl)methyl)benzyl)-1H-imidazo[4,5-d]thieno[3,2-b]pyridin-4-amine (Compound 222)

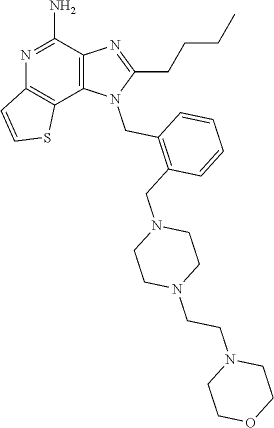 [SMILES: CCCCc1nc2c(N)nc3ccsc3c2n1Cc1ccccc1CN1CCN(CCN2CCOCC2)CC1]

The title compound was prepared according to General Procedure 9 using Compound 5.e (10 mg), DMA and 4-(2-(piperazin-1-yl)ethyl)morpholine. Purification was accomplished by preparative HPLC as described in General Procedure 11, eluting with a 10 to 40% CH₃CN/H₂O+0.1% TFA gradient to give Compound 222 as a white solid (2.7 mg, 11% yield, assumed 4×TFA salt).

LC/MS: Calc'd m/z=547.3 for C₃₀H₄₁N₇OS, found [M+H]⁺=548.3. ¹H NMR (300 MHz, acetonitrile-d₃) δ 7.78 (d, J=5.5 Hz, 1H), 7.58 (d, J=7.6 Hz, 1H), 7.41 (d, J=5.4 Hz, 1H), 7.39 (t, J=7.7 Hz, 1H), 7.23 (t, J=7.7, Hz, 1H), 6.44 (d, J=7.8 Hz, 1H), 5.80 (s, 2H), 4.35 (s, 2H), 3.89 (t, J=4.9 Hz, 4H), 3.31-3.19 (m, 12H), 3.01 (m, 4H), 2.90 (t, J=7.6 Hz, 2H), 1.75 (p, J=7.5 Hz, 2H), 1.39 (h, J=7.3 Hz, 2H), 0.88 (t, J=7.4 Hz, 3H).

### 5.14 4-((2-((4-amino-2-butyl-1H-imidazo[4,5-d]thieno[3,2-b]pyridin-1-yl)methyl)benzyl) amino)butan-1-ol (Compound 223)

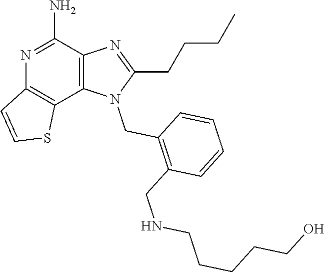 [SMILES: CCCCc1nc2c(N)nc3ccsc3c2n1Cc1ccccc1CNCCCCCO]

The title compound was prepared according to General Procedure 9 using Compound 5.e (10 mg), DMA and 4-aminobutan-1-ol. Purification was accomplished by preparative HPLC as described in General Procedure 11, eluting with a 10 to 40% CH₃CN/H₂O+0.1% TFA gradient to give Compound 223 as a white solid (2.6 mg, 15% yield, assumed 2×TFA salt).

LC/MS: Calc'd m/z=451.2 for C₂₅H₃₃N₅OS, found [M+H]⁺=452.4. ¹H NMR (300 MHz, acetonitrile-d₃) δ 7.77 (d, J=5.5 Hz, 1H), 7.61 (d, J=7.6, Hz, 1H), 7.41 (t, J=7.8 Hz, 1H), 7.40 (d, J=5.3 Hz) 7.22 (t, J=7.8 Hz, 1H), 6.43 (d, J=7.9, Hz, 1H), 5.74 (s, 2H), 4.45 (s, 2H), 3.54 (t, J=6.2 Hz, 2H), 3.22-3.07 (m, 2H), 2.89 (t, J=7.1 Hz, 2H), 1.88-1.66 (m, 4H), 1.64-1.30 (m, 6H), 0.90 (t, J=7.3 Hz, 3H).

### 5.15 (4-(((2-((4-amino-2-butyl-1H-imidazo[4,5-d]thieno[3,2-b]pyridin-1-yl)methyl)benzyl) amino)methyl)phenyl)methanol (Compound 224)

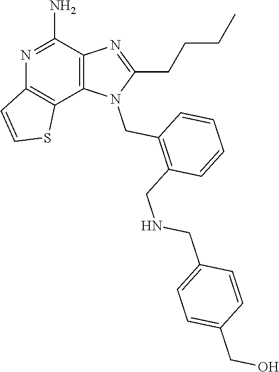 [SMILES: CCCCc1nc2c(N)nc3ccsc3c2n1Cc1ccccc1CNCc1ccc(CO)cc1]

The title compound was prepared according to General Procedure 9 using Compound 5.e (10 mg), DMA and (4-(aminomethyl)phenyl)methanol. Purification was accomplished by preparative HPLC as described in General Procedure 11, eluting with a 10 to 40% CH₃CN/H₂O+0.1% TFA gradient to give Compound 224 as a white solid (2.3 mg, 13% yield, assumed 2×TFA salt).

LC/MS: Calc'd m/z=485.2 for C₂₈H₃₁N₅OS, found [M+H]⁺=486.4. ¹H NMR (300 MHz, acetonitrile-d₃) δ 7.76 (d, J=5.4 Hz, 1H), 7.66-7.48 (m, 3H), 7.48-7.34 (m, 4H), 7.22 (t, J=7.6, 1H), 6.43 (d, J=7.7 Hz, 1H), 5.66 (s, 2H), 4.61 (s, 2H), 4.48 (s, 2H), 4.38 (s, 2H), 2.87 (t, J=7.7 Hz, 2H), 1.75 (p, J=7.5 Hz, 2H), 1.39 (h, J=7.3 Hz, 2H), 0.89 (t, J=7.3 Hz, 3H).

### 5.16 1-((2-((4-amino-2-butyl-1H-imidazo[4,5-d]thieno[3,2-b]pyridin-1-yl)methyl)benzyl) amino)-2-methylpropan-2-ol (Compound 225)

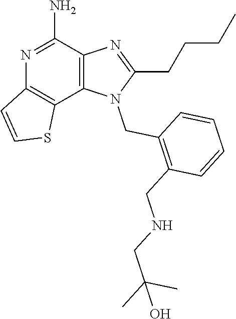 [SMILES: CCCCc1nc2c(N)nc3ccsc3c2n1Cc1ccccc1CNCC(C)(C)O]

The title compound was prepared according to General Procedure 9 using Compound 5.e (10 mg), DMA and 1-amino-2-methylpropan-2-ol. Purification was accomplished by preparative HPLC as described in General Procedure 11, eluting with a 10 to 40% CH₃CN/H₂O+0.1% TFA gradient to give Compound 225 as a white solid (2.2 mg, 13% yield, assumed 2×TFA salt).

LC/MS: Calc'd m/z=437.2 for C₂₄H₃₁N₅OS, found [M+H]⁺=438.2. ¹H NMR (300 MHz, acetonitrile-d₃) δ 7.77 (d, J=5.4 Hz, 1H), 7.66 (d, J=7.7 Hz, 1H), 7.42 (t, J=7.6 Hz, 1H), 7.41 (d, J=5.4 Hz, 1H), 7.24 (t, J=7.7 Hz, 1H), 6.43 (d, J=7.7 Hz, 1H), 5.74 (s, 2H), 4.55 (s, 2H), 2.89 (t, J=7.7 Hz, 2H), 1.75 (p, J=7.0 Hz, 2H), 1.41 (h, J=7.5 Hz, 2H), 1.31 (s, 6H), 0.89 (t, J=7.4 Hz, 3H).

### Example 6: Preparation of Series 6 Compounds

### 6.1 (2-(((6-nitrothieno[3,2-b]pyridin-7-yl)amino)methyl)thiazol-4-yl)methanol (Compound 6.a)

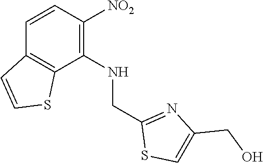 [SMILES: O=[N+]([O-])c1ccc2ccsc2c1NCc1nc(CO)cs1]

The title compound was prepared according to General Procedure 6 starting from Compound 2.a (1.00 g) and (2-(aminomethyl)thiazol-4-yl)methanol(2×HCl salt), using 5 eq. DIPEA in place of potassium carbonate. The crude compound was taken to the next step with no additional purification.

LC/MS: Calc'd m/z=321.0 for C₁₃H₁₁N₃O₃S₂, found [M+H]⁺=323.0

### 6.2 (2-(((6-aminothieno[3,2-b]pyridin-7-yl)amino)methyl)thiazol-4-yl)methanol (Compound 6.b)

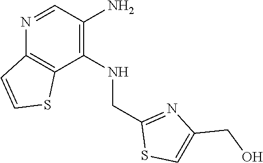 [SMILES: Nc1cnc2ccsc2c1NCc1nc(CO)cs1]

The title compound was prepared according General Procedure 2 starting from crude Compound 6.a. Purification was accomplished as described in General Procedure 11, eluting with a 0 to 20% CH₃CN/H₂O+0.1% TFA gradient to give Compound 6.b as a yellow solid (1.09 g, 45% yield, assumed 2×TFA salt).

LC/MS: Calc'd m/z=291.1 for C₁₃H₁₃N₃OS₂, found [M+H]⁺=292.2.

### 6.3 (2-((2-butyl-1H-imidazo[4,5-d]thieno[3,2-b]pyridin-1-yl)methyl)thiazol-4-yl)methanol (Compound 6.c)

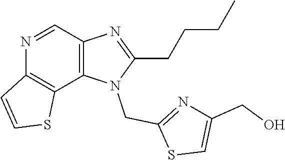 [SMILES: CCCCc1nc2cnc3ccsc3c2n1Cc1nc(CO)cs1]

The title compound was prepared according to General Procedure 7 starting from Compound 6.b (1.09 g). Purification was accomplished as described in General Procedure 11, eluting with a 10 to 50% CH₃CN/H₂O+0.1% TFA gradient to give Compound 6.c as an off-white solid (580 mg, 59% yield, assumed 1×TFA salt).

LC/MS: Calc'd m/z=358.1 for C₁₇H₁₈N₄OS₂, found [M+H]⁺=359.2.

### 6.4 (2-((4-amino-2-butyl-1H-imidazo[4,5-d]thieno[3,2-b]pyridin-1-yl)methyl)thiazol-4-yl)methanol (Compound 228)

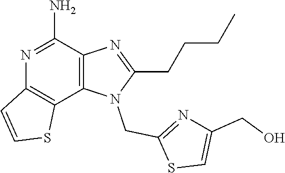 [SMILES: CCCCc1nc2c(N)nc3ccsc3c2n1Cc1nc(CO)cs1]

The title compound was prepared according to General Procedure 4 starting from Compound 6.c (580 mg) using dichloromethane as the solvent. Purification was accomplished as described in General Procedure 11, eluting with eluting with a 10 to 50% CH₃CN/H₂O+0.1% TFA gradient to give Compound 228 as a white solid (128 mg, 21% yield, assumed 1×TFA salt).

LC/MS: Calc'd m/z=373.1 for C₁₇H₁₉N₅OS₂, found [M+H]⁺=374.2. ¹H NMR (300 MHz, DMSO-d₆) δ 7.64 (d, J=5.3 Hz, 1H), 7.38-7.31 (m, 1H), 7.23 (d, J=5.3 Hz, 1H), 5.84 (s, 2H), 4.51 (s, 2H), 3.53 (s, 2H), 3.02-2.89 (m, 2H), 1.73 (p, J=7.6 Hz, 2H), 1.40 (h, J=7.3 Hz, 2H), 0.89 (t, J=7.3 Hz, 3H).

### 6.5 2-butyl-1-((4-(chloromethyl)thiazol-2-yl)methyl)-1H-imidazo[4,5-d]thieno[3,2-b]pyridin-4-amine (Compound 6.e)

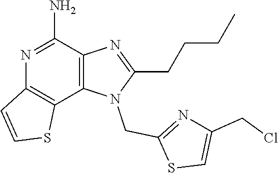 [SMILES: CCCCc1nc2c(N)nc3ccsc3c2n1Cc1nc(CCl)cs1]

The title compound was prepared according to General Procedure 8 starting from Compound 228 (105 mg). The crude compound was taken to the next step with no additional purification.

LC/MS: Calc'd m/z=391.1 for C₁₇H₁₈ClN₅S₂, found [M+H]⁺=392.2.

### 6.6 1-((4-((((1H-pyrrol-3-yl)methyl)amino)methyl)thiazol-2-yl)methyl)-2-butyl-1H-imidazo[4,5-d]thieno[3,2-b]pyridin-4-amine (Compound 231)

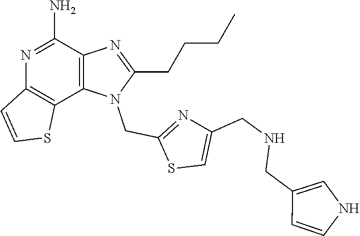 [SMILES: CCCCc1nc2c(N)nc3ccsc3c2n1Cc1nc(CNCc2cc[nH]c2)cs1]

The title compound was prepared according to General Procedure 9 using Compound 6.e (10 mg), DMA and (1H-pyrrol-3-yl)methanamine. Purification was accomplished by preparative HPLC as described in General Procedure 11, eluting with a 10 to 30% CH₃CN/H₂O+0.1% TFA gradient to give Compound 231 as a white solid (6.3 mg, 36% yield, assumed 2×TFA salt).

LC/MS: Calc'd m/z=451.2 for C₂₂H₂₅N₇S₂, found [M+H]⁺=451.8. ¹H NMR (300 MHz, acetonitrile-d₃) δ 9.93 (s, 1H), 7.82 (d, J=5.4 Hz, 1H), 7.63 (s, 1H), 7.41 (d, J=5.4 Hz, 1H), 6.76-6.66 (m, 2H), 5.99 (s, 1H), 5.89 (s, 2H), 4.07 (s, 2H), 3.85 (s, 2H), 2.97 (t, J=7.1 Hz, 1H), 1.77 (p, J=7.5 Hz, 3H), 1.43 (h, J=7.4 Hz, 2H), 0.92 (t, J=7.4 Hz, 4H).

### 6.7 2-butyl-1-((4-((4-(pyridin-4-yl)piperidin-1-yl)methyl)thiazol-2-yl)methyl)-1H-imidazo [4,5-d]thieno[3,2-b]pyridin-4-amine (Compound 232)

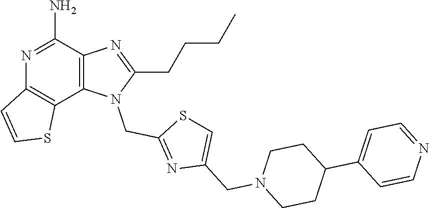 [SMILES: CCCCc1nc2c(N)nc3ccsc3c2n1Cc1nc(CN2CCC(c3ccncc3)CC2)cs1]

The title compound was prepared according to General Procedure 9 using Compound 6.e (10 mg), DMA and 4-(piperidin-4-yl)pyridine. Purification was accomplished by preparative HPLC as described in General Procedure 11, eluting with a 10 to 30% CH₃CN/H₂O+0.1% TFA gradient to give Compound 232 as a white solid (9.4 mg, 43% yield, assumed 3×TFA salt).

LC/MS: Calc'd m/z=517.2 for C₂₇H₃₁N₇S₂, found [M+H]⁺=517.8. ¹H NMR (300 MHz, acetonitrile-d₃) δ 8.67 (d, J=5.9 Hz, 2H), 7.86-7.77 (m, 4H), 7.46-7.38 (m, 1H), 5.92 (s, 2H), 4.26 (s, 2H), 3.11 (q, J=7.5 Hz, 2H), 3.06-2.65 (m, 5H), 1.97-1.73 (m, 2H), 1.43 (h, J=7.5 Hz, 2H), 1.23 (t, J=7.6 Hz, 4H), 0.93 (t, J=7.4 Hz, 3H).

### 6.8 2-butyl-1-((4-((diethylamino)methyl)thiazol-2-yl)methyl)-1H-imidazo[4,5-d]thieno[3,2-b]pyridin-4-amine (Compound 233)

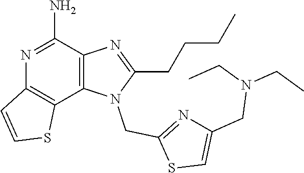 [SMILES: CCCCc1nc2c(N)nc3ccsc3c2n1Cc1nc(CN(CC)CC)cs1]

The title compound was prepared according to General Procedure 9 using Compound 6.e (10 mg), DMA and diethylamine. Purification was accomplished by preparative HPLC as described in General Procedure 11, eluting with a 10 to 30% CH₃CN/H₂O+0.1% TFA gradient to give Compound 233 as a white solid (6.5 mg, 39% yield, assumed 2×TFA salt).

LC/MS: Calc'd m/z=428.2 for C₂₁H₂₈N₆S₂, found [M+H]⁺=428.8. ¹H NMR (300 MHz, acetonitrile-d₃) δ 7.84 (d, J=5.0 Hz, 1H), 7.79 (s, 1H), 7.44 (d, J=5.6 Hz, 1H), 5.90 (s, 2H), 4.19 (s, 2H), 2.98 (t, J=7.7 Hz, 2H), 2.88 (q, J=8.7 Hz, 4H), 1.76 (p, J=7.2 Hz, 2H), 1.41 (h, J=7.4 Hz, 2H), 1.07 (t, J=7.4 Hz, 6H), 0.92 (t, J=7.4 Hz, 3H).

### 6.9 2-butyl-1-((4-((4-(2-morpholinoethyl)piperazin-1-yl)methyl)thiazol-2-yl)methyl)-1H-imidazo[4,5-d]thieno[3,2-b]pyridin-4-amine (Compound 234)

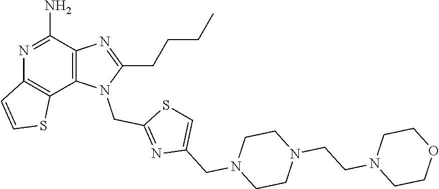 [SMILES: CCCCc1nc2c(N)nc3ccsc3c2n1Cc1nc(CN2CCN(CCN3CCOCC3)CC2)cs1]

The title compound was prepared according to General Procedure 9 using Compound 6.e (10 mg), DMA and 4-(2-(piperazin-1-yl)ethyl)morpholine. Purification was accomplished by preparative HPLC as described in General Procedure 11, eluting with a 10 to 30% CH₃CN/H₂O+0.1% TFA gradient to give Compound 234 as a white solid (13.9 mg, 54% yield, assumed 4×TFA salt).

LC/MS: Calc'd m/z=554.3 for C₂₇H₃₈N₈OS₂, found [M+H]⁺=554.8. ¹H NMR (300 MHz, acetonitrile-d₃) δ 7.85 (d, J=5.4 Hz, 1H), 7.77 (s, 1H), 7.45 (d, J=5.4 Hz, 1H), 5.90 (s, 2H), 4.21 (s, 2H), 3.94-3.83 (m, 4H), 3.32-3.17 (m, 5H), 3.03-2.90 (m, 2H), 2.82-2.67 (m, 3H), 2.69-2.44 (m, 4H), 1.78 (p, J=7.7 Hz, 2H), 1.44 (h, J=7.4 Hz, 2H), 0.94 (t, J=7.5 Hz, 3H).

### 6.10 1-((4-(aminomethyl)thiazol-2-yl)methyl)-2-butyl-1H-imidazo[4,5-d]thieno[3,2-b]pyridin-4-amine (Compound 235)

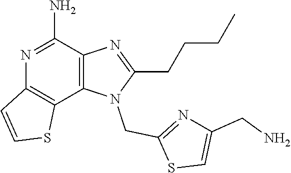 [SMILES: CCCCc1nc2c(N)nc3ccsc3c2n1Cc1nc(CN)cs1]

The title compound was prepared according to General Procedure 9 using Compound 6.e (10 mg), DMA and 0.5 mL 2 M ammonia in methanol. Purification was accomplished by preparative HPLC as described in General Procedure 11, eluting with a 10 to 30% CH₃CN/H₂O+0.1% TFA gradient to give Compound 235 as a white solid (2.9 mg, 19% yield, assumed 2×TFA salt).

LC/MS: Calc'd m/z=372.1 for C₁₇H₂₀N₆S₂, found [M+H]⁺=372.8. ¹H NMR (300 MHz, acetonitrile-d₃) δ 7.86 (d, J=5.1 Hz, 1H), 7.58 (s, 1H), 7.44 (t, J=5.1 Hz, 1H), 5.88 (s, 2H), 4.10 (s, 2H), 2.99 (t, J=8.4 Hz, 2H), 1.79 (p, J=7.9 Hz, 2H), 1.45 (h, J=7.3 Hz, 2H), 0.94 (t, J=7.0 Hz, 3H).

### 6.11 5-(((2-((4-amino-2-butyl-1H-imidazo[4,5-d]thieno[3,2-b]pyridin-1-yl)methyl)thiazol-4-yl)methyl)amino)pentan-1-ol (Compound 236)

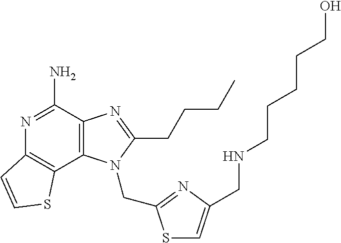 [SMILES: CCCCc1nc2c(N)nc3ccsc3c2n1Cc1nc(CNCCCCCO)cs1]

The title compound was prepared according to General Procedure 9 using Compound 6.e (10 mg), DMA and 5-aminopentan-1-ol. Purification was accomplished by preparative HPLC as described in General Procedure 11, eluting with a 10 to 30% CH₃CN/H₂O+0.1% TFA gradient to give Compound 236 as a white solid (7.7 mg, 44% yield, assumed 2×TFA salt).

LC/MS: Calc'd m/z=458.2 for C₂₂H₃₀N₆OS₂, found [M+H]⁺=458.8. ¹H NMR (300 MHz, acetonitrile-d₃) δ 7.87 (d, J=5.4 Hz, 1H), 7.66 (s, 1H), 7.55-7.32 (m, 1H), 5.89 (s, 2H), 4.12 (s, 2H), 3.45 (t, J=6.7 Hz, 3H), 2.98 (t, J=7.1 Hz, 2H), 2.79-2.70 (m, 2H), 1.78 (p, J=7.8 Hz, 2H), 1.53-1.32 (m, 7H), 1.17 (h, J=7.8 Hz, 2H), 0.94 (t, J=7.4 Hz, 3H).

### 6.12 (4-((((2-((4-amino-2-butyl-1H-imidazo[4,5-d]thieno[3,2-b]pyridin-1-yl)methyl)thiazol-4-yl)methyl)amino)methyl)phenyl)methanol (Compound 237)

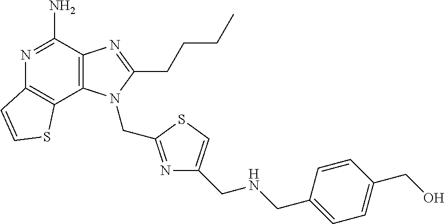 [SMILES: CCCCc1nc2c(N)nc3ccsc3c2n1Cc1nc(CNCc2ccc(CO)cc2)cs1]

The title compound was prepared according to General Procedure 9 using Compound 6.e (10 mg), DMA and 5-aminopentan-1-ol. Purification was accomplished by preparative HPLC as described in General Procedure 11, eluting with a 10 to 30% CH₃CN/H₂O+0.1% TFA gradient to give Compound 237 as a white solid (6.8 mg, 37% yield, assumed 2×TFA salt).

LC/MS: Calc'd m/z=492.2 for C₂₅H₂₈N₆OS₂, found [M+H]⁺=493.2. ¹H NMR (300 MHz, Acetonitrile-d₃) δ 7.81 (d, J=5.4 Hz, 1H), 7.67 (s, 1H), 7.52-7.35 (m, 1H), 7.31 (d, J=7.8 Hz, 2H), 7.10 (d, J=7.7 Hz, 2H), 5.92 (s, 2H), 4.58 (s, 2H), 4.12 (s, 2H), 3.89 (s, 2H), 2.98 (t, J=7.9 Hz, 2H), 1.77 (p, J=7.8 Hz, 3H), 1.42 (h, J=7.9 Hz, 2H), 1.04-0.86 (t, J=7.7 Hz, 4H).

### 6.13 1-(((2-((4-amino-2-butyl-1H-imidazo[4,5-d]thieno[3,2-b]pyridin-1-yl)methyl)thiazol-5-yl)methyl)amino)-2-methylpropan-2-ol (Compound 238)

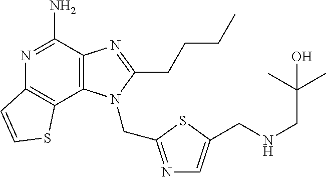 [SMILES: CCCCc1nc2c(N)nc3ccsc3c2n1Cc1ncc(CNCC(C)(C)O)s1]

The title compound was prepared according to General Procedure 9 using Compound 6.e (10 mg), DMA and 1-amino-2-methylpropan-2-ol. Purification was accomplished by preparative HPLC as described in General Procedure 11, eluting with a 10 to 30% CH₃CN/H₂O+0.1% TFA gradient to give Compound 238 as a white solid (4.0 mg, 23% yield, assumed 2×TFA salt).

LC/MS: Calc'd m/z=492.2 for C₂₁H₂₈N₆OS₂, found [M+H]⁺=492.8. ¹H NMR (300 MHz, acetonitrile-d₃) δ 7.85 (d, J=5.1 Hz, 1H), 7.69 (s, 1H), 7.43 (d, J=5.2 Hz, 1H), 5.89 (s, 2H), 4.19 (s, 2H), 2.98 (t, J=7.9 Hz, 2H), 2.71 (s, 2H), 1.78 (p, J=7.8 Hz, 2H), 1.44 (h, J=7.3 Hz, 2H), 1.05 (s, 6H), 0.94 (t, J=7.6 Hz, 3H).

### 6.14 2-butyl-1-((4-(piperazin-1-ylmethyl)thiazol-2-yl)methyl)-1H-imidazo[4,5-d]thieno[3,2-b]pyridin-4-amine (Compound 242)

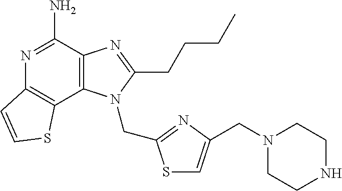 [SMILES: CCCCc1nc2c(N)nc3ccsc3c2n1Cc1nc(CN2CCNCC2)cs1]

The titled compound was prepared according to General Procedure 9 followed by General Procedure 5 using Compound 6.e (10 mg), DMA and tert-butyl piperazine-1-carboxylate. Purification of the intermediate Boc compound was accomplished by preparative HPLC as described in General Procedure 11, eluting with a 10 to 40% CH₃CN/H₂O+0.1% TFA gradient. Purification of Compound 242 was accomplished by preparative HPLC as described in General Procedure 11, eluting with a 10 to 30% CH₃CN/H₂O+0.1% TFA gradient to give Compound 242 as a white solid (6.6 mg, 32% yield, assumed 3×TFA salt).

LC/MS: Calc'd m/z=441.2 for C₂₁H₂₇N₇S, found [M+H]⁺=442.2. ¹H NMR (300 MHz, acetonitrile-d₃) δ 7.84 (d, J=5.3 Hz, 1H), 7.70 (d, J=2.9 Hz, 1H), 7.45 (d, J=5.1 Hz, 1H), 5.90 (s, 2H), 4.12 (s, 2H), 3.21-3.07 (m, 8H), 3.00 (t, J=7.2 Hz, 2H), 1.78 (p, J=7.9 Hz, 3H), 1.51-1.40 (m, 2H), 0.95 (t, J=7.6 Hz, 3H).

### Example 7: Preparation of Series 7 Compounds

### 7.1 (5-(((6-nitrothieno[3,2-b]pyridin-7-yl)amino)methyl)furan-2-yl)methanol (Compound 7.a)

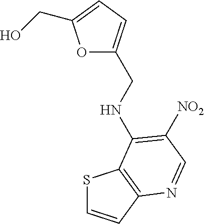 [SMILES: O=[N+]([O-])c1cnc2ccsc2c1NCc1ccc(CO)o1]

The title compound was prepared according to General Procedure 6 starting from Compound 2.a (1.30 g) and (5-(aminomethyl)furan-2-yl)methanol. The crude compound was taken to the next step with no additional purification.

LC/MS: Calc'd m/z=305.1 for C₁₃H₁₁N₃O₄S, found [M+H]⁺=306.0

### 7.2 (5-(((6-aminothieno[3,2-b]pyridin-7-yl)amino)methyl)furan-2-yl)methanol (Compound 7.b)

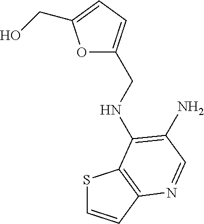 [SMILES: Nc1cnc2ccsc2c1NCc1ccc(CO)o1]

The title compound was prepared according General Procedure 2 starting from crude Compound 7.a. Purification was accomplished as described in General Procedure 11, eluting with 0 to 30% CH₃CN/H₂O+0.1% TFA to give Compound 7.b as a yellow solid (1.60 g, 53% yield, over 2 steps, assumed 2×TFA salt).

LC/MS: Calc'd m/z=275.1 for C₁₃H₁₃N₃O₂S, found [M+H]⁺=276.0.

### 7.3 (5-((2-butyl-1H-imidazo[4,5-d]thieno[3,2-b]pyridin-1-yl)methyl)furan-2-yl)methanol (Compound 7.c)

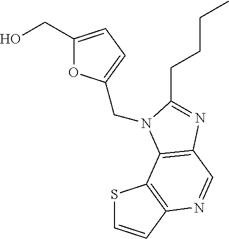 [SMILES: CCCCc1nc2cnc3ccsc3c2n1Cc1ccc(CO)o1]

The title compound was prepared according to General Procedure 7 starting from Compound 7.b (1.61 g). Purification was accomplished as described in General Procedure 11, eluting with 10 to 50% CH₃CN/H₂O+0.1% TFA to give Compound 7.c as an off-white solid, which was dissolved in 1% NH₄OH/MeOH (10 mL) and evaporated to dryness to give Compound 7.c as an off-white solid (920 mg, 84% yield).

LC/MS: Calc'd m/z=358.1 for C₁₇H₁₈N₄OS₂, found [M+H]⁺=359.2.

### 7.4 (5-((4-amino-2-butyl-1H-imidazo[4,5-d]thieno[3,2-b]pyridin-1-yl)methyl)furan-2-yl)methanol (Compound 253)

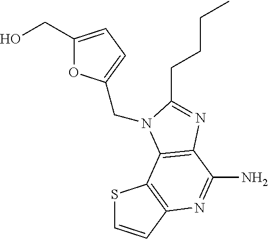 [SMILES: CCCCc1nc2c(N)nc3ccsc3c2n1Cc1ccc(CO)o1]

The title compound was prepared according to General Procedure 4 starting from Compound 7.c (300 mg) and using THF as the solvent. Purification was accomplished as described in General Procedure 11, eluting with 10 to 50% CH₃CN/H₂O+0.1% TFA to give Compound 253 as a white solid (61 mg, 12% yield).

LC/MS: Calc'd m/z=356.1 for C₁₈H₂₀N₄O₂S, found [M+H]⁺=357.2. ¹H NMR (300 MHz, acetonitrile-d₃) δ 7.91 (d, J=5.5 Hz, 1H), 7.45 (d, J=5.5 Hz, 1H), 6.40 (d, J=3.2 Hz, 1H), 6.24 (d, J=3.2 Hz, 1H), 5.53 (s, 2H), 4.36 (s, 2H), 3.03 (t, J=7.5 Hz, 2H), 1.83 (p, J=7.5 Hz, 2H), 1.57-1.38 (m, 2H), 0.97 (t, J=7.4 Hz, 3H).

### Example 8: Preparation of Series 8 Compounds

### 8.1 tert-butyl(4-((7-bromo-2-butyl-1H-imidazo[4,5-d]thieno[3,2-b]pyridin-1-yl)methyl) benzyl)carbamate (Compound 8.a)

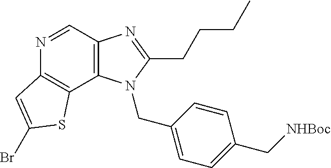 [SMILES: CCCCc1nc2cnc3cc(Br)sc3c2n1Cc1ccc(CNC(=O)OC(C)(C)C)cc1]

To a stirring solution of Compound 2.d (1.00 g, 2.22 mmol) in 20 mL acetic acid was added sodium acetate (910 mg, 11.1 mmol) followed by bromine (0.170 mL, 3.33 mmol). The reaction mixture was allowed to stir for 16 h at rt. The reaction mixture was concentrated under vacuum, carefully quenched with 1 M NaHCO₃ in water, then extracted twice with dichloromethane. The organic extract was dried over sodium sulfate then concentrated in vacuo. Purification of Compound 8.a was accomplished by reverse phase chromatography as described in General Procedure 11, eluting with a 10 to 60% CH₃CN/H₂O+0.1% TFA gradient to give Compound 8.a as a white solid (220 mg, 15% yield, assumed 1×TFA salt).

LC/MS: Calc'd m/z=528.1 for C₂₅H₂₉BrN₄O₂S, found [M+H]⁺=529.2.

### 8.2 tert-butyl(4-((2-butyl-7-phenyl-1H-imidazo[4,5-d]thieno[3,2-b]pyridin-1-yl)methyl) benzyl)carbamate (Compound 8.b)

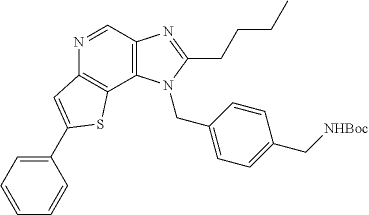 [SMILES: CCCCc1nc2cnc3cc(-c4ccccc4)sc3c2n1Cc1ccc(CNC(=O)OC(C)(C)C)cc1]

To a stirring solution of Compound 8.a (50 mg, 0.094 mmol) in toluene (0.5 mL) and ethanol (0.5 mL) was added phenyl boronic acid (18 mg, 0.151 mmol) followed by 2 M Na₂CO₃ in water (0.2 mL). Pd(PPh₃)₄ (5 mg, 0.005 mmol) was added and the reaction mixture was heated at 90° C. for 3 h. The reaction mixture was diluted with water then extracted twice with ethyl acetate. The combined organic extracts were dried over sodium sulfate then concentrated in vacuo. Purification was accomplished by reverse phase chromatography as described in General Procedure 11, eluting with a 10 to 60% CH₃CN/H₂O+0.1% TFA gradient to give Compound 8.b as a white solid (25 mg, 42% yield, assumed 1×TFA salt).

LC/MS: Calc'd m/z=526.2 for C₃₁H₃₄N₄O₂S, found [M+H]⁺=527.4.

### 8.3 1-(4-(aminomethyl)benzyl)-2-butyl-7-phenyl-1H-imidazo[4,5-d]thieno[3,2-b]pyridin-4-amine (Compound 209)

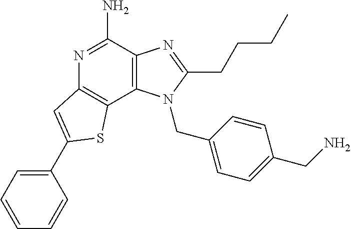 [SMILES: CCCCc1nc2c(N)nc3cc(-c4ccccc4)sc3c2n1Cc1ccc(CN)cc1]

The title compound was prepared according to General Procedure 4 followed by General Procedure 5 starting from Compound 8.b (25 mg) using dichloromethane as the solvent. Purification was accomplished as described in General Procedure 11, eluting with eluting with 10 to 45% CH₃CN/H₂O+0.1% TFA to give Compound 209 as a white solid (3.2 mg, 10% yield, assumed 2×TFA salt).

LC/MS: Calc'd m/z=441.2 for C₂₆H₂₇N₅S, found [M+H]⁺=442.2. ¹H NMR (300 MHz, acetonitrile-d₃) δ 7.75-7.65 (m, 3H), 7.53-7.36 (m, 3H), 7.20 (d, J=8.1 Hz, 2H), 5.66 (s, 2H), 5.46 (s, 1H), 4.05 (s, 2H), 2.89 (t, J=7.7 Hz, 2H), 1.75 (p, J=7.7 Hz, 2H), 1.41 (h, J=7.5 Hz, 2H), 0.90 (t, J=7.4 Hz, 3H).

### 8.4 tert-butyl(4-((2-butyl-7-methyl-1H-imidazo[4,5-d]thieno[3,2-b]pyridin-1-yl)methyl) benzyl)carbamate (Compound 8.c)

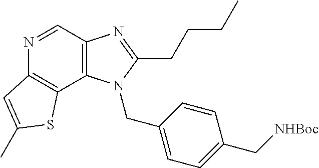 [SMILES: CCCCc1nc2cnc3cc(C)sc3c2n1Cc1ccc(CNC(=O)OC(C)(C)C)cc1]

To a stirring solution of Compound 8.a (100 mg, 0.189 mmol) in toluene (1 mL) and ethanol (0.6 mL) was added methyl boronic acid (18 mg, 0.302 mmol) followed by 2 M Na₂CO₃ in water (0.4 mL). Pd(PPh₃)₄ (11 mg, 0.009 mmol) was added and the reaction mixture was heated at 80° C. for 1 h. The reaction mixture was diluted with water then extracted twice with ethyl acetate. The combined organic extracts were dried over sodium sulfate then concentrated in vacuo. Purification was accomplished by reverse phase chromatography as described in General Procedure 11, eluting with a 10 to 60% CH₃CN/H₂O+0.1% TFA gradient to give Compound 8.c as a white solid (8.0 mg, 4% yield, assumed 1×TFA salt).

LC/MS: Calc'd m/z=464.2 for C₂₆H₃₂N₄O₂S, found [M+H]⁺=465.4.

### 8.5 1-(4-(aminomethyl)benzyl)-2-butyl-7-methyl-1H-imidazo[4,5-d]thieno[3,2-b]pyridin-4-amine (Compound 254)

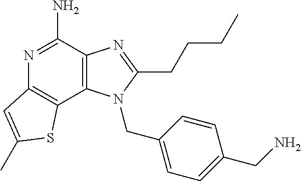 [SMILES: CCCCc1nc2c(N)nc3cc(C)sc3c2n1Cc1ccc(CN)cc1]

The title compound was prepared according to General Procedure 4 using dichloromethane as the solvent starting from Compound 8.c (8.0 mg) followed by General Procedure 5. Purification was accomplished as described in General Procedure 11, eluting with 10 to 40% CH₃CN/H₂O+0.1% TFA to give Compound 254 as a white solid (3.3 mg, 31% yield, assumed 2×TFA salt).

LC/MS: Calc'd m/z=379.2 for C₂₆H₂₇N₅S, found [M+H]⁺=380.2. ¹H NMR (300 MHz, acetonitrile-d₃) δ 7.40 (d, J=8.1 Hz, 2H), 7.28-7.04 (m, 3H), 5.59 (s, 3H), 4.06 (s, 2H), 2.92-2.81 (m, 2H), 2.56 (d, J=1.2 Hz, 3H), 1.73 (p, J=7.4 Hz, 2H), 1.39 (h, J=7.4 Hz, 2H), 0.89 (t, J=7.3 Hz, 3H).

### 8.6 tert-butyl (tert-butoxycarbonyl)(1-(4-(((tert-butoxycarbonyl)amino)methyl)benzyl)-2-butyl-1H-imidazo[4,5-d]thieno[3,2-b]pyridin-4-yl)carbamate (Compound 8.d)

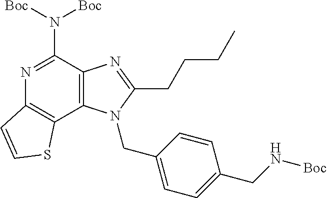 [SMILES: CCCCc1nc2c(N(C(=O)OC(C)(C)C)C(=O)OC(C)(C)C)nc3ccsc3c2n1Cc1ccc(CNC(=O)OC(C)(C)C)cc1]

To a solution of Compound 2.e (50 mg, 0.107 mmol) in 1 mL DMF was added DIPEA (56 μL, 0.322 mmol) followed by di-tert-butyl dicarbonate (74 μL, 0.322 mmol) and DMAP (1.3 mg, 0.011 mmol). The reaction mixture was stirred for 16 h then concentrated in vacuo. Purification was accomplished as described in General Procedure 11, eluting with a 20 to 80% CH₃CN/H₂O+0.1% TFA gradient to give Compound 8.d as a white solid (25 mg, 35% yield, assumed 1×TFA salt).

LC/MS: Calc'd m/z=665.3 for C₃₅H₄₇N₅O₆S, found [M+H]⁺=666.6.

### 8.7 1-(4-(aminomethyl)benzyl)-7-bromo-2-butyl-1H-imidazo[4,5-d]thieno[3,2-b]pyridin-4-amine (Compound 245)

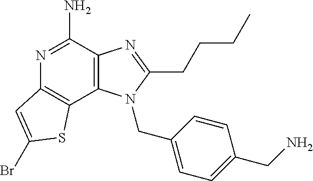 [SMILES: CCCCc1nc2c(N)nc3cc(Br)sc3c2n1Cc1ccc(CN)cc1]

To a stirring solution of Compound 8.d (25 mg, 0.038 mmol) in 2 mL acetic acid was added sodium acetate (15 mg, 0.188 mmol) followed by bromine (3 μL, 0.056 mmol). The reaction mixture was allowed to stir for 16 h. The reaction mixture was concentrated under vacuum, carefully quenched with 1 M NaHCO₃ in water, then extracted with dichloromethane. The organic extract was dried over sodium sulfate then concentrated in vacuo. To a stirring solution of the crude intermediate in 0.8 mL DCM was added 0.2 mL TFA. The solution was stirred for 1 h then concentrated in vacuo. Purification was accomplished by reverse phase chromatography as described in General Procedure 11, eluting with a 20 to 55% CH₃CN/H₂O+0.1% TFA gradient to give Compound 245 as a white solid (1.1 mg, 4% yield, assumed 2×TFA salt).

LC/MS: Calc'd m/z=443.1 for C₂₀H₂₂BrN₅S, found [M+H]⁺=444.2. ¹H NMR (300 MHz, acetonitrile-d₃) δ 9.96 (s, 1H), 7.88 (d, J=8.2 Hz, 2H), 7.50 (s, 1H), 7.28 (d, J=7.8 Hz, 3H), 5.65 (s, 2H), 2.90 (t, J=7.7 Hz, 2H), 1.74 (p, J=7.7 Hz, 3H), 1.39 (h, J=7.4 Hz, 2H), 0.89 (t, J=7.4 Hz, 3H).

### Example 9: Preparation of Series 9 Compounds

### 9.1 tert-butyl(4-((2-(ethoxymethyl)-1H-imidazo[4,5-d]thieno[3,2-b]pyridin-1-yl)methyl) benzyl)carbamate (Compound 9.a)

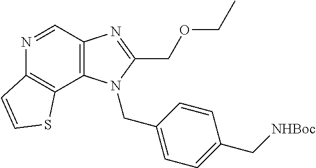 [SMILES: CCOCc1nc2cnc3ccsc3c2n1Cc1ccc(CNC(=O)OC(C)(C)C)cc1]

To a solution of Compound 2.c (400 mg, 0.653 mmol, 2×TFA salt) in 5 mL dichloromethane was added ethoxy-acetyl chloride (74 μL, 0.718 mmol) followed by triethylamine (275 μL, 1.96 mmol). Upon completion, the reaction was concentrated in vacuo then dissolved in ethanol (10 mL). To the stirring solution was added a solution of sodium hydroxide (131 mg, 3.26 mmol) dissolved in 1.3 mL water. Upon completion, the reaction was partially concentrated in vacuo, diluted with water, then extracted twice with ethyl acetate. The combined organic extracts were dried over sodium sulfate then concentrated in vacuo to give Compound 9.a as an off-white solid (280 mg, 95% yield).

LC/MS: Calc'd m/z=452.2 for C₂₄H₂₈N₄O₃S, found [M+H]⁺=453.2.

### 9.2 tert-butyl (4-((4-amino-2-(ethoxymethyl)-1H-imidazo[4,5-d]thieno[3,2-b]pyridin-1-yl)methyl)benzyl)carbamate (Compound 9.b)

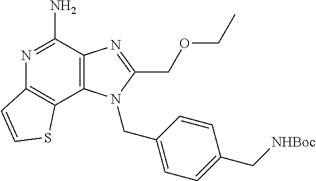 [SMILES: CCOCc1nc2c(N)nc3ccsc3c2n1Cc1ccc(CNC(=O)OC(C)(C)C)cc1]

The title compound was prepared according to General Procedure 4 starting from Compound 9.a (230 mg) using 1,2-dichloroethane as the solvent. The crude product was taken to the next step with no additional purification.

LC/MS: Calc'd m/z=467.2 for C₂₅H₃₁N₅O₂S, found [M+H]⁺=468.2.

### 9.3 1-(4-(aminomethyl)benzyl)-2-(ethoxymethyl)-1H-imidazo[4,5-d]thieno[3,2-b]pyridin-4-amine (Compound 284)

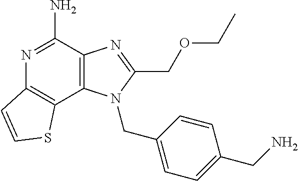 [SMILES: CCOCc1nc2c(N)nc3ccsc3c2n1Cc1ccc(CN)cc1]

The title compound was prepared according to General Procedure 5 starting from Compound 9.b (10 mg, 0.021 mmol). Purification was accomplished by preparative HPLC as described in General Procedure 11, eluting with 0 to 30% CH₃CN/H₂O+0.1% TFA to give Compound 284 as a white solid (2.8 mg, 22% yield, assumed 2×TFA salt).

LC/MS: Calc'd m/z=367.2 for C₁₉H₂₁N₅OS, found [M+H]⁺=368.2. ¹H NMR (300 MHz, methanol-d₄) δ 7.91 (d, J=5.5 Hz, 1H), 7.47 (d, J=8.4 Hz, 3H), 7.44 (d, J=5.5 Hz, 1H), 7.27 (d, J=8.0 Hz, 2H), 5.84 (s, 2H), 4.85 (s, 3H), 4.11 (s, 2H), 3.60 (q, J=7.0 Hz, 2H), 1.10 (t, J=7.0 Hz, 3H).

### 9.4 tert-butylethyl((1-(4-(hydroxymethyl)benzyl)-1H-imidazo[4,5-d]thieno[3,2-b]pyridin-2-yl)methyl)carbamate (Compound 9.c)

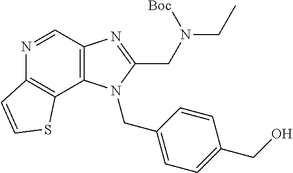 [SMILES: CCN(Cc1nc2cnc3ccsc3c2n1Cc1ccc(CO)cc1)C(=O)OC(C)(C)C]

To a solution of Compound 2.g (500 mg, 1.752 mmol) in 1 mL DMF was added triethylamine (0.739 mL, 5.26 mmol), [(tert-butoxycarbonyl)(ethyl)amino]acetic acid (356 mg, 1.75 mmol) followed by DMAP (21 mg, 0.18 mmol) then HATU (733 mg, 1.97 mmol). Upon completion, the reaction was concentrated in vacuo then dissolved in ethyl acetate. The organic layer was washed once with water then concentrated in vacuo. To the crude solid was added ethanol (2 mL). To the stirring solution was added a solution of sodium hydroxide (350 mg, 8.76 mmol) dissolved in 3.1 mL water and the mixture was heated at 90° C. Upon completion, the reaction was partially concentrated in vacuo, diluted with water, then extracted twice with ethyl acetate. The combined organic extracts were dried over sodium sulfate then concentrated in vacuo to give Compound 9.c as an off-white solid (442 mg, 56% yield).

LC/MS: Calc'd m/z=452.2 for C₂₄H₂₈N₄O₃S, found [M+H]⁺=453.2.

### 9.5 tert-butyl ((4-amino-1-(4-(hydroxymethyl)benzyl)-1H-imidazo[4,5-d]thieno[3,2-b]pyridin-2-yl)methyl)(ethyl)carbamate (Compound 9.d)

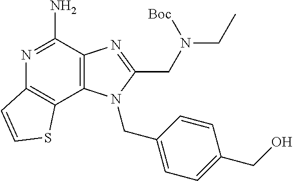 [SMILES: CCN(Cc1nc2c(N)nc3ccsc3c2n1Cc1ccc(CO)cc1)C(=O)OC(C)(C)C]

The title compound was prepared according to General Procedure 4 starting from Compound 9.c (442 mg) using 1,2-dichloroethane as the solvent. The crude product was taken to the next step with no additional purification.

LC/MS: Calc'd m/z=467.2 for C₂₄H₂₉N₅O₃S, found [M+H]⁺=468.2.

### 9.6 tert-butyl ((4-amino-1-(4-(bromomethyl)benzyl)-1H-imidazo[4,5-d]thieno[3,2-b]pyridin-2-yl)methyl)(ethyl)carbamate (Compound 9.e)

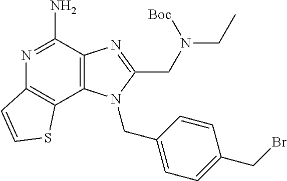 [SMILES: CCN(Cc1nc2c(N)nc3ccsc3c2n1Cc1ccc(CBr)cc1)C(=O)OC(C)(C)C]

To a solution of Compound 9.d (100 mg, 0.214 mmol) in 5 mL THF was added triphenylphosphine (84 mg, 0.321 mmol) followed by N-bromosuccinimide (57 mg, 0.321 mmol). Upon completion, the reaction mixture was concentrated in vacuo then columned by normal phase chromatography using a 0-100% DCM/acetone gradient. The combined fractions were concentrated in vacuo to give Compound 9.e as an off-white solid (89 mg, 79% yield).

LC/MS: Calc'd m/z=529.1 for C₂₄H₂₈BrN₅O₂S, found [M+H]⁺=530.2.

### 9.7 1-(4-(aminomethyl)benzyl)-2-((ethylamino)methyl)-1H-imidazo[4,5-d]thieno[3,2-b]pyridin-4-amine (Compound 267)

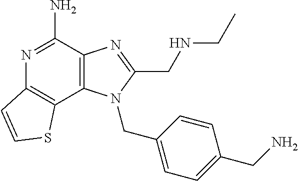 [SMILES: CCNCc1nc2c(N)nc3ccsc3c2n1Cc1ccc(CN)cc1]

The title compound was prepared according to General Procedure 9 followed by General Procedure 5 starting from Compound 9.e (10 mg) and 1M ammonia in methanol. Purification was accomplished by preparative HPLC as described in General Procedure 11, eluting with 0 to 30% CH₃CN/H₂O+0.1% TFA to give Compound 267 as a white solid (1.9 mg, 6% yield assumed 3×TFA salt).

LC/MS: Calc'd m/z=366.2 for C₁₉H₂₂N₆S, found [M+H]⁺=366.8. ¹H NMR (300 MHz, methanol-d₄) δ 7.95 (d, J=5.4 Hz, 1H), 7.50 (d, J=8.0 Hz, 2H), 7.46 (d, J=5.5 Hz, 1H), 7.27 (d, J=7.7 Hz, 2H), 5.81 (s, 2H), 4.67 (s, 2H), 4.12 (s, 2H), 3.36 (q, J=7.3 Hz, 2H), 1.44 (t, J=7.3 Hz, 3H).

### 9.8 2-((ethylamino)methyl)-1-(4-(piperazin-1-ylmethyl)benzyl)-1H-imidazo[4,5-d]thieno [3,2-b]pyridin-4-amine (Compound 268)

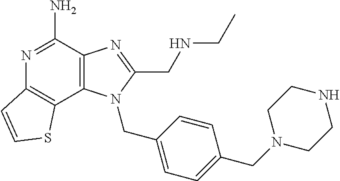 [SMILES: CCNCc1nc2c(N)nc3ccsc3c2n1Cc1ccc(CN2CCNCC2)cc1]

The title compound was prepared according to General Procedure 9 followed by General Procedure 5 starting from Compound 9.e (30 mg) and tert-butyl piperazine-1-carboxylate. Purification was accomplished by preparative HPLC as described in General Procedure 11, eluting with 0 to 20% CH₃CN/H₂O+0.1% TFA to give Compound 268 as a white solid (3.1 mg, 6% yield, assumed 4×TFA salt).

LC/MS: Calc'd m/z=435.2 for C₂₃H₂₉N₇S, found [M+H]⁺=436.2. ¹H NMR (300 MHz, methanol-d₄) δ 7.98 (d, J=5.5 Hz, 1H), 7.48 (d, J=5.5 Hz, 1H), 7.42 (d, J=8.0 Hz, 2H), 7.19 (d, J=8.0 Hz, 2H), 5.77 (s, 2H), 4.66 (s, 2H), 3.69 (s, 2H), 3.38 (q, J=7.3 Hz, 2H), 3.28-3.21 (m, 4H), 2.79-2.69 (m, 4H), 1.42 (d, J=7.3 Hz, 3H).

### 9.9 1-(4-((diethylamino)methyl)benzyl)-2-((ethylamino)methyl)-1H-imidazo[4,5-d]thieno [3,2-b]pyridin-4-amine (Compound 269)

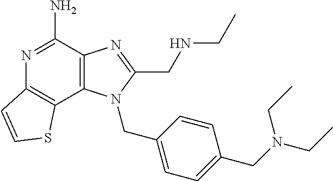 [SMILES: CCNCc1nc2c(N)nc3ccsc3c2n1Cc1ccc(CN(CC)CC)cc1]

The title compound was prepared according to General Procedure 9 followed by General Procedure 5 starting from Compound 9.e (30 mg) and diethylamine. Purification of the intermediate Boc-compound was accomplished by preparative HPLC as described in General Procedure 11, eluting with a 10 to 40% CH₃CN/H₂O+0.1% TFA gradient to give Compound 269 as a white solid (8.4 mg, 18% yield, assumed 3×TFA salt).

LC/MS: Calc'd m/z=422.2 for C₂₃H₃₀N₆S, found [M+H]⁺=423.4. ¹H NMR (300 MHz, methanol-d₄) δ 7.96 (d, J=5.5 Hz, 1H), 7.62-7.51 (m, 2H), 7.48 (d, J=5.5 Hz, 1H), 7.36-7.27 (m, 2H), 5.83 (s, 2H), 4.70 (s, 2H), 4.34 (s, 2H), 3.39 (q, J=7.3 Hz, 2H), 3.19 (qd, J=7.3, 3.4 Hz, 4H), 1.45 (t, J=7.3 Hz, 3H), 1.32 (t, J=7.3 Hz, 6H).

### 9.10 (4-((2-(ethoxymethyl)-1H-imidazo[4,5-d]thieno[3,2-b]pyridin-1-yl)methyl)phenyl) methanol (Compound 9.f)

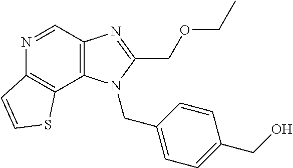 [SMILES: CCOCc1nc2cnc3ccsc3c2n1Cc1ccc(CO)cc1]

To a solution of Compound 2.g (810 mg, 2.84 mmol) in 5 mL dichloromethane was added ethoxy-acetyl chloride (614 μL, 5.96 mmol) followed by triethylamine (1.2 mL, 8.51 mmol). Upon completion, the reaction was concentrated in vacuo then dissolved in ethanol (20 mL). To the stirring solution was added a solution of sodium hydroxide (568 mg, 14.2 mmol) dissolved in 10 mL water. Upon completion, the reaction was partially concentrated in vacuo, diluted with water, then extracted twice with ethyl acetate. The combined organic extracts were dried over sodium sulfate then concentrated in vacuo to give Compound 9.f as an off-white solid (530 mg, 52% yield).

LC/MS: Calc'd m/z=353.1 for C₁₉H₁₉N₃O₂S, found [M+H]⁺=354.2.

### 9.11 (4-((4-amino-2-(ethoxymethyl)-1H-imidazo[4,5-d]thieno[3,2-b]pyridin-1-yl)methyl)phenyl)methanol (Compound 9.g)

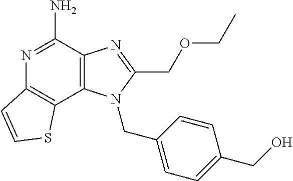 [SMILES: CCOCc1nc2c(N)nc3ccsc3c2n1Cc1ccc(CO)cc1]

The title compound was prepared according to General Procedure 4 starting from Compound 9.f (530 mg) using dichloromethane as the solvent. Purification was accomplished as described in General Procedure 11, eluting with eluting with a 10 to 50% CH₃CN/H₂O+0.1% TFA gradient to give Compound 9.g as an off-white solid (150 mg, 23% yield, assumed 1×TFA salt).

LC/MS: Calc'd m/z=368.1 for C₁₉H₂₀N₄O₂S, found [M+H]⁺=369.2.

### 9.12 1-(4-(chloromethyl)benzyl)-2-(ethoxymethyl)-1H-imidazo[4,5-d]thieno[3,2-b]pyridin-4-amine (Compound 9.h)

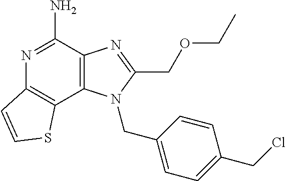 [SMILES: CCOCc1nc2c(N)nc3ccsc3c2n1Cc1ccc(CCl)cc1]

The title compound was prepared according to General Procedure 8 starting from Compound 9.g (150 mg). The crude compound was taken to the next step with no additional purification.

LC/MS: Calc'd m/z=386.1 for C₁₉H₁₉ClN₄OS, found [M+H]⁺=387.2.

### 9.13 2-(ethoxymethyl)-1-(4-(piperazin-1-ylmethyl)benzyl)-1H-imidazo[4,5-d]thieno[3,2-b]pyridin-4-amine (Compound 289)

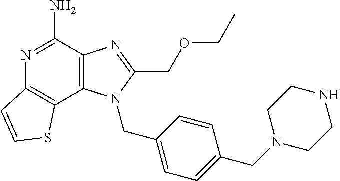 [SMILES: CCOCc1nc2c(N)nc3ccsc3c2n1Cc1ccc(CN2CCNCC2)cc1]

The title compound was prepared according to General Procedure 9 followed by General Procedure 5, starting from Compound 9.h (10 mg) and tert-butyl piperazine-1-carboxylate. Purification of the intermediate Boc-compound was accomplished by preparative HPLC as described in General Procedure 11, eluting with a 10 to 40% CH₃CN/H₂O+0.1% TFA gradient to give Compound 289 as a white solid (4.7 mg, 23% yield, assumed 3×TFA salt).

LC/MS: Calc'd m/z=436.2 for C₂₃H₂₈N₆OS, found [M+H]⁺=437.4. ¹H NMR (300 MHz, methanol-d4) δ 7.92 (d, J=5.5 Hz, 1H), 7.44 (d, J=5.5 Hz, 1H), 7.40 (d, J=8.0 Hz, 2H), 7.19 (d, J=7.9 Hz, 2H), 5.81 (s, 2H), 4.84 (s, 2H), 3.76 (s, 2H), 3.59 (q, J=7.0 Hz, 2H), 3.32-3.23 (m, 4H), 2.87-2.78 (m, 4H), 1.10 (t, J=7.0 Hz, 3H).

### 9.14 1-(4-((diethylamino)methyl)benzyl)-2-(ethoxymethyl)-1H-imidazo[4,5-d]thieno[3,2-b]pyridin-4-amine (Compound 290)

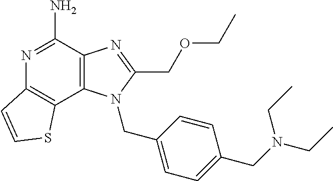 [SMILES: CCOCc1nc2c(N)nc3ccsc3c2n1Cc1ccc(CN(CC)CC)cc1]

The title compound was prepared according to General Procedure 9, starting from Compound 9.h (10 mg) and diethylamine. Purification was accomplished by preparative HPLC as described in General Procedure 11, eluting with a 10 to 40% CH₃CN/H₂O+0.1% TFA gradient to give Compound 290 as a white solid (7.9 mg, 47% yield, assumed 2×TFA salt).

LC/MS: Calc'd m/z=423.2 for C₂₃H₂₉N₅OS, found [M+H]⁺=424.2. 1H NMR (300 MHz, methanol-d4) δ 7.91 (d, J=5.4 Hz, 1H), 7.58-7.48 (m, 2H), 7.44 (d, J=5.4 Hz, 1H), 7.31 (d, J=8.2 Hz, 2H), 5.86 (s, 2H), 4.86 (s, 2H), 4.34 (s, 2H), 3.60 (q, J=7.0 Hz, 2H), 3.19 (qd, J=7.2, 2.5 Hz, 4H), 1.32 (t, J=7.3 Hz, 6H), 1.07 (t, J=7.0 Hz, 3H).

### 9.15 1-(4-(((3,3-difluorocyclobutyl)amino)methyl)benzyl)-2-(ethoxymethyl)-1H-imidazo[4,5-d]thieno[3,2-b]pyridin-4-amine (Compound 291)

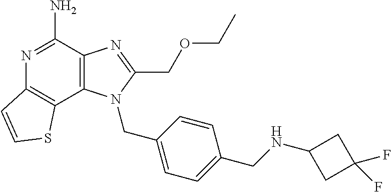 [SMILES: CCOCc1nc2c(N)nc3ccsc3c2n1Cc1ccc(CNC2CC(F)(F)C2)cc1]

The title compound was prepared according to General Procedure 9, starting from Compound 9.h (10 mg) and 3,3-difluorocyclobutan-1-amine. Purification was accomplished by preparative HPLC as described in General Procedure 11, eluting with a 10 to 40% CH₃CN/H₂O+0.1% TFA gradient to give Compound 291 as a white solid (5.9 mg, 33% yield, assumed 2×TFA salt).

LC/MS: Calc'd m/z=457.2 for C₂₃H₂₅F₂N₅OS, found [M+H]⁺=458.2. ¹H NMR (300 MHz, methanol-d₄) δ 7.90 (d, J=5.5 Hz, 1H), 7.51 (d, J=8.3 Hz, 2H), 7.43 (d, J=5.5 Hz, 1H), 7.29 (d, J=8.1 Hz, 2H), 5.85 (s, 2H), 4.86 (s, 2H), 4.20 (s, 2H), 3.87-3.68 (m, 1H), 3.61 (q, J=7.0 Hz, 2H), 3.09-2.92 (m, 2H), 2.92-2.72 (m, 2H), 1.09 (t, J=7.0 Hz, 3H).

### 9.16 (2S,3S)-2-((4-((4-amino-2-(ethoxymethyl)-1H-imidazo[4,5-d]thieno[3,2-b]pyridin-1-yl)methyl)benzyl)amino)-3-methylpentan-1-ol (Compound 292)

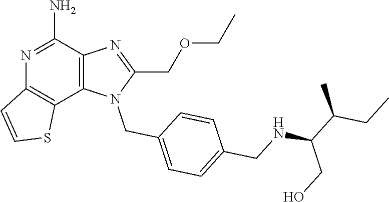 [SMILES: CCOCc1nc2c(N)nc3ccsc3c2n1Cc1ccc(CN[C@H](CO)[C@@H](C)CC)cc1]

The title compound was prepared according to General Procedure 9 starting from Compound 9.h (10 mg) and L-isoleucinol. Purification was accomplished by preparative HPLC as described in General Procedure 11, eluting with a 10 to 40% CH₃CN/H₂O+0.1% TFA gradient to give Compound 292 as a white solid (6.0 mg, 33% yield, assumed 2×TFA salt).

LC/MS: Calc'd m/z=467.2 for C₂₅H₃₃N₅O₂S, found [M+H]⁺=468.4. ¹H NMR (300 MHz, methanol-d₄) δ 7.90 (d, J=5.5 Hz, 1H), 7.59-7.50 (m, 2H), 7.44 (d, J=5.5 Hz, 1H), 7.29 (d, J=8.2 Hz, 2H), 5.85 (s, 2H), 4.86 (s, 1H), 4.52-4.10 (m, 2H), 3.86 (dd, J=12.1, 3.9 Hz, 1H), 3.76 (dd, J=12.1, 7.1 Hz, 1H), 3.61 (q, J=7.0 Hz, 2H), 3.11 (dt, J=7.0, 4.2 Hz, 1H), 1.83 (p, J=6.0 Hz, 1H), 1.56-1.17 (m, 2H), 1.11 (t, J=7.0 Hz, 3H), 0.96 (d, J=6.9 Hz, 3H), 0.87 (t, J=7.4 Hz, 3H).

### 9.17 1-(4-((((1H-pyrrol-3-yl)methyl)amino)methyl)benzyl)-2-(ethoxymethyl)-1H-imidazo [4,5-d]thieno[3,2-b]pyridin-4-amine (Compound 293)

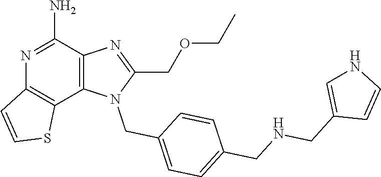 [SMILES: CCOCc1nc2c(N)nc3ccsc3c2n1Cc1ccc(CNCc2cc[nH]c2)cc1]

The title compound was prepared according to General Procedure 9 starting from Compound 9.h (10 mg) and (1H-pyrrol-3-yl)methanamine. Purification was accomplished by preparative HPLC as described in General Procedure 11, eluting with a 10 to 40% CH₃CN/H₂O+0.1% TFA gradient to give Compound 293 as a white solid (5.4 mg, 31% yield, assumed 2×TFA salt).

LC/MS: Calc'd m/z=446.2 for C₂₄H₂₆N₆OS, found [M+H]⁺=447.2. ¹H NMR (300 MHz, methanol-d₄) δ 7.90 (d, J=5.5 Hz, 1H), 7.49-7.41 (m, 3H), 7.27 (d, J=8.1 Hz, 2H), 6.91 (q, J=2.0 Hz, 1H), 6.81 (td, J=2.7, 1.9 Hz, 1H), 6.21 (td, J=2.6, 1.6 Hz, 1H), 5.85 (s, 2H), 4.88 (s, 2H), 4.15 (s, 2H), 4.09 (s, 2H), 3.60 (q, J=7.0 Hz, 2H), 1.09 (t, J=7.0 Hz, 3H).

### Example 10: Preparation of Drug-Linkers

### 10.1 2,3,5,6-tetrafluorophenyl 3-(2-(2-(2-(2,5-dioxo-2,5-dihydro-1H-pyrrol-1-yl)ethoxy) ethoxy)ethoxy)propanoate (Compound 10.a; MT-OTfp)

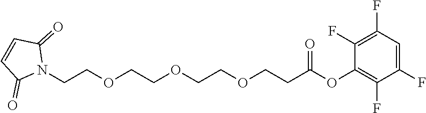 [SMILES: O=C(CCOCCOCCOCCN1C(=O)C=CC1=O)Oc1c(F)c(F)cc(F)c1F]

The title compound was prepared according to the procedure described in International Patent Publication No. WO 2017/054080.

LC/MS: Calc'd m/z=449.4 for C₁₉H₁₉F₄O₇, found [M+H]⁺=450.4. 10.2 tert-butyl ((S)-1-(((S)-1-((4-(hydroxymethyl)phenyl)amino)-1-oxo-5-ureidopentan-2-yl) amino)-3-methyl-1-oxobutan-2-yl)carbamate (Compound 10.b; Boc-VC-PAB-OH)

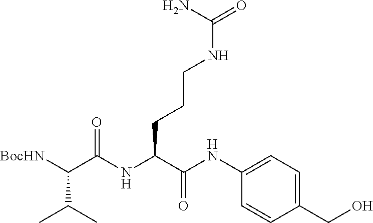 [SMILES: CC(C)[C@H](NC(=O)OC(C)(C)C)C(=O)N[C@@H](CCCNC(N)=O)C(=O)Nc1ccc(CO)cc1]

The title compound was prepared according to the procedure described in International Patent Publication No. WO 2005/112919.

LC/MS: Calc'd m/z=479.3 for C₂₃H₃₇N₅O₆, found [M+H]⁺=480.4, [M-Boc, +H]⁺=380.2.

### 10.3 (S)-2-((S)-2-amino-3-methylbutanamido)-N-(4-(hydroxymethyl)phenyl)-5-ureidopentanamide (Compound 10.c; VC-PAB-OH)

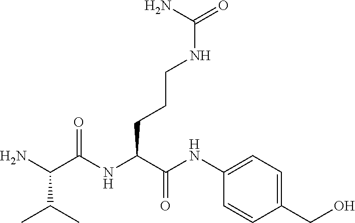 [SMILES: CC(C)[C@H](N)C(=O)N[C@@H](CCCNC(N)=O)C(=O)Nc1ccc(CO)cc1]

The title compound was prepared according to the procedure described in International Patent Publication No. WO 2017/214282.

LC/MS: Calc'd m/z=379.2 for C₁₈H₂₉N₅O₄, found [M+H]⁺=380.2.

### 10.4 (S)-2-((S)-1-(2,5-dioxo-2,5-dihydro-1H-pyrrol-1-yl)-14-isopropyl-12-oxo-3,6,9-trioxa-13-azapentadecan-15-amido)-N-(4-(hydroxymethyl)phenyl)-5-ureidopentanamide (Compound 10.d; MT-VC-PAB-OH)

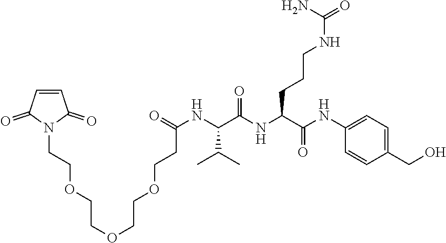 [SMILES: CC(C)[C@H](NC(=O)CCOCCOCCOCCN1C(=O)C=CC1=O)C(=O)N[C@@H](CCCNC(N)=O)C(=O)Nc1ccc(CO)cc1]

To a solution of Compound 10.c (740 mg, 1.95 mmol) in 5 mL DMF was added Compound 10.a (876 mg, 1.95 mmol) followed by DIPEA (1.4 mL, 7.8 mmol). Upon completion, the reaction was adjusted to pH 1 with 1 M aqueous HCl. Purification was accomplished by reverse phase chromatography as described in General Procedure 11, eluting with a 10 to 50% CH₃CN/H₂O+0.1% TFA gradient to give Compound 10.d as a white solid (960 mg, 79% yield).

LC/MS: Calc'd m/z=662.3 for C₃₁H₄₆N₆O₁₀, found [M+H]⁺=663.2.

### 10.5 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (4-nitrophenyl) carbonate (Compound 10.e)

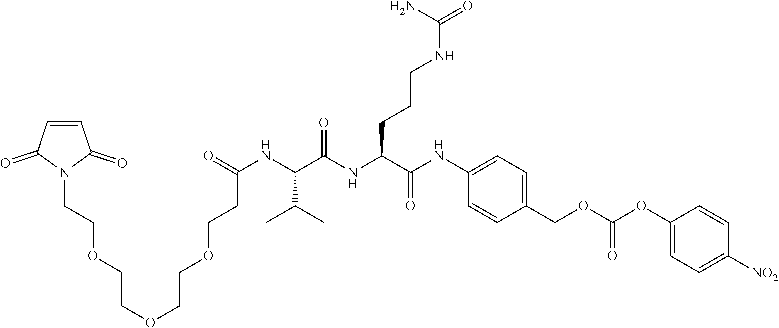 [SMILES: CC(C)[C@H](NC(=O)CCOCCOCCOCCN1C(=O)C=CC1=O)C(=O)N[C@@H](CCCNC(N)=O)C(=O)Nc1ccc(COC(=O)Oc2ccc([N+](=O)[O-])cc2)cc1]

To a solution of Compound 10.d (910 mg, 1.37 mmol) in 5 mL DMF was added bis(4-nitrophenyl) carbonate (459 mg, 1.51 mmol) followed by DIPEA (0.72 mL, 2.75 mmol). Upon completion, the reaction mixture was adjusted to pH 1 with 1 M aqueous HCl. Purification was accomplished by reverse phase chromatography as described in General Procedure 11, eluting with a 10 to 60% CH₃CN/H₂O+0.1% TFA gradient to give Compound 10.e as a white solid (1.04 g, 92% yield).

LC/MS: Calc'd m/z=827.3 for C₃₈H₄₉N₇O₁₄, found [M+H]⁺=828.3.

### 10.6 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (2-(4-(4-((4-amino-2-butyl-1H-imidazo[4,5-d]thieno[3,2-b]pyridin-1-yl)methyl)benzyl)piperazin-1-yl)ethyl)carbamate (MT-VC-PABC-Compound 111)

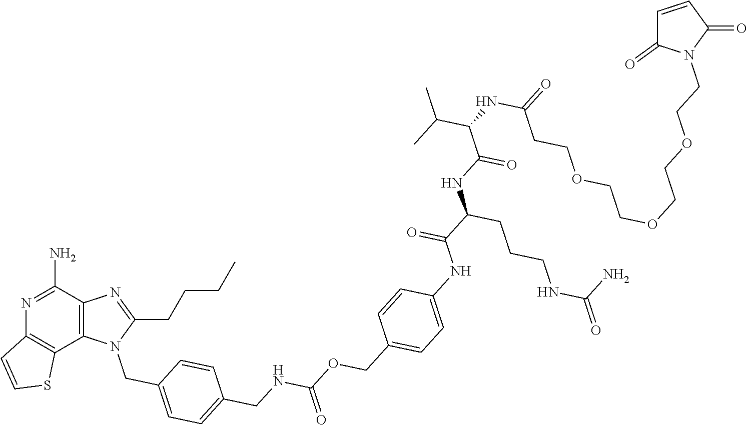 [SMILES: CCCCc1nc2c(N)nc3ccsc3c2n1Cc1ccc(CNC(=O)OCc2ccc(NC(=O)[C@H](CCCNC(N)=O)NC(=O)[C@@H](NC(=O)CCOCCOCCOCCN3C(=O)C=CC3=O)C(C)C)cc2)cc1]

The title compound was prepared according to General Procedure 10 using Compound 111 (11 mg, assumed 2×TFA salt). Purification was accomplished by reverse phase chromatography as described in General Procedure 11, eluting with a 5 to 50% CH₃CN/H₂O+0.1% TFA gradient to give MT-VC-PABC-Compound 111 as a white solid (8.2 mg, 26% yield, assumed 1×TFA salt).

LC/MS: Calc'd m/z=1053.5 for C₅₂H₆₇N₁₁O₁₁S, found [M+H]⁺=1054.6, [M+2H]²⁺=528.0. ¹H NMR (300 MHz, acetonitrile-d₃) δ 7.79 (d, J=5.4 Hz, 1H), 7.57 (d, J=8.1 Hz, 1H), 7.41 (d, J=5.3 Hz, 1H), 7.25 (t, J=8.5 Hz, 3H), 7.05 (d, J=8.0 Hz, 2H), 6.77 (s, 2H), 5.60 (s, 2H), 4.99 (s, 2H), 4.45 (dd, J=9.5, 4.5 Hz, 1H), 4.23 (s, 2H), 4.14 (d, J=6.5 Hz, 1H), 3.83-3.65 (m, 2H), 3.64-3.54 (m, 3H), 3.50 (q, J=2.5 Hz, 6H), 3.11 (dt, J=11.2, 6.8 Hz, 2H), 2.53 (t, J=6.0 Hz, 2H), 2.11 (q, J=6.8 Hz, 1H), 1.81-1.67 (m, 2H), 1.52 (d, J=7.4 Hz, 1H), 1.45-1.34 (m, 2H), 0.99-0.85 (m, 9H).

### 10.7 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (2-(4-(4-((4-amino-2-butyl-1H-imidazo[4,5-d]thieno[3,2-b]pyridin-1-yl)methyl)benzyl)piperazin-1-yl)ethyl)carbamate (MT-VC-PABC-Compound 166)

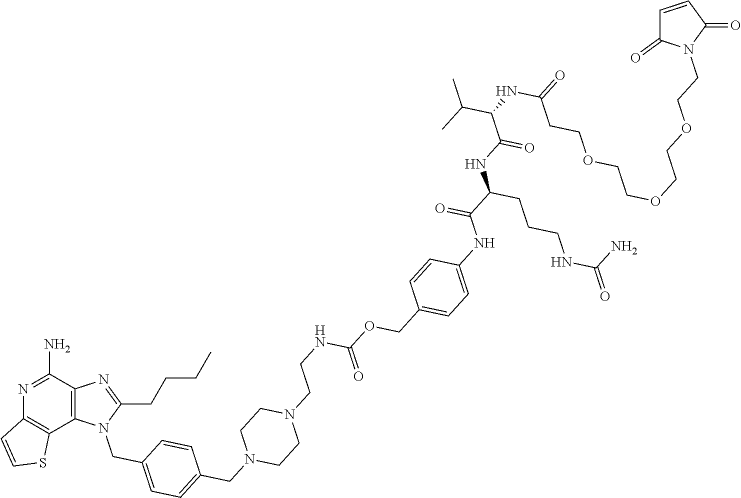 [SMILES: CCCCc1nc2c(N)nc3ccsc3c2n1Cc1ccc(CN2CCN(CCNC(=O)OCc3ccc(NC(=O)[C@H](CCCNC(N)=O)NC(=O)[C@@H](NC(=O)CCOCCOCCOCCN4C(=O)C=CC4=O)C(C)C)cc3)CC2)cc1]

The title compound was prepared according to General Procedure 10 using Compound 166 (29 mg, assumed 3×TFA salt). Purification was accomplished by reverse phase chromatography as described in General Procedure 11, eluting with a 25 to 55% CH₃CN/H₂O+0.1% TFA gradient to give MT-VC-PABC-Compound 166 as a white solid (10.1 mg, 19% yield, assumed 3×TFA salt).

LC/MS: Calc'd m/z=1165.6 for C₅₈H₇₉N₁₃O₁₁S, found [M+H]⁺=1166.8, [M+2H]²⁺=584.0. ¹H NMR (300 MHz, acetonitrile-d₃) δ 7.79 (d, J=5.5 Hz, 1H), 7.68-7.53 (m, 2H), 7.43 (d, J=5.5 Hz, 1H), 7.39 (d, J=8.0 Hz, 2H), 7.30 (d, J=8.4 Hz, 2H), 7.21-7.11 (m, 2H), 6.78 (s, 2H), 5.64 (s, 2H), 5.01 (s, 2H), 4.42 (dd, J=9.6, 4.5 Hz, 1H), 4.12 (d, J=6.4 Hz, 1H), 3.95 (s, 2H), 3.76-3.65 (m, 2H), 3.65-3.54 (m, 3H), 3.50 (s, 4H), 3.39 (t, J=5.9 Hz, 2H), 3.20 (s, 4H), 3.08-2.99 (m, 6H), 2.52 (t, J=6.0 Hz, 2H), 2.09 (q, J=6.7 Hz, 1H), 1.75 (p, J=7.7 Hz, 2H), 1.51 (dt, J=14.5, 7.0 Hz, 2H), 1.39 (dt, J=14.6, 7.4 Hz, 2H), 0.97-0.84 (m, 9H).

### Example 11: In Vitro Agonism of Tlr7 and Tlr8

The ability of the compounds of Formula I to agonise TLR7 and TLR8 was assessed by a reporter gene assay as well as IL-6 release from peripheral blood mononuclear cells (PBMCs) as described below.

### Methods

### Reporter Gene Assay

3×10⁴ HEK-Blue™ TLR7 or 3×10⁴ TLR8 reporter cells (InvivoGen, San Diego, CA) were treated with titrated (4-fold) amounts of test compound. Compounds were dissolved in DMSO. Starting compound concentration was 15 μM. A 4-fold, 12-point titration curve was performed in cell culture medium for detection of secreted embryonic alkaline phosphatase (SEAP) (HEK-Blue™ Detection; InvivoGen, San Diego, CA). Cells were incubated overnight at 37° C. and 5% CO₂, then absorbance (620 and 655 nm) was read on a Synergy™ H1 microplate reader.

### PBMC Assay

Whole peripheral blood was obtained from healthy donors. PBMCs were isolated from peripheral blood by density gradient centrifugation. Briefly, 15 mL of whole blood was diluted with an equal volume of wash buffer (1×PBS, 2% fetal bovine serum (FBS)), overlaid on 15 mL of Lymphoprep™ (#07801; STEMCELL Technologies, Vancouver, Canada) and centrifuged at 1200×G for 10 minutes in SepMate™ tubes (#15450, STEMCELL Technologies). The plasma layer containing PBMCs was poured into a new tube and washed twice. Isolated PBMCs were either used fresh or thawed from previously frozen. 1×10⁵ PBMCs were cultured with titrating concentrations of compounds of Formula I overnight followed by assaying for cytokines by homogeneous time resolved fluorescence (HTRF). Compounds were dissolved in DMSO. Starting compound concentration was 15 μM. A 4-fold, 12-point titration curve was performed in cell culture medium (RPMI, 10% FBS). Cells were incubated overnight at 37° C. and 5% CO₂ followed by assaying for the release of relevant cytokines (IL-6, TNF-α) by HTRF in the supernatants.

### Controls and Analyses

Each assay included a positive control (Compound C-8 from International Patent Publication No. WO 2017/072662; shown below) and a negative control (growth medium). Dose response curves were plotted on PRISM (GraphPad Software, San Diego, CA) and EC₅₀ values were calculated as the concentration of the compound required to produce 50% maximal effect (IL-6 induction or NF-κB activation measured by SEAP for PBMCs and HEK Blue TLR7/8 assays, respectively).

Structure of compound C-8 from International Patent Publication No. WO 2017/072662:

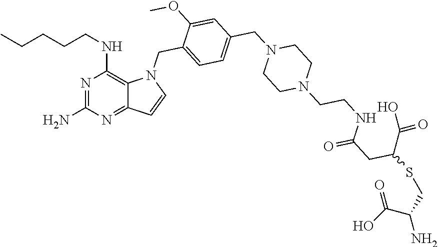 [SMILES: CCCCCNc1nc(N)nc2ccn(Cc3ccc(CN4CCN(CCNC(=O)CC(SC[C@H](N)C(=O)O)C(=O)O)CC4)cc3OC)c12]

### Results

The results of the Reporter Gene Assay (TLR7 and TLR8) and the ability of the tested compounds to induce of the production of cytokines in the PBMC assay are shown in Tables 11.1 and 11.2.

TABLE 11.1
Agonism of TLR7 and TLR8 by Compounds of Formula I*

| | EC₅₀/nM | | |
|---|---|---|---|
| Compound | TLR7 | TLR8 | IL-6 |
| 112 | 200 | >1000 | 850 |
| 111 | 7.6 | 410 | 5.0 |
| 141 | 370 | >1000 | >1000 |
| 144 | 2.6 | 617 | 9.6 |
| 145 | 27 | >1000 | 44 |
| 146 | 4.3 | >1000 | 15 |
| 147 | 3.5 | 930 | 17 |
| 148 | 3.8 | 990 | 19 |
| 149 | 2.8 | 410 | 14 |
| 150 | 1.8 | 460 | 7.7 |
| 151 | 3.8 | >1000 | 20 |
| 152 | 67 | >1000 | 59 |
| 153 | 85 | >1000 | 420 |
| 154 | 3.3 | >1000 | 10 |
| 155 | 444 | >1000 | 110 |
| 156 | 6.6 | >1000 | 20 |
| 157 | 4.8 | >1000 | 7.4 |
| 158 | 20 | >1000 | 92 |
| 159 | 8.9 | >1000 | 16 |
| 160 | 8.7 | >1000 | 32 |
| 161 | 13 | >1000 | 14 |
| 162 | 3.0 | >1000 | 11 |
| 163 | 5.0 | >1000 | 25 |
| 164 | >1000 | >1000 | >1000 |
| 165 | 15 | >1000 | 65 |
| 166 | 8.8 | >1000 | 50 |
| 167 | 76 | >1000 | 200 |
| 168 | 3.4 | 578 | 15 |
| 172 | 27 | >1000 | 580 |
| 173 | >1000 | >1000 | >1000 |
| 174 | 21 | >1000 | 78 |
| 175 | 12 | 380 | 99 |
| 176 | 33 | >1000 | 58 |
| 177 | 24 | 570 | 270 |
| 178 | 37 | >1000 | >1000 |
| 179 | 12 | 260 | 76 |
| 180 | 83 | >1000 | 610 |
| 182 | 24 | >1000 | 160 |
| 183 | 34 | >1000 | >1000 |
| 185 | 89 | >1000 | >1000 |
| 196 | >1000 | >1000 | >1000 |
| 197 | 63 | 490 | 290 |
| 198 | 220 | >1000 | 280 |
| 199 | 130 | 430 | 330 |
| 200 | 550 | >1000 | >1000 |
| 201 | 82 | 72 | 58 |
| 202 | 330 | 950 | 750 |
| 203 | 290 | 120 | 160 |
| 204 | 68 | 310 | 200 |
| 205 | 110 | 190 | 160 |
| 206 | 280 | >1000 | >1000 |
| 207 | 480 | >1000 | >1000 |
| 210 | >1000 | >1000 | >1000 |
| 214 | 63 | >1000 | 750 |
| 215 | >1000 | >1000 | >1000 |
| 216 | >1000 | >1000 | >1000 |
| 217 | >1000 | >1000 | >1000 |
| 218 | >1000 | >1000 | >1000 |
| 219 | >1000 | >1000 | >1000 |
| 220 | >1000 | >1000 | >1000 |
| 221 | >1000 | >1000 | >1000 |
| 222 | >1000 | >1000 | >1000 |
| 223 | >1000 | >1000 | >1000 |
| 224 | >1000 | >1000 | >1000 |
| 225 | >1000 | >1000 | >1000 |
| 228 | 220 | >1000 | >1000 |
| 229 | 420 | >1000 | >1000 |
| 231 | 110 | 580 | 920 |
| 232 | 260 | >1000 | >1000 |
| 233 | 140 | 880 | 630 |
| 234 | 270 | >1000 | >1000 |
| 235 | 190 | 770 | 700 |
| 236 | 310 | 73 | 310 |
| 237 | 190 | >1000 | 500 |
| 238 | 420 | >1000 | >1000 |
| 241 | >1000 | >1000 | >1000 |
| 242 | 150 | >1000 | >1000 |
| 245 | >1000 | >1000 | >1000 |
| 253 | >1000 | >1000 | >1000 |
| 254 | 20 | 540 | 30 |
| 260 | 89 | >1000 | 719 |
| 261 | 106 | >1000 | 602 |
| 262 | 95 | >1000 | >1000 |
| 263 | 53 | >1000 | 853 |
| 264 | 83 | >1000 | 153 |
| 265 | 20 | >1000 | 434 |
| 267 | >1000 | >1000 | >1000 |
| 268 | >1000 | >1000 | >1000 |
| 269 | 186 | >1000 | 557 |
| 272 | 33 | >1000 | N.D. |
| 275 | >1000 | >1000 | N.D. |
| 277 | <1 | 404 | N.D. |
| 279 | <1 | >1000 | N.D. |
| 284 | 11 | 1992 | N.D. |
| 286 | 58 | >1000 | N.D. |
| 289 | 9 | >1000 | N.D. |
| 290 | 4 | 1028 | N.D. |
| 291 | 14 | 1127 | N.D. |
| 292 | 3 | 674 | N.D. |
| 293 | 7 | >1000 | N.D. |
| 295 | >1000 | >1000 | N.D. |
| 296 | <1 | 134 | N.D. |
| 297 | 676 | >1000 | N.D. |
| 298 | 159 | >1000 | N.D. |
| 299 | 446 | >1000 | N.D. |
| 300 | >1000 | >1000 | N.D. |
| 301 | 348 | >1000 | N.D. |
| 303 | 703 | >1000 | N.D. |
| 305 | 108 | >1000 | N.D. |

*Reported values are an average of an experiment run in quadruplicate
^{§} N.D. = not determined

TABLE 11.2
Induction of TNF-α by Compounds of Formula I

| | EC₅₀/nM |
|---|---|
| Compound | TNF-α |
| 111 | 223 |
| 149 | 155 |
| 154 | 59 |
| 161 | 117 |
| 166 | >1000 |
| 185 | >1000 |
| 201 | 125 |
| 235 | >1000 |
| 236 | >1000 |
| 254 | 112 |
| 267 | >1000 |
| 284 | 271 |

### Example 12: Preparation of Antibody-Drug Conjugates

### 12.1 Trastuzumab-MTvcPABC-Compound 111

This conjugate was prepared following General Procedure 13, using Trastuzumab as the antibody, 2.2 equivalent of TCEP, 12 equivalents of drug-linker, and performing the conjugation and purification in 10 mM sodium acetate, pH 4.5.

### 12.2 Trastuzumab-MTvcPABC-Compound 166

This conjugate was prepared following General Procedure 13, using Trastuzumab as the antibody, 2.2 equivalent of TCEP, 12 equivalents of drug-linker, and performing the conjugation and purification in phosphate buffered saline, pH 7.4.

### Results

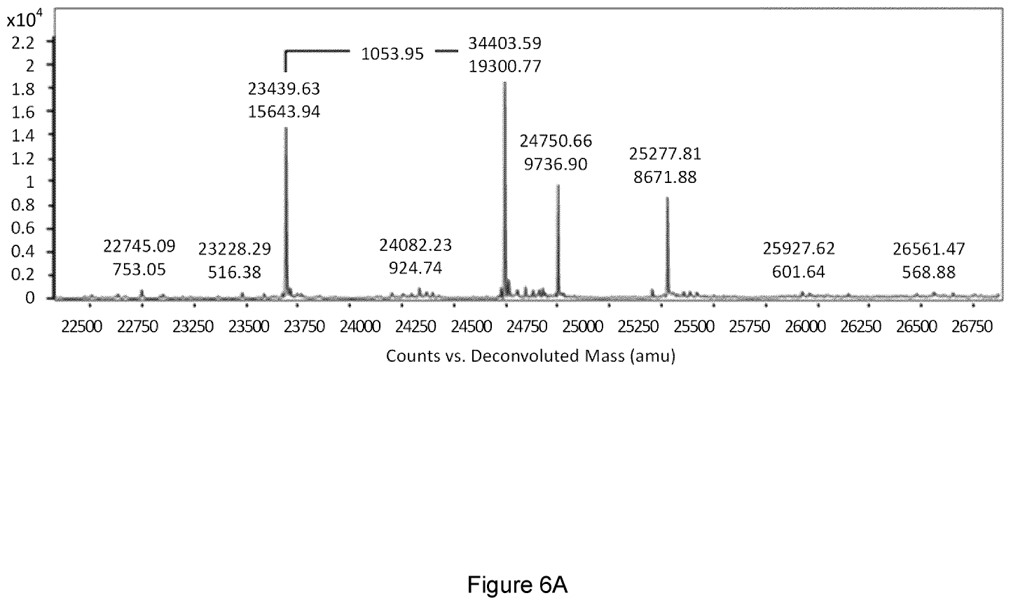

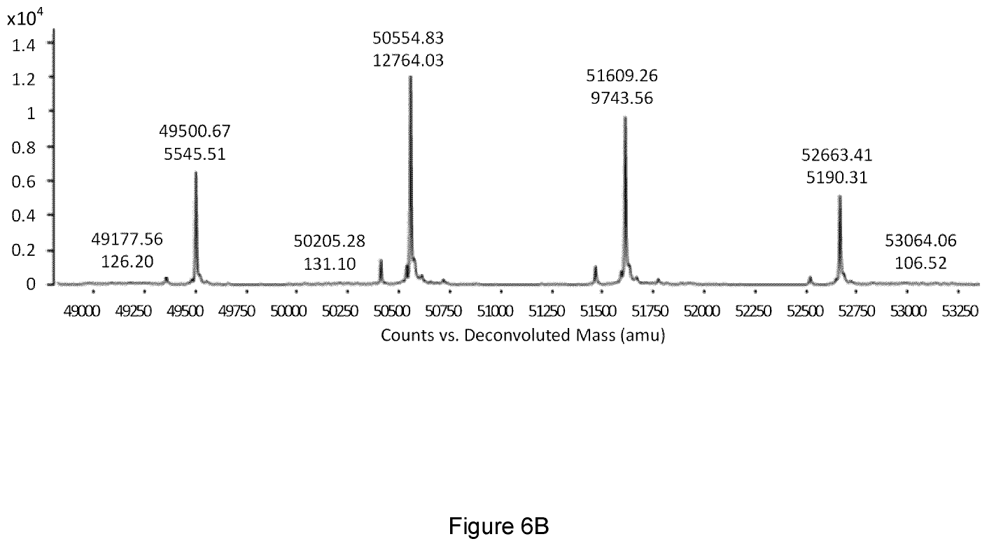

FIGS. 6A and 6B show the deconvoluted spectra obtained from the antibody-drug conjugate Trastuzumab-MTvcPABC-Compound 111 light chain and heavy chains, respectively.

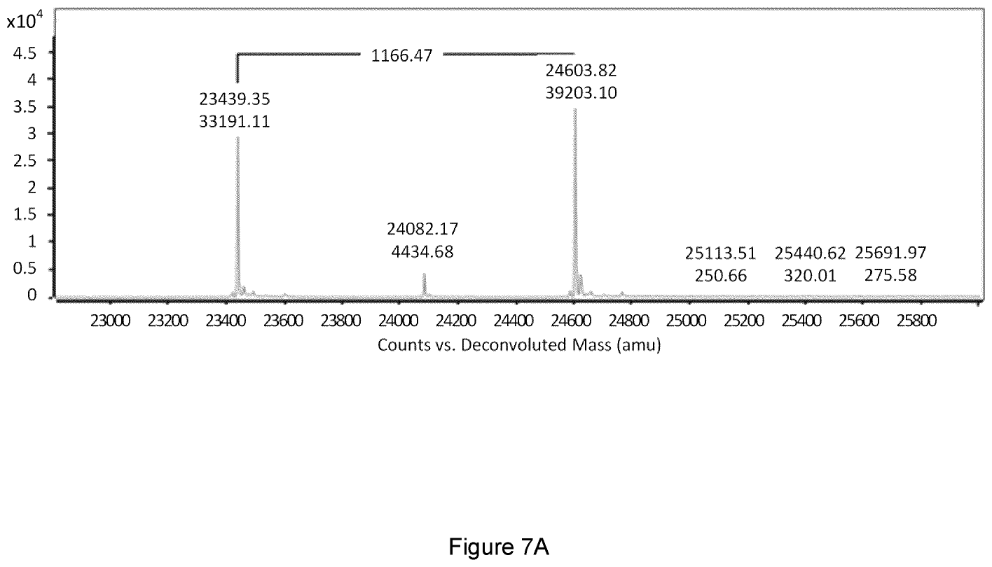

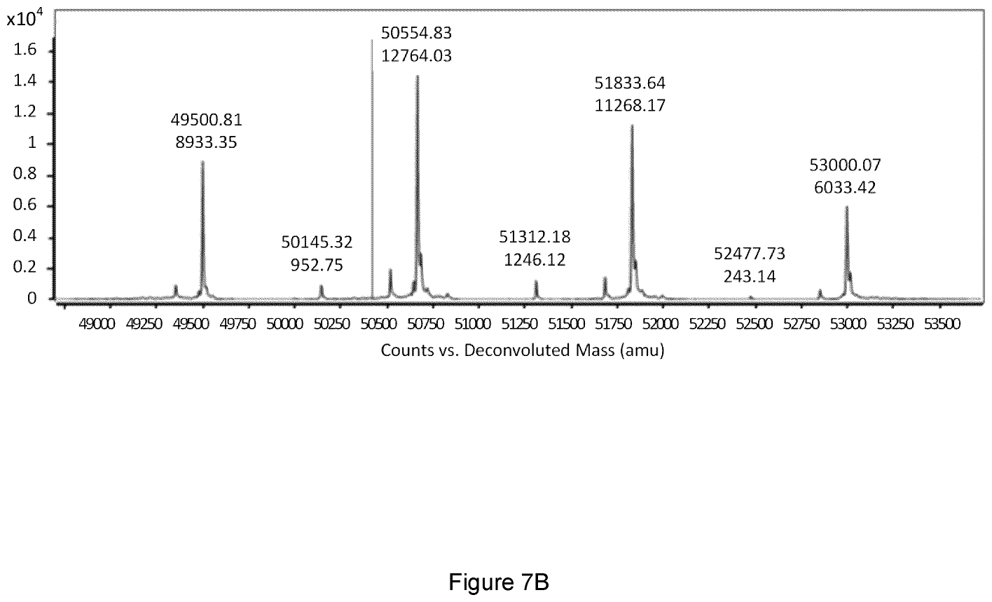

FIGS. 7A and 7B show the deconvoluted spectra obtained from conjugate Trastuzumab-MTvcPABC-Compound 166 light chain and heavy chains, respectively. Analysis of data indicated an average DAR between 3.6-4.2 for both antibody-drug conjugates.

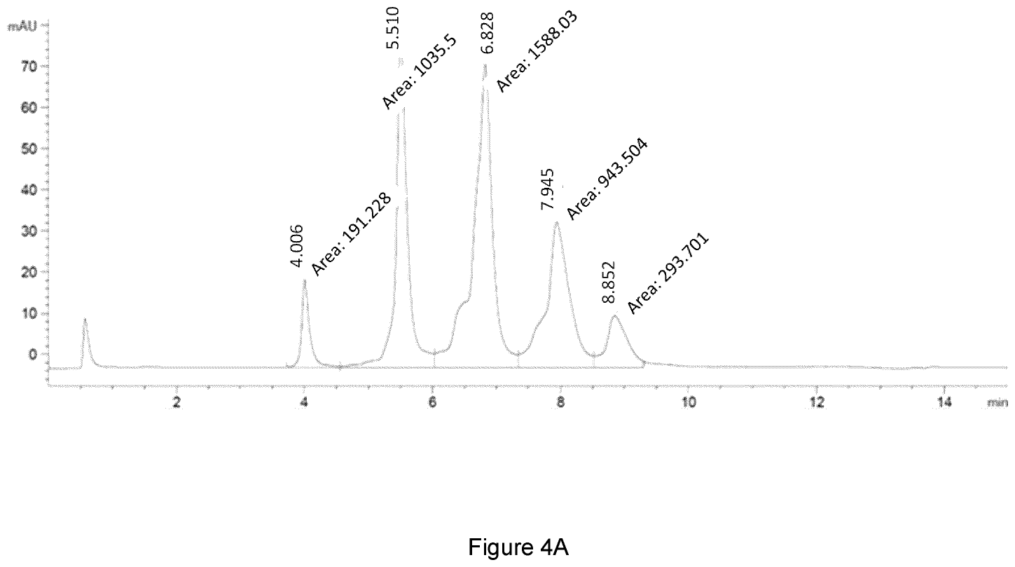

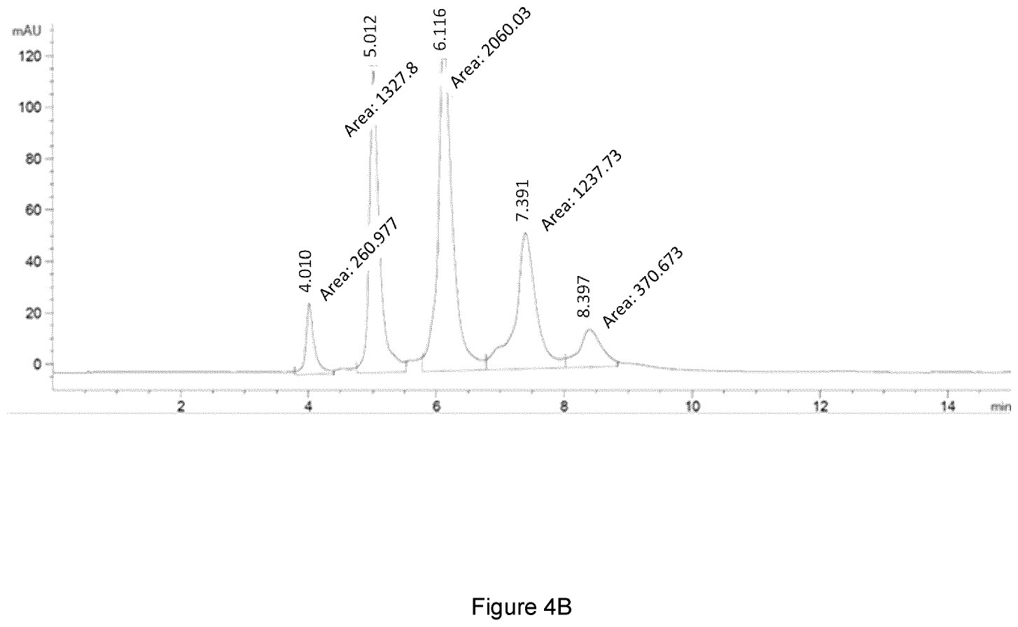

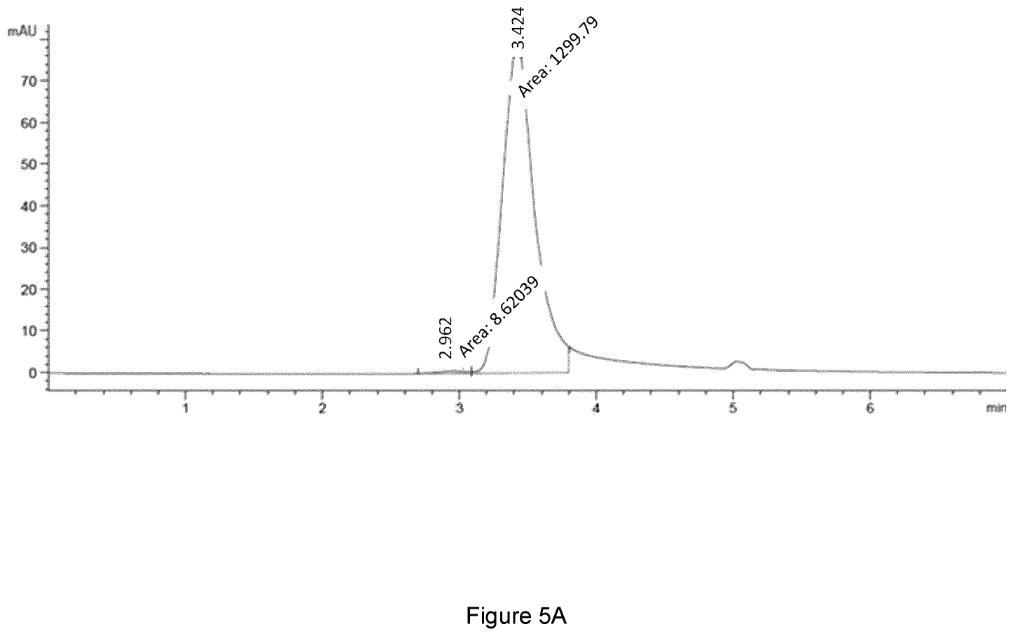

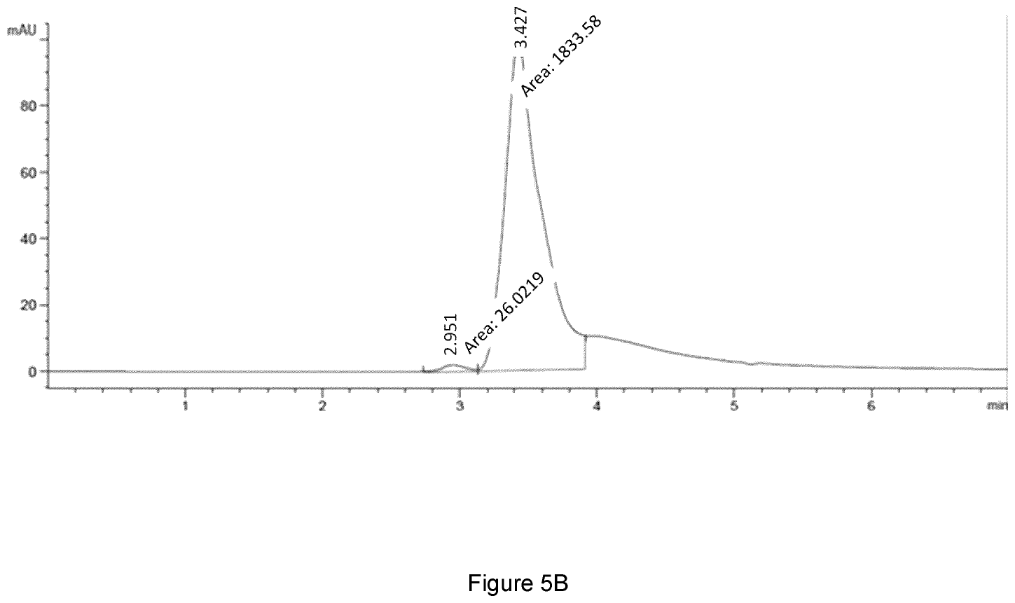

FIGS. 4A and 4B show the HIC chromatograms indicating the distribution of DAR species for the antibody-drug conjugates Trastuzumab-MTvcPABC-Compound 111 and Trastuzumab-MTvcPABC-Compound 166, respectively. FIGS. 5A and 5B show the chromatograms obtained from size exclusion chromatography (SEC) analysis, indicating the presence of minimal amounts of aggregated species in either preparation of antibody-drug conjugates Trastuzumab-MTvcPABC-Compound 111 or Trastuzumab-MTvcPABC-Compound 166.

### Example 13: In Vivo Activity of Antibody-Drug Conjugates

This experiment was performed to assess the anti-tumor activity of the antibody-drug conjugates Trastuzumab-MTvcPABC-Compound 111 and Trastuzumab-MTvcPABC-Compound 166 (see Example 12) in the NCI-N₈₇ xenograft model of gastric cancer (HER2 high).

Tumor cell suspensions (10⁷ cells in a 1:1 mix of PBS and matrigel) were implanted subcutaneously into balb/c nude mice. When mean tumor volume reached ˜165 mm³, the animals were randomly assigned to groups (n=6 per group) and treated as shown in Table 13.1 below. Tumor volume and body weight were measured twice weekly with a study duration of 44 days.

TABLE 13.1
In vivo Study Design

| | Active Agent | Admin. | Admin. |
|---|---|---|---|
| Agent | Dosage (mg/kg) | Route | Time point |
| Vehicle | 0 | iv | Day 0 |
| Trastuzumab-MTvcPABC- | 3 | iv | Day 0 |
| Compound 111 | | | |
| Trastuzumab-MTvcPABC- | 3 | iv | Day 0 |
| Compound 166 | | | |

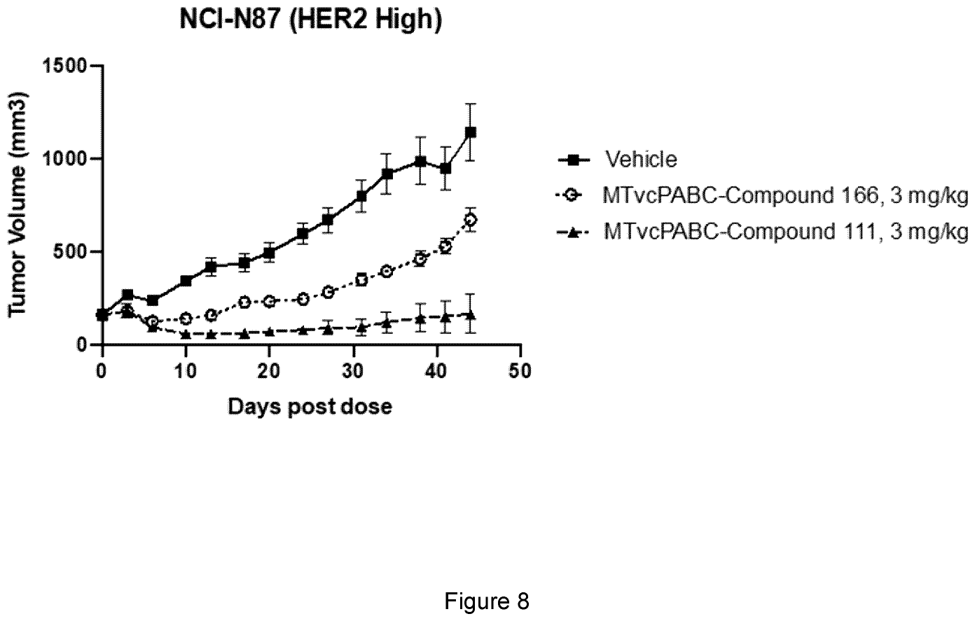

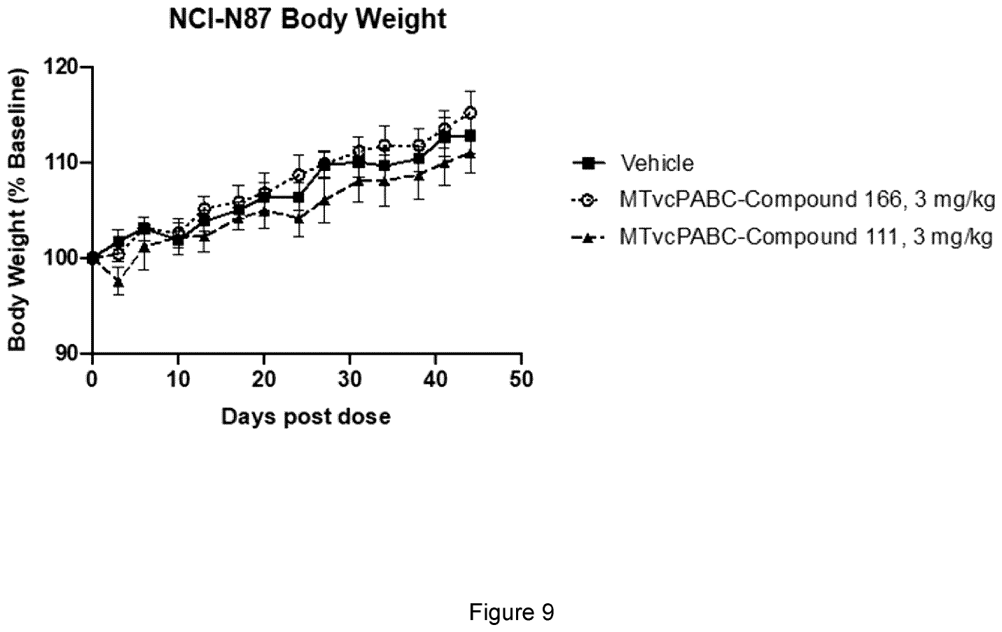

The results are shown in FIGS. 8 and 9. Both Compound 111 and Compound 166 conjugated ADCs significantly inhibited the tumor growth rate at a single dose (administered i.v. at day 0) at 3 mg/kg compared to vehicle control (p<0.01) (FIG. 8), with no significant effect on body weight (FIG. 9).

Plural instances may be provided for components, operations, or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

All publications, patents, and patent applications mentioned in this specification are herein incorporated by reference in their entireties to the same extent as if each individual publication, patent, or patent application was specifically and individually indicated to be incorporated by reference. In the event of a conflict between a term herein and a term in an incorporated reference, the term herein controls.

What is claimed is:

1. A compound having Formula I: 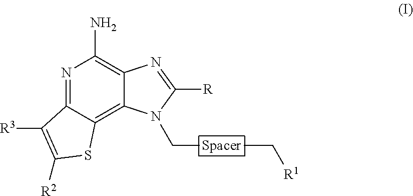
or a pharmaceutically acceptable salt thereof,
wherein:
R is H, C₁-C₆ alkyl, CH₂SR¹⁵ or CH₂OR¹⁵;
R¹ is —OH, —NR⁴R⁵, —OR¹⁰, SR¹¹ or 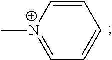
R² and R³ are each independently H or optionally substituted C₁-C₆ alkyl;
Spacer is —(CH₂)ₙ—, 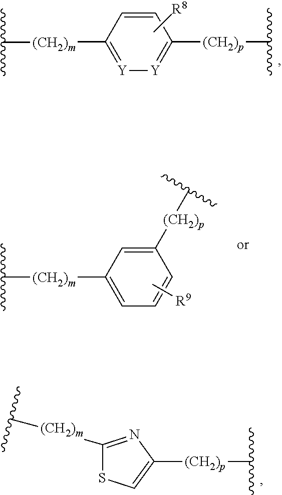
wherein n is an integer between 3 and 10; each m is independently an integer between 0 and 4; each p is independently an integer between 0 and 4; and Y is CH or N;
R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;
R⁸ and R⁹ are each independently H, NR¹³R¹⁴, halo, optionally substituted C₁-C₄ alkoxy or optionally substituted C₁-C₄ alkyl;
R¹⁰ and R¹¹ are each independently optionally substituted C₁-C₆ alkyl, optionally substituted aryl or optionally substituted C₃-C₇ cycloalkyl;
R¹³ and R¹⁴ are each independently H or optionally substituted C₁-C₄ alkyl; and
R¹⁵ is C₃-C₄ cycloalkyl or C₁-C₄ alkyl optionally substituted with one or more halo.

2. The compound according to claim 1, wherein R is C₁-C₆ alkyl.

3. The compound according to claim 1 or claim 2, wherein R is C₂-C₄ alkyl.

4. The compound according to claim 1, wherein R is CH₂OR¹⁵.

5. The compound according to claim 4, wherein R¹⁵ is C₁-C₂ alkyl.

6. The compound according to any one of claims 1-5, wherein R⁸ and R⁹ are each H or halo.

7. The compound according to any one of claims 1-6, wherein Spacer is 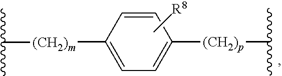
m is an integer between 0 and 4; p is an integer between 0 and 4; R⁸ is H, NR¹³R¹⁴, halo, optionally substituted C₁-C₄ alkoxy or optionally substituted C₁-C₄ alkyl, and wherein R¹³ and R¹⁴ are each independently H or C₁-C₄ alkyl.

8. The compound according to any one of claims 1-7, wherein m is 0.

9. The compound according to any one of claims 1-8, wherein p is 0.

10. The compound according to any one of claims 1-6, wherein Spacer is

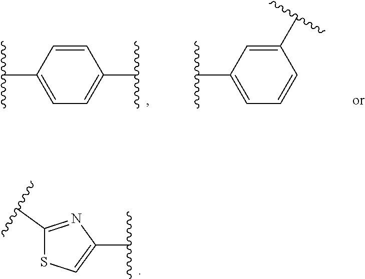 [SMILES: *c1ccc(*)cc1 ; *c1cccc(*)c1 ; *c1csc(*)n1]

11. The compound according to any one of claims 1-6, wherein n is an integer between 3 and 5.

12. The compound according to any one of claims 1-11, wherein R¹ is —OH, —NR⁴R⁵, —OR¹⁰, SR¹¹, 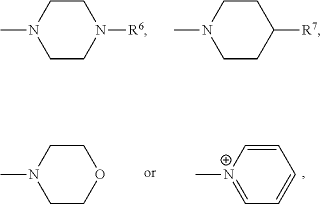
and wherein
R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl;
R⁶ and R⁷ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; and
R¹⁰ and R¹¹ are each independently optionally substituted C₁-C₆ alkyl, optionally substituted aryl or optionally substituted C₃-C₇ cycloalkyl.

13. The compound according to claim 12, wherein R¹ is —OH, —NR⁴R⁵,

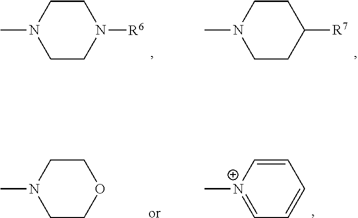

14. The compound according to claim 12 or claim 13, wherein R⁶ is H, optionally substituted C₁-C₄ alkoxycarbonyl, optionally substituted C₁-C₄ alkyl, optionally substituted C₁-C₄ aminoalkyl or optionally substituted C₃-C₇ heterocyclyl-C₁-C₄ alkyl.

15. The compound according to claim 12 or claim 13, wherein R⁷ is H, optionally substituted C₁-C₄ alkyl, optionally substituted C₁-C₄ aminoalkyl or optionally substituted heteroaryl.

16. The compound according to any one of claims 1-13, wherein R⁴ is H, optionally substituted C₁-C₄ alkyl, optionally substituted C₁-C₄ carboxyalkyl or optionally substituted C₁-C₄ aminoalkyl.

17. The compound according to any one of claims 1-13, or 16, wherein R⁵ is H, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl.

18. The compound according to any one of claims 1-17, wherein R² and R³ are each independently H or C₁-C₄ alkyl.

19. The compound according to any one of claims 1-18, wherein R³ is H.

20. The compound according to any one of claims 1-19, wherein R² is C₁-C₄ alkyl, and R³ is H.

21. The compound according to any one of claims 1-18, wherein R² and R³ are each H.

22. The compound according to claim 1, wherein R² and R³ are each independently H or C₁-C₆ alkyl, R⁸ and R⁹ are each independently H, NR¹³R¹⁴, halo, C₁-C₄ alkoxy or C₁-C₄ alkyl, and R¹³ and R¹⁴ are each independently H or C₁-C₄ alkyl.

23. The compound according to claim 1, having Formula II: 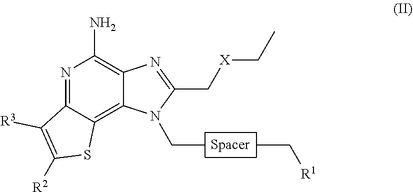
wherein:
X is —CH₂— or —O—;
R¹ is —OH, —NR⁴R⁵, —OR¹⁰, SR¹¹ or 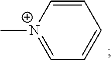
R² and R³ are each independently H or optionally substituted C₁-C₆ alkyl;
Spacer is —(CH₂)ₙ—, 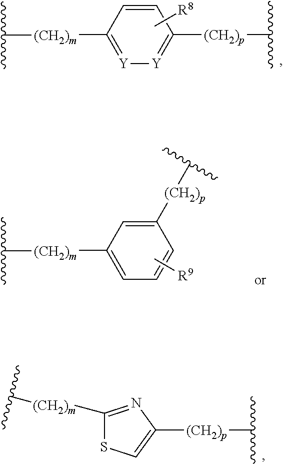
wherein n is an integer between 3 and 10; each m is independently an integer between 0 and 4; each p is independently an integer between 0 and 4; and Y is CH or N;
R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;
R⁸ and R⁹ are each independently H, NR¹³R¹⁴, halo, optionally substituted C₁-C₄ alkoxy or optionally substituted C₁-C₄ alkyl;
R¹⁰ and R¹¹ are each independently optionally substituted C₁-C₆ alkyl, optionally substituted aryl or optionally substituted C₃-C₇ cycloalkyl; and
R¹³ and R¹⁴ are each independently H or optionally substituted C₁-C₄ alkyl.

24. The compound according to claim 1, having Formula III: 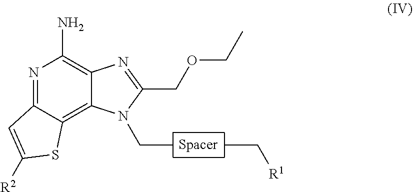
wherein:
R¹ is —OH, —NR⁴R⁵, —OR¹⁰, SR¹¹ or 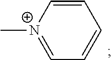
R² is H or optionally substituted C₁-C₆ alkyl;
Spacer is —(CH₂)ₙ—, 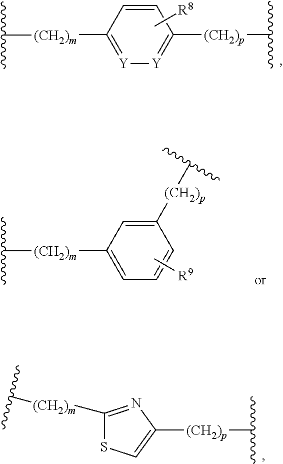
wherein n is an integer between 3 and 10; each m is independently an integer between 0 and 4; each p is independently an integer between 0 and 4; and Y is CH or N;
R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;
R⁸ and R⁹ are each independently H, NR¹³R¹⁴, halo, optionally substituted C₁-C₄ alkoxy or optionally substituted C₁-C₄ alkyl;
R¹⁰ and R¹¹ are each independently optionally substituted C₁-C₆ alkyl, optionally substituted aryl or optionally substituted C₃-C₇ cycloalkyl; and
R¹³ and R¹⁴ are each independently H or optionally substituted C₁-C₄ alkyl.

25. The compound according to claim 1, having Formula IV: 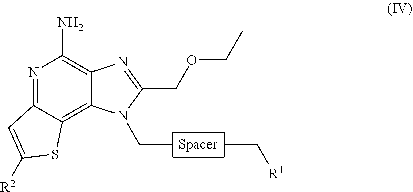
wherein:
R¹ is —OH, —NR⁴R⁵, —OR¹⁰, SR¹¹ or 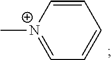
R² is H or optionally substituted C₁-C₆ alkyl, and
Spacer is —(CH₂)ₙ—, 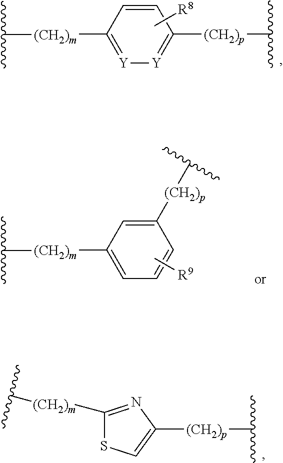
wherein n is an integer between 3 and 10; each m is independently an integer between 0 and 4; each p is independently an integer between 0 and 4; and Y is CH or N;
R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;
R⁸ and R⁹ are each independently H, NR¹³R¹⁴, halo, optionally substituted C₁-C₄ alkoxy or optionally substituted C₁-C₄ alkyl;
R¹⁰ and R¹¹ are each independently optionally substituted C₁-C₆ alkyl, optionally substituted aryl or optionally substituted C₃-C₇ cycloalkyl; and
R¹³ and R¹⁴ are each independently H or optionally substituted C₁-C₄ alkyl.

26. The compound according to claim 1, having Formula V: 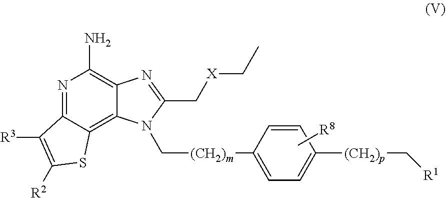
wherein:
X is —CH₂— or —O—;
R¹ is —OH, —NR⁴R⁵, —OR¹⁰, SR¹¹ or 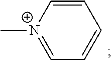
R² and R³ are each independently H or optionally substituted C₁-C₆ alkyl;
R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;
R⁸ is H, NR¹³R¹⁴, halo, optionally substituted C₁-C₄ alkoxy or optionally substituted C₁-C₄ alkyl;
R¹⁰ and R¹¹ are each independently optionally substituted C₁-C₆ alkyl, optionally substituted aryl or optionally substituted C₃-C₇ cycloalkyl;
R¹³ and R¹⁴ are each independently H or optionally substituted C₁-C₄ alkyl;
each m is independently an integer between 0 and 4; and
each p is independently an integer between 0 and 4.

27. The compound according to claim 1, having Formula VI: 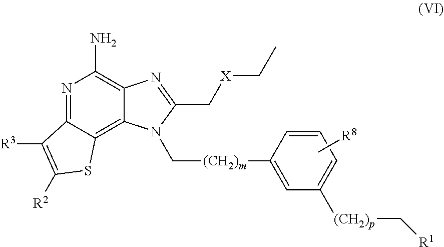
wherein:
X is —CH₂— or —O—;
R¹ is —OH, —NR⁴R⁵, —OR¹⁰, SR¹¹ or 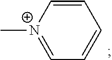
R² and R³ are each independently H or optionally substituted C₁-C₆ alkyl;
R⁴ and R⁵ are each independently H, optionally substituted C₁-C₄ alkoxycarbonyl, optionally substituted C₁-C₆ alkyl, optionally substituted C₁-C₄ amidoalkyl, optionally substituted C₁-C₄ aminoalkyl, optionally substituted aryl, optionally substituted aryl-C₁-C₄ alkyl, optionally substituted C₁-C₄ carboxyalkyl, optionally substituted heteroaryl, optionally substituted heteroaryl-C₁-C₄ alkyl, optionally substituted C₃-C₇ cycloalkyl, optionally substituted C₃-C₇ cycloalkyl-C₁-C₄ alkyl, optionally substituted C₃-C₇ heterocyclyl or optionally substituted C₃-C₇ heterocyclyl-C₁-C₄ alkyl; or R⁴ and R⁵ together with the N atom to which they are attached form a four- to ten-membered optionally substituted heterocycle;
R⁹ is H, NR¹³R¹⁴, halo, optionally substituted C₁-C₄ alkoxy or optionally substituted C₁-C₄ alkyl;
R¹⁰ and R¹¹ are each independently optionally substituted C₁-C₆ alkyl, optionally substituted aryl or optionally substituted C₃-C₇ cycloalkyl;
R¹³ and R¹⁴ are each independently H or optionally substituted C₁-C₄ alkyl;
each m is independently an integer between 0 and 4; and
each p is independently an integer between 0 and 4.

28. A compound selected from the following compounds, or a pharmaceutically acceptable salt thereof:

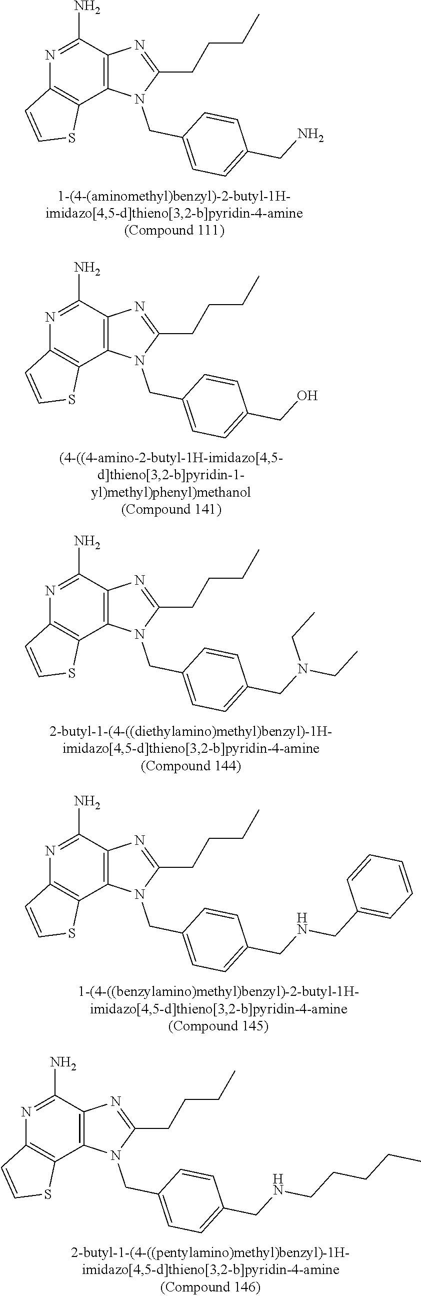 [SMILES: CCCCc1nc2c(N)nc3ccsc3c2n1Cc1ccc(CN)cc1 ; CCCCc1nc2c(N)nc3ccsc3c2n1Cc1ccc(CO)cc1 ; CCCCc1nc2c(N)nc3ccsc3c2n1Cc1ccc(CN(CC)CC)cc1 ; CCCCc1nc2c(N)nc3ccsc3c2n1Cc1ccc(CNCc2ccccc2)cc1 ; CCCCCNCc1ccc(Cn2c(CCCC)nc3c(N)nc4ccsc4c32)cc1]

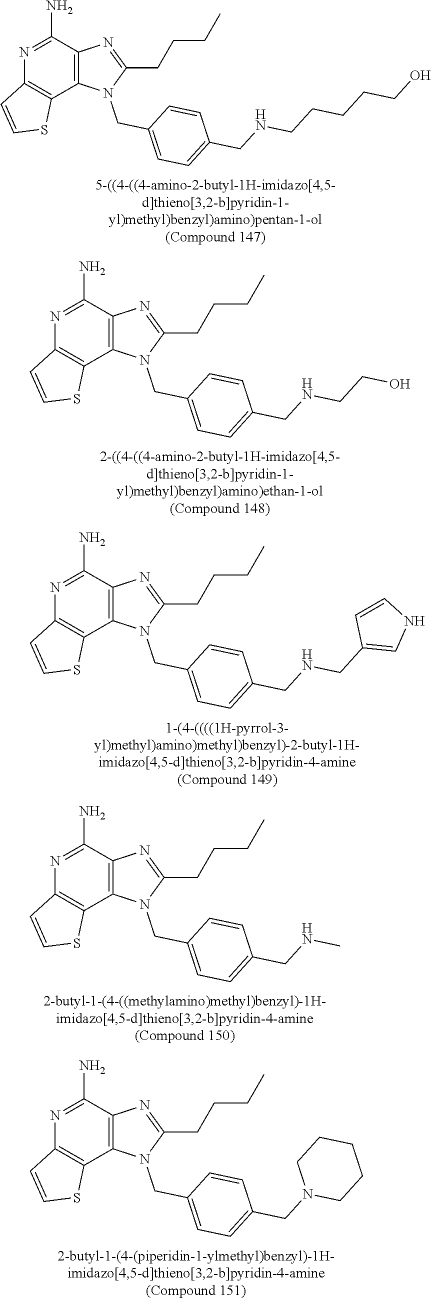 [SMILES: CCCCc1nc2c(N)nc3ccsc3c2n1Cc1ccc(CNCCCCCO)cc1 ; CCCCc1nc2c(N)nc3ccsc3c2n1Cc1ccc(CNCCO)cc1 ; CCCCc1nc2c(N)nc3ccsc3c2n1Cc1ccc(CNCc2cc[nH]c2)cc1 ; CCCCc1nc2c(N)nc3ccsc3c2n1Cc1ccc(CNC)cc1 ; CCCCc1nc2c(N)nc3ccsc3c2n1Cc1ccc(CN2CCCCC2)cc1]

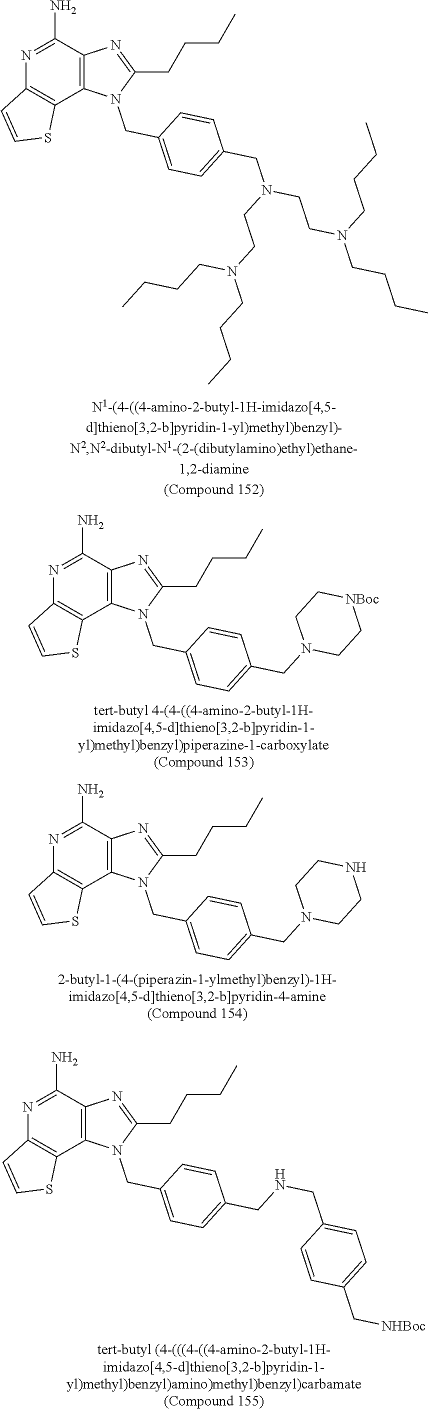 [SMILES: CCCCc1nc2c(N)nc3ccsc3c2n1Cc1ccc(CN(CCN(CCCC)CCCC)CCN(CCCC)CCCC)cc1 ; CCCCc1nc2c(N)nc3ccsc3c2n1Cc1ccc(CN2CCN(C(=O)OC(C)(C)C)CC2)cc1 ; CCCCc1nc2c(N)nc3ccsc3c2n1Cc1ccc(CN2CCNCC2)cc1 ; CCCCc1nc2c(N)nc3ccsc3c2n1Cc1ccc(CNCc2ccc(CNC(=O)OC(C)(C)C)cc2)cc1]

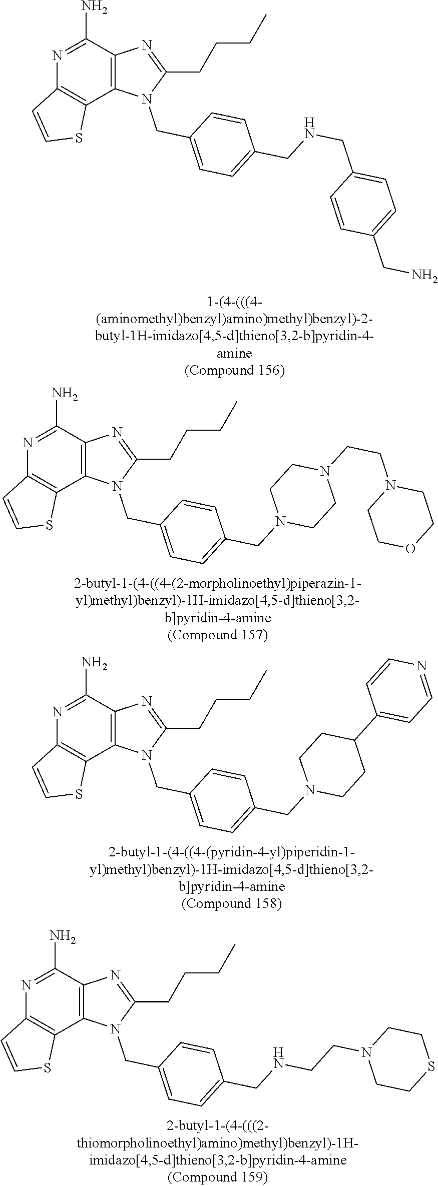 [SMILES: CCCCc1nc2c(N)nc3ccsc3c2n1Cc1ccc(CNCc2ccc(CN)cc2)cc1 ; CCCCc1nc2c(N)nc3ccsc3c2n1Cc1ccc(CN2CCN(CCN3CCOCC3)CC2)cc1 ; CCCCc1nc2c(N)nc3ccsc3c2n1Cc1ccc(CN2CCC(c3ccncc3)CC2)cc1 ; CCCCc1nc2c(N)nc3ccsc3c2n1Cc1ccc(CNCCN2CCSCC2)cc1]

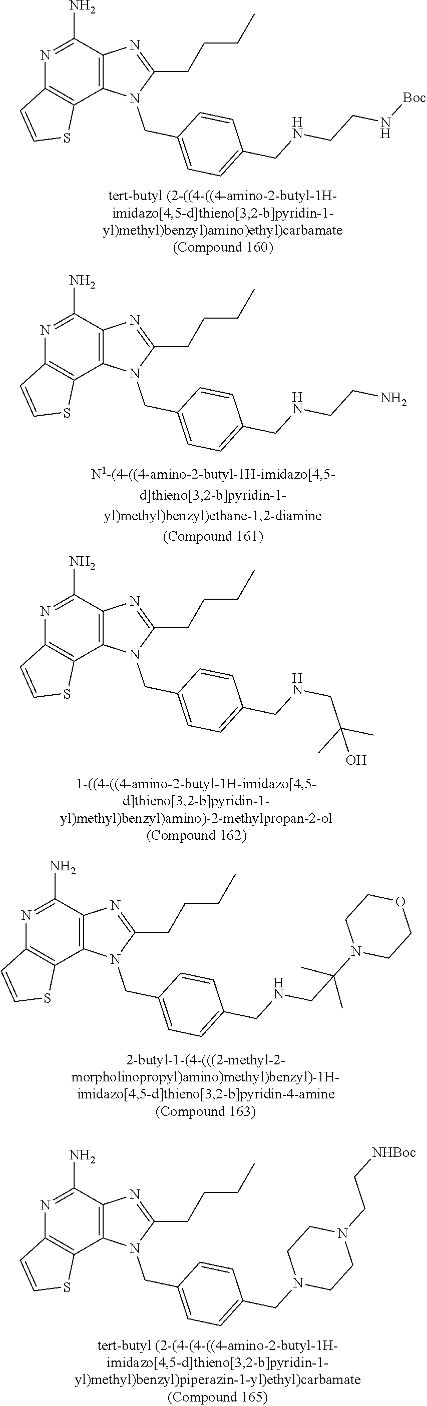 [SMILES: CCCCc1nc2c(N)nc3ccsc3c2n1Cc1ccc(CNCCNC(=O)OC(C)(C)C)cc1 ; CCCCc1nc2c(N)nc3ccsc3c2n1Cc1ccc(CNCCN)cc1 ; CCCCc1nc2c(N)nc3ccsc3c2n1Cc1ccc(CNCC(C)(C)O)cc1 ; CCCCc1nc2c(N)nc3ccsc3c2n1Cc1ccc(CNCC(C)(C)N2CCOCC2)cc1 ; CCCCc1nc2c(N)nc3ccsc3c2n1Cc1ccc(CN2CCN(CCNC(=O)OC(C)(C)C)CC2)cc1]

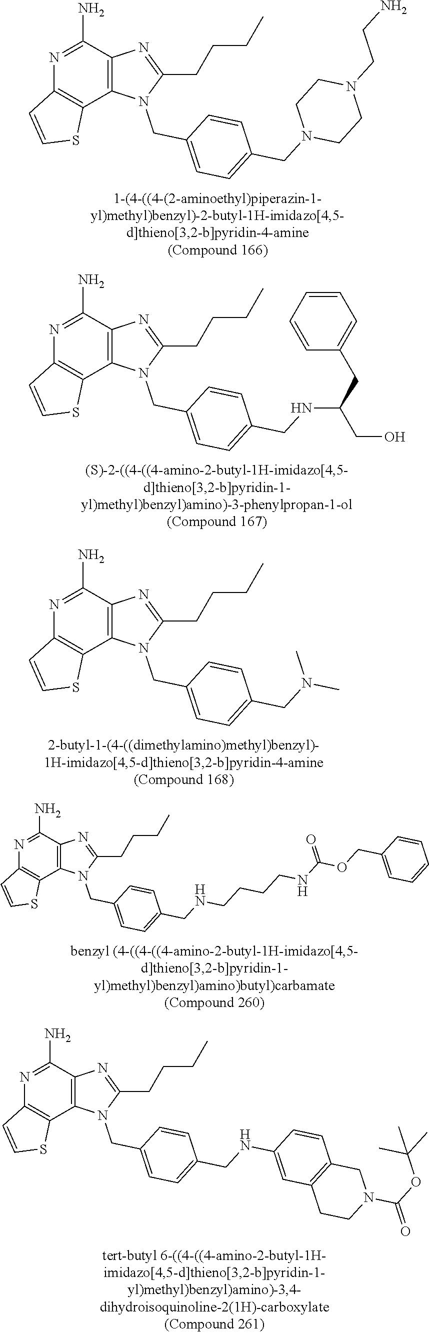 [SMILES: CCCCc1nc2c(N)nc3ccsc3c2n1Cc1ccc(CN2CCN(CCN)CC2)cc1 ; CCCCc1nc2c(N)nc3ccsc3c2n1Cc1ccc(CN[C@H](CO)Cc2ccccc2)cc1 ; CCCCc1nc2c(N)nc3ccsc3c2n1Cc1ccc(CN(C)C)cc1 ; CCCCc1nc2c(N)nc3ccsc3c2n1Cc1ccc(CNCCCCNC(=O)OCc2ccccc2)cc1 ; CCCCc1nc2c(N)nc3ccsc3c2n1Cc1ccc(CNc2ccc3c(c2)CCN(C(=O)OC(C)(C)C)C3)cc1]

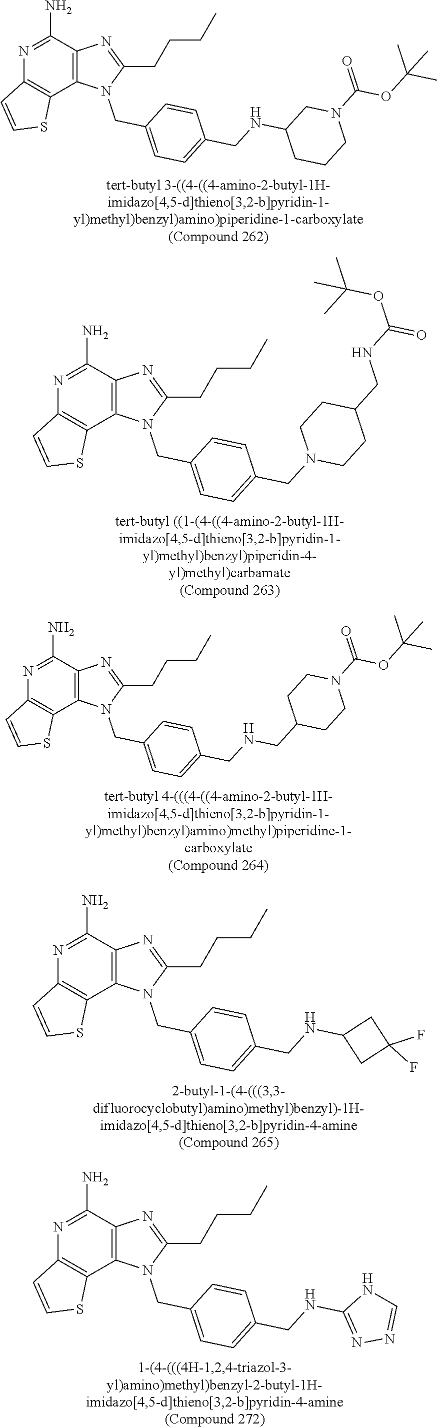 [SMILES: CCCCc1nc2c(N)nc3ccsc3c2n1Cc1ccc(CNC2CCCN(C(=O)OC(C)(C)C)C2)cc1 ; CCCCc1nc2c(N)nc3ccsc3c2n1Cc1ccc(CN2CCC(CNC(=O)OC(C)(C)C)CC2)cc1 ; CCCCc1nc2c(N)nc3ccsc3c2n1Cc1ccc(CNCC2CCN(C(=O)OC(C)(C)C)CC2)cc1 ; CCCCc1nc2c(N)nc3ccsc3c2n1Cc1ccc(CNC2CC(F)(F)C2)cc1 ; CCCCc1nc2c(N)nc3ccsc3c2n1Cc1ccc(CNc2nnc[nH]2)cc1]

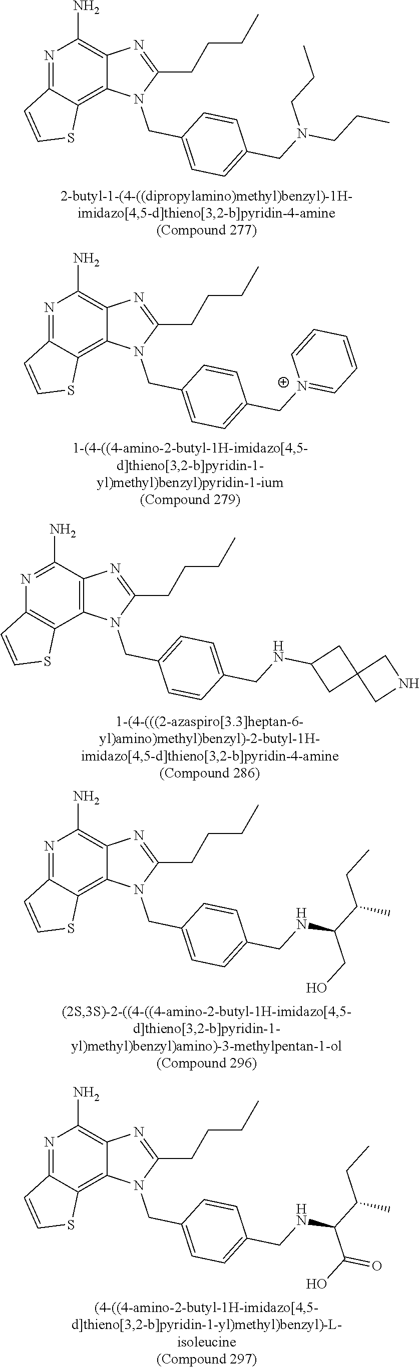 [SMILES: CCCCc1nc2c(N)nc3ccsc3c2n1Cc1ccc(CN(CCC)CCC)cc1 ; CCCCc1nc2c(N)nc3ccsc3c2n1Cc1ccc(C[n+]2ccccc2)cc1 ; CCCCc1nc2c(N)nc3ccsc3c2n1Cc1ccc(CNC2CC3(CNC3)C2)cc1 ; CCCCc1nc2c(N)nc3ccsc3c2n1Cc1ccc(CN[C@H](CO)[C@@H](C)CC)cc1 ; CCCCc1nc2c(N)nc3ccsc3c2n1Cc1ccc(CN[C@H](C(=O)O)[C@@H](C)CC)cc1]

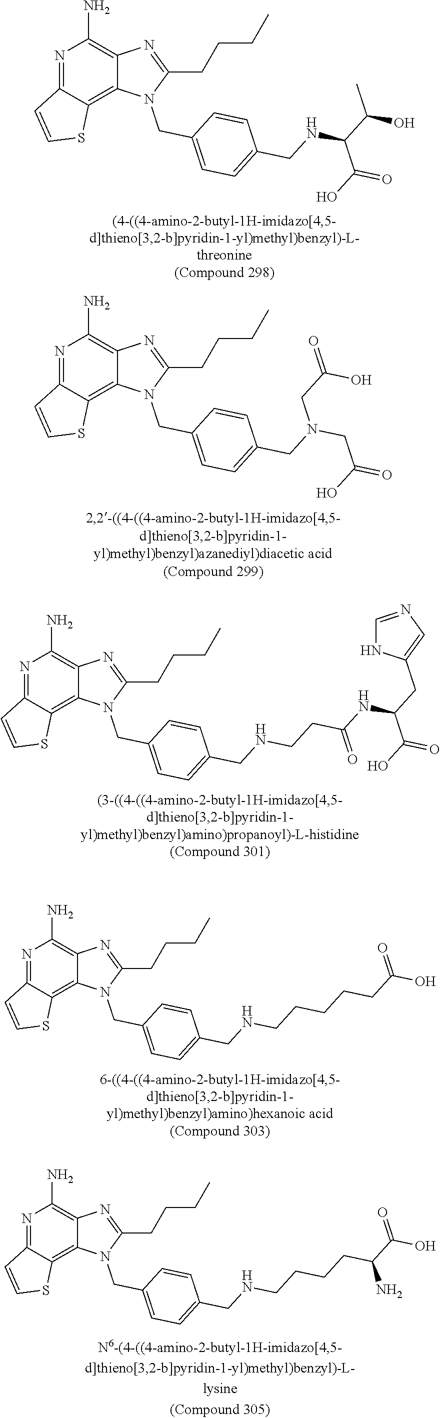 [SMILES: CCCCc1nc2c(N)nc3ccsc3c2n1Cc1ccc(CN[C@H](C(=O)O)[C@@H](C)O)cc1 ; CCCCc1nc2c(N)nc3ccsc3c2n1Cc1ccc(CN(CC(=O)O)CC(=O)O)cc1 ; CCCCc1nc2c(N)nc3ccsc3c2n1Cc1ccc(CNCCC(=O)N[C@@H](Cc2cnc[nH]2)C(=O)O)cc1 ; CCCCc1nc2c(N)nc3ccsc3c2n1Cc1ccc(CNCCCCCC(=O)O)cc1 ; CCCCc1nc2c(N)nc3ccsc3c2n1Cc1ccc(CNCCCC[C@H](N)C(=O)O)cc1]

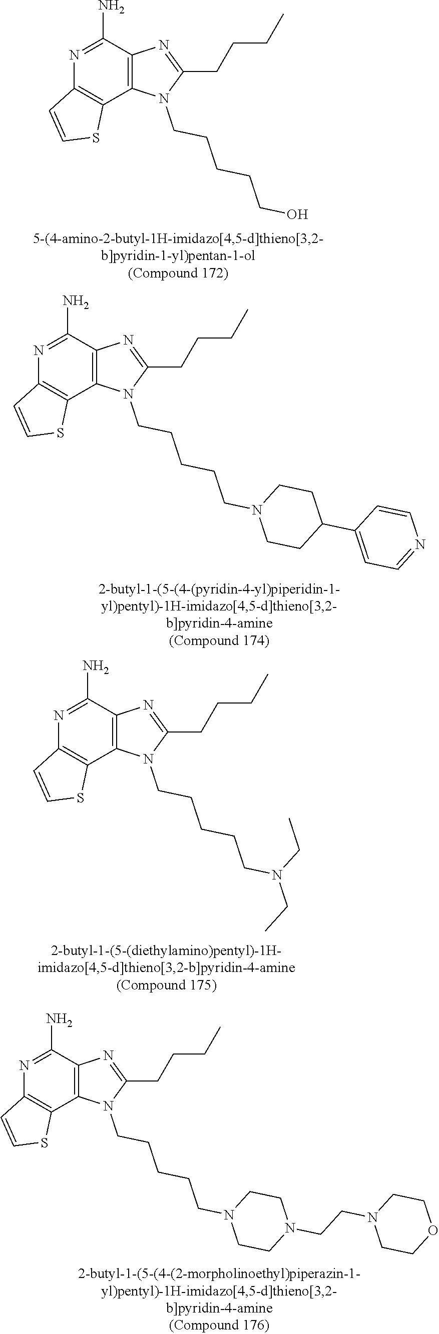 [SMILES: CCCCc1nc2c(N)nc3ccsc3c2n1CCCCCO ; CCCCc1nc2c(N)nc3ccsc3c2n1CCCCCN1CCC(c2ccncc2)CC1 ; CCCCc1nc2c(N)nc3ccsc3c2n1CCCCCN(CC)CC ; CCCCc1nc2c(N)nc3ccsc3c2n1CCCCCN1CCN(CCN2CCOCC2)CC1]

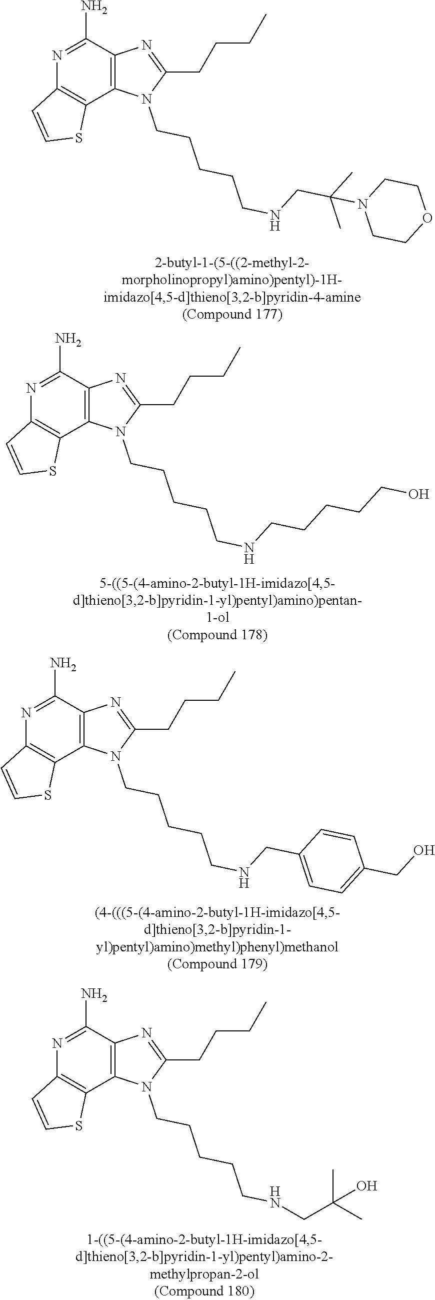 [SMILES: CCCCc1nc2c(N)nc3ccsc3c2n1CCCCCNCC(C)(C)N1CCOCC1 ; CCCCc1nc2c(N)nc3ccsc3c2n1CCCCCNCCCCCO ; CCCCc1nc2c(N)nc3ccsc3c2n1CCCCCNCc1ccc(CO)cc1 ; CCCCc1nc2c(N)nc3ccsc3c2n1CCCCCNCC(C)(C)O]

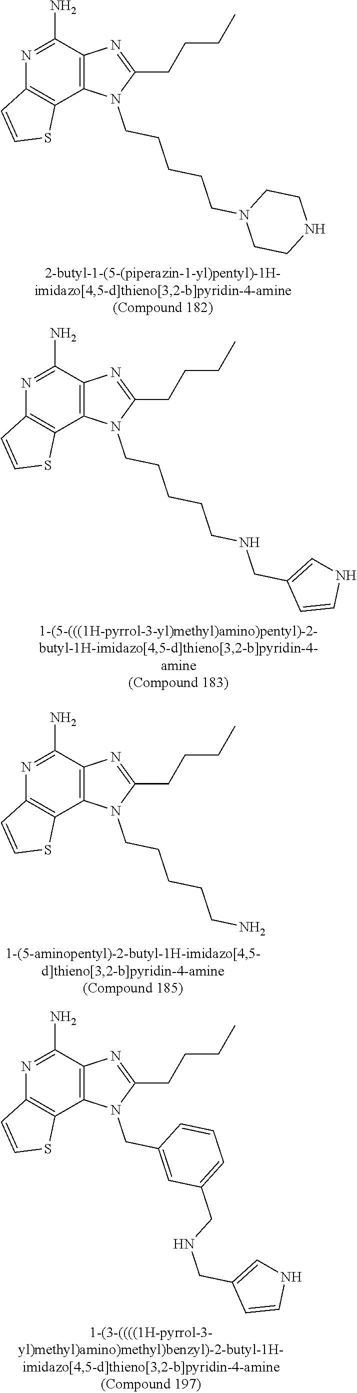 [SMILES: CCCCc1nc2c(N)nc3ccsc3c2n1CCCCCN1CCNCC1 ; CCCCc1nc2c(N)nc3ccsc3c2n1CCCCCNCc1cc[nH]c1 ; CCCCc1nc2c(N)nc3ccsc3c2n1CCCCCN ; CCCCc1nc2c(N)nc3ccsc3c2n1Cc1cccc(CNCc2cc[nH]c2)c1]

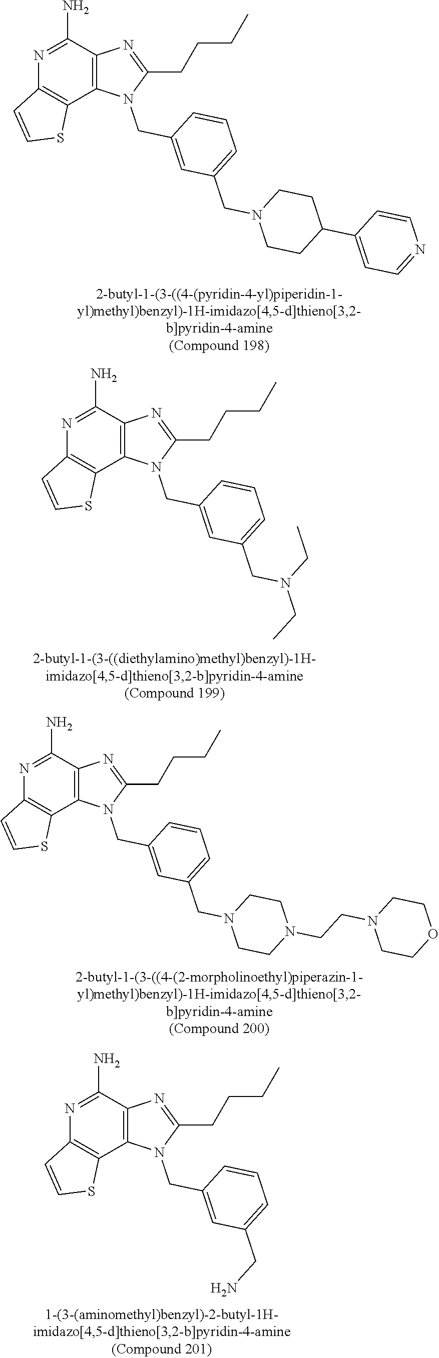 [SMILES: CCCCc1nc2c(N)nc3ccsc3c2n1Cc1cccc(CN2CCC(c3ccncc3)CC2)c1 ; CCCCc1nc2c(N)nc3ccsc3c2n1Cc1cccc(CN(CC)CC)c1 ; CCCCc1nc2c(N)nc3ccsc3c2n1Cc1cccc(CN2CCN(CCN3CCOCC3)CC2)c1 ; CCCCc1nc2c(N)nc3ccsc3c2n1Cc1cccc(CN)c1]

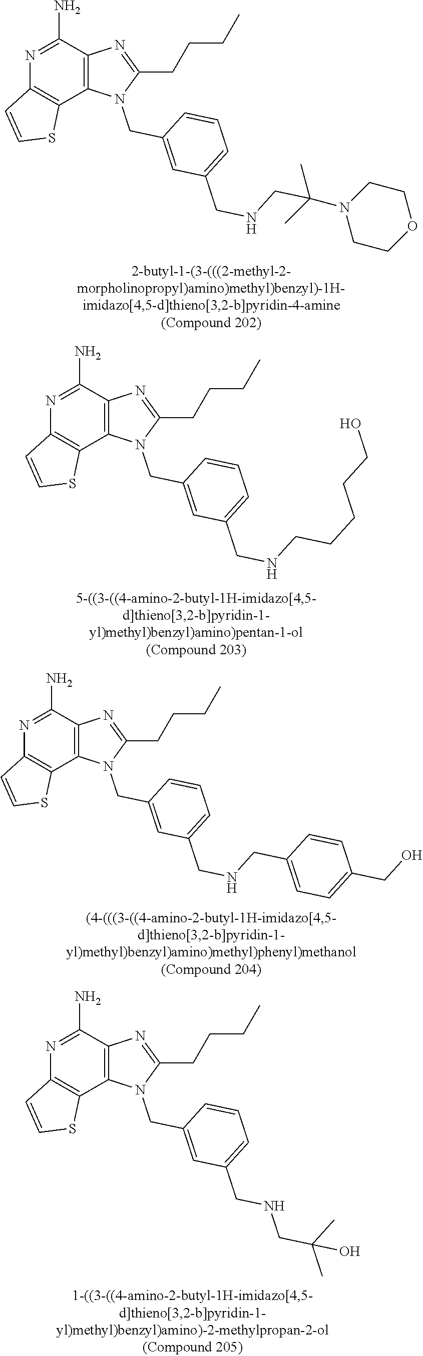 [SMILES: CCCCc1nc2c(N)nc3ccsc3c2n1Cc1cccc(CNCC(C)(C)N2CCOCC2)c1 ; CCCCc1nc2c(N)nc3ccsc3c2n1Cc1cccc(CNCCCCCO)c1 ; CCCCc1nc2c(N)nc3ccsc3c2n1Cc1cccc(CNCc2ccc(CO)cc2)c1 ; CCCCc1nc2c(N)nc3ccsc3c2n1Cc1cccc(CNCC(C)(C)O)c1]

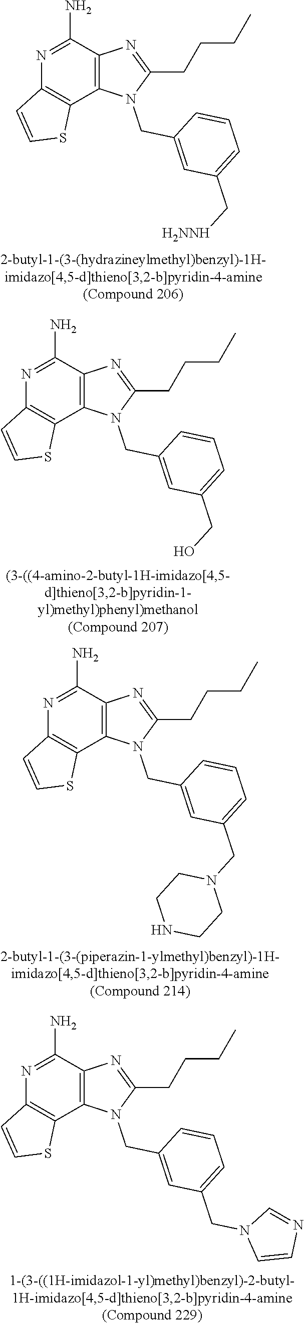

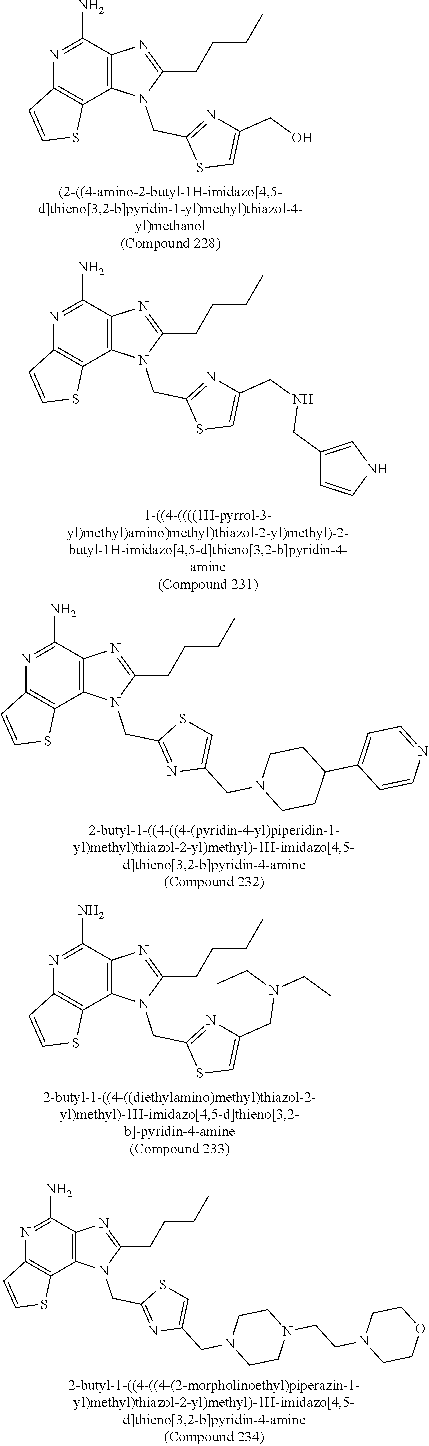 [SMILES: CCCCc1nc2c(N)nc3ccsc3c2n1Cc1nc(CO)cs1 ; CCCCc1nc2c(N)nc3ccsc3c2n1Cc1nc(CNCc2cc[nH]c2)cs1 ; CCCCc1nc2c(N)nc3ccsc3c2n1Cc1nc(CN2CCC(c3ccncc3)CC2)cs1 ; CCCCc1nc2c(N)nc3ccsc3c2n1Cc1nc(CN(CC)CC)cs1 ; CCCCc1nc2c(N)nc3ccsc3c2n1Cc1nc(CN2CCN(CCN3CCOCC3)CC2)cs1]

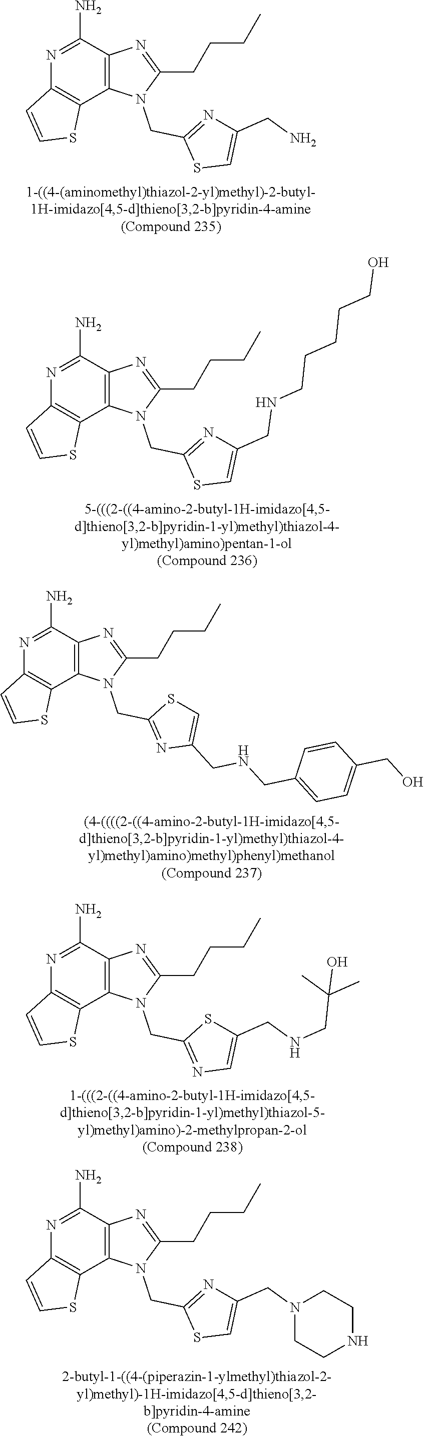 [SMILES: CCCCc1nc2c(N)nc3ccsc3c2n1Cc1nc(CN)cs1 ; CCCCc1nc2c(N)nc3ccsc3c2n1Cc1nc(CNCCCCCO)cs1 ; CCCCc1nc2c(N)nc3ccsc3c2n1Cc1nc(CNCc2ccc(CO)cc2)cs1 ; CCCCc1nc2c(N)nc3ccsc3c2n1Cc1ncc(CNCC(C)(C)O)s1 ; CCCCc1nc2c(N)nc3ccsc3c2n1Cc1nc(CN2CCNCC2)cs1]

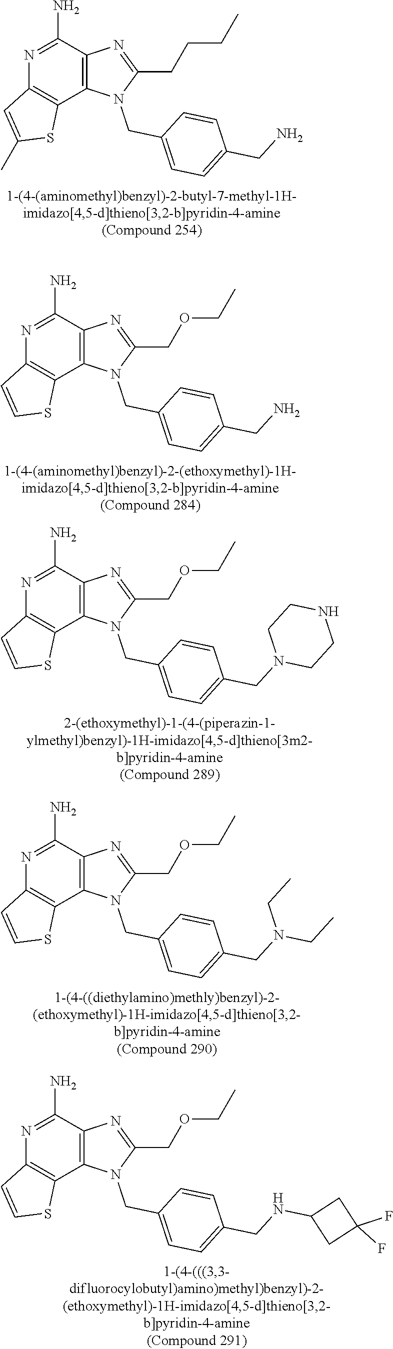 [SMILES: CCCCc1nc2c(N)nc3cc(C)sc3c2n1Cc1ccc(CN)cc1 ; CCOCc1nc2c(N)nc3ccsc3c2n1Cc1ccc(CN)cc1 ; CCOCc1nc2c(N)nc3ccsc3c2n1Cc1ccc(CN2CCNCC2)cc1 ; CCOCc1nc2c(N)nc3ccsc3c2n1Cc1ccc(CN(CC)CC)cc1 ; CCOCc1nc2c(N)nc3ccsc3c2n1Cc1ccc(CNC2CC(F)(F)C2)cc1]

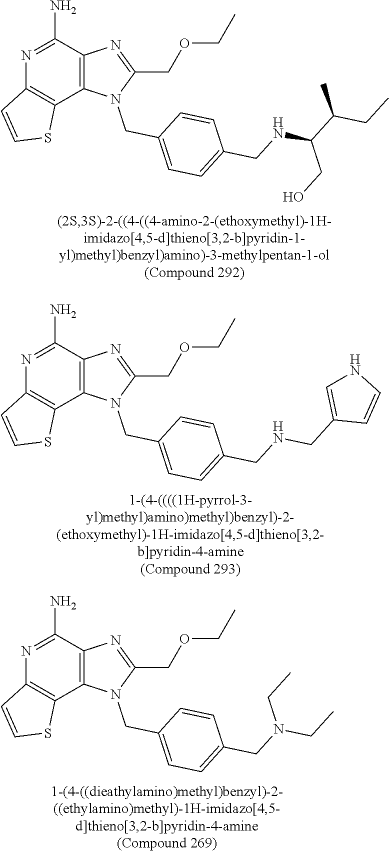 [SMILES: CCOCc1nc2c(N)nc3ccsc3c2n1Cc1ccc(CN[C@H](CO)[C@@H](C)CC)cc1 ; CCOCc1nc2c(N)nc3ccsc3c2n1Cc1ccc(CNCc2cc[nH]c2)cc1 ; CCOCc1nc2c(N)nc3ccsc3c2n1Cc1ccc(CN(CC)CC)cc1]

29. The compound according to any one of claims 1-28, wherein the compound has an EC₅₀ value for agonizing TLR7 of <500 nM, <250 nM, or <100 nM.

30. A pharmaceutical composition comprising a compound according to any one of claims 1-29, or a pharmaceutically acceptable salt thereof, and a pharmaceutically acceptable carrier or diluent.

31. A method of agonizing a TLR, the method comprising contacting a cell that expresses the TLR with a compound according to any one of claims 1-29, or a pharmaceutically acceptable salt thereof, thereby agonizing the TLR.

32. The method of claim 31, wherein the TLR is a TLR7, a TLR8, or a combination thereof.

33. A method of stimulating an immune response in a subject in need thereof, the method comprising administering to the subject an effective amount of a compound according to any one of claims 1-29, or a pharmaceutically acceptable salt thereof.

34. The method of claim 33, wherein the compound agonizes a TLR in the subject, thereby stimulating the immune response in the subject.

35. A method of treating a cancer in a subject in need thereof, the method comprising administering to the subject an effective amount of a compound according to any one of claims 1-29, or a pharmaceutically acceptable salt thereof.

36. The method of claim 35, wherein the compound agonizes a TLR in the subject, thereby treating the cancer in the subject.

37. A conjugate having Formula X:
T-(L-(D)ᵣ)_{q} (X)
wherein:
T is a targeting moiety;
L is a linker;
D is a compound according to any one of claims 1-120;
q is a value from about 1 to about 8, and
r is an integer from 1 to 4.

38. The conjugate according to claim 37, wherein q is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

39. The conjugate according to any one of claims 37-38, wherein T is an antibody or antigen-binding antibody fragment.

40. A pharmaceutical composition comprising a conjugate according to any one of claims 37-39, and a pharmaceutically acceptable carrier or diluent.

41. A method of agonizing a TLR, the method comprising contacting a cell that expresses the TLR with a conjugate according to any one of claims 37-39, thereby agonizing the TLR.

42. The method of claim 41, wherein the TLR is a TLR7, a TLR8, or a combination thereof.

43. A method of stimulating an immune response in a subject in need thereof, the method comprising administering to the subject an effective amount of a conjugate according to any one of claims 37-39.

44. The method of claim 43, wherein the compound agonizes a TLR in the subject, thereby stimulating the immune response in the subject.

45. A method of treating a cancer in a subject in need thereof, the method comprising administering to the subject an effective amount of a conjugate according to any one of claims 37-39.

46. The method of claim 45, wherein the compound agonizes a TLR in the subject, thereby treating the cancer in the subject.

47. A compound according to any one of claims 1-29 for use in therapy.

48. A compound according to any one of claims 1-29, or a pharmaceutically acceptable salt thereof, for use to stimulate an immune response in a subject in need thereof.

49. Use of a compound according to any one of claims 1-29, or a pharmaceutically acceptable salt thereof, in the manufacture of a medicament for stimulating an immune response in a subject in need thereof.

50. A compound according to any one of claims 1-29, or a pharmaceutically acceptable salt thereof, for use to treat a cancer in a subject in need thereof.

51. Use of a compound according to any one of claims 1-29, or a pharmaceutically acceptable salt thereof, in the manufacture of a medicament for treating a cancer in a subject in need thereof.

52. A conjugate according to any one of claims 37-39 for use to stimulate an immune response in a subject in need thereof.

53. Use of a conjugate according to any one of claims 37-39 in the manufacture of a medicament for stimulating an immune response in a subject in need thereof.

54. A conjugate according to any one of claims 37-39 for use to treat a cancer in a subject in need thereof.

55. Use of a conjugate according to any one of claims 37-39 in the manufacture of a medicament for treating a cancer in a subject in need thereof.
